(19) United States
(12) Patent Application Publication
(10) Pub. No.: US 2023/0144824 A1
(43) Pub. Date: May 11, 2023

(54) 1H-PYRAZOLO[4,3-d]PYRIMIDINE COMPOUNDS AS TOLL-LIKE RECEPTOR 7 (TLR7) AGONISTS

(71) Applicant: BRISTOL-MYERS SQUIBB COMPANY, Princeton, NJ (US)

(72) Inventors: Liqi HE, San Jose, CA (US); Ashvinikumar V. GAVAI, Princeton Junction, NJ (US); Sanjeev GANGWAR, Foster City, CA (US); Qiang CONG, Palo Alto, CA (US); Yam B. POUDEL, Fremont, CA (US); Prasanna SIVAPRAKASAM, Lawrenceville, NJ (US); Christine M. TARBY, Lawrenceville, NJ (US); Patrice GILL, Levittown, PA (US); Andrew F. DONNELL, Branchburg, NJ (US); Murugaiah ANDAPPAN MURUGAIAH SUBBAIAH, Bommasandra Bangalore (IN)

(21) Appl. No.: 17/793,248

(22) PCT Filed: Jan. 26, 2021
(86) PCT No.: PCT/US2021/014983

§ 371 Date: Jul. 15, 2022

Related U.S. Application Data
(60) Provisional application No. 63/058,144, filed on Jul. 29, 2020.
(60) Provisional application No. 62/966,137, filed on Jan. 27, 2020.

(51) Int. Cl. C07D 487/04

(52) U.S. Cl. CPC C07D 487/04

(57) ABSTRACT

Compounds according to formula I or II are useful as agonists of Toll-like receptor 7 (TLR7). Such compounds can be used in cancer treatment, especially in combination with an anti-cancer immunotherapy agent, or as a vaccine adjuvant.

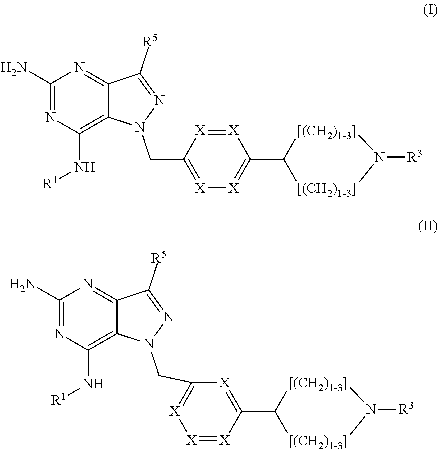

### CROSS-REFERENCE TO RELATED APPLICATIONS

This application claims the benefit under 35 U.S.C. § 119(e) of U.S. Provisional Application Ser. No. 63/058,144, filed Jul. 29, 2020, and U.S. Provisional Application Ser. No. 62/966,137, filed Jan. 27, 2020; the disclosures of which are incorporated herein by reference.

### BACKGROUND OF THE DISCLOSURE

This disclosure relates to Toll-like receptor 7 (“TLR7”) agonists and conjugates thereof, and methods for the preparation and use of such agonists and their conjugates.

Toll-like receptors (“TLRs”) are receptors that recognize pathogen-associated molecular patterns (“PAMPs”), which are small molecular motifs conserved in certain classes of pathogens. TLRs can be located either on a cell's surface or intracellularly. Activation of a TLR by the binding of its cognate PAMP signals the presence of the associated pathogen inside the host—i.e., an infection—and stimulates the host's immune system to fight the infection. Humans have 10 TLRs, named TLR1, TLR2, TLR3, and so on.

The activation of a TLR—with TLR7 being the most studied—by an agonist can have a positive effect on the action of vaccines and immunotherapy agents in treating a variety of conditions other than actual pathogen infection, by stimulating the immune response overall. Thus, there is considerable interest in the use of TLR7 agonists as vaccine adjuvants or as enhancers in cancer immunotherapy. See, for example, Vasilakos and Tomai 2013, Sato-Kaneko et al. 2017, Smits et al. 2008, and Ota et al. 2019.

TLR7, an intracellular receptor located on the membrane of endosomes, recognizes PAMPs associated with single-stranded RNA viruses. Its activation induces secretion of Type I interferons such as IFNα and IFNβ (Lund et al. 2004). TLR7 has two binding sites, one for single stranded RNA ligands (Berghofer et al. 2007) and one for small molecules such as guanosine (Zhang et al. 2016).

TLR7 can bind to, and be activated by, guanosine-like synthetic agonists such as imiquimod, resiquimod, and gardiquimod, which are based on a 1H-imidazo[4,5-c]quinoline scaffold. For a review of small-molecule TLR7 agonists, see Cortez and Va 2018.

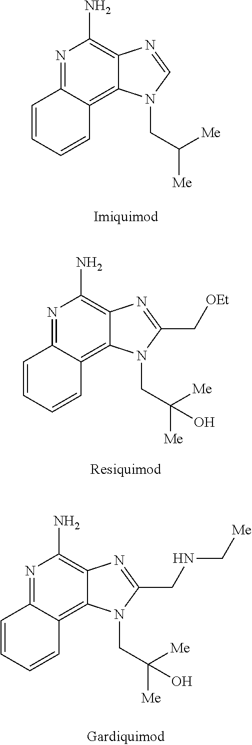 [SMILES: CC(C)Cn1cnc2c(N)nc3ccccc3c21 ; CCOCc1nc2c(N)nc3ccccc3c2n1CC(C)(C)O ; CCNCc1nc2c(N)nc3ccccc3c2n1CC(C)(C)O]

Synthetic TLR7 agonists based on a pteridinone molecular scaffold are also known, as exemplified by vesatolimod (Desai et al. 2015).

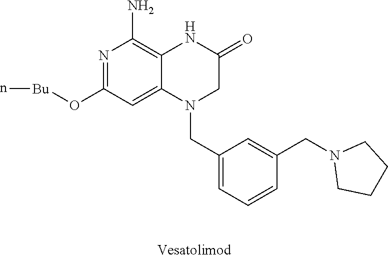

Other synthetic TLR7 agonists based on a purine-like scaffold have been disclosed, frequently according to the general formula (A):

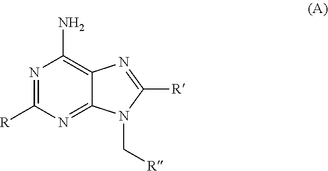

where R, R′, and R″ are structural variables, with R″ typically containing an unsubstituted or substituted aromatic or heteroaromatic ring.

Disclosures of bioactive molecules having a purine-like scaffold and their uses in treating conditions such as fibrosis, inflammatory disorders, cancer, or pathogenic infections include: Akinbobuyi et al. 2015 and 2016; Barberis et al. 2012; Carson et al. 2014; Ding et al. 2016, 2017a, and 2017b; Graupe et al. 2015; Hashimoto et al. 2009; He et al. 2019a and 2019b; Holldack et al. 2012; Isobe et al. 2009a and 2012; Poudel et al. 2019a and 2019b; Pryde 2010; and Young et al. 2019.

The group R″ can be pyridyl: Bonfanti et al. 2015a and 2015b; Halcomb et al. 2015; Hirota et al. 2000; Isobe et al. 2002, 2004, 2006, 2009a, 2009b, 2011, and 2012; Kasibhatla et al. 2007; Koga-Yamakawa et al. 2013; Musmuca et al. 2009; Nakamura 2012; Ogita et al. 2007; and Yu et al. 2013.

There are disclosures of related molecules in which the 6,5-fused ring system of formula (A)—a pyrimidine six member ring fused to an imidazole five member ring—is modified. (a) Dellaria et al. 2007, Jones et al. 2010 and 2012, and Pilatte et al. 2017 disclose compounds in which the pyrimidine ring is replaced by a pyridine ring. (b) Chen et al. 2011, Coe et al. 2017, Poudel et al. 2020a and 2020b, and Zhang et al. 2018 disclose compounds in which the imidazole ring is replaced by a pyrazole ring. (c) Cortez et al. 2017 and 2018; Li et al. 2018; and McGowan et al. 2016a, 2016b, and 2017 disclose compounds in which the imidazole ring is replaced by a pyrrole ring.

Bonfanti et al. 2015b and 2016 and Purandare et al. 2019 disclose TLR7 modulators in which the two rings of a purine moiety are spanned by a macrocycle:

A TLR7 agonist can be conjugated to a partner molecule, which can be, for example, a phospholipid, a poly(ethylene glycol) (“PEG”), an antibody, or another TLR (commonly TLR2). Exemplary disclosures include: Carson et al. 2013, 2015, and 2016, Chan et al. 2009 and 2011, Cortez et al. 2017, Gadd et al. 2015, Lioux et al. 2016, Maj et al. 2015, Vernejoul et al. 2014, and Zurawski et al. 2012. A frequent conjugation site is at the R″ group of formula (A).

Jensen et al. 2015 discloses the use of cationic lipid vehicles for the delivery of TLR7 agonists.

Some TLR7 agonists, including resiquimod are dual TLR7/TLR8 agonists. See, for example, Beesu et al. 2017, Embrechts et al. 2018, Lioux et al. 2016, and Vernejoul et al. 2014.

Full citations for the documents cited herein by first author or inventor and year are listed at the end of this specification.

### BRIEF SUMMARY OF THE DISCLOSURE

This specification relates to compounds having a 1H-pyrazolo[4,3d]pyrimidine aromatic system, having activity as TLR7 agonists.

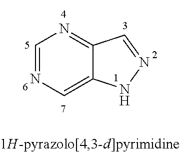

In one aspect, there is provided a compound with a structure according to formula (I) or (II)

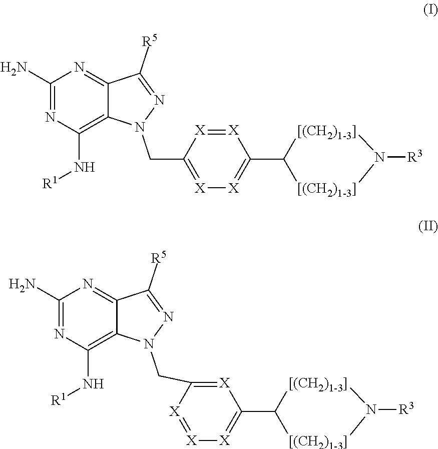

wherein
each X is independently N or CR²;
R¹ is H,
   (C₁-C₅ alkyl),
   (C₂-C₅ alkenyl),
   (C₁-C₈ alkanediyl)₀₋₁(C₃-C₆ cycloalkyl),
   (C₁-C₈ alkanediyl)₀₋₁(C₅-C₁₀ spiroalkyl),
   (C₂-C₈ alkanediyl)OH,
   (C₂-C₈ alkanediyl)O(C₁-C₃ alkyl),
   (C₁-C₄ alkanediyl)₀₋₁(5-6 membered heteroaryl),
   (C₁-C₄ alkanediyl)₀₋₁phenyl,
   (C₁-C₄ alkanediyl)CF₃,
   (C₂-C₈ alkanediyl)N[C(═O)](C₁-C₃ alkyl), or
   (C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN; or
   [C(═O)]₀₋₁NR^{x}R^{y};
R³ is [C(═O)]₀₋₁H,
   [C(═O)]₀₋₁(C₁-C₅ alkyl),
   [C(═O)]₀₋₁(C₁-C₄ alkanediyl)₀₋₁(C₃-C₅ cycloalkyl),
   [C(═O)]₀₋₁(C₁-C₄ alkanediyl)₀₋₁(C₄-C₁₀ bicycloalkyl),
   [C(═O)]₀₋₁(C₁-C₄ alkanediyl)₀₋₁(C₅-C₁₀ spiroalkyl),
   [C(═O)]₀₋₁(C₁-C₄ alkanediyl)₀₋₁O(C₁-C₃ alkyl),
   [C(═O)]₀₋₁(C₁-C₄ alkanediyl)₀₋₁OH,
   [C(═O)]₀₋₁(C₁-C₄ alkanediyl)₀₋₁NR^{x}R^{y},

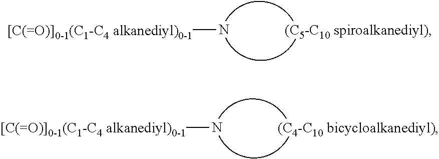

(C₁-C₄ alkanediyl)(C═O)NR^{x}R^{y},
(SO₂)(C₁-C₅ alkyl),
(SO₂)(C₁-C₄ alkanediyl)₀₋₁(C₃-C₅ cycloalkyl),
(SO₂)(C₁-C₄ alkanediyl)₀₋₁(C₄-C₁₀ bicycloalkyl),
(SO₂)(C₁-C₄ alkanediyl)₀₋₁(C₅-C₁₀ spiroalkyl),
a 6-membered aromatic or heteroaromatic moiety, or
a 5-membered heteroaromatic moiety;
R⁵ is H, C₁-C₅ alkyl, C₂-C₅ alkenyl, C₃-C₆ cycloalkyl, halo, O(C₁-C₅ alkyl),
   (C₁-C₄ alkanediyl)OH, (C₁-C₄ alkanediyl)O(C₁-C₃ alkyl), phenyl, NH(C₁-C₅ alkyl), 5 or 6 membered heteroaryl,

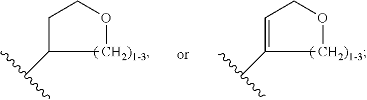

and
R^{x} and R^{y} are independently H or C₁-C₃ alkyl or R^{x} and R^{y} combine with the nitrogen to which they are bonded to form a 3- to 7-membered ring
wherein in R¹, R², R³, and R⁵
   an alkyl, cycloalkyl, alkanediyl, bicycloalkyl, spiroalkyl, cyclic amine, 6-membered aromatic or heteroaromatic moiety, 5-membered heteroaromatic moiety or a moiety of the formula

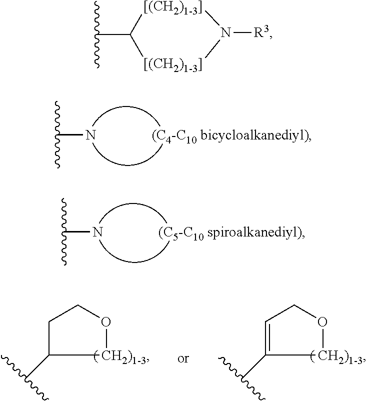

is optionally substituted with one or more substituents selected from OH, halo, CN,
(C₁-C₃ alkyl), O(C₁-C₃ alkyl), C(═O)(C₁-C₃ alkyl), SO₂(C₁-C₃ alkyl), NR^{x}R^{y},
(C₁-C₄ alkanediyl)OH, (C₁-C₄ alkanediyl)O(C₁-C₃ alkyl);
and
an alkyl, alkanediyl, cycloalkyl, bicycloalkyl, spiroalkyl, or a moiety of the formula

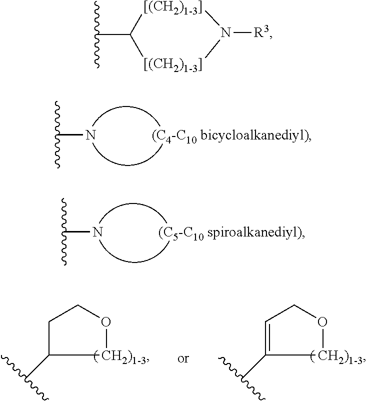

may have a CH₂ group replaced by O, SO₂, CF₂, C(═O), NH,
N[C(═O)]₀-1(C₁-C₃ alkyl),
N[C(═O)]₀-1(C₁-C₄ alkanediyl)₀₋₁CF₃,
or
N[C(═O)]₀-1(C₁-C₄ alkanediyl)₀₋₁(C₃-C₅ cycloalkyl).

Compounds disclosed herein have activity as TLR7 agonists and some can be conjugated to an antibody for targeted delivery to a target tissue or organ of intended action. They can also be PEGylated, to modulate their pharmaceutical properties.

Compounds disclosed herein, or their conjugates or their PEGylated derivatives, can be used in the treatment of a subject suffering from a condition amenable to treatment by activation of the immune system, by administering to such subject a therapeutically effective amount of such a compound or a conjugate thereof or a PEGylated derivative thereof, especially in combination with a vaccine or a cancer immunotherapy agent.

### DETAILED DESCRIPTION OF THE DISCLOSURE

### Compounds

In another aspect, this disclosure provides a compound having a structure according to formula (I′) or (II′), where R¹, R⁵, R³, and X are as defined in respect of formula (I):

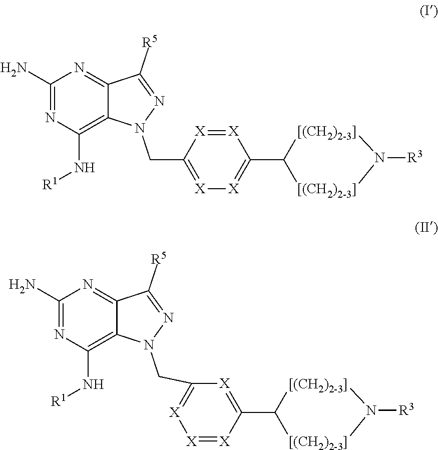

In another aspect, this disclosure provides a compound having a structure according to formula (I″) or (II″), where R¹, R⁵, R³, and X are as defined in respect of formula (I):

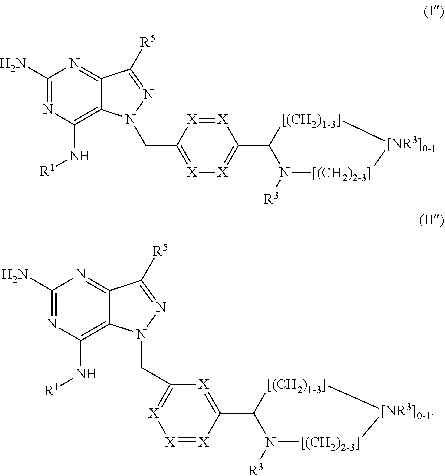

In compounds of formula (I), (I′), or (I″), embodiments of the moiety

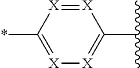

include

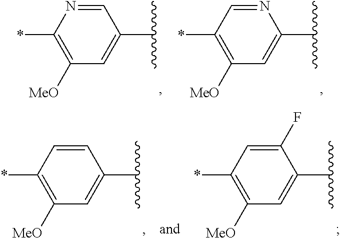

where the asterisk * denotes the position of bonding towards the pyrazolo-pyrimidine moiety and the wriggly line [symbol] denotes the position of bonding to the group W, with the first embodiment being a preferred one.

In compounds of formula (II), (II′), and (II″) a preferred embodiment of the moiety

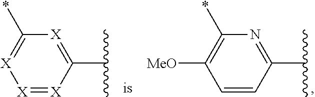

where the asterisk * denotes the position of bonding towards the pyrazolo-pyrimidine moiety and the wriggly line [symbol] denotes the position of bonding to the group W.

Embodiments of the group R⁵ include H (preferably), cyclopropyl, Cl and Me.

Embodiments of the group R¹ include

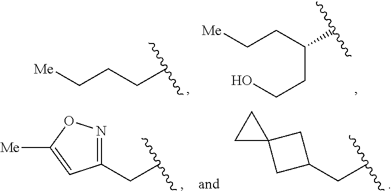

Additional embodiments of the group R¹ include:

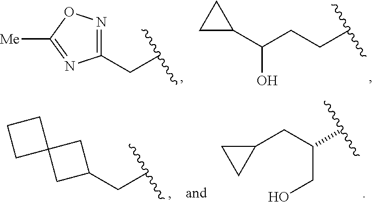 [SMILES: *Cc1noc(C)n1 ; *CCC(O)C1CC1 ; *CC1CC2(CCC2)C1 ; *[C@H](CO)CC1CC1]

Embodiments of the group

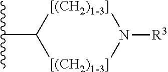

include:

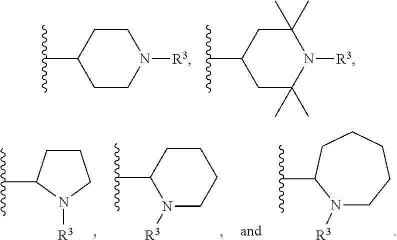

An embodiment of the group

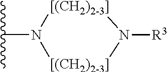

is

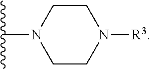

An embodiment of the group

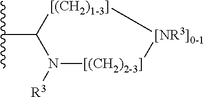

is

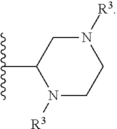

Embodiments of the group R³ include:

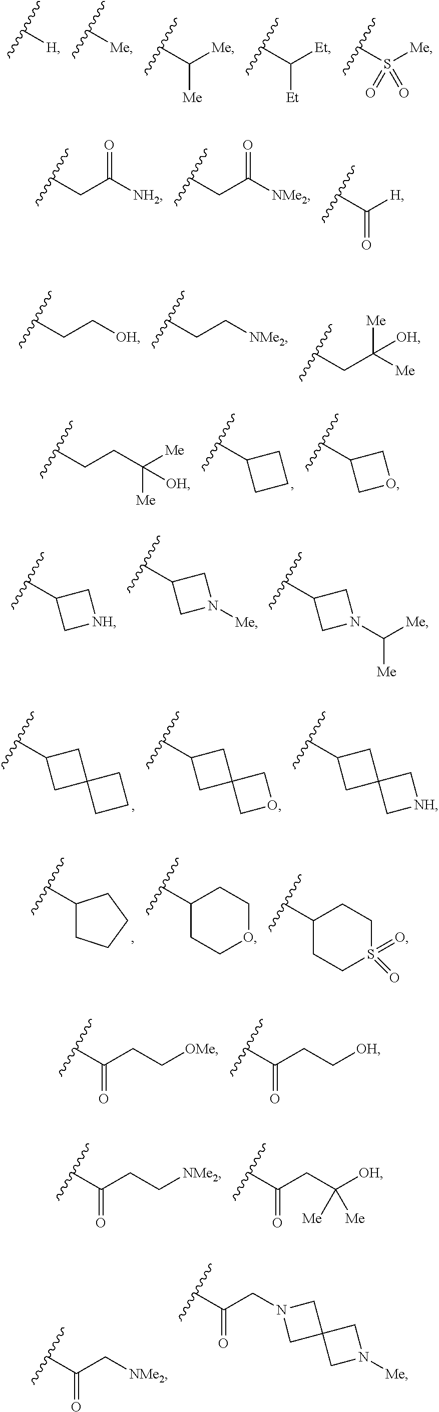

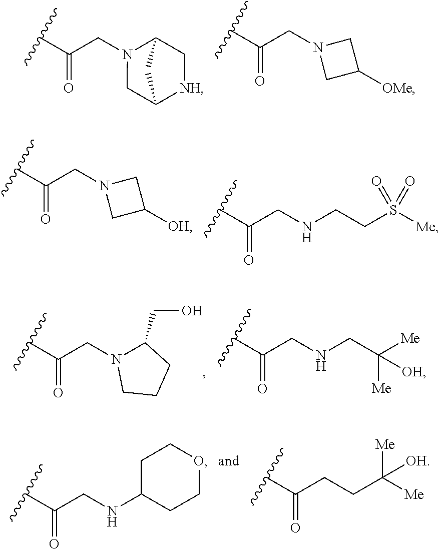

In one aspect, compounds of this disclosure are according to formula (Ia), wherein R¹, R³, and R⁵ are as defined in respect of formula (I).

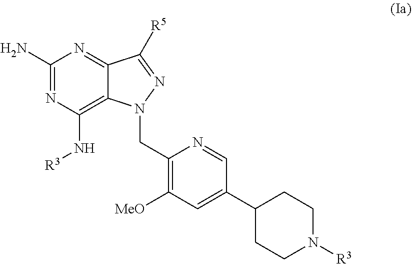

In another aspect, there is provided a compound according to formula (Ia), wherein

### R¹ is

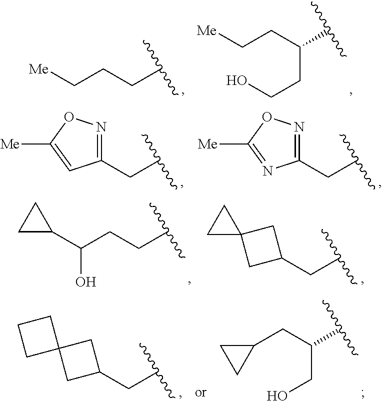

### R³ is H, Me,

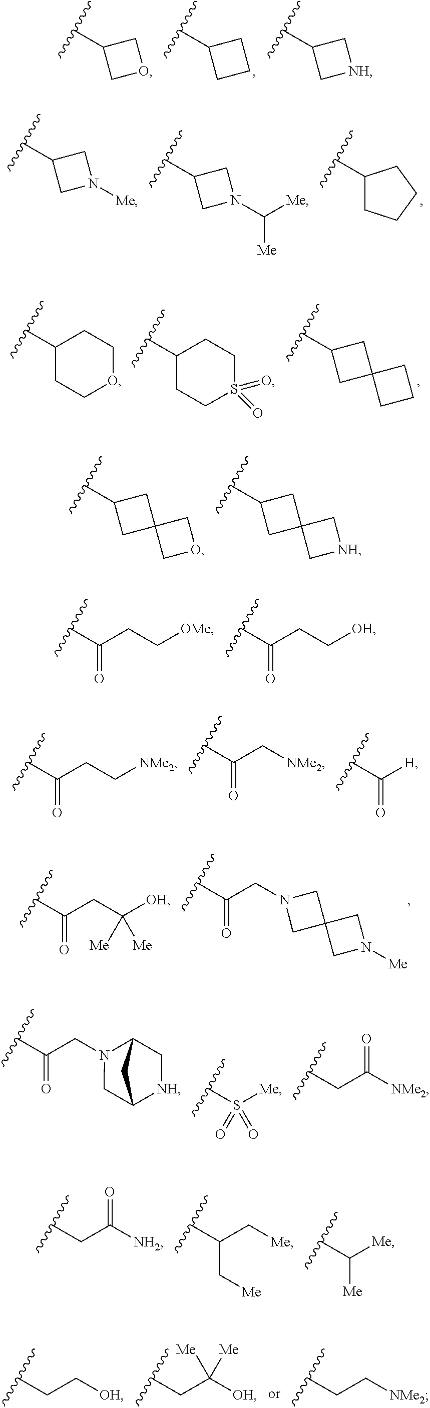

and
R⁵ is H, Me, cyclopropyl, or Cl.

In one aspect, this disclosure provides a compound according to formula (Ib)

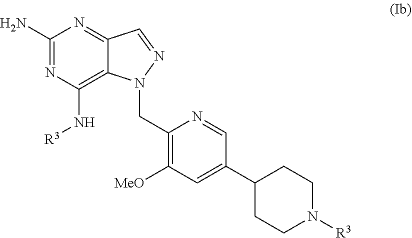

wherein

### R¹ is

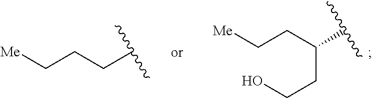 [SMILES: *CCCC ; *[C@@H](CCC)CCO]

and

### R³ is H,

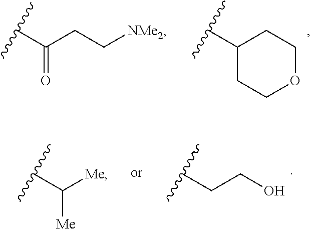

In one aspect, this disclosure provides a compound having a structure according to formula (I′a)

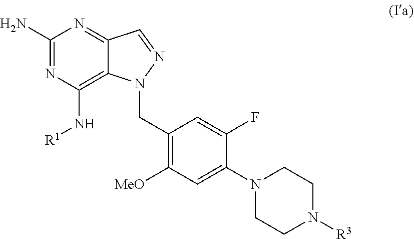

wherein R¹ and R³ are as defined in respect of formula (I). Preferably, in compounds of formula (I′a) R¹ is

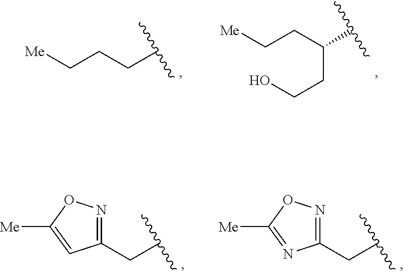 [SMILES: *CCCC ; *[C@@H](CCC)CCO ; *Cc1cc(C)on1 ; *Cc1noc(C)n1]

and more preferably

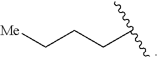 [SMILES: *CCCC]

Examples of compounds according to formula (I′a) are those wherein R¹ is

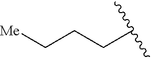 [SMILES: *CCCC]

### and R³ is H,

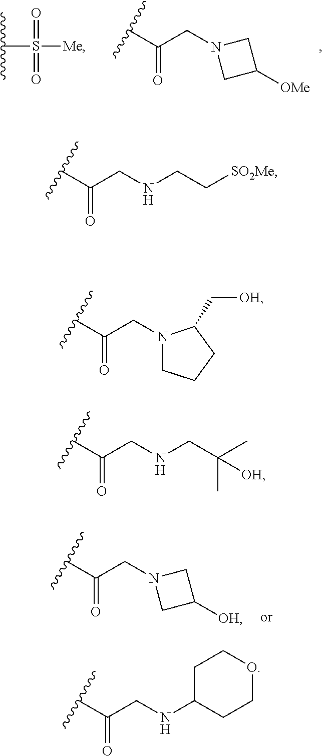

In one aspect, this disclosure provides a compound having a structure according to formula (I″a)

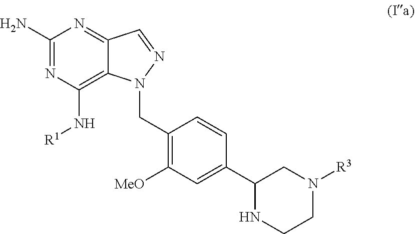

wherein R¹ and R³ are as defined in respect of formula (I).

Examples of compounds according to formula (I″a) are those wherein

### R¹ is

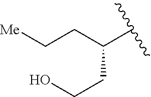 [SMILES: *[C@@H](CCC)CCO]

and

### R³ is

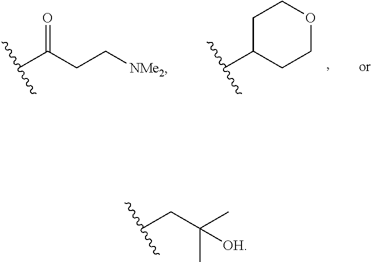

In one aspect, this disclosure provides a compound having a structure according to formula (IIa)

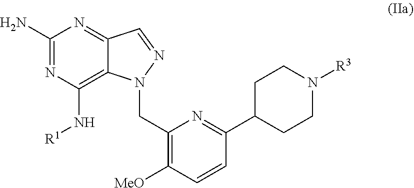

wherein R¹ and R³ are as defined in respect of formula (II).

Preferably, R¹ is

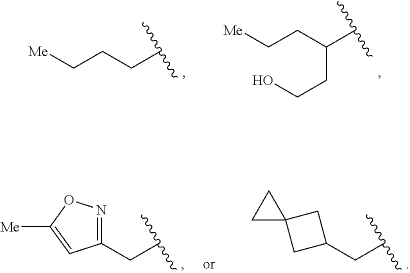 [SMILES: *CCCC ; *C(CCC)CCO ; *Cc1cc(C)on1 ; *CC1CC2(CC2)C1]

R² preferably is OMe, O(cyclopropyl), or OCHF₂, more preferably OMe.

In one aspect, R⁵ is H.

In one embodiment of compounds according to formula (Ia):
R¹ is

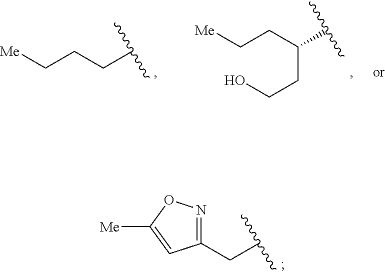 [SMILES: *CCCC ; *[C@@H](CCC)CCO ; *Cc1cc(C)on1]

R³ is a C₃-C₈ cycloalkyl moiety, wherein one CH₂ group in the cycloalkyl moiety is optionally replaced by O, SO₂, NH, or N(C₁-C₃ alkyl), as in, for example,

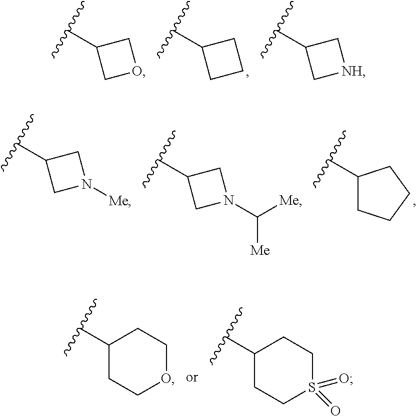

and
R⁵ is H or Cl (preferably H).

Specific examples of compounds disclosed herein are shown in the following Table A. The table also provides data relating to biological activity: human TLR7 agonism reporter assay and/or induction of the CD69 gene in human whole blood, determined per the procedure provided hereinbelow. The right-most column contains analytical data (mass spectrum, HPLC retention time, and NMR). In one embodiment, a compound of this disclosure has (a) a human TLR7 (hTLR7) Reporter Assay EC₅₀ value of less than 1,000 nM and (b) a human whole blood (hWB) CD69 induction EC₅₀ value of less than 1,000 nM. (Where an assay was performed multiple times, the reported value is an average.)

TABLE A

| | | hTLR7 | hWB | Mass spectrum; LC/MS Retention |
|---|---|---|---|---|
| | | Agonism | CD69 | Time (Procedure A unless noted |
| Cpd | | EC₅₀ | EC₅₀ | otherwise); ¹H NMR (500 MHz, |
| No. | Structure | (nM) | (nM) | DMSO-d₆ unless noted otherwise) |
| 101 | 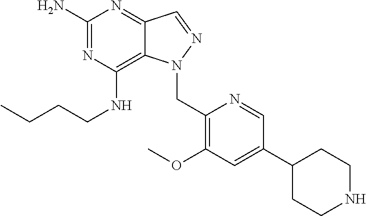 [SMILES: CCCCNc1nc(N)nc2cnn(Cc3ncc(C4CCNCC4)cc3OC)c12] | 56.6 | 9.3 | LC/MS [M + H]⁺ = 411.2 RT (min) = 1.06 δ 8.00-7.84 (m, 2H), 7.45 (s, 1H), 7.38 (s, 1H), 5.64 (s, 2H), 5.57 (s, 2H), 3.88 (s, 3H), 3.60-3.54 (m, 2H), 3.16-3.05 (m, 2H), 2.79- 2.62 (m, 3H), 1.81-1.73 (m, 2H), 1.71-1.56 (m, 4H), 1.36 (dq, J = 14.9, 7.3 Hz, 2H), 0.91 (t, J = 7.4 Hz, 3H) |
| | N7-butyl-1-{[3-methoxy-5-(piperidin-4- | | | |
| | yl)pyridin-2-yl]methyl}-1H-pyrazolo[4,3- | | | |
| | d]pyrimidine-5,7-diamine | | | |
| 102 | 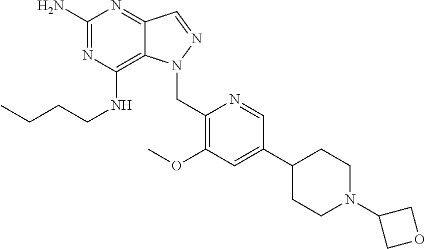 [SMILES: CCCCNc1nc(N)nc2cnn(Cc3ncc(C4CCN(C5COC5)CC4)cc3OC)c12] | 119.6 | 87.8 | LC/MS [M + H]⁺ = 467.3 RT (min) = 1.10 δ 8.07 (br t, J = 4.3 Hz, 1H), 7.99 (s, 1H), 7.49-7.43 (m, 2H), 5.75 (br s, 2H), 5.65 (s, 2H), 4.55 (t, J = 6.4 Hz, 2H), 4.48-4.39 (m, 2H), 3.90 (s, 3H), 3.61-3.30 (m, 2H), 2.80 (br d, J = 10.4 Hz, 2H), 2.63-2.57 (m, 2H), 1.90-1.79 (m, 2H), 1.79-1.69 (m, 4H), 1.63 (quin, J = 7.2 Hz, 2H), 1.43- 1.31 (m, 2H), 0.93 (t, J = 7.4 Hz, 3H) |
| | N7-butyl-1-({3-methoxy-5-[1-(oxetan-3- | | | |
| | yl)piperidin-4-yl]pyridin-2-yl}methyl)-1H- | | | |
| | pyrazolo[4,3-d]pyrimidine-5,7-diamine | | | |
| 103 | 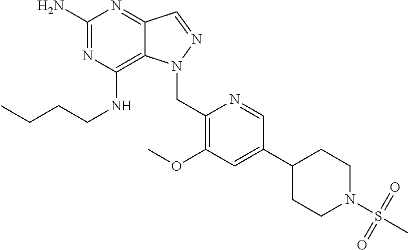 [SMILES: CCCCNc1nc(N)nc2cnn(Cc3ncc(C4CCN(S(C)(=O)=O)CC4)cc3OC)c12] | 204.1 | 561.6 | LC/MS [M + H]⁺ = 489.2 RT (min) = 1.27 δ 8.03-7.89 (m, 2H), 7.47 (s, 1H), 7.44 (s, 1H), 5.64 (s, 2H), 5.58 (s, 2H), 3.88 (s, 3H), 3.71-3.55 (m, 2H), 3.51-3.38 (m, 2H), 2.88 (s, 3H), 2.80 (br t, J = 11.1 Hz, 2H), 2.76- 2.68 (m, 1H), 1.89-1.83 (m, 2H), 1.80-1.67 (m, 2H), 1.62 (quin, J = 7.2 Hz, 2H), 1.44-1.26 (m, 2H), 0.91 (t, J = 7.3 Hz, 3H) |
| | N7-butyl-1-{[5-(1-methanesulfonylpiperi- | | | |
| | din-4-yl)-3-methoxypyridin-2-yl]methyl}- | | | |
| | 1H-pyrazolo[4,3-d]pyrimidine-5,7- | | | |
| | diamine | | | |
| 104 | 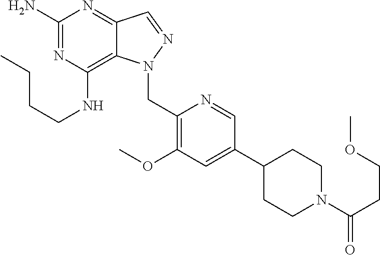 [SMILES: CCCCNc1nc(N)nc2cnn(Cc3ncc(C4CCN(C(=O)CCOC)CC4)cc3OC)c12] | 445.3 | 213.0 | LC/MS [M + H]⁺ = 497.0 RT (min) = 1.39 δ 8.15-8.02 (m, 1H), 7.98 (s, 1H), 7.48 (s, 1H), 7.41 (s, 1H), 5.64 (s, 2H), 4.59-4.44 (m, 1H), 4.00 (br d, J = 13.4 Hz, 1H), 3.88 (s, 3H), 3.50- 3.41 (m, 2H), 3.23 (s, 3H), 3.08 (br t, J = 12.7 Hz, 1H), 2.91-2.79 (m, 1H), 2.64-2.56 (m, 7H), 1.78 (br t, J = 12.8 Hz, 2H), 1.69-1.56 (m, 3H), 1.56-1.43 (m, 1H), 1.41-1.29 (m, 2H), 0.92 (t, J = 7.3 Hz, 3H) |
| | 1-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-3- | | | |
| | methoxypropan-1-one | | | |
| 105 | 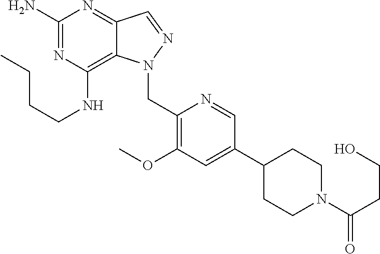 [SMILES: CCCCNc1nc(N)nc2cnn(Cc3ncc(C4CCN(C(=O)CCO)CC4)cc3OC)c12] | 817.1 | 159.0 | LC/MS [M + H] = 482.9 RT (min) = 1.26 δ 7.98 (s, 2H), 7.46 (s, 1H), 7.42 (s, 1H), 5.64 (s, 2H), 5.57 (s, 2H), 4.55 (br d, J = 12.8 Hz, 1H), 4.01 (br d, J = 13.1 Hz, 1H), 3.88 (s, 3H), 3.65 (t, J = 6.6 Hz, 1H), 3.60-3.41 (m, 4H), 3.09 (br t, J = 12.1 Hz, 1H), 2.91- 2.79 (m, 1H), 2.59 (br s, 2H), 1.85- 1.70 (m, 2H), 1.63 (dt, J = 14.6, 7.3 Hz, 3H), 1.57-1.46 (m, 1H), 1.42- 1.29 (m, 2H), 0.92 (t, J = 7.3 Hz, 3H) |
| | 1-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-3- | | | |
| | hydroxypropan-1-one | | | |
| 106 | 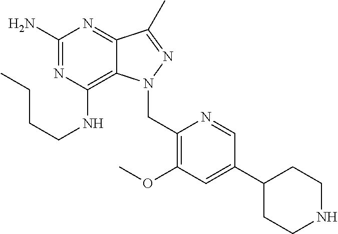 [SMILES: CCCCNc1nc(N)nc2c(C)nn(Cc3ncc(C4CCNCC4)cc3OC)c12] | 549.3 | 150.2 | LC/MS [M + H]⁺ = 425.1 RT (min) = 1.13 δ 7.97 (s, 1H), 7.91-7.82 (m, 1H), 7.39 (s, 1H), 5.61 (br s, 2H), 5.58 (s, 2H), 3.92 (s, 3H), 3.15 (br d, J = 12.2 Hz, 2H), 2.80-2.65 (m, 3H), 2.21 (s, 3H), 1.82-1.73 (m, 2H), 1.72- 1.56 (m, 4H), 1.43-1.33 (m, 2H), 0.94 (t, J = 7.3 Hz, 3H) |
| | N7-butyl-1-{[3-methoxy-5-(piperidin-4- | | | |
| | yl)pyridin-2-yl]methyl}-3-methyl-1H- | | | |
| | pyrazolo[4,3-d]pyrimidine-5,7-diamine | | | |
| 107 | 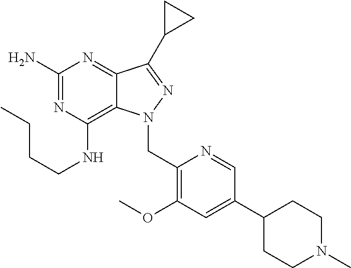 [SMILES: CCCCNc1nc(N)nc2c(C3CC3)nn(Cc3ncc(C4CCN(C)CC4)cc3OC)c12] | 383.1 | 444.2 | LC/MS [M + H]⁺ = 465.3 RT (min) = 1.25 δ 7.96 (s, 1H), 7.85-7.76 (m, 1H), 7.40 (s, 1H), 5.53 (br s, 4H), 3.89 (s, 3H), 2.86 (br d, J = 11.0 Hz, 2H), 2.19 (s, 3H), 2.03-1.89 (m, 2H), 1.84 (br s, 1H), 1.77-1.65 (m, 3H), 1.64-1.53 (m, 2H), 1.39-1.29 (m, 2H), 0.95 (br d, J = 3.1 Hz, 2H), 0.91 (br t, J = 7.3 Hz, 2H), 0.84 (br dd, J = 8.2, 2.1 Hz, 2H) |
| | N7-butyl-3-cyclopropyl-1-{[3-methoxy-5- | | | |
| | (1-methylpiperidin-4-yl)pyridin-2- | | | |
| | yl]methyl}-1H-pyrazolo[4,3-d]pyrimidine- | | | |
| | 5,7-diamine | | | |
| 108 | 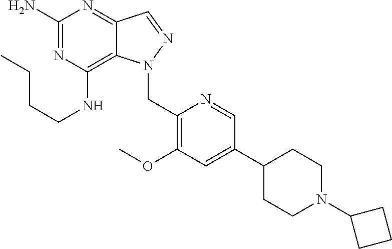 [SMILES: CCCCNc1nc(N)nc2cnn(Cc3ncc(C4CCN(C5CCC5)CC4)cc3OC)c12] | 51.9 | 4.8 | LC/MS [M + H]⁺ = 465.0 RT (min) = 1.20 δ 8.03 (br s, 1H), 7.99 (s, 1H), 7.48 (s, 1H), 7.42 (s, 1H), 5.65 (s, 2H), 5.60 (br s, 2H), 3.89 (s, 3H), 3.47 (br d, J = 4.6 Hz, 2H), 2.91 (br d, J = 11.9 Hz, 3H), 2.79-2.68 (m, 1H), 2.06-1.95 (m, 2H), 1.88-1.54 (m, 12H), 1.44-1.28 (m, 2H), 0.93 (br t, J = 7.2 Hz, 3H) |
| | N7-butyl-1-{[5-(1-cyclobutylpiperidin-4- | | | |
| | yl)-3-methoxypyridin-2-yl]methyl}-1H- | | | |
| | pyrazolo[4,3-d]pyrimidine-5,7-diamine | | | |
| 109 | 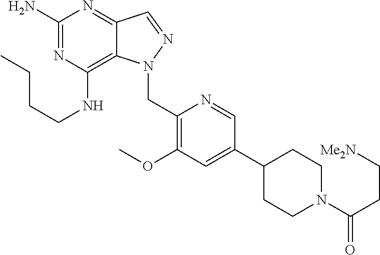 [SMILES: CCCCNc1nc(N)nc2cnn(Cc3ncc(C4CCN(C(=O)CCN(C)C)CC4)cc3OC)c12] | 214.7 | 14.5 | LC/MS [M + H]⁺ = 510.3 RT (min) = 1.19 δ 9.38 (br t, J = 4.7 Hz, 1H), 7.99 (s, 1H), 7.74 (s, 1H), 7.46 (s, 1H), 5.80 (s, 2H), 4.56 (br d, J = 11.9 Hz, 1H), 3.98 (br d, J = 12.8 Hz, 1H), 3.91 (s, 3H), 3.59 (br s, 2H), 3.37-3.22 (m, 2H), 3.21-3.08 (m, 1H), 2.97- 2.85 (m, 3H), 2.82 (br s, 6H), 2.69 (br t, J = 11.9 Hz, 1H), 1.88-1.75 (m, 2H), 1.74-1.60 (m, 3H), 1.60- 1.48 (m, 1H), 1.40-1.29 (m, 2H), 0.92 (br t, J = 7.0 Hz, 3H) |
| | 1-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-3- | | | |
| | (dimethylamino)propan-1-one | | | |
| 110 | 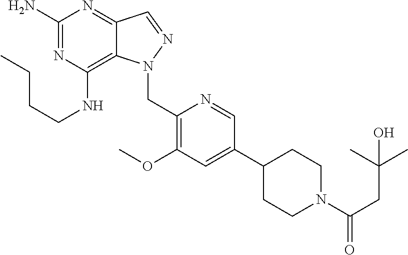 [SMILES: CCCCNc1nc(N)nc2cnn(Cc3ncc(C4CCN(C(=O)CC(C)(C)O)CC4)cc3OC)c12] | 342.9 | 382.3 | LC/MS [M + H]⁺ = 511.2 RT (min) = 1.49 δ 8.05 (br s, 1H), 7.99 (s, 1H), 7.49 (s, 1H), 7.42 (s, 1H), 5.66 (br s, 4H), 4.61 (br d, J = 12.8 Hz, 1H), 4.12 (br d, J = 11.9 Hz, 1H), 3.89 (s, 3H), 3.59 (br s, 1H), 3.53-3.42 (m, 1H), 3.16- 3.04 (m, 1H), 2.94-2.82 (m, 1H), 2.69-2.59 (m, 2H), 2.50-2.39 (m, 2H), 1.80 (brd, J = 11.3 Hz, 2H), 1.71-1.47 (m, 4H), 1.42-1.31 (m, 2H), 1.20 (s, 6H), 0.93 (br t, J = 7.3 Hz, 3H) |
| | 1-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-3- | | | |
| | hydroxy-3-methylbutan-1-one | | | |
| 111 | 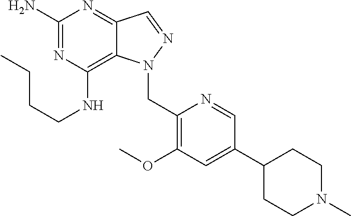 [SMILES: CCCCNc1nc(N)nc2cnn(Cc3ncc(C4CCN(C)CC4)cc3OC)c12] | 65.3 | 5.4 | LC/MS [M + H]⁺ = 425.1 RT (min) = 1.13 δ 8.09-7.96 (m, 2H), 7.48 (s, 1H), 7.41 (s, 1H), 5.72-5.56 (m, 4H), 3.89 (s, 3H), 3.52-3.36 (m, 2H), 3.00-2.86 (m, 2H), 2.26 (s, 3H), 2.15-1.99 (m, 2H), 1.83-1.66 (m, 4H), 1.67-1.58 (m, 2H), 1.44- 1.29 (m, 2H), 0.93 (br t, J = 7.2 Hz, 3H) |
| | N7-butyl-1-{[3-methoxy-5-(1-methyl- | | | |
| | piperidin-4-yl)pyridin-2-yl]methyl}-1H- | | | |
| | pyrazolo[4,3-d]pyrimidine-5,7-diamine | | | |
| 112 | 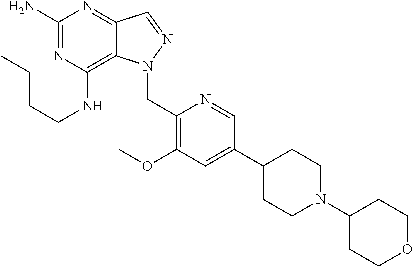 [SMILES: CCCCNc1nc(N)nc2cnn(Cc3ncc(C4CCN(C5CCOCC5)CC4)cc3OC)c12] | 34.0 | 5.9 | LC/MS [M + H]⁺ = 495.2 RT (min) = 1.10 δ 8.08-8.02 (m, 1H), 7.98 (s, 1H), 7.48 (s, 1H), 7.41 (s, 1H), 5.69 (br s, 2H), 5.65 (s, 2H), 3.95-3.84 (m, 7H), 3.29 (br t, J = 11.6 Hz, 2H), 3.07 (br d, J = 9.8 Hz, 2H), 2.68-2.58 (m, 2H), 2.35-2.25 (m, 2H), 1.85- 1.66 (m, 6H), 1.63 (quin, J = 7.2 Hz, 2H), 1.53-1.41 (m, 2H), 1.41- 1.31 (m, 2H), 0.92 (t, J = 7.3 Hz, 3H) |
| | N7-butyl-1-({3-methoxy-5-[1-(oxan-4- | | | |
| | yl)piperidin-4-yl]pyridin-2-yl}methyl)-1H- | | | |
| | pyrazolo[4,3-d]pyrimidine-5,7-diamine | | | |
| 113 | 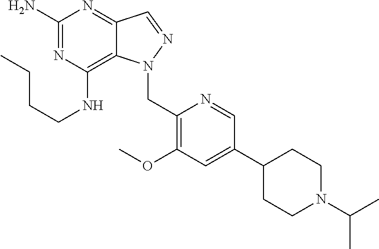 [SMILES: CCCCNc1nc(N)nc2cnn(Cc3ncc(C4CCN(C(C)C)CC4)cc3OC)c12] | 47.2 | 9.4 | LC/MS [M + H]⁺ = 452.1 RT (min) = 1.17 δ 7.99 (s, 2H), 7.47 (s, 1H), 7.43 (s, 1H), 5.65 (s, 2H), 5.60 (s, 2H), 3.90 (s, 3H), 2.90 (br d, J = 10.1 Hz, 2H), 2.77-2.68 (m, 1H), 2.60-2.57 (m, 1H), 2.22 (br t, J = 10.7 Hz, 2H), 1.81- 1.59 (m, 6H), 1.44-1.33 (m, 2H), 1.00 (d, J = 6.4 Hz, 6H), 0.94 (t, J = 7.3 Hz, 3H). |
| | N7-butyl-1-({3-methoxy-5-[1-(propan-2- | | | |
| | yl)piperidin-4-yl]pyridin-2-yl}methyl)-1H- | | | |
| | pyrazolo[4,3-d]pyrimidine-5,7-diamine | | | |
| 114 | 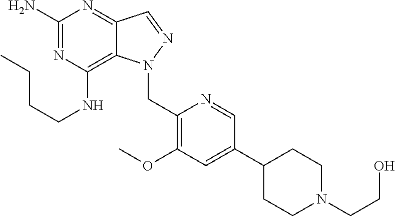 [SMILES: CCCCNc1nc(N)nc2cnn(Cc3ncc(C4CCN(CCO)CC4)cc3OC)c12] | 49.9 | 3.6 | LC/MS [M + H]⁺ = 455.2 RT (min) = 1.11 δ 7.99 (s, 2H), 7.48 (s, 1H), 7.43 (s, 1H), 5.66 (s, 2H), 5.62 (s, 2H), 3.90 (s, 3H), 3.55 (br t, J = 6.0 Hz, 1H), 3.51-3.40 (m, 2H), 3.06-2.96 (m, 2H), 2.66-2.58 (m, 1H), 2.49 (br t, J = 6.0 Hz, 2H), 2.14 (br d, J = 4.0 Hz, 2H), 1.79-1.69 (m, 4H), 1.69- 1.59 (m, 2H), 1.44-1.33 (m, 2H), 0.94 (t, J = 7.3 Hz, 3H) |
| | 2-[4-(6-{[5-amino-7-(butylamino)-1H-py- | | | |
| | razolo[4,3-d]pyrimidin-1-yl]methyl}-5- | | | |
| | methoxypyridin-3-yl)piperidin-1- | | | |
| | yl]ethan-1-ol | | | |
| 115 | 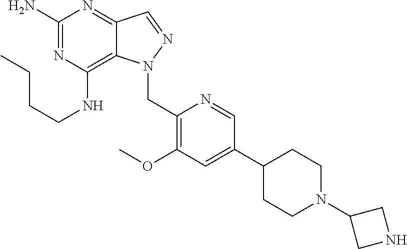 [SMILES: CCCCNc1nc(N)nc2cnn(Cc3ncc(C4CCN(C5CNC5)CC4)cc3OC)c12] | 447.0 | 64.7 | LC/MS [M + H]⁺ = 466.2 RT (min) = 1.01 δ 7.99 (s, 1H), 7.96-7.88 (m, 1H), 7.47 (s, 1H), 7.43 (s, 1H), 5.65 (s, 2H), 5.60 (s, 2H), 3.90 (s, 3H), 3.76- 3.58 (m, 2H), 2.85 (br d, J = 10.4 Hz, 2H), 2.65-2.57 (m, 2H), 1.90- 1.83 (m, 2H), 1.83-1.55 (m, 6H), 1.44-1.33 (m, 2H), 0.93 (t, J = 7.3 Hz, 3H) |
| | 1-({5-[1-(azetidin-3-yl)piperidin-4-yl]-3- | | | |
| | methoxypyridin-2-yl}methyl)-N7-butyl- | | | |
| | 1H-pyrazolo[4,3-d]pyrimidine-5,7- | | | |
| | diamine | | | |
| 116 | 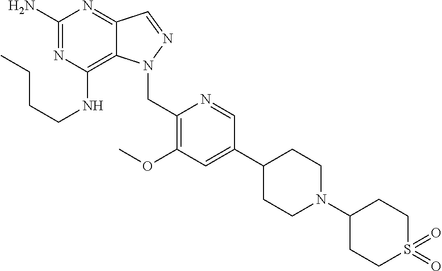 [SMILES: CCCCNc1nc(N)nc2cnn(Cc3ncc(C4CCN(C5CCS(=O)(=O)CC5)CC4)cc3OC)c12] | 84.6 | 10.5 | LC/MS [M + H]⁺ = 543.0 RT (min) = 1.10 δ 8.07 (br d, J = 5.3 Hz, 1H), 7.96 (s, 1H), 7.46 (s, 1H), 7.38 (s, 1H), 5.68- 5.56 (m, 4H), 3.87 (s, 3H), 3.45 (br d, J = 5.6 Hz, 2H), 3.16-3.02 (m, 4H), 2.89 (br d, J = 10.5 Hz, 2H), 2.71-2.62 (m, 1H), 2.60-2.56 (m, 1H), 2.26 (br t, J = 10.6 Hz, 2H), 2.06- 1.94 (m, 4H), 1.79-1.71 (m, 2H), 1.71-1.54 (m, 4H), 1.41-1.27 (m, 2H), 0.90 (t, J = 7.4 Hz, 3H) |
| | 4-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-1λ⁶- | | | |
| | thiane-1,1-dione | | | |
| 117 | 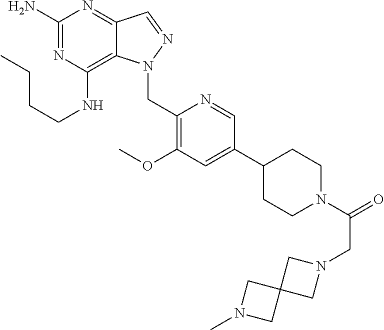 [SMILES: CCCCNc1nc(N)nc2cnn(Cc3ncc(C4CCN(C(=O)CN5CC6(CN(C)C6)C5)CC4)cc3OC)c12] | 855.5 | 312.6 | LC/MS [M + H]⁺ = 563.2 RT (min) = 1.11 δ 8.06-7.86 (m, 2H), 7.47 (s, 1H), 7.42 (s, 1H), 5.65 (s, 2H), 5.59 (s, 2H), 3.89 (s, 3H), 3.30-3.21 (m, 4H), 3.19 (s, 2H), 3.08-2.92 (m, 2H), 2.91-2.79 (m, 1H), 2.17 (s, 3H), 1.91 (s, 2H), 1.85-1.72 (m, 2H), 1.69-1.47 (m, 4H), 1.44- 1.31 (m, 2H), 0.93 (t, J = 7.2 Hz, 3H). |
| | 1-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-2-{6- | | | |
| | methyl-2,6-diazaspiro[3.3]heptan-2- | | | |
| | yl}ethan-1-one | | | |
| 118 | 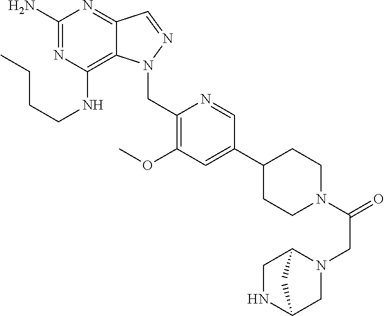 [SMILES: CCCCNc1nc(N)nc2cnn(Cc3ncc(C4CCN(C(=O)CN5C[C@@H]6C[C@H]5CN6)CC4)cc3OC)c12] | 715.9 | 890.0 | LC/MS [M + H]⁺ = 549.3 RT (min) = 1.11 δ 8.02-7.86 (m, 2H), 7.45 (s, 1H), 7.37 (s, 1H), 5.63 (s, 2H), 5.55 (s, 2H), 3.88 (s, 3H), 3.82-3.70 (m, 4H), 3.53-3.30 (m,4H), 3.26- 3.18 (m, 1H), 3.10-3.01 (m, 1H), 2.98-2.90 (m, 1H), 2.91-2.78 (m, 2H), 2.71-2.58 (m, 2H), 1.89 (br d, J = 8.4 Hz, 1H), 1.80-1.76 (m, 1H), 1.71-1.41 (m, 6H), 1.41-1.25 (m, 2H), 0.89 (t, J = 7.3 Hz, 3H) |
| | 1-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-2- | | | |
| | [(1S,4S)-2,5-diazabicyclo[2.2.1]heptan-2- | | | |
| | yl]ethan-1-one | | | |
| 119 | 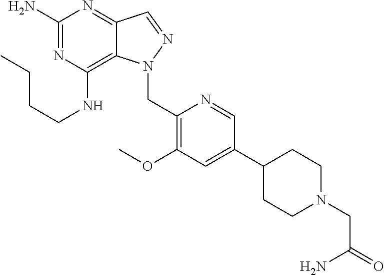 [SMILES: CCCCNc1nc(N)nc2cnn(Cc3ncc(C4CCN(CC(N)=O)CC4)cc3OC)c12] | 164.7 | 5.1 | LC/MS [M + H]⁺ = 468.1 RT (min) = 0.81 δ 9.30-9.11 (m, 1H), 7.96 (br d, J = 8.3 Hz, 2H), 7.72 (s, 1H), 7.69 (br s, 1H), 7.42 (br s, 1H), 5.80 (s, 2H), 3.96-3.89 (m, 3H), 3.82-3.28 (m, 6H), 3.20-3.02 (m, 1H), 2.96- 2.80 (m, 1H), 2.12-1.87 (m, 4H), 1.68-1.55 (m, 2H), 1.40-1.22 (m, 2H), 0.90 (t, J = 7.3 Hz, 3H), |
| | 2-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1- | | | |
| | yl]acetamide | | | |
| 120 | 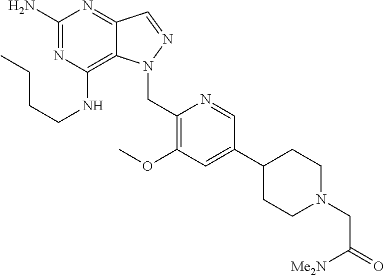 [SMILES: CCCCNc1nc(N)nc2cnn(Cc3ncc(C4CCN(CC(=O)N(C)C)CC4)cc3OC)c12] | 79.1 | 2.0 | LC/MS [M + H]⁺ = 496.1 RT (min) = 0.86 δ 8.04-7.87 (m, 2H), 7.45 (s, 1H), 7.41 (s, 1H), 5.63 (s, 2H), 5.56 (s, 2H), 3.88 (s, 3H), 3.50 (br s, 3H), 3.13 (s, 2H), 3.03 (s, 3H), 2.93 (br d, J = 11.6 Hz, 2H), 2.81 (s, 3H), 2.20- 2.04 (m, 2H), 1.76-1.66 (m, 4H), 1.62 (quin, J = 7.2 Hz, 2H), 1.44- 1.28 (m, 2H), 0.92 (t, J = 7.3 Hz, 3H) |
| | 2-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-N,N- | | | |
| | dimethylacetamide | | | |
| 121 | 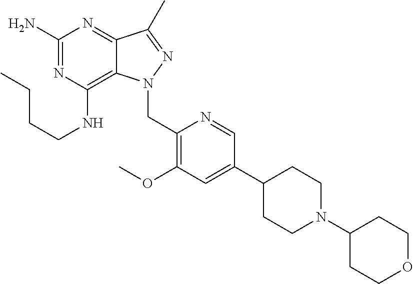 [SMILES: CCCCNc1nc(N)nc2c(C)nn(Cc3ncc(C4CCN(C5CCOCC5)CC4)cc3OC)c12] | 602.0 | 121.6 | LC/MS [M + H]⁺ = 509.2 RT (min) = 0.900 δ 8.07-7.96 (m, 1H), 7.96 (s, 1H), 7.40 (s, 1H), 5.87-5.67 (m, 2H), 5.58 (s, 2H), 3.95-3.82 (m, 5H), 3.53-3.40 (m, 2H), 3.33-3.23 (m, 1H), 2.68-2.61 (m, 1H), 2.20 (s, 3H), 1.89-1.67 (m, 6H), 1.61 (quin, J = 7.2 Hz, 2H), 1.56-1.42 (m, 2H), 1.40-1.28 (m, 2H), 0.91 (t, J = 7.3 Hz, 3H) |
| | N7-butyl-1-({3-methoxy-5-[1-(oxan-4- | | | |
| | yl)piperidin-4-yl]pyridin-2-yl}methyl)-3- | | | |
| | methyl-1H-pyrazolo[4,3-d]pyrimidine- | | | |
| | 5,7-diamine | | | |
| 122 | 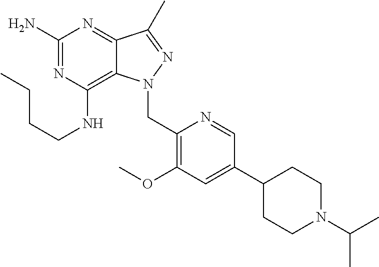 [SMILES: CCCCNc1nc(N)nc2c(C)nn(Cc3ncc(C4CCN(C(C)C)CC4)cc3OC)c12] | 192.8 | 18.7 | LC/MS [M + H]⁺ = 467.3 RT (min) = 0.92 δ 7.98-7.86 (m, 2H), 7.38 (s, 1H), 5.55 (br s, 4H), 3.88 (s, 3H), 3.56- 3.52 (m, 2H), 3.48-3.34 (m, 1H), 2.88 (br d, J = 11.5 Hz, 2H), 2.76- 2.66 (m, 1H), 2.26-2.11 (m, 5H), 1.77-1.52 (m, 6H), 1.40-1.27 (m, 2H), 0.98 (d, J = 6.6 Hz, 6H), 0.90 (t, J = 7.3 Hz, 3H) |
| | N7-butyl-1-({3-methoxy-5-[1-(propan-2- | | | |
| | yl)piperidin-4-yl]pyridin-2-yl}methyl)-3- | | | |
| | methyl-1H-pyrazolo[4,3-d]pyrimidine- | | | |
| | 5,7-diamine | | | |
| 123 | 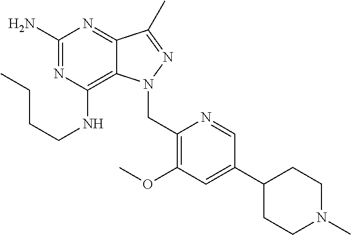 [SMILES: CCCCNc1nc(N)nc2c(C)nn(Cc3ncc(C4CCN(C)CC4)cc3OC)c12] | 491.0 | 40.2 | LC/MS [M + H]⁺ = 439.3 RT (min) = 0.87 δ 8.10 (br dd, J = 6.1, 3.1 Hz, 1H), 7.97 (s, 1H), 7.41 (s, 1H), 6.14- 5.96 (m, 2H), 5.62 (s, 2H), 3.92 (s, 3H), 2.87-2.72 (m, 2H), 2.70- 2.60 (m, 2H), 2.23 (s, 3H), 2.02- 1.79 (m, 4H), 1.62 (quin, J = 7.0 Hz, 2H), 1.35 (dq, J = 14.8, 7.5 Hz, 2H), 0.92 (br t, J = 7.5 Hz, 3H) |
| | N7-butyl-1-{[3-methoxy-5-(1-methylpiper- | | | |
| | idin-4-yl)pyridin-2-yl]methyl}-3-methyl- | | | |
| | 1H-pyrazolo[4,3-d]pyrimidine-5,7- | | | |
| | diamine | | | |
| 124 | 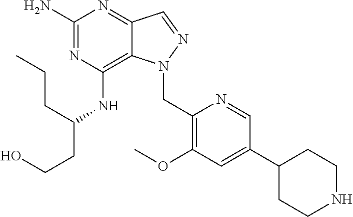 | 188.5 | 56.9 | LC/MS [M + H]⁺ = 455.5 RT (min) = 0.93 δ 8.29 (s, 1H), 7.95 (s, 1H), 7.76- 7.68 (m, 1H), 7.47 (s, 1H), 5.69- 5.58 (m, 4H), 4.46-4.36 (m, 1H), 3.89 (s, 3H), 3.37-3.30 (m, 2H), 2.91 (q, J = 12.5 Hz, 4H), 1.94 (br d, J = 1.4 Hz, 1H), 1.88-1.49 (m, 9H), 1.40-1.23 (m, 2H), 0.87 (t, J = 7.4 Hz, 3H) |
| | (3S)-3-[(5-amino-1-{[3-methoxy-5- | | | |
| | (piperidin-4-yl)pyridin-2-yl]methyl}-1H- | | | |
| | pyrazolo[4,3-d]pyrimidin-7- | | | |
| | yl)amino]hexan-1-ol | | | |
| 125 | 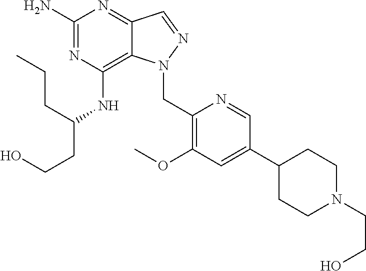 | 57.8 | 10.2 | LC/MS [M + H]⁺ = 499.0 RT (min) = 1.09 δ 7.96 (s, 1H), 7.82 (br d, J = 8.1 Hz, 1H), 7.46 (s, 1H), 7.42 (br s, 1H), 5.72-5.44 (m, 4H), 4.40 (br d, J = 6.6 Hz, 1H), 3.88 (s, 3H), 3.53- 3.50 (m, 4H), 3.01-2.91 (m, 2H), 2.62-2.55 (m, 3H), 2.42 (t, J = 6.3 Hz, 2H), 2.12-2.01 (m, 2H), 1.84- 1.63 (m, 6H), 1.63-1.48 (m, 2H), 1.41-1.16 (m, 2H), 0.87 (t, J = 7.3 Hz, 3H) |
| | (3S)-3-{[5-amino-1-({5-[1-(2-hydroxy- | | | |
| | ethyl)piperidin-4-yl]-3-methoxypyridin-2- | | | |
| | yl}methyl)-1H-pyrazolo[4,3-d]pyrimidin- | | | |
| | 7-yl]amino}hexan-1-ol | | | |
| 126 | 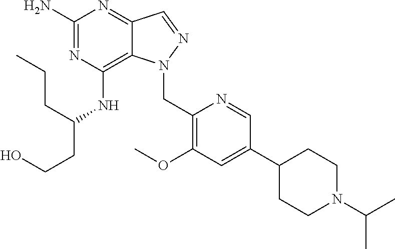 | 15.5 | 2.0 | LC/MS [M + H]⁺ = 497.2 RT (min) = 1.10 δ 7.95 (s, 1H), 7.83 (br d, J = 7.8 Hz, 1H), 7.46 (s, 1H), 7.41 (s, 1H), 5.72- 5.45 (m, 4H), 4.48-4.34 (m, 1H), 3.88 (s, 3H), 3.81-3.66 (m, 2H), 2.91 (br d, J = 10.9 Hz, 2H), 2.80- 2.70 (m, 1H), 2.62-2.55 (m, 2H), 2.25 (br t, J = 10.6 Hz, 2H), 1.85- 1.62 (m, 6H), 1.62-1.52 (m, 2H), 1.39-1.20 (m, 2H), 1.00 (d, J = 6.6 Hz, 6H), 0.86 (t, J = 7.3 Hz, 3H) |
| | (3S)-3-{[5-amino-1-({3-methoxy-5-[1- | | | |
| | (propan-2-yl)piperidin-4-yl]pyridin-2- | | | |
| | yl}methyl)-1H-pyrazolo[4,3-d]pyrimidin- | | | |
| | 7-yl]amino}hexan-1-ol | | | |
| 127 | 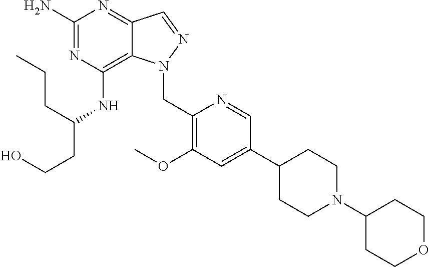 | 12.8 | 2.7 | LC/MS [M + H]⁺ = 539.3 RT (min) = 1.06 δ 7.97 (s, 1H), 7.89 (br d, J = 7.6 Hz, 1H), 7.48 (s, 1H), 7.43 (s, 1H), 5.75- 5.41 (m, 4H), 4.42 (br d, J = 5.8 Hz, 1H), 3.90 (s, 3H), 3.29 (br t, J = 11.3 Hz, 2H), 3.06 (br d, J = 9.2 Hz, 2H), 2.68-2.58 (m, 1H), 2.34-2.21 (m, 2H), 1.84-1.65 (m, 8H), 1.65- 1.53 (m, 2H), 1.53-1.40 (m, 2H), 1.38-1.24 (m, 2H), 0.88 (br t, J = 7.3 Hz, 3H) |
| | (3S)-3-{[5-amino-1-({3-methoxy-5-[1- | | | |
| | (oxan-4-yl)piperidin-4-yl]pyridin-2- | | | |
| | yl}methyl)-1H-pyrazolo[4,3-d]pyrimidin- | | | |
| | 7-yl]amino}hexan-1-ol | | | |
| 128 | 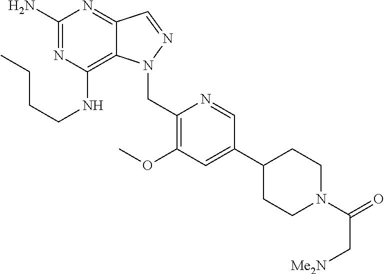 [SMILES: CCCCNc1nc(N)nc2cnn(Cc3ncc(C4CCN(C(=O)CN(C)C)CC4)cc3OC)c12] | 48.4 | 3.5 | LC/MS [M + H]⁺ = 496.0 RT (min) = 1.16 δ 8.04-7.85 (m, 2H), 7.46 (s, 1H), 7.42 (br s, 1H), 5.64 (s, 2H), 5.61 (br s, 2H), 4.55-4.39 (m, 1H), 4.21- 4.07 (m, 1H), 3.88 (d, J = 1.6 Hz, 3H), 3.48 (br s, 4H), 3.12-3.02 (m, 1H), 2.90-2.80 (m, 1H), 2.66- 2.57 (m, 1H), 2.21 (s, 6H), 1.79 (br d, J = 11.8 Hz, 2H), 1.70-1.58 (m, 3H), 1.57-1.46 (m, 1H), 1.41- 1.30 (m, 2H), 0.92 (td, J = 7.4, 1.7 Hz, 3H) |
| | 1-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-2- | | | |
| | (dimethylamino)ethan-1-one | | | |
| 129 | 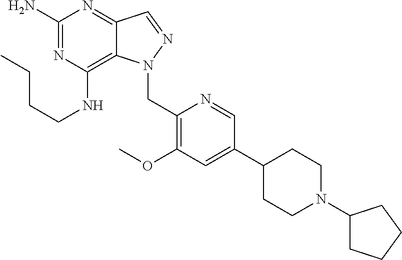 [SMILES: CCCCNc1nc(N)nc2cnn(Cc3ncc(C4CCN(C5CCCC5)CC4)cc3OC)c12] | 27.3 | 7.6 | LC/MS [M + H]⁺ = 479.3 RT (min) = 1.22 δ 8.17 (s, 1H), 8.02-7.98 (m, 1H), 7.46 (s, 1H), 7.42 (s, 1H), 5.67 (br d, J = 11.5 Hz, 2H), 5.64 (s, 1H), 3.89 (s, 3H), 3.20-3.08 (m, 2H), 2.76- 2.58 (m, 3H), 2.27-2.14 (m, 2H), 1.90-1.70 (m, 7H), 1.62 (quin, J = 7.1 Hz, 5H), 1.56-1.47 (m, 2H), 1.45-1.34 (m, 4H), 0.92 (t, J = 7.4 Hz, 3H) |
| | N7-butyl-1-{[5-(1-cyclopentylpiperidin-4- | | | |
| | yl)-3-methoxypyridin-2-yl]methyl}-1H- | | | |
| | pyrazolo[4,3-d]pyrimidine-5,7-diamine | | | |
| 130 | 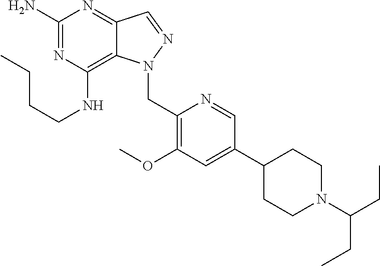 [SMILES: CCCCNc1nc(N)nc2cnn(Cc3ncc(C4CCN(C(CC)CC)CC4)cc3OC)c12] | 119.0 | | LC/MS [M + H]⁺ = 481.3 RT (min) = 1.24 δ 7.99 (s, 2H), 7.47 (s, 1H), 7.41 (s, 1H), 5.65 (s, 2H), 5.59 (s, 2H), 3.90 (s, 3H), 2.83-2.75 (m, 2H), 2.37 (br t, J = 11.0 Hz, 2H), 2.24-2.15 (m, 1H), 1.74 (br s, 2H), 1.70-1.59 (m, 4H), 1.49 (dt, J = 13.9, 7.1 Hz, 2H), 1.42-1.34 (m, 2H), 1.28 (dt, J = 14.0, 7.3 Hz, 2H), 0.98-0.82 (m, 9H) |
| | N7-butyl-1-({3-methoxy-5-[1-(pentan-3- | | | |
| | yl)piperidin-4-yl]pyridin-2-yl}methyl)-1H- | | | |
| | pyrazolo[4,3-d]pyrimidine-5,7-diamine | | | |
| 131 | 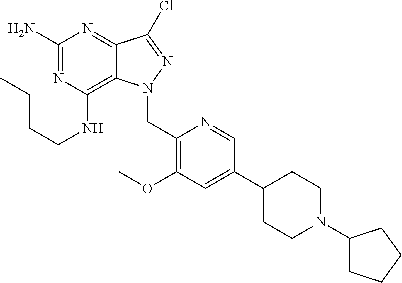 [SMILES: CCCCNc1nc(N)nc2c(Cl)nn(Cc3ncc(C4CCN(C5CCCC5)CC4)cc3OC)c12] | 594.9 | 971.8 | LC/MS [M + H]⁺ = 513.0 RT (min) = 1.29 δ 7.98 (s, 1H), 7.94 (br t, J = 5.2 Hz, 1H), 7.44 (s, 1H), 5.91 (s, 2H), 5.63 (s, 2H), 3.96-3.82 (m, 3H), 3.07 (br d, J = 10.1 Hz, 2H), 2.64-2.54 (m, 2H), 2.09-1.96 (m, 2H), 1.87- 1.77 (m, 2H), 1.77-1.69 (m, 3H), 1.62 (quin, J = 6.9 Hz, 4H), 1.56- 1.44 (m, 2H), 1.41-1.28 (m, 4H), 0.92 (t, J = 7.3 Hz, 3H) |
| | N7-butyl-3-chloro-1-{[5-(1- | | | |
| | cyclopentylpiperidin-4-yl)-3- | | | |
| | methoxypyridin-2-yl]methyl}-1H- | | | |
| | pyrazolo[4,3-d]pyrimidine-5,7-diamine | | | |
| 132 | 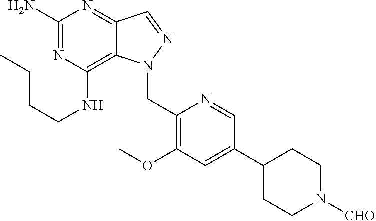 [SMILES: CCCCNc1nc(N)nc2cnn(Cc3ncc(C4CCN(C=O)CC4)cc3OC)c12] | 180.0 | 194.7 | LC/MS [M + H]⁺ = 438.9 RT (min) = 1.33 δ 8.02 (s, 2H), 7.98 (s, 1H), 7.47 (s, 1H), 7.44 (s, 1H), 5.72 (br s, 2H), 5.64 (s, 2H), 4.31 (br d, J = 13.8 Hz, 1H), 3.88 (s, 3H), 3.83-3.74 (m, 1H), 3.51 (br s, 2H), 3.19-3.07 (m, 1H), 2.94-2.79 (m, 1H), 2.67 (td, J = 12.8, 2.6 Hz, 1H), 1.79 (br t, J = 16.0 Hz, 2H), 1.68-1.47 (m, 4H), 1.36 (dq, J = 14.9, 7.5 Hz, 2H), 0.92 (t, J = 7.4 Hz, 3H) |
| | 4-(6-{[5-amino-7-(butylamino)-1H- | | | |
| | pyrazolo[4,3-d]pyrimidin-1-yl]methyl}-5- | | | |
| | methoxypyridin-3-yl)piperidine-1- | | | |
| | carbaldehyde | | | |
| 133 | 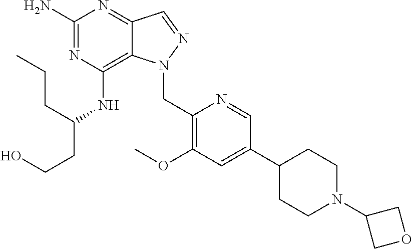 | 7.6 | 5.4 | LC/MS [M + H]⁺ = 511.2 RT (min) = 1.06 δ 8.07 (br s, 1H), 7.95 (s, 1H), 7.49 (s, 1H), 7.40 (s, 1H), 5.79 (br s, 2H), 5.68-5.50 (m, 2H), 4.53 (t, J = 6.6 Hz, 2H), 4.48-4.34 (m, 3H), 3.87 (s, 3H), 3.52-3.37 (m, 3H), 2.77 (br d, J = 11.3 Hz, 2H), 2.65-2.55 (m, 2H), 1.90-1.81 (m, 2H), 1.83- 1.62 (m, 6H), 1.64-1.49 (m, 2H), 1.26 (td, J = 14.8, 7.2 Hz, 2H), 0.84 (t, J = 7.3 Hz, 3H) |
| | (3S)-3-{[5-amino-1-({3-methoxy-5-[1- | | | |
| | (oxetan-3-yl)piperidin-4-yl]pyridin-2- | | | |
| | yl}methyl)-1H-pyrazolo[4,3-d]pyrimidin- | | | |
| | 7-yl]amino}hexan-1-ol | | | |
| 134 | 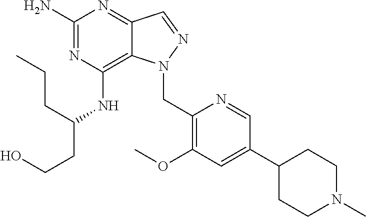 | 14.1 | 2.6 | LC/MS [M + H]⁺ = 469.0 RT (min) = 1.06 δ 7.97 (s, 1H), 7.87 (br d, J = 8.5 Hz, 1H), 7.48 (s, 1H), 7.42 (s, 1H), 5.79- 5.51 (m, 4H), 4.48-4.30 (m, 1H), 3.89 (s, 3H), 3.51-3.35 (m, 2H), 3.02 (br d, J = 11.3 Hz, 2H), 2.70- 2.60 (m, 1H), 2.35 (s, 3H), 2.26 (br t, J = 10.7 Hz, 2H), 1.86-1.64 (m, 6H), 1.58 (dt, J = 13.9, 7.1 Hz, 2H), 1.39-1.20 (m, 2H), 0.87 (t, J = 7.3 Hz, 3H) |
| | (3S)-3-[(5-amino-1-{[3-methoxy-5-(1- | | | |
| | methylpiperidin-4-yl)pyridin-2- | | | |
| | yl]methyl}-1H-pyrazolo[4,3-d]pyrimidin- | | | |
| | 7-yl)amino]hexan-1-ol | | | |
| 135 | 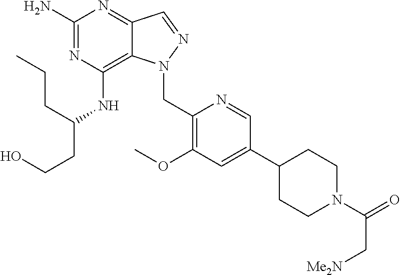 | 41.4 | 11.7 | LC/MS [M + H]⁺ = 540.2 RT (min) = 1.12 δ 7.98 (s, 1H), 7.85 (br d, J = 7.9 Hz, 1H), 7.49 (s, 1H), 7.45 (s, 1H), 5.79- 5.16 (m, 4H), 4.58-4.46 (m, 1H), 4.47-4.37 (m, 1H), 4.19-4.08 (m, 1H), 3.90 (s, 3H), 3.23 (br d, J = 4.9 Hz, 1H), 3.13-3.03 (m, 1H), 2.94- 2.82 (m, 1H), 2.64 (br t, J = 12.1 Hz, 1H), 2.27 (s, 6H), 1.86-1.69 (m, 4H), 1.68-1.48 (m, 4H), 1.32 (td, J = 14.0, 6.9 Hz, 2H), 0.89 (br t, J = 7.3 Hz, 3H) |
| | 1-(4-{6-[(5-amino-7-{[(3S)-1-hydroxy- | | | |
| | hexan-3-yl]amino}-1H-pyrazolo[4,3-d]pyri- | | | |
| | midin-1-yl)methyl]-5-methoxypyridin-3- | | | |
| | yl}piperidin-1-yl)-2-(dimethylamino)- | | | |
| | ethan-1-one | | | |
| 136 | 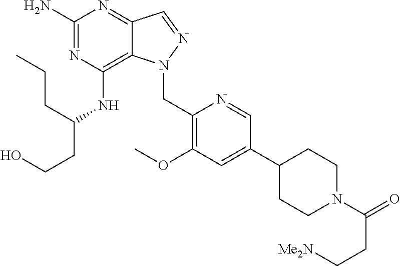 | 196.6 | 42.3 | LC/MS [M + H]⁺ = 554.3 RT (min) = 1.14 δ 7.94 (s, 1H), 7.86 (br d, J = 8.6 Hz, 1H), 7.46 (s, 1H), 7.39 (s, 1H), 5.66- 5.56 (m, 2H), 5.55 (br s, 2H), 4.50 (br d, J = 11.9 Hz, 1H), 4.42-4.30 (m, 1H), 3.88-3.85 (m, 7H), 3.50- 3.39 (m, 2H), 3.09 (br t, J = 12.3 Hz, 1H), 2.93-2.81 (m, 1H), 2.72- 2.63 (m, 2H), 2.60-2.57 (m, 1H), 2.32-2.22 (m, 6H), 1.83-1.67 (m, 4H), 1.64-1.40 (m, 4H), 1.26 (td, J = 14.8, 7.0 Hz, 2H), 0.84 (t, J = 7.3 Hz, 3H) |
| | 1-(4-{6-[(5-amino-7-{[(3S)-1- | | | |
| | hydroxyhexan-3-yl]amino}-1H- | | | |
| | pyrazolo[4,3-d]pyrimidin-1-yl)methyl]-5- | | | |
| | methoxypyridin-3-yl}piperidin-1-yl)-3- | | | |
| | (dimethylamino)propan-1-one | | | |
| 137 | 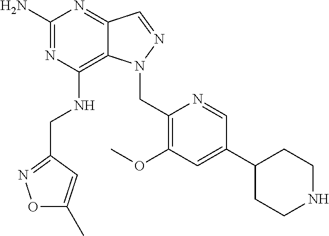 [SMILES: COc1cc(C2CCNCC2)cnc1Cn1ncc2nc(N)nc(NCc3cc(C)on3)c21] | 357.4 | 117.9 | LC/MS [M + H]⁺ = 450.1 RT (min) = 0.98 δ 7.83 (s, 1H), 7.52 (s, 1H), 7.37 (s, 1H), 6.20 (s, 1H), 5.73 (br s, 2H), 5.68 (s, 2H), 4.72 (br d, J = 3.4 Hz, 2H), 3.89 (s, 3H), 3.26-3.03 (m, 2H), 2.81-2.67 (m, 3H), 2.38 (s, 3H), 1.86-1.72 (m, 2H), 1.71- 1.62 (m, 2H) |
| | 1-{[3-methoxy-5-(piperidin-4-yl)pyridin- | | | |
| | 2-yl]methyl}-N7-[(5-methyl-1,2-oxazol-3- | | | |
| | yl)methyl]-1H-pyrazolo[4,3-d]pyrimidine- | | | |
| | 5,7-diamine | | | |
| 138 | 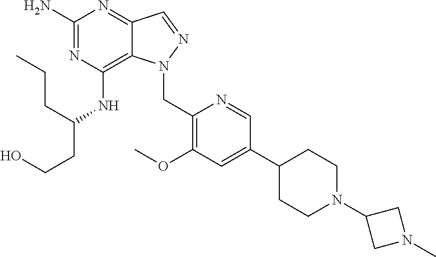 | 26.7 | 5.6 | LC/MS [M + H]⁺ = 524.4 RT (min) = 1.01 δ 7.98 (s, 1H), 7.83 (br d, J = 7.9 Hz, 1H), 7.52-7.42 (m, 2H), 5.72- 5.57 (m, 4H), 4.47-4.38 (m, 1H), 3.98-3.82 (m, 4H), 3.62-3.49 (m, 1H), 3.03-2.96 (m, 2H), 2.95- 2.87 (m, 1H), 2.81 (br d, J = 11.3 Hz, 2H), 2.64-2.58 (m, 1H), 2.36 (s, 3H), 1.89-1.64 (m, 6H), 1.64- 1.53 (m, 2H), 1.40-1.24 (m, 2H), 0.89 (br t, J = 7.5 Hz, 3H) |
| | (3S)-3-{[5-amino-1-({3-methoxy-5-[1-(1- | | | |
| | methylazetidin-3-yl)piperidin-4- | | | |
| | yl]pyridin-2-yl}methyl)-1H-pyrazolo[4,3- | | | |
| | d]pyrimidin-7-yl]amino}hexan-1-ol | | | |
| 139 | 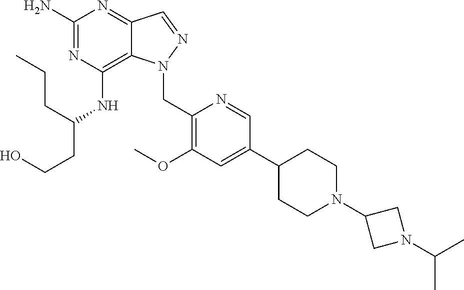 | 8.1 | 1.1 | LC/MS [M + H]⁺ = 552.1 RT (min) = 1.05 δ 8.00 (s, 1H), 7.86 (br d, J = 7.6 Hz, 1H), 7.50 (s, 1H), 7.47 (s, 1H), 5.74- 5.55 (m, 4H), 4.53-4.35 (m, 1H), 3.92 (s, 3H), 2.97-2.77 (m, 5H), 2.49-2.38 (m, 1H), 1.92-1.51 (m, 12H), 1.42-1.27 (m, 2H), 0.97- 0.88 (m, 9H) |
| | (3S)-3-({5-amino-1-[(3-methoxy-5-{1-[1- | | | |
| | (propan-2-yl)azetidin-3-yl]piperidin-4- | | | |
| | yl}pyridin-2-yl)methyl]-1H-pyrazolo[4,3- | | | |
| | d]pyrimidin-7-yl}amino)hexan-1-ol | | | |
| 140 | 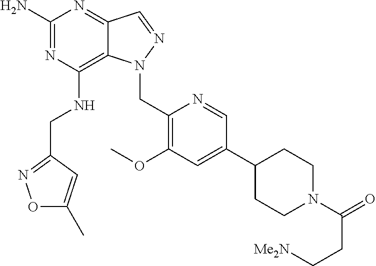 [SMILES: COc1cc(C2CCN(C(=O)CCN(C)C)CC2)cnc1Cn1ncc2nc(N)nc(NCc3cc(C)on3)c21] | 530.4 | 127.8 | LC/MS [M + H]⁺ = 549.0 RT (min) = 1.06 δ 7.88 (s, 1H), 7.55 (s, 1H), 7.45 (s, 1H), 6.24 (s, 1H), 5.78 (s, 2H), 5.71 (s, 2H), 4.75 (br d, J = 4.9 Hz, 2H), 4.59 (br d, J = 12.5 Hz, 1H), 4.05 (br d, J = 12.2 Hz, 1H), 3.92 (s, 3H), 3.21- 3.06 (m, 1H), 2.88 (br t, J = 12.1 Hz, 1H), 2.76 (br d, J = 6.4 Hz, 2H), 2.65 (br d, J = 7.6 Hz, 2H), 2.41 (s, 3H), 2.39 (s, 6H), 1.90-1.76 (m, 2H), 1.72-1.61 (m, 1H), 1.54 (br dd, J = 12.8, 3.4 Hz, 1H) |
| | 1-(4-{6-[(5-amino-7-{[(5-methyl-1,2- | | | |
| | oxazol-3-yl)methyl]amino}-1H- | | | |
| | pyrazolo[4,3-d]pyrimidin-1-yl)methyl]-5- | | | |
| | methoxypyridin-3-yl}piperidin-1-yl)-3- | | | |
| | (dimethylamino)propan-1-one | | | |
| 141 | 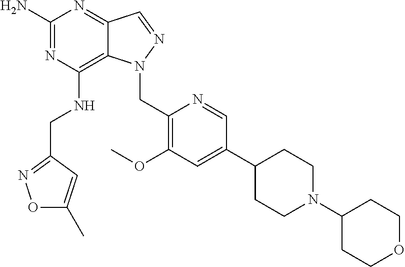 [SMILES: COc1cc(C2CCN(C3CCOCC3)CC2)cnc1Cn1ncc2nc(N)nc(NCc3cc(C)on3)c21] | 73.2 | 16.2 | LC/MS [M + H]⁺ = 534.2 RT (min) = 1.03 δ 8.58 (br s, 1H), 7.83 (s, 1H), 7.51 (s, 1H), 7.40 (s, 1H), 6.19 (s, 1H), 5.72 (s, 2H), 5.66 (s, 2H), 4.71 (br d, J = 5.5 Hz, 2H), 3.96-3.78 (m, 5H), 3.29 (br t, J = 11.0 Hz, 2H), 3.00 (br d, J = 10.4 Hz, 2H), 2.49-2.41 (m, 1H), 2.38 (s, 3H), 2.20 (br t, J = 11.0 Hz, 2H), 1.79-1.61 (m, 6H), 1.51-1.33 (m, 2H) |
| | 1-({3-methoxy-5-[1-(oxan-4-yl)piperidin- | | | |
| | 4-yl]pyridin-2-yl}methyl)-N7-[(5-methyl- | | | |
| | 1,2-oxazol-3-yl)methyl]-1H-pyrazolo[4,3- | | | |
| | d]pyrimidine-5,7-diamine | | | |
| 142 | 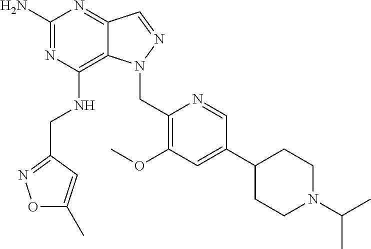 [SMILES: COc1cc(C2CCN(C(C)C)CC2)cnc1Cn1ncc2nc(N)nc(NCc3cc(C)on3)c21] | 43.3 | 5.6 | LC/MS [M + H]⁺ = 492.3 RT (min) = 1.09 δ 7.82 (s, 1H), 7.51 (s, 1H), 7.36 (s, 1H), 6.17 (s, 1H), 5.70 (br s, 2H), 5.66 (s, 2H), 4.71 (br d, J = 5.2 Hz, 2H), 3.88 (br d, J = 15.9 Hz, 3H), 3.01 (br d, J = 10.1 Hz, 2H), 2.93- 2.84 (m, 1H), 2.70-2.57 (m, 2H), 2.45-2.28 (m, 5H), 1.80 (br d, J = 10.4 Hz, 2H), 1.75-1.63 (m, 2H), 1.05 (br d, J = 6.4 Hz, 6H) |
| | 1-({3-methoxy-5-[1-(propan-2- | | | |
| | yl)piperidin-4-yl]pyridin-2-yl}methyl)-N7- | | | |
| | [(5-methyl-1,2-oxazol-3-yl)methyl]-1H- | | | |
| | pyrazolo[4,3-d]pyrimidine-5,7-diamine | | | |
| 143 | 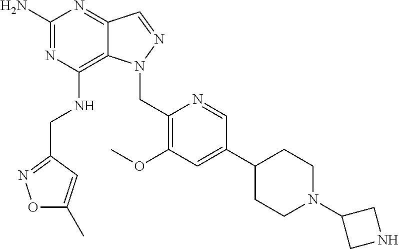 [SMILES: COc1cc(C2CCN(C3CNC3)CC2)cnc1Cn1ncc2nc(N)nc(NCc3cc(C)on3)c21] | 294.8 | 264.8 | LC/MS [M + H]⁺ = 505.1 RT (min) = 0.92 δ 9.90-9.74 (m, 1H), 7.85 (s, 1H), 7.78 (s, 1H), 7.43 (s, 1H), 6.27 (s, 1H), 5.82 (s, 2H), 4.84 (br d, J = 5.8 Hz, 2H), 4.10 (br d, J = 5.5 Hz, 3H), 3.90 (s, 3H), 2.83-2.68 (m, 1H), 2.40 (s, 3H), 1.92 (br d, J = 11.6 Hz, 2H), 1.86-1.70 (m, 2H) |
| | 1-({5-[1-(azetidin-3-yl)piperidin-4-yl]-3- | | | |
| | methoxypyridin-2-yl}methyl)-N7-[(5- | | | |
| | methyl-1,2-oxazol-3-yl)methyl]-1H- | | | |
| | pyrazolo[4,3-d]pyrimidine-5,7-diamine | | | |
| 144 | 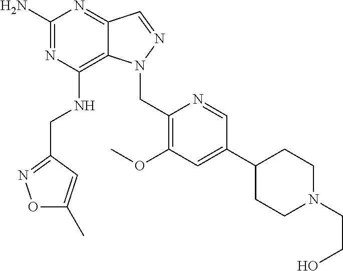 [SMILES: COc1cc(C2CCN(CCO)CC2)cnc1Cn1ncc2nc(N)nc(NCc3cc(C)on3)c21] | 77.0 | 8.7 | LC/MS [M + H]⁺ = 494.2 RT (min) = 1.02 δ 7.83 (s, 1H), 7.51 (s, 1H), 7.39 (s, 1H), 6.19 (s, 1H), 5.74 (s, 2H), 5.67 (s, 2H), 4.79-4.59 (m, 2H), 3.88 (s, 3H), 3.06 (br d, J = 10.4 Hz, 2H), 2.37 (s, 3H), 2.27-2.09 (m, 2H), 1.81-1.68 (m, 4H) |
| | 2-(4-{6-[(5-amino-7-{[(5-methyl-1,2-oxa- | | | |
| | zol-3-yl)methyl]amino}-1H-pyrazolo[4,3- | | | |
| | d]pyrimidin-1-yl)methyl]-5-methoxypy- | | | |
| | ridin-3-yl}piperidin-1-yl)ethan-1-ol | | | |
| 145 | 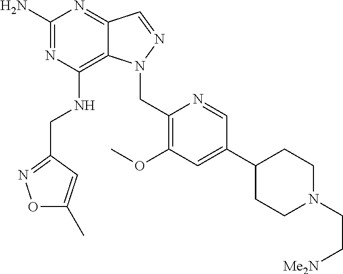 [SMILES: COc1cc(C2CCN(CCN(C)C)CC2)cnc1Cn1ncc2nc(N)nc(NCc3cc(C)on3)c21] | 100.9 | | LC/MS [M + H]⁺ = 521.7 RT (min) = 0.70 δ 8.72-8.38 (m, 1H), 7.82 (s, 1H), 7.50 (s, 1H), 7.36 (s, 1H), 6.18 (s, 1H), 5.72 (br s, 2H), 5.65 (s, 2H), 4.70 (br d, J = 5.4 Hz, 2H), 3.86 (s, 3H), 3.05 (br d, J = 10.3 Hz, 2H), 2.79 (br s, 2H), 2.61-2.56 (m, 3H), 2.46 (s, 6H), 2.36 (s, 3H), 2.21- 2.09 (m, 2H), 1.81-1.61 (m, 4H) |
| | 1-[(5-{1-[2-(dimethylamino)ethyl]- | | | |
| | piperidin-4-yl}-3-methoxypyridin-2- | | | |
| | yl)methyl]-N7-[(5-methyl-1,2-oxazol-3- | | | |
| | yl)methyl]-1H-pyrazolo[4,3-d]pyrimidine- | | | |
| | 5,7-diamine | | | |
| 146 | 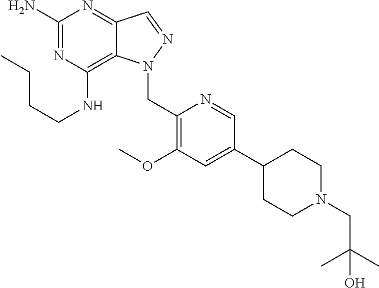 [SMILES: CCCCNc1nc(N)nc2cnn(Cc3ncc(C4CCN(CC(C)(C)O)CC4)cc3OC)c12] | 28.5 | 25.8 | LC/MS [M + H]⁺ = 482.9 RT (min) = 1.13 δ 8.04 (br d, J = 2.7 Hz, 1H), 7.97 (s, 1H), 7.46 (s, 1H), 7.39 (br s, 1H), 5.63 (br s, 4H), 3.88 (s, 3H), 3.11- 2.97 (m, 2H), 2.33-2.15 (m, 4H), 1.91 (s, 2H), 1.81-1.57 (m, 6H), 1.41-1.30 (m, 2H), 1.09 (s, 6H), 0.91 (br t, J = 7.2 Hz, 3H) |
| | 1-[4-(6-{[5-amino-7-(butylamino)-1H- | | | |
| | pyrazolo[4,3-d]pyrimidin-1-yl]methyl}-5- | | | |
| | methoxypyridin-3-yl)piperidin-1-yl]-2- | | | |
| | methylpropan-2-ol | | | |
| 147 | 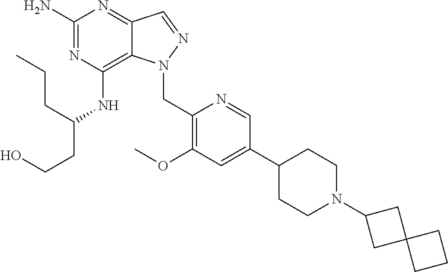 | 15.7 | 2.1 | LC/MS [M + H]⁺ = 549.0 RT (min) = 1.26 δ 7.96 (s, 1H), 7.85 (br d, J = 7.9 Hz, 1H), 7.47 (s, 1H), 7.43 (s, 1H), 5.71- 5.50 (m, 4H), 4.48-4.33 (m, 1H), 3.89 (s, 3H), 3.55-3.36 (m, 1H), 2.87 (br d, J = 10.7 Hz, 2H), 2.17- 2.05 (m, 2H), 1.98 (br t, J = 7.0 Hz, 2H), 1.88-1.54 (m, 14H), 1.42- 1.21 (m, 2H), 0.87 (t, J = 7.3 Hz, 3H) |
| | (3S)-3-[(5-amino-1-{[3-methoxy-5-(1- | | | |
| | {spiro[3.3]heptan-2-yl}piperidin-4- | | | |
| | yl)pyridin-2-yl]methyl}-1H-pyrazolo[4,3- | | | |
| | d]pyrimidin-7-yl)amino]hexan-1-ol | | | |
| 148 | 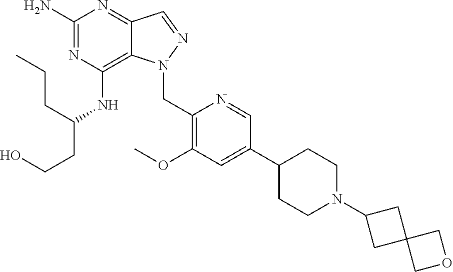 | 14.1 | 0.49 | LC/MS [M + H]⁺ = 550.9 RT (min) = 1.06 δ 7.96 (s, 1H), 7.85 (br d, J = 7.9 Hz, 1H), 7.47 (s, 1H), 7.42 (s, 1H), 5.71- 5.47 (m, 4H), 4.58 (s, 2H), 4.48- 4.44 (m, 2H), 4.43-4.38 (m, 1H), 3.88 (s, 3H), 3.54-3.38 (m, 1H), 2.87 (br d, J = 11.0 Hz, 2H), 2.37- 2.25 (m, 2H), 1.97-1.93 (m, 2H), 1.84-1.50 (m, 10H), 1.41-1.19 (m, 2H), 0.87 (t, J = 7.3 Hz, 3H) |
| | (3S)-3-[(5-amino-1-{[3-methoxy-5-(1-{2- | | | |
| | oxaspiro[3.3]heptan-6-yl}piperidin-4- | | | |
| | yl)pyridin-2-yl]methyl}-1H-pyrazolo[4,3- | | | |
| | d]pyrimidin-7-yl)amino]hexan-1-ol | | | |
| 149 | 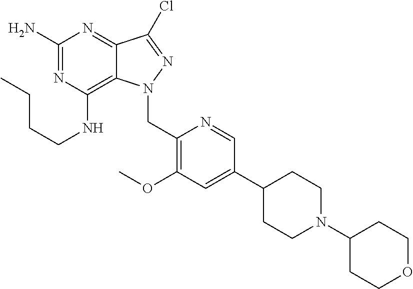 [SMILES: CCCCNc1nc(N)nc2c(Cl)nn(Cc3ncc(C4CCN(C5CCOCC5)CC4)cc3OC)c12] | 869.2 | 590.1 | LC/MS [M + H]⁺ = 529.1 RT (min) = 1.25 δ 7.97 (s, 1H), 7.96-7.86 (m, 1H), 7.43 (s, 1H), 5.91 (s, 2H), 5.62 (s, 2H), 3.96-3.82 (m, 7H), 3.29 (br t, J = 10.8 Hz, 2H), 3.02 (br d, J = 11.3 Hz, 2H), 2.50-2.40 (m, 2H), 2.22 (br t, J = 11.0 Hz, 2H), 1.81-1.64 (m, 6H), 1.61 (quin, J = 7.2 Hz, 2H), 1.51-1.39 (m, 2H), 1.39-1.27 (m, 2H), 0.91 (t, J = 7.3 Hz, 3H) |
| | N7-butyl-3-chloro-1-({3-methoxy-5-[1- | | | |
| | (oxan-4-yl)piperidin-4-yl]pyridin-2- | | | |
| | yl}methyl)-1H-pyrazolo[4,3-d]pyrimidine- | | | |
| | 5,7-diamine | | | |
| 150 | 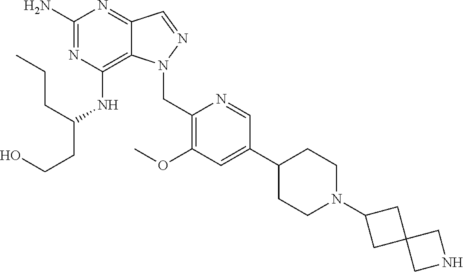 | 88.8 | 44.1 | LC/MS [M + H]⁺ = 550.7 RT (min) = 0.78 δ 7.94 (s, 1H), 7.83 (br d, J = 5.5 Hz, 1H), 7.46 (s, 1H), 7.39 (s, 1H), 5.69- 5.46 (m, 4H), 4.39 (br dd, J = 7.0, 3.8 Hz, 1H), 3.86 (s, 3H), 3.82- 3.74 (m, 2H), 3.50-3.37 (m, 2H), 2.83 (br d, J = 10.9 Hz, 2H), 2.62- 2.55 (m, 2H), 2.28 (br d, J = 8.4 Hz, 2H), 1.90 (br t, J = 8.9 Hz, 2H), 1.75- 1.51 (m, 10H), 1.38-1.16 (m, 2H), 0.85 (t, J = 7.3 Hz, 3H) |
| | (3S)-3-[(5-amino-1-{[5-(1-{2-azaspiro- | | | |
| | [3.3]heptan-6-yl}piperidin-4-yl)-3-methoxy- | | | |
| | pyridin-2-yl]methyl}-1H-pyrazolo[4,3- | | | |
| | d]pyrimidin-7-yl)amino]hexan-1-ol | | | |
| 151 | 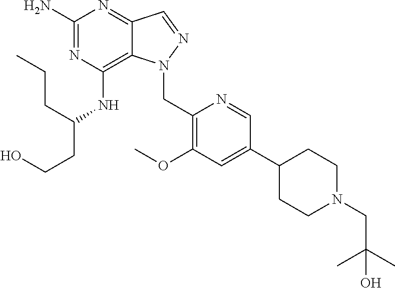 | 15.2 | 0.32 | LC/MS [M + H]⁺ = 526.9 RT (min) = 1.08 δ 7.98 (s, 1H), 7.86 (br d, J = 8.9 Hz, 1H), 7.47 (s, 1H), 7.43 (s, 1H), 5.70- 5.49 (m, 4H), 4.52-4.32 (m, 1H), 3.90 (s, 3H), 3.05 (br d, J = 11.0 Hz, 2H), 2.29-2.16 (m, 4H), 1.86- 1.65 (m, 6H), 1.64-1.51 (m, 2H), 1.32 (td, J = 14.0, 7.2 Hz, 2H), 1.11 (s, 6H), 0.89 (br t, J = 7.3 Hz, 3H) |
| | (3S)-3-{[5-amino-1-({5-[1-(2-hydroxy-2- | | | |
| | methylpropyl)piperidin-4-yl]-3-methoxy- | | | |
| | pyridin-2-yl}methyl)-1H-pyrazolo[4,3- | | | |
| | d]pyrimidin-7-yl]amino}hexan-1-ol | | | |
| 152 | 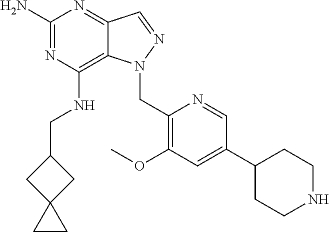 [SMILES: COc1cc(C2CCNCC2)cnc1Cn1ncc2nc(N)nc(NCC3CC4(CC4)C3)c21] | 110.7 | | LC/MS [M + H]⁺ = 449.1 RT (min) = 1.20 δ 9.24 (br t, J = 5.2 Hz, 1H), 7.95 (s, 1H), 7.73 (s, 1H), 7.41 (s, 1H), 5.80 (s, 2H), 3.91 (s, 3H), 3.80-3.68 (m, 2H), 3.52-3.30 (m, 1H), 3.08- 2.86 (m, 3H), 2.85-2.71 (m, 1H), 2.18-2.03 (m, 2H), 2.00-1.78 (m, 6H), 0.40 (s, 4H) |
| | 1-{[3-methoxy-5-(piperidin-4-yl)pyridin- | | | |
| | 2-yl]methyl}-N7-({spiro[2.3]hexan-5- | | | |
| | yl}methyl)-1H-pyrazolo[4,3-d]pyrimidine- | | | |
| | 5,7-diamine | | | |
| 153 | 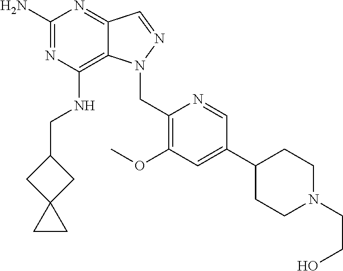 [SMILES: COc1cc(C2CCN(CCO)CC2)cnc1Cn1ncc2nc(N)nc(NCC3CC4(CC4)C3)c21] | 86.8 | | LC/MS [M + H]⁺ = 493.2 RT (min) = 1.18 δ 8.00 (br d, J = 2.9 Hz, 1H), 7.96 (s, 1H), 7.46 (s, 1H), 7.38 (s, 1H), 5.68- 5.54 (m, 4H), 3.87 (s, 3H), 3.58 (br s, 2H), 2.97 (br d, J = 11.5 Hz, 2H), 2.82-2.70 (m, 1H), 2.43 (br t, J = 6.2 Hz, 2H), 2.16-2.02 (m, 4H), 1.87 (br dd, J = 11.8, 6.1 Hz, 2H), 1.70 (br dd, J = 7.8, 3.0 Hz, 4H), 0.38 (s, 4H) |
| | 2-{4-[6-({5-amino-7-[({spiro[2.3]hexan-5- | | | |
| | yl}methyl)amino]-1H-pyrazolo[4,3-d]pyri- | | | |
| | midin-1-yl}methyl)-5-methoxypyridin-3- | | | |
| | yl]piperidin-1-yl}ethan-1-ol | | | |
| 154 | 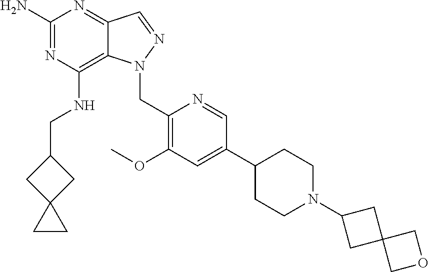 [SMILES: COc1cc(C2CCN(C3CC4(COC4)C3)CC2)cnc1Cn1ncc2nc(N)nc(NCC3CC4(CC4)C3)c21] | 34.0 | 7.1 | LC/MS [M + H]⁺ = 545.3 RT (min) = 1.34 δ 8.01 (br d, J = 3.8 Hz, 1H), 7.95 (s, 1H), 7.46 (s, 1H), 7.38 (br s, 1H), 5.62 (s, 4H), 4.57 (s, 2H), 4.44 (s, 2H), 3.86 (s, 3H), 2.85 (br d, J = 10.9 Hz, 2H), 2.80-2.69 (m, 1H), 2.32 (br t, J = 9.7 Hz, 2H), 2.11 (br dd, J = 10.2, 9.1 Hz, 2H), 1.96-1.91 (m, 2H), 1.87 (br dd, J = 11.1, 6.0 Hz, 2H), 1.81-1.57 (m, 6H), 0.38 (s, 4H) |
| | 1-{[3-methoxy-5-(1-{2-oxaspiro- | | | |
| | [3.3]heptan-6-yl}piperidin-4-yl)pyridin-2- | | | |
| | yl]methyl}-N7-({spiro[2.3]hexan-5- | | | |
| | yl}methyl)-1H-pyrazolo[4,3-d]pyrimidine- | | | |
| | 5,7-diamine | | | |
| 155 | 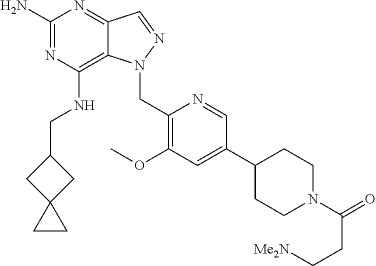 [SMILES: COc1cc(C2CCN(C(=O)CCN(C)C)CC2)cnc1Cn1ncc2nc(N)nc(NCC3CC4(CC4)C3)c21] | 199.4 | | LC/MS [M + H]⁺ = 548.2 RT (min) = 1.38 δ 7.98 (s, 2H), 7.46 (s, 1H), 7.42 (s, 1H), 5.66 (br s, 2H), 5.64 (s, 2H), 4.55 (br d, J = 12.6 Hz, 1H), 4.00 (br d, J = 12.9 Hz, 1H), 3.88 (s, 3H), 3.62 (dd, J = 7.2, 5.5 Hz, 2H), 3.10 (br t, J = 12.6 Hz, 1H), 2.91-2.82 (m, 1H), 2.81-2.68 (m, 3H), 2.62-2.58 (m, 2H), 2.34 (s, 6H), 2.18-2.09 (m, 2H), 1.89 (br dd, J = 12.2, 6.1 Hz, 2H), 1.84-1.72 (m, 2H), 1.70- 1.58 (m, 2H), 1.58-1.47 (m, 1H), 0.40 (s, 4H) |
| | 1-{4-[6-({5-amino-7-[({spiro[2.3]hexan-5- | | | |
| | yl}methyl)amino]-1H-pyrazolo[4,3- | | | |
| | d]pyrimidin-1-yl}methyl)-5- | | | |
| | methoxypyridin-3-yl]piperidin-1-yl}-3- | | | |
| | (dimethylamino)propan-1-one | | | |
| 156 | 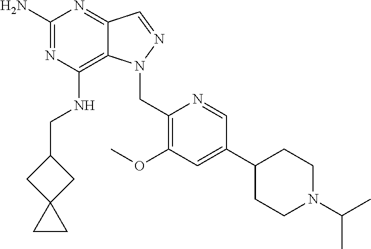 [SMILES: COc1cc(C2CCN(C(C)C)CC2)cnc1Cn1ncc2nc(N)nc(NCC3CC4(CC4)C3)c21] | 40.9 | | LC/MS [M + H]⁺ = 491.0 RT (min) = 1.34 δ 9.24 (br s, 1H), 7.95 (s, 1H), 7.74 (s, 1H), 7.41 (s, 1H), 5.81 (s, 2H), 3.92 (s, 3H), 3.75 (br d, J = 6.1 Hz, 2H), 3.15-3.02 (m, 2H), 3.02- 2.89 (m, 1H), 2.86-2.73 (m, 1H), 2.17-2.08 (m, 2H), 2.08-1.93 (m, 4H), 1.88 (br dd, J = 11.3, 5.8 Hz, 2H), 1.29 (br d, J = 6.4 Hz, 6H), 0.40 (s, 4H) |
| | 1-({3-methoxy-5-[1-(propan-2- | | | |
| | yl)piperidin-4-yl]pyridin-2-yl}methyl)-N7- | | | |
| | ({spiro[2.3]hexan-5-yl}methyl)-1H- | | | |
| | pyrazolo[4,3-d]pyrimidine-5,7-diamine | | | |
| 157 | 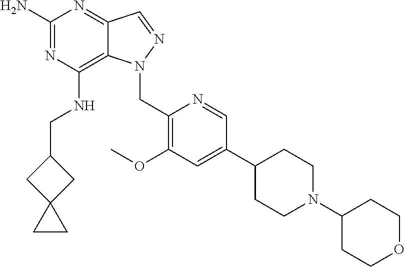 [SMILES: COc1cc(C2CCN(C3CCOCC3)CC2)cnc1Cn1ncc2nc(N)nc(NCC3CC4(CC4)C3)c21] | 50.8 | 37.0 | LC/MS [M + H]⁺ = 533.2 RT (min) = 1.27 δ 8.03 (br s, 1H), 7.95 (s, 1H), 7.46 (s, 1H), 7.37 (s, 1H), 3.93-3.79 (m, 5H), 3.68-3.65 (m, 2H), 3.26 (br t, J = 11.3 Hz, 2H), 3.05-2.94 (m, 2H), 2.81-2.70 (m, 1H), 2.49-2.39 (m, 1H), 2.20 (br t, J = 10.6 Hz, 2H), 2.15- 2.06 (m, 2H), 1.86 (br dd, J = 11.9, 6.3 Hz, 2H), 1.79-1.58 (m, 6H), 1.49-1.36 (m, 2H), 0.37 (s, 4H) |
| | 1-({3-methoxy-5-[1-(oxan-4-yl)piperidin- | | | |
| | 4-yl]pyridin-2-yl}methyl)-N7- | | | |
| | ({spiro[2.3]hexan-5-yl}methyl)-1H- | | | |
| | pyrazolo[4,3-d]pyrimidine-5,7-diamine | | | |
| 158 | 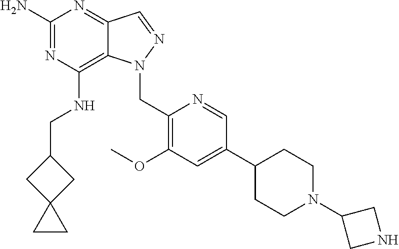 [SMILES: COc1cc(C2CCN(C3CNC3)CC2)cnc1Cn1ncc2nc(N)nc(NCC3CC4(CC4)C3)c21] | 223.6 | 69.4 | LC/MS [M + H]⁺ = 504.2 RT (min) = 1.15 δ 8.04 (br s, 1H), 7.97 (s, 1H), 7.49 (s, 1H), 7.39 (br s, 1H), 5.65 (s, 2H), 3.78 (br s, 3H), 3.66-3.53 (m, 2H), 2.83 (br d, J = 10.7 Hz, 2H), 2.80- 2.68 (m, 2H), 2.13 (br t, J = 9.9 Hz, 2H), 1.96-1.83 (m, 4H), 1.82- 1.74 (m, 6H), 1.72-1.63 (m, 2H), 0.39 (s, 4H). |
| | 1-({5-[1-(azetidin-3-yl)piperidin-4-yl]-3- | | | |
| | methoxypyridin-2-yl}methyl)-N7- | | | |
| | ({spiro[2.3]hexan-5-yl}methyl)-1H- | | | |
| | pyrazolo[4,3-d]pyrimidine-5,7-diamine | | | |
| 159 | 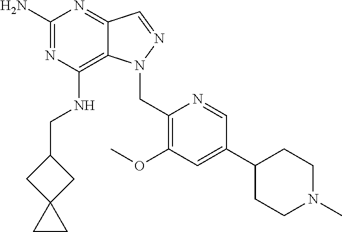 [SMILES: COc1cc(C2CCN(C)CC2)cnc1Cn1ncc2nc(N)nc(NCC3CC4(CC4)C3)c21] | 62.0 | 4.1 | LC/MS [M + H]⁺ = 463.2 RT (min) = 1.24 δ 8.03-7.90 (m, 2H), 7.46 (s, 1H), 7.39 (s, 1H), 5.73-5.49 (m, 4H), 3.87 (s, 3H), 3.61 (br dd, J = 7.0, 5.4 Hz, 2H), 2.93-2.82 (m, 2H), 2.81- 2.73 (m, 1H), 2.21 (s, 3H), 2.16- 2.08 (m, 2H), 2.04-1.95 (m, 2H), 1.88 (br dd, J = 11.8, 6.0 Hz, 2H), 1.78-1.65 (m, 4H), 0.38 (s, 4H). |
| | 1-{[3-methoxy-5-(1-methylpiperidin-4- | | | |
| | yl)pyridin-2-yl]methyl}-N7- | | | |
| | ({spiro[2.3]hexan-5-yl}methyl)-1H- | | | |
| | pyrazolo[4,3-d]pyrimidine-5,7-diamine | | | |
| 160 | 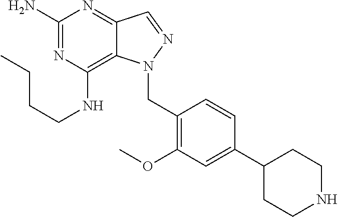 [SMILES: CCCCNc1nc(N)nc2cnn(Cc3ccc(C4CCNCC4)cc3OC)c12] | 13.0 | — | LC/MS [M + H]⁺ = 410.0 RT (min) = 1.10 δ 6.89 (s, 1H), 6.69 (br d, J = 7.9 Hz, 1H), 6.45 (br d, J = 7.6 Hz, 1H), 6.39 (br t, J = 5.2 Hz, 1H), 5.61 (s, 2H), 5.58 (s, 2H), 3.84 (s, 2H), 3.13- 2.92 (m, 2H), 2.60 (br d, J = 11.3 Hz, 2H), 1.68 (br d, J = 12.8 Hz, 2H), 1.61-1.48 (m, 2H), 1.49-1.39 (m, 2H), 1.24-1.09 (m, 2H), 0.84 (t, J = 7.3 Hz, 3H) |
| | N7-butyl-1-{[2-methoxy-4-(piperidin-4- | | | |
| | yl)phenyl]methyl}-1H-pyrazolo[4,3- | | | |
| | d]pyrimidine-5,7-diamine | | | |
| 161 | 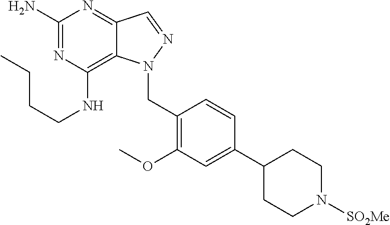 [SMILES: CCCCNc1nc(N)nc2cnn(Cc3ccc(C4CCN(S(C)(=O)=O)CC4)cc3OC)c12] | 107.4 | 36 | LC/MS [M + H]⁺ = 488.1 RT (min) = 1.55 δ 7.55 (s, 1H), 6.95 (s, 1H), 6.73 (d, J = 7.7 Hz, 1H), 6.43 (br d, J = 7.8 Hz, 2H), 5.67 (s, 2H), 5.59 (s, 2H), 3.85 (s, 3H), 3.66 (br d, J = 12.2 Hz, 2H), 2.89 (s, 3H), 2.78 (br t, J = 11.1 Hz, 2H), 2.64-2.57 (m, 1H), 1.83 (br d, J = 12.5 Hz, 2H), 1.75-1.62 (m, 2H), 1.47 (quin, J = 7.2 Hz, 2H), 1.24- 1.13 (m, 2H), 0.84 (t, J = 7.4 Hz, 3H) |
| | N7-butyl-1-{[4-(1-methanesulfonyl- | | | |
| | piperidin-4-yl)-2-methoxyphenyl]- | | | |
| | methyl}-1H-pyrazolo[4,3-d]pyrimidine- | | | |
| | 5,7-diamine | | | |
| 162 | 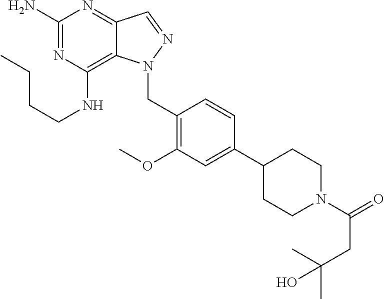 [SMILES: CCCCNc1nc(N)nc2cnn(Cc3ccc(C4CCN(C(=O)CC(C)(C)O)CC4)cc3OC)c12] | 12.2 | — | LC/MS [M + H]⁺ = 510.1 RT (min) = 1.43 δ 7.56 (s, 1H), 6.89 (s, 1H), 6.70 (br d, J = 8.2 Hz, 1H), 6.57-6.40 (m, 2H), 5.73 (br s, 2H), 5.58 (s, 2H), 4.57 (br d, J = 13.4 Hz, 1H), 4.13- 4.00 (m, 1H), 3.82 (s, 3H), 3.63 (br d, J = 2.7 Hz, 2H), 3.46-3.32 (m, 2H), 3.06 (br t, J = 13.0 Hz, 1H), 2.82- 2.69 (m, 1H), 2.47 (br d, J = 4.0 Hz, 2H), 1.75 (br d, J = 13.1 Hz, 2H), 1.54 (br s, 1H), 1.50-1.40 (m, 3H), 1.22-1.11 (m, 8H), 0.82 (t, J = 7.3 Hz, 3H) |
| | 1-[4-(4-{[5-amino-7-(butylamino)-1H- | | | |
| | pyrazolo[4,3-d]pyrimidin-1-yl]methyl}-3- | | | |
| | methoxyphenyl)piperidin-1-yl]-3- | | | |
| | hydroxy-3-methylbutan-1-one | | | |
| 163 | 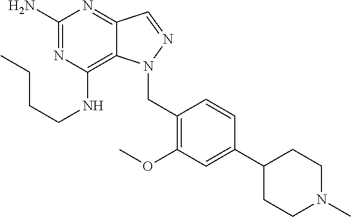 [SMILES: CCCCNc1nc(N)nc2cnn(Cc3ccc(C4CCN(C)CC4)cc3OC)c12] | 5.0 | 0.065 | LC/MS [M + H]⁺ = 424.2 RT (min) = 1.13 δ 7.54 (s, 1H), 6.91 (s, 1H), 6.70 (d, J = 7.1 Hz, 1H), 6.56-6.21 (m, 2H), 5.64 (s, 2H), 5.58 (s, 2H), 3.84 (s, 3H), 2.85 (br d, J = 10.9 Hz, 2H), 2.42 (br t, J = 4.5 Hz, 1H), 2.19 (s, 3H), 2.00-1.93 (m, 2H), 1.74- 1.56 (m, 4H), 1.46 (quin, J = 7.2 Hz, 2H), 1.24-1.08 (m, 2H), 0.83 (t, J = 7.3 Hz, 3H) |
| | N7-butyl-1-{[2-methoxy-4-(1- | | | |
| | methylpiperidin-4-yl)phenyl]methyl}-1H- | | | |
| | pyrazolo[4,3-d]pyrimidine-5,7-diamine | | | |
| 164 | 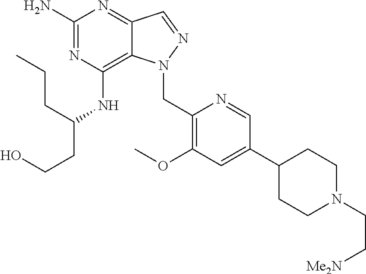 | 41.0 | 3.9 | LC/MS [M + H]⁺ = 526.3 RT (min) = 1.10 δ 7.99 (s, 1H), 7.83 (br d, J = 7.9 Hz, 1H), 7.52-7.40 (m, 2H), 5.75- 5.49 (m, 4H), 4.43 (br d, J = 7.9 Hz, 1H), 3.91 (s, 3H), 3.00 (br d, J = 11.3 Hz, 2H), 2.41 (br dd, J = 10.2, 4.4 Hz, 4H), 2.18 (s, 6H), 2.09-1.99 (m, 2H), 1.88-1.76 (m, 2H), 1.74 (br s, 4H), 1.65-1.54 (m, 2H), 1.42- 1.26 (m, 2H), 0.89 (t, J = 7.3 Hz, 3H) |
| | (3S)-3-({5-amino-1-[(5-{1-[2-(dimethyl- | | | |
| | amino)ethyl]piperidin-4-yl}-3-methoxy- | | | |
| | pyridin-2-yl)methyl]-1H-pyrazolo[4,3- | | | |
| | d]pyrimidin-7-yl}amino)hexan-1-ol | | | |
| 165 | 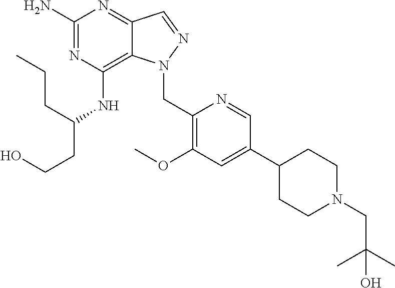 | 168.5 | 140.7 | LC/MS [M + H]⁺ = 483.3 RT (min) = 1.28 δ 7.64 (s, 1H), 7.47 (d, J = 8.5 Hz, 1H), 7.22 (d, J = 8.5 Hz, 1H), 5.92- 5.71 (m, 2H), 4.49-4.25 (m, 1H), 3.85 (s, 3H), 2.81-2.69 (m, 2H), 1.78-1.63 (m, 6H), 1.57-1.41 (m, 2H), 1.29-1.12 (m, 6H), 1.15- 1.03 (m, 2H), 0.79 (br t, J = 7.2 Hz, 3H) |
| | (3S)-3-{[5-amino-1-({6-[1-(2-hydroxy-2- | | | |
| | methylpropyl)piperidin-4-yl]-3-methoxy- | | | |
| | pyridin-2-yl}methyl)-1H-pyrazolo[4,3- | | | |
| | d]pyrimidin-7-yl]amino}hexan-1-ol | | | |
| 166 | 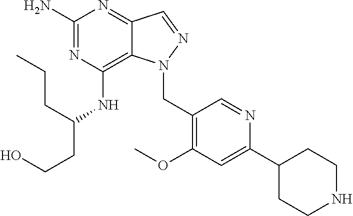 | 45.5 | 22.5 | LC/MS [M + H]⁺ = 455.3 RT (min) = 0.98 ¹H NMR (400 MHz, DMSO-d6) δ = 7.55 (s, 1H), 7.50 (s, 1H), 6.91 (s, 1H), 6.04 (d, J = 8.3 Hz, 1H), 5.69- 5.60 (m, 3H), 5.58-5.50 (m, 1H), 4.48-4.31 (m, 2H), 3.86 (s, 3H), 3.17 (s, 2H), 3.05-3.00 (m,2H), 2.74-2.67 (m, 1H), 2.63-2.54 (m, 2H), 1.89 (s, 2H), 1.74-1.54 (m, 6H), 1.49-1.40 (m, 2H), 1.15- 1.02 (m, 2H), 0.78 (t, J = 7.3 Hz, 3H) |
| | (3S)-3-[(5-amino-1-{[4-methoxy-6- | | | |
| | (piperidin-4-yl)pyridin-3-yl]methyl}-1H- | | | |
| | pyrazolo[4,3-d]pyrimidin-7- | | | |
| | yl)amino]hexan-1-ol | | | |
| 167 | 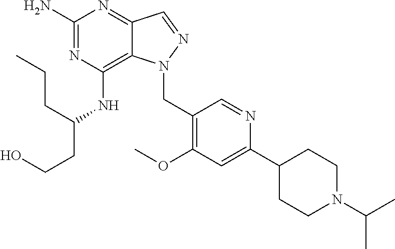 | 9.4 | 0.17 | LC/MS [M + H]⁺ = 497.4 RT (min) = 1.00 ¹H NMR (400 MHz, DMSO-d6) δ = 12.55-12.19 (m, 1H), 9.11-8.96 (m, 1H), 7.99-7.94 (m, 1H), 7.91- 7.81 (m, 2H), 7.78-7.74 (m, 1H), 7.24-7.21 (m, 1H), 7.11-7.08 (m, 1H), 6.99-6.94 (m, 1H), 5.81- 5.63 (m, 2H), 4.62-4.55 (m, 1H), 3.84-3.79 (m, 3H), 3.47 (br d, J = 6.6 Hz, 6H), 3.17 (s, 1H), 3.13- 2.94 (m, 2H), 2.11-1.96 (m, 3H), 1.83-1.73 (m, 2H), 1.67-1.50 (m, 2H), 1.31-1.16 (m, 8H), 0.88- 0.81 (m, 3H) |
| | (3S)-3-{[5-amino-1-({4-methoxy-6-[1-(propan- | | | |
| | 2-yl)piperidin-4-yl]pyridin-3-yl}methyl)-1H- | | | |
| | pyrazolo[4,3-d]pyrimidin-7-yl]amino}hexan-1- | | | |
| | ol | | | |
| 168 | 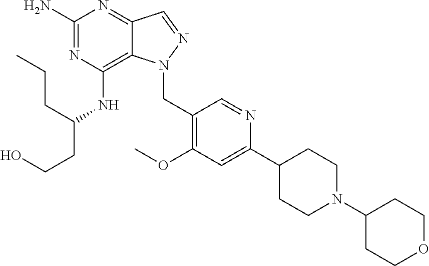 | 6.8 | 0.47 | LC/MS [M + H]⁺= 539.4 RT (min) = 1.07 ¹H NMR (400 MHz, DMSO-d6) δ = 7.55 (s, 1H), 7.49 (s, 1H), 6.93 (s, 1H), 6.05 (d, J = 8.6 Hz, 1H), 5.71- 5.59 (m, 3H), 5.59-5.47 (m, 1H), 4.45 (br s, 1H), 4.41-4.29 (m, 1H), 3.91-3.84 (m, 5H), 3.27 (br s, 4H), 2.97 (br d, J = 12.0 Hz, 2H), 2.61- 2.53 (m, 1H), 2.45-2.38 (m, 1H), 2.24-2.12 (m, 2H), 1.91 (s, 4H), 1.79-1.56 (m, 8H), 1.49-1.38 (m, 4H), 1.16-1.05 (m, 2H), 0.78 (t, J = 7.3 Hz, 3H) |
| | (3S)-3-{[5-amino-1-({4-methoxy-6-[1-(oxan-4- | | | |
| | yl)piperidin-4-yl]pyridin-3-yl}methyl)-1H- | | | |
| | pyrazolo[4,3-d]pyrimidin-7-yl]amino}hexan-1- | | | |
| | ol | | | |
| 169 | 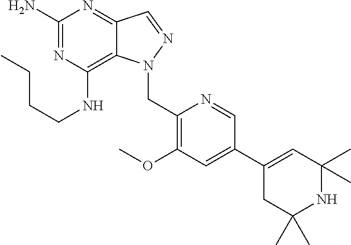 [SMILES: CCCCNc1nc(N)nc2cnn(Cc3ncc(C4=CC(C)(C)NC(C)(C)C4)cc3OC)c12] | 495.6 | 120.6 | LC/MS [M + H]⁺ = 465.3 RT (min) = 1.27 δ 8.12 (s, 1H), 7.76 (br s, 1H), 7.47 (s, 2H), 6.26 (s, 1H), 5.68 (s, 2H), 5.59 (s, 2H), 3.93 (s, 3H), 3.56- 3.30 (m, 2H), 2.31 (s, 2H), 1.67- 1.57 (m, 2H), 1.37-1.31 (m, 2H), 1.28 (s, 6H), 1.21 (s, 6H), 0.91 (t, J = 7.5 Hz, 3H) |
| | N7-butyl-1-({5-methoxy-2′,2′,6′,6′- | | | |
| | tetramethyl-1′,2′,3′,6′-tetrahydro-[3,4′- | | | |
| | bipyridine]-6-yl}methyl)-1H-pyrazolo[4,3- | | | |
| | d]pyrimidine-5,7-diamine | | | |
| 170 | 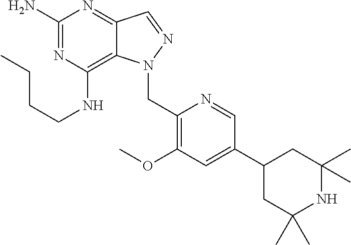 [SMILES: CCCCNc1nc(N)nc2cnn(Cc3ncc(C4CC(C)(C)NC(C)(C)C4)cc3OC)c12] | 208.6 | 42.6 | LC/MS [M + H]⁺ = 467.0 RT (min) = 1.22 δ 8.02 (s, 2H), 7.47 (s, 1H), 7.43 (s, 1H), 5.65 (s, 2H), 5.60 (s, 2H), 3.90 (s, 3H), 3.29-2.96 (m, 1H), 1.70- 1.55 (m, 4H), 1.50-1.31 (m, 4H), 1.27 (s, 6H), 1.13 (s, 6H), 0.94 (t, J = 7.3 Hz, 3H). |
| | N7-butyl-1-{[3-methoxy-5-(2,2,6,6- | | | |
| | tetramethylpiperidin-4-yl)pyridin-2- | | | |
| | yl]methyl}-1H-pyrazolo[4,3-d]pyrimidine- | | | |
| | 5,7-diamine | | | |
| 171 | 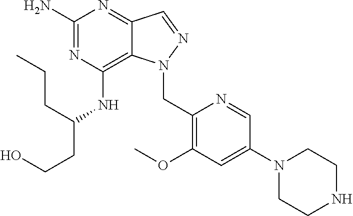 | 308.6 | 55.5 | LC/MS [M + H]⁺ = 456.2 RT (min) = 1.07 δ 9.49-9.34 (m, 1H), 7.79 (br d, J = 2.1 Hz, 1H), 7.75-7.56 (m, 2H), 7.13 (d, J = 1.9 Hz, 1H), 5.66 (br d, J = 2.7 Hz, 2H), 4.65-4.50 (m, 1H), 3.89 (s, 3H), 3.56-3.41 (m, 2H), 3.23 (br s, 2H), 1.92-1.74 (m, 2H), 1.67 (q, J = 7.2 Hz, 2H), 1.40-1.25 (m, 2H), 0.90 (br t, J = 7.3 Hz, 3H) |
| | (3S)-3-[(5-amino-1-{[3-methoxy-5- | | | |
| | (piperazin-1-yl)pyridin-2-yl]methyl}-1H- | | | |
| | pyrazolo[4,3-d]pyrimidin-7- | | | |
| | yl)amino]hexan-1-ol | | | |
| 172 | 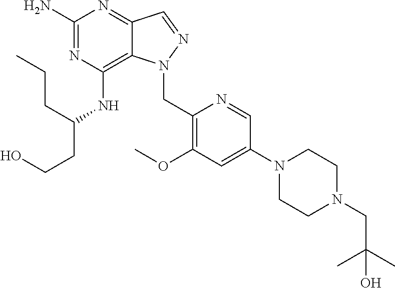 | 19.9 | 10.8 | LC/MS [M + H]⁺ = 527.9 RT (min) = 1.08 δ 8.03 (br d, J = 6.0 Hz, 1H), 7.97 (d, J = 1.2 Hz, 1H), 7.50 (s, 1H), 7.43 (s, 1H), 5.89 (br s, 1H), 5.72-5.53 (m, 2H), 4.50-4.38 (m, 1H), 3.90 (s, 3H), 3.58-3.38 (m, 1H), 2.44- 2.25 (m, 2H), 1.87-1.65 (m, 6H), 1.64-1.51 (m, 2H), 1.31 (td, J = 14.1, 7.0 Hz, 2H), 1.13 (s, 6H), 0.88 (t, J = 7.3 Hz, 3H) |
| | (3S)-3-{[5-amino-1-({5-[4-(2-hydroxy-2- | | | |
| | methylpropyl)piperazin-1-yl]-3-methoxy- | | | |
| | pyridin-2-yl}methyl)-1H-pyrazolo[4,3- | | | |
| | d]pyrimidin-7-yl]amino}hexan-1-ol | | | |
| 173 | 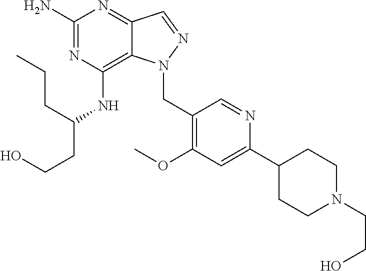 | 89.9 | 4.8 | LC/MS [M + H]⁺ = 499.4 RT (min) = 0.81 ¹H NMR (400 MHz, DMSO-d6) δ = 7.77 (s, 1H), 7.69 (s, 1H), 6.98 (s, 3H), 5.76-5.60 (m, 2H), 5.28 (br d, J = 1.7 Hz, 1H), 4.57-4.44 (m, 2H), 3.83 (s, 3H), 3.78-3.71 (m, 2H), 3.62-3.50 (m, 2H), 3.45-3.41 (m, 2H), 3.19-3.15 (m, 2H), 3.10- 2.88 (m, 4H), 2.01 (br s, 4H), 1.79- 1.68 (m, 2H), 1.61-1.49 (m, 2H), 1.27-1.10 (m, 3H), 0.83 (t, J = 7.3 Hz, 3H) |
| | (3S)-3-{[5-amino-1-({6-[1-(2-hydroxy- | | | |
| | ethyl)piperidin-4-yl]-4-methoxypyridin-3- | | | |
| | yl}methyl)-1H-pyrazolo[4,3-d]pyrimidin- | | | |
| | 7-yl]amino}hexan-1-ol | | | |
| 174 | 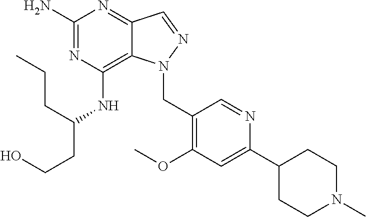 | 11.0 | 2.9 | LC/MS [M + H]⁺ = 469.3 RT (min) = 1.05 ¹H NMR (400 MHz, DMSO-d₆) δ = 7.56 (s, 1H), 7.50 (s, 1H), 6.94 (s, 1H), 6.06 (d, J = 8.3 Hz, 1H), 5.70- 5.62 (m, 3H), 5.59-5.51 (m, 1H), 4.49-4.43 (m, 1H), 4.41-4.33 (m, 1H), 3.86 (s, 3H), 3.41-3.38 (m, 2H), 2.90-2.81 (m, 2H), 2.59- 2.54 (m, 1H), 2.01-1.88 (m, 3H), 1.78-1.58 (m, 6H), 1.48-1.39 (m, 2H), 1.16-1.03 (m, 2H), 0.79 (t, J = 7.4 Hz, 3H) |
| | (3S)-3-[(5-amino-1-{[4-methoxy-6-(1- | | | |
| | methylpiperidin-4-yl)pyridin-3- | | | |
| | yl]methyl}-1H-pyrazolo[4,3-d]pyrimidin- | | | |
| | 7-yl)amino]hexan-1-ol | | | |
| 175 | 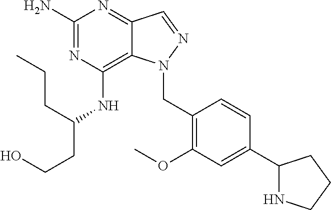 | 8.8 | 0.91 | LC/MS [M + H]⁺ = 440.24 RT (min) = 1.12 δ 7.59-7.54 (m, 1H), 7.13-7.07 (m, 1H), 6.89-6.79 (m, 1H), 6.45- 6.37 (m, 1H), 5.72-5.65 (m, 1H), 5.65-5.61 (m, 2H), 5.59-5.52 (m, 1H), 4.37-4.27 (m, 1H), 4.16- 4.05 (m, 1H), 3.89-3.83 (m, 3H), 3.33-3.26 (m, 1H), 3.12-3.03 (m, 1H), 3.02-2.90 (m, 1H), 2.55- 2.54 (m, 3H), 2.20-2.06 (m, 1H), 1.87-1.73 (m, 2H), 1.68-1.32 (m, 6H), 1.16-1.00 (m, 2H), 0.82- 0.73 (m, 3H) |
| | (3S)-3-[(5-amino-1-{[2-methoxy-4- | | | |
| | (pyrrolidin-2-yl)phenyl]methyl}-1H- | | | |
| | pyrazolo[4,3-d]pyrimidin-7- | | | |
| | yl)amino]hexan-1-ol | | | |
| 176 | 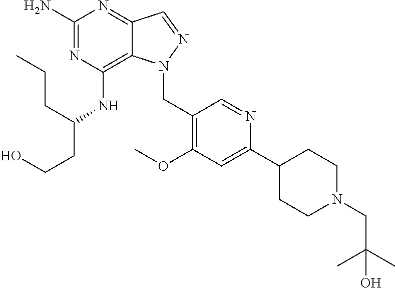 | 4.8 | 0.21 | LC/MS [M + H]⁺ = 527.4 RT (min) = 1.05 ¹H NMR (400 MHz, DMSO-d₆) δ = 7.55 (s, 1H), 7.52 (s, 1H), 6.92 (s, 1H), 6.04 (d, J = 8.3 Hz, 1H), 5.68- 5.51 (m, 4H), 4.47-4.33 (m, 2H), 3.86 (s, 3H), 3.38 (br d, J = 1.5 Hz, 2H), 3.02 (br d, J = 11.5 Hz, 2H), 2.54 (s, 1H), 2.25-2.16 (m, 4H), 1.91 (s, 2H), 1.82-1.58 (m, 6H), 1.48-1.42 (m, 2H), 1.15-1.05 (m, 9H), 0.79 (t, J = 7.3 Hz, 3H) |
| | (3S)-3-{[5-amino-1-({6-[1-(2-hydroxy-2- | | | |
| | methylpropyl)piperidin-4-yl]-4-methoxy- | | | |
| | pyridin-3-yl}methyl)-1H-pyrazolo[4,3- | | | |
| | d]pyrimidin-7-yl]amino}hexan-1-ol | | | |
| 177 | 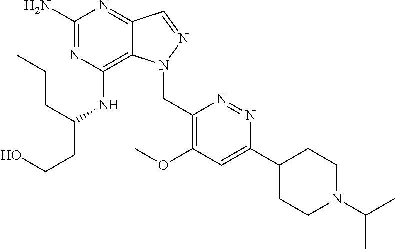 | 578.7 | 9.9 | LC/MS [M + H]⁺ = 498.4 RT (min) = 0.92 ¹H NMR (400 MHz, DMSO-d6) δ = 7.49 (s, 1H), 7.32 (d, J = 8.3 Hz, 1H), 7.25 (s, 1H), 5.81 (d, J = 3.4 Hz, 2H), 5.57 (s, 2H), 4.43-4.30 (m, 2H), 3.93 (s, 3H), 3.43 (br s, 2H), 2.95-2.88 (m, 2H), 2.83- 2.69 (m, 2H), 2.30-2.18 (m, 2H), 1.91 (s, 2H), 1.87-1.67 (m, 6H), 1.60-1.48 (m, 2H), 1.32-1.17 (m, 2H), 0.99 (d, J = 6.6 Hz, 6H), 0.84 (t, J = 7.3 Hz, 3H) |
| | (3S)-3-{[5-amino-1-({4-methoxy-6-[1- | | | |
| | (propan-2-yl)piperidin-4-yl]pyridazin-3- | | | |
| | yl}methyl)-1H-pyrazolo[4,3-d]pyrimidin- | | | |
| | 7-yl]amino}hexan-1-ol | | | |
| 178 | 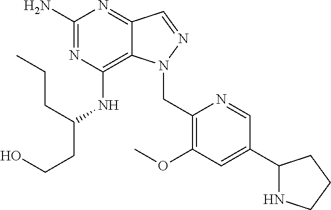 | 278.9 | 2.0 | LC/MS [M + H]⁺ = 441.4 RT (min) = 1.03/B δ 9.82-9.67 (m, 1H), 9.14-8.97 (m, 1H), 8.89 (br t, J = 7.6 Hz, 1H), 8.10 (s, 1H), 7.87 (br s, 2H), 7.73 (s, 2H), 5.84 (s, 2H), 4.63 (br d, J = 1.9 Hz, 1H), 4.57 (br dd, J = 13.3, 6.2 Hz, 1H), 3.93 (s, 3H), 3.49-3.45 (m, 2H), 2.41-2.34 (m, 1H), 2.17- 2.01 (m, 3H), 1.80 (br d, J = 5.8 Hz, 2H), 1.65-1.58 (m, 2H), 1.36- 1.23 (m, 2H), 0.92-0.84 (m, 3H) |
| | (3S)-3-[(5-amino-1-{[3-methoxy-5- | | | |
| | (pyrrolidin-2-yl)pyridin-2-yl]methyl}-1H- | | | |
| | pyrazolo[4,3-d]pyrimidin-7- | | | |
| | yl)amino]hexan-1-ol | | | |
| 179 | 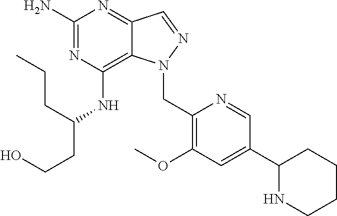 | 234.9 | 2.8 | LC/MS [M + H]⁺ = 455.3 RT (min) = 1.08/B δ 8.04 (d, J = 1.1 Hz, 1H), 7.89 (br t, J = 9.3 Hz, 1H), 7.51 (s, 1H), 7.45 (s, 1H), 5.66-5.57 (m, 2H), 5.56 (s, 2H), 4.45-4.36 (m, 1H), 3.87 (s, 3H), 3.65-3.60 (m, 1H), 3.07- 3.01 (m, 1H), 2.68-2.60 (m, 1H), 1.82-1.75 (m, 2H), 1.74-1.67 (m, 2H), 1.62-1.52 (m, 3H), 1.46- 1.26 (m, 5H), 0.88 (t, J = 7.3 Hz, 3H) |
| | (3S)-3-[(5-amino-1-{[3-methoxy-5- | | | |
| | (piperidin-2-yl)pyridin-2-yl]methyl}-1H- | | | |
| | pyrazolo[4,3-d]pyrimidin-7- | | | |
| | yl)amino]hexan-1-ol | | | |
| 180 | 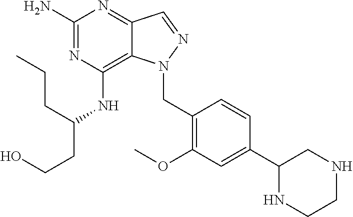 | 67.9 | 7.2 | LC/MS [M + H]⁺ = 455.36 RT (min) = 1.02 δ 7.61-7.52 (m, 1H), 7.16-7.05 (m, 1H), 6.91-6.76 (m, 1H), 6.49- 6.37 (m, 1H), 5.75-5.50 (m, 5H), 4.38-4.25 (m, 1H), 4.15-4.06 (m, 1H), 3.90-3.79 (m, 3H), 3.34- 3.23 (m, 1H), 3.13-3.01 (m, 1H), 3.01-2.91 (m, 1H), 2.21-2.05 (m, 1H), 1.93-1.88 (m, 1H), 1.87- 1.72 (m, 2H), 1.70-1.29 (m, 5H), 1.15-0.99 (m, 2H), 0.84-0.70 (m, 3H) |
| | (3S)-3-[(5-amino-1-{[2-methoxy-4- | | | |
| | (piperazin-2-yl)phenyl]methyl}-1H- | | | |
| | pyrazolo[4,3-d]pyrimidin-7- | | | |
| | yl)amino]hexan-1-ol | | | |
| 181 | 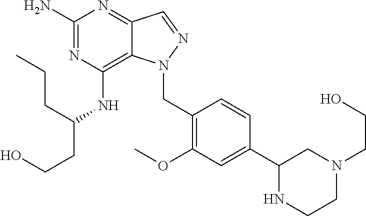 | 139.5 | 4.57 | LC/MS [M + H]⁺ = 499.0 RT (min) = 1.13 δ 7.82-7.77 (m, 1H), 7.76-7.70 (m, 1H), 7.28-7.21 (m, 1H), 7.06- 6.97 (m, 1H), 6.90-6.79 (m, 1H), 5.86-5.68 (m, 2H), 4.64-4.50 (m, 1H), 4.50-4.37 (m, 1H), 3.91- 3.73 (m, 3H), 3.70-3.61 (m, 1H), 3.46-3.22 (m, 2H), 3.07-2.75 (m, 3H), 1.83-1.68 (m, 2H), 1.66- 1.49 (m, 2H), 1.29-1.12 (m, 2H), 0.92-0.80 (m, 3H) |
| | (3S)-3-{[5-amino-1-({4-[4-(2- | | | |
| | hydroxyethyl)piperazin-2-yl]-2- | | | |
| | methoxyphenyl}methyl)-1H- | | | |
| | pyrazolo[4,3-d]pyrimidin-7- | | | |
| | yl]amino}hexan-1-ol | | | |
| 182 | 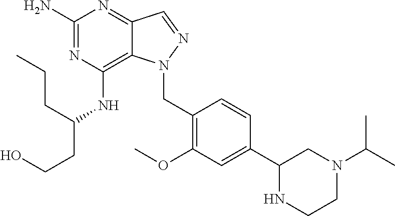 | 6.8 | 0.28 | LC/MS [M + H]⁺ = 497.2 RT (min) = 1.24 δ 7.60-7.54 (m, 1H), 7.14-7.05 (m, 1H), 6.90-6.82 (m, 1H), 6.47- 6.37 (m, 1H), 5.72-5.49 (m, 4H), 4.38-4.26 (m, 1H), 3.89-3.82 (m, 2H), 3.36-3.23 (m, 2H), 2.99- 2.88 (m, 1H), 2.82-2.72 (m, 1H), 2.71-2.59 (m, 3H), 2.23-2.14 (m, 1H), 2.03-1.93 (m, 1H), 1.70- 1.59 (m, 1H), 1.53-1.31 (m, 4H), 1.16-1.00 (m, 2H), 0.98-0.90 (m, 6H), 0.83-0.73 (m, 3H) |
| | (3S)-3-{[5-amino-1-({2-methoxy-4-[4- | | | |
| | (propan-2-yl)piperazin-2- | | | |
| | yl]phenyl}methyl)-1H-pyrazolo[4,3- | | | |
| | d]pyrimidin-7-yl]amino}hexan-1-ol | | | |
| 183 | 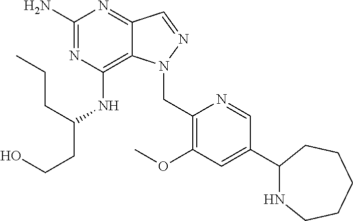 | 89.9 | 2.8 | LC/MS [M + H]⁺ = 469.2 RT (min) = 1.29 δ 9.31-9.10 (m, 2H), 9.08-9.01 (m, 1H), 8.11 (s, 1H), 7.84 (br s, 2H), 7.72 (s, 1H), 7.69 (s, 1H), 5.81 (s, 2H), 4.60-4.53 (m, 1H), 4.51- 4.45 (m, 1H), 3.92 (s, 3H), 3.36- 3.27 (m, 1H), 3.21-3.13 (m, 1H), 2.16-2.05 (m, 1H), 1.99-1.74 (m, 7H), 1.69-1.57 (m, 4H), 1.37- 1.24 (m, 2H), 0.91-0.85 (m, 3H) |
| | (3S)-3-[(5-amino-1-{[5-(azepan-2-yl)-3- | | | |
| | methoxypyridin-2-yl]methyl}-1H-pyrazo- | | | |
| | lo[4,3-d]pyrimidin-7-yl)amino]hexan-1-ol | | | |
| 184 | 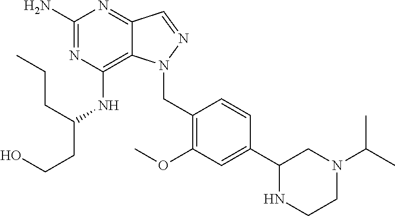 | 6.5 | 0.15 | LC/MS [M + H]⁺ = 497.2 RT (min) = 1.14 δ 7.61-7.53 (m, 1H), 7.16-7.09 (m, 1H), 6.88-6.80 (m, 1H), 6.45- 6.36 (m, 1H), 5.75-5.49 (m, 4H), 4.38-4.26 (m, 1H), 3.91-3.82 (m, 3H), 3.35-3.24 (m, 2H), 3.02- 2.93 (m, 1H), 2.82-2.73 (m, 1H), 2.71-2.59 (m, 3H), 2.26-2.17 (m, 1H), 2.05-1.96 (m, 1H), 1.70- 1.60 (m, 1H), 1.53-1.31 (m, 3H), 1.14-0.99 (m, 2H), 0.96-0.89 (m, 6H), 0.82-0.73 (m, 3H) |
| | (3S)-3-{[5-amino-1-({2-methoxy-4-[4- | | | |
| | (propan-2-yl)piperazin-2- | | | |
| | yl]phenyl}methyl)-1H-pyrazolo[4,3- | | | |
| | d]pyrimidin-7-yl]amino}hexan-1-ol | | | |
| 185 | 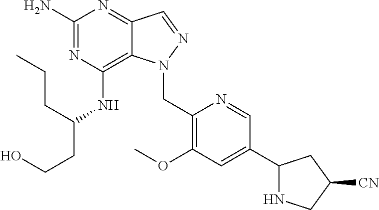 | 398.8 | 27.8 | LC/MS [M + H]⁺ = 465.9 RT (min) = 1.34 δ 8.93 (br d, J = 8.3 Hz, 0.75H), 8.90- 8.85 (m, 0.25H), 8.12 (s, 0.75H), 8.08 (s, 0.25H), 7.86-7.77 (m, 2H), 7.77-7.70 (m, 2H), 5.82 (br s, 2H), 4.87 (br dd, J = 10.6, 7.0 Hz, 0.75H), 4.68-4.63 (m, 0.25H), 4.60-4.52 (m, 1H), 3.93 (s, 0.25H), 3.92 (s, 0.75H) 3.85-3.75 (m, 2H), 2.85- 2.78 (m, 0.25H), 2.73-2.65 (m, 0.75H), 1.85-1.73 (m, 2H), 1.65- 1.57 (m, 2H), 1.34-1.22 (m, 2H), 0.87 (t, J = 7.3 Hz, 3H) |
| | (3R)-5-{6-[(5-amino-7-{[(3S)-1- | | | |
| | hydroxyhexan-3-yl]amino}-1H- | | | |
| | pyrazolo[4,3-d]pyrimidin-1-yl)methyl]-5- | | | |
| | methoxypyridin-3-yl}pyrrolidine-3- | | | |
| | carbonitrile | | | |
| 186 | 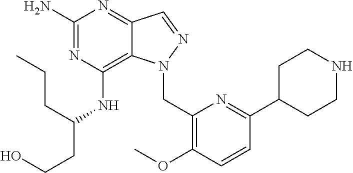 | 1,650 | 242 | LC/MS [M + H]⁺ = 454.9 RT (min) = 1.07 δ 7.54 (s, 1H), 7.49 (d, J = 8.5 Hz, 1H), 7.22 (d, J = 8.2 Hz, 1H), 6.45- 6.35 (m, 1H), 5.88-5.72 (m, 3H), 5.72-5.65 (m, 1H), 4.37 (br dd, J = 12.2, 5.8 Hz, 1H), 3.87 (s, 3H), 3.20 (br d, J = 11.9 Hz, 1H), 2.91 (br t, J = 12.7 Hz, 2H), 2.83-2.74 (m, 1H), 1.85 (br d, J = 12.8 Hz, 2H), 1.75-1.55 (m, 4H), 1.50-1.42 (m, 2H), 1.17-1.07 (m, 2H), 0.80 (br t, J = 7.2 Hz, 3H). |
| | (3S)-3-[(5-amino-1-{[3-methoxy-6- | | | |
| | (piperidin-4-yl)pyridin-2-yl]methyl}-1H- | | | |
| | pyrazolo[4,3-d]pyrimidin-7- | | | |
| | yl)amino]hexan-1-ol | | | |
| 187 | 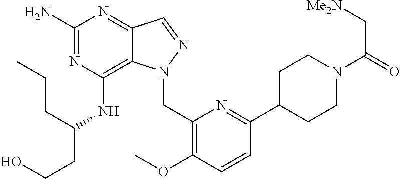 | 402 | 80.5 | LC/MS [M + H]⁺ = 540.3 RT (min) = 1.07 δ 7.51 (s, 1H), 7.44 (br d, J = 8.5 Hz, 1H), 7.19 (dd, J = 8.7, 2.8 Hz, 1H), 6.62 (br t, J = 7.2 Hz, 1H), 5.83-5.73 (m, 1H), 5.65 (br dd, J = 16.7, 7.1 Hz, 1H), 4.39-4.31 (m, 1H), 4.25 (br dd, J = 9.5, 5.4 Hz, 1H), 3.88- 3.79 (m, 4H), 2.99 (br t, J = 12.3 Hz, 1H), 2.79-2.70 (m, 1H), 2.69-2.60 (m, 1H), 2.38 (br d, J = 3.7 Hz, 6H), 1.76-1.58 (m, 4H), 1.54-1.41 (m, 2H), 1.40-1.30 (m, 1H), 1.29- 1.19 (m, 1H), 1.13 (td, J = 15.0, 7.7 Hz, 2H), 0.84-0.71 (m, 3H). |
| | 1-(4-{6-[(5-amino-7-{[(3S)-1- | | | |
| | hydroxyhexan-3-yl]amino}-1H- | | | |
| | pyrazolo[4,3-d]pyrimidin-1-yl)methyl]-5- | | | |
| | methoxypyridin-2-yl}piperidin-1-yl)-2- | | | |
| | (dimethylamino)ethan-1-one | | | |
| 188 | 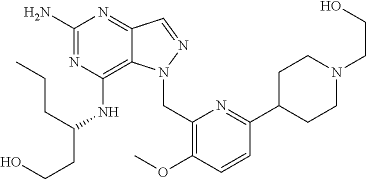 | 290 | 83.7 | LC/MS [M + H]⁺ = 498.9 RT (min) = 1.04 δ 7.52 (s, 1H), 7.46 (d, J = 8.6 Hz, 1H), 7.21 (d, J = 8.5 Hz, 1H), 6.65 (br d, J = 5.4 Hz, 1H), 5.80-5.61 (m, 2H), 4.34 (br dd, J = 12.6, 5.4 Hz, 1H), 3.85 (s, 4H), 3.60 (br t, J = 5.8 Hz, 2H), 3.13-2.95 (m, 2H), 2.78- 2.64 (m, 2H), 2.67-2.54 (m, 2H), 2.49-2.33 (m, 2H), 1.80-1.46 (m, 8H), 1.20-1.05 (m, 2H), 0.80 (br t, J = 7.3 Hz, 3H) |
| | (3S)-3-{[5-amino-1-({6-[1-(2-hydroxy- | | | |
| | ethyl)piperidin-4-yl]-3-methoxypyridin-2- | | | |
| | yl}methyl)-1H-pyrazolo[4,3-d]pyrimidin- | | | |
| | 7-yl]amino}hexan-1-ol | | | |
| 189 | 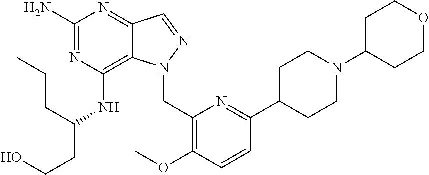 | 85.3 | 9.8 | LC/MS [M + H]⁺ = 538.9 RT (min) = 1.06 δ 7.50 (s, 1H), 7.46 (d, J = 8.5 Hz, 1H), 7.24 (d, J = 8.5 Hz, 1H), 6.91- 6.70 (m, 1H), 5.80-5.53 (m, 4H), 4.40-4.28 (m, 1H), 3.96-3.78 (m, 5H), 3.28 (br t, J = 11.7 Hz, 1H), 3.01- 2.88 (m, 1H), 2.62-2.53 (m, 2H), 2.41-2.23 (m, 2H), 1.80-1.61 (m, 6H), 1.61-1.40 (m, 6H), 1.28- 1.17 (m, 2H), 0.83 (t, J = 7.3 Hz, 3H). |
| | (3S)-3-{[5-amino-1-({3-methoxy-6-[1- | | | |
| | (oxan-4-yl)piperidin-4-yl]pyridin-2- | | | |
| | yl}methyl)-1H-pyrazolo[4,3-d]pyrimidin- | | | |
| | 7-yl]amino}hexan-1-ol | | | |
| 190 | 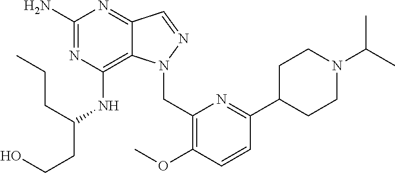 | 175 | 26.7 | LC/MS [M + H]⁺ = 497.0 RT (min) = 1.06 δ 7.51 (s, 1H), 7.47 (d, J = 8.5 Hz, 1H), 7.23 (d, J = 8.6 Hz, 1H), 6.68- 6.47 (m, 1H), 5.87-5.52 (m, 4H), 4.40-4.26 (m, 1H), 3.85 (s, 3H), 2.76-2.55 (m, 2H), 1.87-1.75 (m, 2H), 1.75-1.54 (m, 4H), 1.49 (br dd, J = 7.2, 6.3 Hz, 2H), 1.21-1.12 (m, 2H), 1.10 (br d, J = 6.5 Hz, 6H), 0.81 (br t, J = 7.3 Hz, 3H). |
| | (3S)-3-{[5-amino-1-({3-methoxy-6-[1- | | | |
| | (propan-2-yl)piperidin-4-yl]pyridin-2- | | | |
| | yl}methyl)-1H-pyrazolo[4,3-d]pyrimidin- | | | |
| | 7-yl]amino}hexan-1-ol | | | |
| 191 | 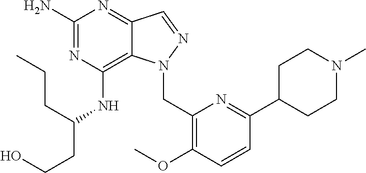 | 221 | 35.8 | LC/MS [M + H]⁺ = 469.3 RT (min) = 1.06 δ 7.69 (s, 1H), 7.48 (br d, J = 8.5 Hz, 1H), 7.24 (br d, J = 8.5 Hz, 1H), 5.99- 5.72 (m, 2H), 4.50-4.37 (m, 1H), 3.87 (s, 3H), 3.03-2.89 (m, 1H), 2.70 (br s, 3H), 1.91-1.79 (m, 2H), 1.73-1.53 (m, 4H), 1.47 (br d, J = 3.7 Hz, 2H), 1.16-1.00 (m, 2H), 0.79 (br t, J = 7.0 Hz, 3H) |
| | (3S)-3-[(5-amino-1-{[3-methoxy-6-(1- | | | |
| | methylpiperidin-4-yl)pyridin-2- | | | |
| | yl]methyl}-1H-pyrazolo[4,3-d]pyrimidin- | | | |
| | 7-yl)amino]hexan-1-ol | | | |
| 192 | 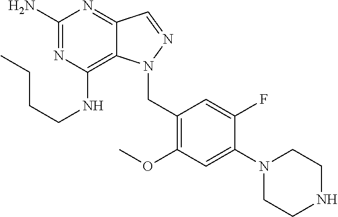 [SMILES: CCCCNc1nc(N)nc2cnn(Cc3cc(F)c(N4CCNCC4)cc3OC)c12] | 193 | 58.2 | LC/MS [M + H]⁺ = 429.1 RT (min) = 1.15 δ 9.54-9.23 (m, 1H), 7.97 (s, 1H), 7.87 (br s, 2H), 7.01 (br d, J = 12.5 Hz, 1H), 6.69 (br d, J = 7.0 Hz, 1H), 5.41 (s, 2H), 3.82 (s, 3H), 3.26 (s, 4H), 1.68-1.51 (m, 2H), 1.41- 1.23 (m, 2H), 0.91 (br t, J = 7.3 Hz, 3H) |
| | N7-butyl-1-{[5-fluoro-2-methoxy-4- | | | |
| | (piperazin-1-yl)phenyl]methyl}-1H- | | | |
| | pyrazolo[4,3-d]pyrimidine-5,7-diamine | | | |
| 193 | 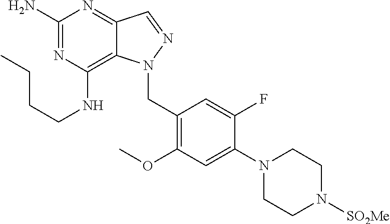 [SMILES: CCCCNc1nc(N)nc2cnn(Cc3cc(F)c(N4CCN(S(C)(=O)=O)CC4)cc3OC)c12] | 84.9 | 529 | LC/MS [M + H]⁺ = 507.1 RT (min) = 1.55 δ 9.40 (br t, J = 5.6 Hz, 1H), 7.97 (br s, 1H), 7.84 (br s, 2H), 6.99 (br d, J = 12.5 Hz, 1H), 6.70 (br d, J = 7.3 Hz, 1H), 5.41 (s, 2H), 3.82 (s, 3H), 3.60- 3.40 (m, 2H), 3.27 (br d, J = 4.3 Hz, 4H), 3.16 (br s, 4H), 2.94 (s, 3H), 1.69-1.51 (m, 2H), 1.40-1.25 (m, 2H), 0.91 (t, J = 7.5 Hz, 3H) |
| | N7-butyl-1-{[5-fluoro-4-(4- | | | |
| | methanesulfonylpiperazin-1-yl)-2- | | | |
| | methoxyphenyl]methyl}-1H- | | | |
| | pyrazolo[4,3-d]pyrimidine-5,7-diamine | | | |
| 194 | 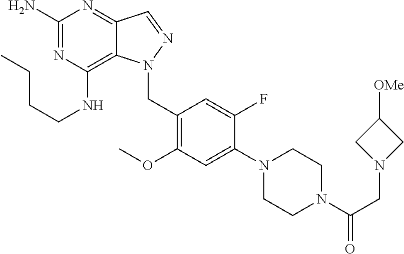 [SMILES: CCCCNc1nc(N)nc2cnn(Cc3cc(F)c(N4CCN(C(=O)CN5CC(OC)C5)CC4)cc3OC)c12] | 63.1 | 37.1 | LC/MS [M + H]⁺ = 556.3 RT (min) = 1.30 δ 9.41 (br t, J = 5.6 Hz, 1H), 7.97 (s, 1H), 7.90 (br s, 2H), 7.00 (br d, J = 12.5 Hz, 1H), 6.65 (br d, J = 7.3 Hz, 1H), 5.41 (s, 2H), 4.44 (br s, 3H), 3.99-3.89 (m, 1H), 3.81 (s, 3H), 3.62 (br s, 1H), 3.25 (s, 2H), 3.11 (br s, 1H), 3.06 (br s, 2H), 1.60 (br t, J = 7.3 Hz, 2H), 1.39-1.22 (m, 2H), 0.91 (t, J = 7.3 Hz, 3H). |
| | 1-[4-(4-{[5-amino-7-(butylamino)-1H- | | | |
| | pyrazolo[4,3-d]pyrimidin-1-yl]methyl}-2- | | | |
| | fluoro-5-methoxyphenyl)piperazin-1-yl]- | | | |
| | 2-(3-methoxyazetidin-1-yl)ethan-1-one | | | |
| 195 | 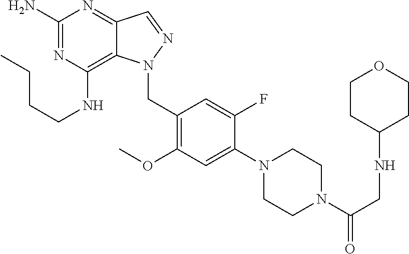 [SMILES: CCCCNc1nc(N)nc2cnn(Cc3cc(F)c(N4CCN(C(=O)CNC5CCOCC5)CC4)cc3OC)c12] | 53.3 | 128 | LC/MS [M + H]⁺ = 570.2 RT (min) = 1.37 δ 9.41 (s, 1H), 7.96 (s, 1H), 7.89 (br d, J = 2.4 Hz, 2H), 7.01 (d, J = 12.8 Hz, 1H), 6.65 (br d, J = 7.3 Hz, 1H), 5.41 (s, 2H), 4.13 (br s, 2H), 3.93 (br d, J = 8.2 Hz, 2H), 3.81 (s, 3H), 3.68 (br d, J = 1.2 Hz, 1H), 3.35-3.21 (m, 2H), 3.08 (br d, J = 18.6 Hz, 3H), 1.97 (br d, J = 11.9 Hz, 2H), 1.71- 1.49 (m, 4H), 1.39-1.26 (m, 2H), 0.90 (t, J = 7.3 Hz, 3H). |
| | 1-[4-(4-{[5-amino-7-(butylamino)-1H- | | | |
| | pyrazolo[4,3-d]pyrimidin-1-yl]methyl}-2- | | | |
| | fluoro-5-methoxyphenyl)piperazin-1-yl]- | | | |
| | 2-[(oxan-4-yl)amino]ethan-1-one | | | |
| 196 | 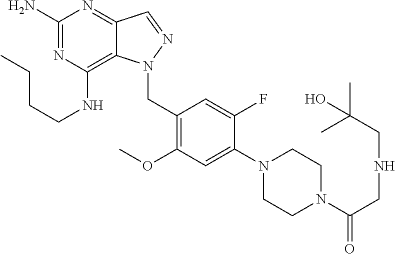 [SMILES: CCCCNc1nc(N)nc2cnn(Cc3cc(F)c(N4CCN(C(=O)CNCC(C)(C)O)CC4)cc3OC)c12] | 131 | 81.0 | LC/MS [M + H]⁺ = 558.2 RT (min) = 1.24 δ 7.89-7.74 (m, 1H), 7.70 (s, 1H), 6.82 (br d, J = 12.5 Hz, 1H), 6.65 (br d, J = 7.2 Hz, 1H), 5.83 (br s, 1H), 5.34 (s, 2H), 3.82 (s, 3H), 3.67- 3.54 (m, 2H), 3.11-2.95 (m, 4H), 2.43 (s, 2H), 1.65-1.51 (m, 2H), 1.41-1.25 (m, 2H), 1.10 (s, 6H), 0.90 (br t, J = 7.4 Hz, 3H) |
| | 1-[4-(4-{[5-amino-7-(butylamino)-1H- | | | |
| | pyrazolo[4,3-d]pyrimidin-1-yl]methyl}-2- | | | |
| | fluoro-5-methoxyphenyl)piperazin-1-yl]- | | | |
| | 2-[(2-hydroxy-2- | | | |
| | methylpropyl)amino]ethan-1-one | | | |
| 197 | 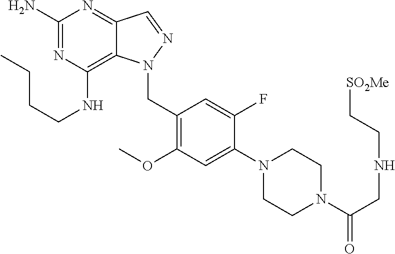 [SMILES: CCCCNc1nc(N)nc2cnn(Cc3cc(F)c(N4CCN(C(=O)CNCCS(C)(=O)=O)CC4)cc3OC)c12] | 320 | 1,000 | LC/MS [M + H]⁺ = 591.9 RT (min) = 1.20 δ 9.42 (br t, J = 5.8 Hz, 1H), 7.97 (s, 1H), 7.90 (br d, J = 1.5 Hz, 2H), 7.01 (d, J = 12.5 Hz, 1H), 6.66 (br d, J = 7.0 Hz, 1H), 5.42 (s, 2H), 4.19 (s, 2H), 3.81 (s, 3H), 3.68 (br s, 1H), 3.59 (br t, J = 7.5 Hz, 1H), 3.52 (br s, 1H), 3.13 (s, 5H), 3.07 (br s, 2H), 1.67- 1.53 (m, 2H), 1.40-1.27 (m, 2H), 0.91 (t, J = 7.3 Hz, 3H). |
| | 1-[4-(4-{[5-amino-7-(butylamino)-1H- | | | |
| | pyrazolo[4,3-d]pyrimidin-1-yl]methyl}-2- | | | |
| | fluoro-5-methoxyphenyl)piperazin-1-yl]- | | | |
| | 2-[(2-methanesulfonylethyl)- | | | |
| | amino]ethan-1-one | | | |
| 198 | 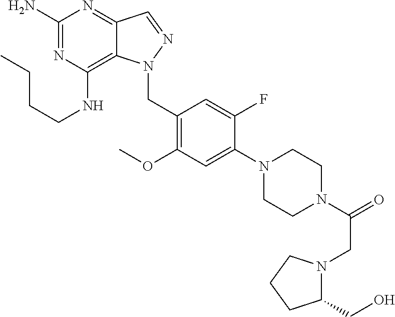 [SMILES: CCCCNc1nc(N)nc2cnn(Cc3cc(F)c(N4CCN(C(=O)CN5CCC[C@H]5CO)CC4)cc3OC)c12] | 48.2 | 50.9 | LC/MS [M + H]⁺ = 570.3 RT (min) = 1.22 δ 7.72 (br d, J = 5.3 Hz, 1H), 7.68 (s, 1H), 6.80 (br d, J = 12.5 Hz, 1H), 6.64 (br d, J = 7.4 Hz, 1H), 5.33 (s, 2H), 3.82 (s, 3H), 3.73-3.45 (m, 3H), 3.14-2.83 (m, 6H), 2.36- 2.23 (m, 1H), 1.86-1.75 (m, 1H), 1.74-1.40 (m, 5H), 1.39-1.27 (m, 2H), 0.90 (br t, J = 7.3 Hz, 3H). |
| | 1-[4-(4-{[5-amino-7-(butylamino)-1H- | | | |
| | pyrazolo[4,3-d]pyrimidin-1-yl]methyl}-2- | | | |
| | fluoro-5-methoxyphenyl)piperazin-1-yl]- | | | |
| | 2-[(2S)-2-(hydroxymethyl)pyrrolidin-1- | | | |
| | yl]ethan-1-one | | | |
| 199 | 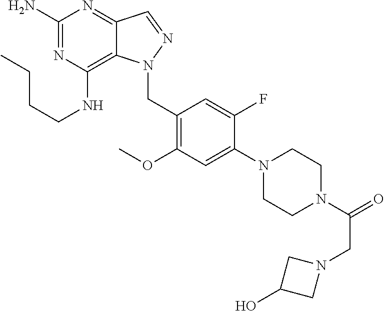 [SMILES: CCCCNc1nc(N)nc2cnn(Cc3cc(F)c(N4CCN(C(=O)CN5CC(O)C5)CC4)cc3OC)c12] | 339 | 1,000 | LC/MS [M + H]⁺ = 542.2 RT (min) = 1.20 δ 9.43 (br t, J = 5.8 Hz, 1H), 8.01- 7.96 (m, 1H), 7.93 (br s, 2H), 7.00 (d, J = 12.5 Hz, 1H), 6.65 (br d, J = 7.3 Hz, 1H), 5.41 (s, 2H), 4.48-4.33 (m, 4H), 3.81 (s, 3H), 3.63 (br s, 2H), 3.54-3.31 (m, 4H), 3.11 (br s, 2H), 3.06 (br d, J = 4.0 Hz, 2H), 3.00 (s, 2H), 1.65-1.54 (m, 2H), 1.38- 1.27 (m, 2H), 0.91 (t, J = 7.4 Hz, 3H). |
| | 1-[4-(4-{[5-amino-7-(butylamino)-1H- | | | |
| | pyrazolo[4,3-d]pyrimidin-1-yl]methyl}-2- | | | |
| | fluoro-5-methoxyphenyl)piperazin-1-yl]- | | | |
| | 2-(3-hydroxyazetidin-1-yl)ethan-1-one | | | |
| 200 | 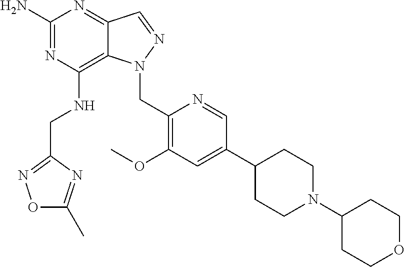 [SMILES: COc1cc(C2CCN(C3CCOCC3)CC2)cnc1Cn1ncc2nc(N)nc(NCc3noc(C)n3)c21] | 116.8 | | LC/MS [M + H]⁺ = 535.3 RT (min) = 1.01 δ 7.92 (d, J = 1.2 Hz, 1H), 7.50 (s, 1H), 7.39 (s, 1H), 5.65 (br d, J = 4.6 Hz, 3H), 4.83 (br d, J = 4.3 Hz, 2H), 3.95-3.83 (m, 3H), 3.63-3.50 (m, 1H), 3.27 (br t, J = 11.7 Hz, 1H), 3.00 (br d, J = 9.5 Hz, 2H), 2.57 (s, 3H), 2.51 (br s, 4H), 2.48-2.37 (m, 1H), 2.21 (br t, J = 10.5 Hz, 2H), 1.83- 1.59 (m, 5H), 1.44 (br dd, J = 11.6, 4.3 Hz, 2H) |
| | 1-({3-methoxy-5-[1-(oxan-4-yl)piperidin- | | | |
| | 4-yl]pyridin-2-yl}methyl)-N7-[(5-methyl- | | | |
| | 1,2,4-oxadiazol-3-yl)methyl]-1H- | | | |
| | pyrazolo[4,3-d]pyrimidine-5,7-diamine | | | |
| 201 | 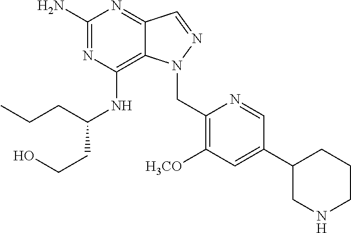 [SMILES: CCC[C@@H](CCO)Nc1nc(N)nc2cnn(Cc3ncc(C4CCCNC4)cc3OC)c12] | 255.5 | | LC/MS [M + H]⁺ = 454.9 RT (min) = 1.08 δ 9.24-9.10 (m, 1H), 9.03-8.87 (m, 1H), 8.72-8.49 (m, 1H), 8.04- 7.93 (m, 1H), 7.75-7.67 (m, 1H), 7.58-7.49 (m, 1H), 5.86-5.71 (m, 2H), 4.63-4.48 (m, 1H), 4.02- 3.79 (m, 3H), 3.65-3.43 (m, 3H), 3.38-3.27 (m, 2H), 3.20-2.82 (m, 3H), 1.98-1.70 (m, 6H), 1.69- 1.56 (m, 2H), 1.38-1.18 (m, 2H), 0.93-0.83 (m, 3H) |
| | (3S)-3-[(5-amino-1-{[3-methoxy-5- | | | |
| | (piperidin-3-yl)pyridin-2-yl]methyl}-1H- | | | |
| | pyrazolo[4,3-d]pyrimidin-7- | | | |
| | yl)amino]hexan-1-ol | | | |
| 202 | 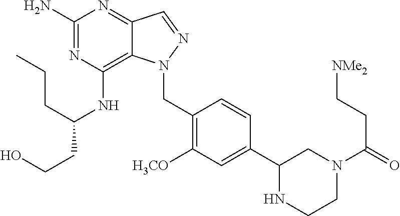 [SMILES: CCC[C@@H](CCO)Nc1nc(N)nc2cnn(Cc3ccc(C4CN(C(=O)CCN(C)C)CCN4)cc3OC)c12] | 495.6 | | LC/MS [M + H]⁺ = 554.3 RT (min) = 0.97 δ 7.94-7.83 (m, 1H), 7.83-7.72 (m, 2H), 7.72-7.61 (m, 1H), 7.35- 7.24 (m, 1H), 7.11-7.01 (m, 1H), 6.96-6.88 (m, 1H), 6.85-6.76 (m, 1H), 5.86-5.73 (m, 2H), 4.67- 4.41 (m, 3H), 4.31-4.22 (m, 1H), 4.14-3.98 (m, 1H), 3.85-3.76 (m, 3H), 3.49-3.23 (m, 2H), 3.03- 2.95 (m, 1H), 2.78 (br s, 3H), 2.52 (br s, 6H), 1.84-1.71 (m, 3H), 1.70- 1.49 (m, 3H), 1.33-1.10 (m, 3H), 0.93-0.79 (m, 4H) |
| | 1-(3-{4-[(5-amino-7-{[(3S)-1- | | | |
| | hydroxyhexan-3-yl]amino}-1H- | | | |
| | pyrazolo[4,3-d]pyrimidin-1-yl)methyl]-3- | | | |
| | methoxyphenyl}piperazin-1-yl)-3- | | | |
| | (dimethylamino)propan-1-one | | | |
| 203 | 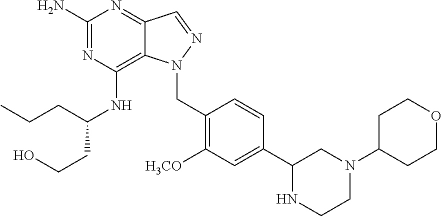 [SMILES: CCC[C@@H](CCO)Nc1nc(N)nc2cnn(Cc3ccc(C4CN(C5CCOCC5)CCN4)cc3OC)c12] | 35.0 | | LC/MS [M + H]⁺ = 539.3 RT (min) = 1.06 δ 7.95-7.86 (m, 1H), 7.82-7.76 (m, 1H), 7.75-7.68 (m, 1H), 7.33- 7.23 (m, 2H), 7.09-7.00 (m, 2H), 6.88-6.77 (m, 1H), 5.84-5.68 (m, 3H), 4.63-4.52 (m, 2H), 4.51- 4.39 (m, 1H), 4.00-3.87 (m, 3H), 3.85-3.76 (m, 3H), 3.36-3.20 (m, 2H), 3.05-2.79 (m, 3H), 1.88- 1.67 (m, 5H), 1.67-1.44 (m, 5H), 1.31-1.12 (m, 3H), 0.93-0.78 (m, 4H) |
| | (3S)-3-{[5-amino-1-({2-methoxy-4-[4- | | | |
| | (oxan-4-yl)piperazin-2-yl]phenyl}methyl)- | | | |
| | 1H-pyrazolo[4,3-d]pyrimidin-7- | | | |
| | yl]amino}hexan-1-ol | | | |
| 204 | 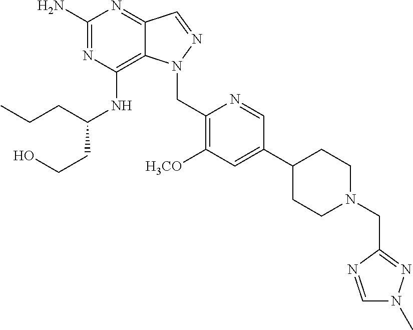 [SMILES: CCC[C@@H](CCO)Nc1nc(N)nc2cnn(Cc3ncc(C4CCN(Cc5ncn(C)n5)CC4)cc3OC)c12] | 91.1 | | LC/MS [M + H]⁺ = 550.0 RT (min) = 1.09 δ 9.23-9.13 (m, 1H), 8.64-8.51 (m, 1H), 7.98-7.90 (m, 1H), 7.74- 7.67 (m, 1H), 7.48-7.38 (m, 1H), 5.81-5.72 (m, 2H), 4.61-4.49 (m, 1H), 4.37-4.26 (m, 1H), 3.96- 3.85 (m, 5H), 3.83-3.63 (m, 1H), 3.62-3.50 (m, 1H), 3.10-2.97 (m, 1H), 2.98-2.81 (m, 5H), 2.03- 1.86 (m, 4H), 1.86-1.75 (m, 2H), 1.69-1.58 (m, 2H), 1.38-1.21 (m, 2H), 0.92-0.82 (m, 3H) |
| | (3S)-3-({5-amino-1-[(3-methoxy-5-{1-[(1- | | | |
| | methyl-1H-1,2,4-triazol-3- | | | |
| | yl)methyl]piperidin-4-yl}pyridin-2- | | | |
| | yl)methyl]-1H-pyrazolo[4,3-d]pyrimidin- | | | |
| | 7-yl}amino)hexan-1-ol | | | |
| 205 | 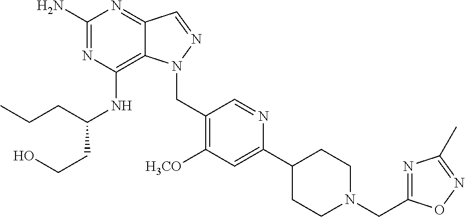 [SMILES: CCC[C@@H](CCO)Nc1nc(N)nc2cnn(Cc3cnc(C4CCN(Cc5nc(C)no5)CC4)cc3OC)c12] | 28.1 | | LC/MS [M + H]⁺ = 551.0 RT (min) = 1.20 δ 9.38-9.20 (m, 1H), 7.95 (s, 1H), 7.71 (s, 2H), 7.44 (s, 2H), 5.75 (s, 2H), 4.69-4.51 (m, 3H), 4.14- 3.96 (m, 2H), 3.93-3.85 (m, 3H), 3.56-3.43 (m, 3H), 3.10-2.97 (m, 2H), 2.96-2.80 (m, 1H), 2.41- 2.37 (m, 3H), 2.04-1.86 (m, 4H), 1.85-1.74 (m, 2H), 1.67-1.56 (m, 2H), 1.36-1.18 (m,2H), 0.91- 0.78 (m, 3H) |
| | (3S)-3-({5-amino-1-[(3-methoxy-5-{1-[(3- | | | |
| | methyl-1,2,4-oxadiazol-5- | | | |
| | yl)methyl]piperidin-4-yl}pyridin-2- | | | |
| | yl)methyl]-1H-pyrazolo[4,3-d]pyrimidin- | | | |
| | 7-yl}amino)hexan-1-ol | | | |
| 206 | 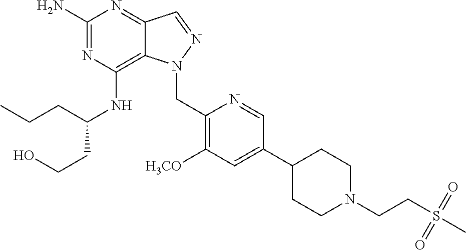 [SMILES: CCC[C@@H](CCO)Nc1nc(N)nc2cnn(Cc3ncc(C4CCN(CCS(C)(=O)=O)CC4)cc3OC)c12] | 74.9 | | LC/MS [M + H]⁺ = 561.0 RT (min) = 1.08 δ 8.01-7.92 (m, 1H), 7.85-7.75 (m, 1H), 7.51-7.38 (m, 2H), 5.70- 5.49 (m, 4H), 4.48-4.35 (m, 1H), 3.95-3.85 (m, 3H), 3.34-3.24 (m, 1H), 3.07-2.96 (m, 4H), 2.78- 2.70 (m, 2H), 2.64-2.57 (m, 1H), 2.11-2.01 (m, 2H), 1.93-1.87 (m, 1H), 1.84-1.51 (m, 8H), 1.39- 1.22 (m, 2H), 0.93-0.82 (m, 3H) |
| | (3S)-3-{[5-amino-1-({5-[1-(2- | | | |
| | methanesulfonylethyl)piperidin-4-yl]-3- | | | |
| | methoxypyridin-2-yl}methyl)-1H- | | | |
| | pyrazolo[4,3-d]pyrimidin-7- | | | |
| | yl]amino}hexan-1-ol | | | |
| 207 | 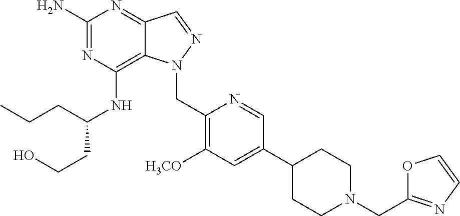 [SMILES: CCC[C@@H](CCO)Nc1nc(N)nc2cnn(Cc3ncc(C4CCN(Cc5ncco5)CC4)cc3OC)c12] | 25.1 | | LC/MS [M + H]⁺ = 536.3 RT (min) = 1.09 δ 8.09-8.03 (m, 1H), 7.99-7.94 (m, 1H), 7.90-7.83 (m, 1H), 7.49- 7.45 (m, 1H), 7.45-7.40 (m, 1H), 7.20-7.14 (m, 1H), 5.69-5.53 (m, 4H), 4.47-4.35 (m, 1H), 3.93- 3.84 (m, 3H), 3.71-3.64 (m, 1H), 2.99-2.87 (m, 2H), 2.24-2.11 (m, 2H), 1.84-1.66 (m, 7H), 1.63- 1.50 (m, 2H), 1.39-1.24 (m, 2H), 0.92-0.78 (m, 3H) |
| | (3S)-3-({5-amino-1-[(3-methoxy-5-{1- | | | |
| | [(1,3-oxazol-2-yl)methyl]piperidin-4- | | | |
| | yl}pyridin-2-yl)methyl]-1H-pyrazolo[4,3- | | | |
| | d]pyrimidin-7-yl}amino)hexan-1-ol | | | |
| 208 | 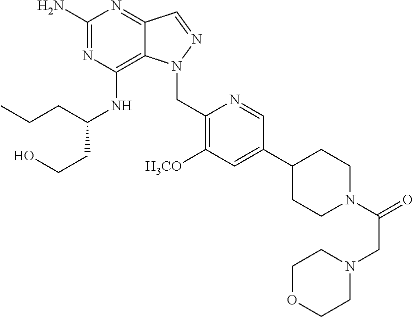 [SMILES: CCC[C@@H](CCO)Nc1nc(N)nc2cnn(Cc3ncc(C4CCN(C(=O)CN5CCOCC5)CC4)cc3OC)c12] | 167.9 | | LC/MS [M + H]⁺ = 582.0 RT (min) = 1.39 δ 7.96 (s, 1H), 7.84 (br s, 1H), 7.47 (s, 1H), 7.45-7.41 (m, 1H), 5.71- 5.58 (m, 4H), 4.50 (br d, J = 14.0 Hz, 2H), 4.45-4.31 (m, 1H), 4.16 (br d, J = 12.2 Hz, 1H), 3.94-3.84 (m, 4H), 3.57 (br s, 1H), 3.25 (br d, J = 13.1 Hz, 1H), 3.13-3.03 (m, 2H), 2.94- 2.75 (m, 2H), 2.67-2.57 (m, 1H), 2.50-2.26 (m, 4H), 1.86-1.45 (m, 8H), 1.39-1.22 (m, 2H), 0.92- 0.81 (m, 3H) |
| | 1-(4-{6-[(5-amino-7-{[(3S)-1- | | | |
| | hydroxyhexan-3-yl]amino}-1H- | | | |
| | pyrazolo[4,3-d]pyrimidin-1-yl)methyl]-5- | | | |
| | methoxypyridin-3-yl}piperidin-1-yl)-2- | | | |
| | (morpholin-4-yl)ethan-1-one | | | |
| 209 | 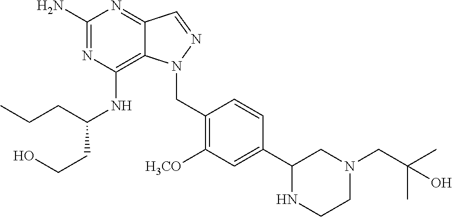 [SMILES: CCC[C@@H](CCO)Nc1nc(N)nc2cnn(Cc3ccc(C4CN(CC(C)(C)O)CCN4)cc3OC)c12] | 12.3 | | LC/MS [M + H]⁺ = 527.3 RT (min) = 1.05 δ 7.62-7.52 (m, 1H), 7.17-7.05 (m, 1H), 6.89-6.75 (m, 1H), 6.47- 6.34 (m, 1H), 5.76-5.43 (m, 5H), 4.37-4.23 (m, 1H), 3.89-3.79 (m, 2H), 3.71-3.53 (m, 1H), 3.35- 3.21 (m, 2H), 2.94-2.70 (m, 4H), 2.30-1.99 (m, 4H), 1.71-1.55 (m, 1H), 1.54-1.27 (m, 3H), 1.14- 0.92 (m, 8H), 0.82-0.69 (m, 3H) |
| | (3S)-3-{[5-amino-1-({4-[4-(2-hydroxy-2- | | | |
| | methylpropyl)piperazin-2-yl]-2- | | | |
| | methoxyphenyl}methyl)-1H- | | | |
| | pyrazolo[4,3-d]pyrimidin-7- | | | |
| | yl]amino}hexan-1-ol | | | |
| 210 | 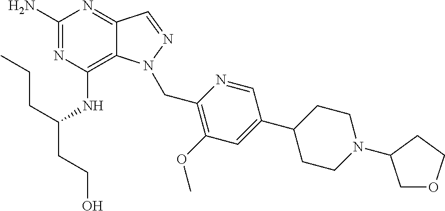 [SMILES: CCC[C@@H](CCO)Nc1nc(N)nc2cnn(Cc3ncc(C4CCN(C5CCOC5)CC4)cc3OC)c12] | 9.05 | | LC/MS m/z = 525.4 [M + H]+ RT (min) = 2.678 (Method C) 1H NMR (400 MHz, methanol-d4) δ = 8.03 (s, 1H), 7.51 (s, 1H), 7.42- 7.39 (m, 1H), 5.76-5.64 (m, 2H), 4.62-4.52 (m, 1H), 3.98-3.92 (m, 5H), 3.83-3.75 (m, 1H), 3.69- 3.63 (m, 3H), 3.19-3.11 (m, 2H), 3.10-3.05 (m, 1H), 2.98-2.92 (m, 1H), 2.26 (br d, J = 11.5 Hz, 1H), , 2.01-2.01 (m, 2H), 1.92-1.80 (m, 8H), 1.94-1.79 (m, 1H), 1.49- 1.43 (m, 2H), 0.99 (t, J = 7.3 Hz, 3H). |
| | (3S)-3-{[5-amino-1-({3-methoxy-5-[1- | | | |
| | (oxolan-3-yl)piperidin-4-yl]pyridin-2- | | | |
| | yl}methyl)-1H-pyrazolo[4,3-d]pyrimidin- | | | |
| | 7-yl]amino}hexan-1-ol | | | |
| 211 | 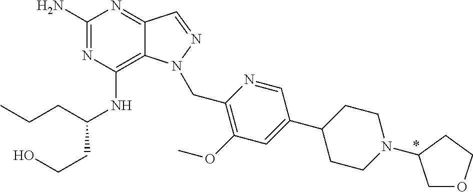 [SMILES: CCC[C@@H](CCO)Nc1nc(N)nc2cnn(Cc3ncc(C4CCN(C5CCOC5)CC4)cc3OC)c12] | 122.98 | | LC/MS m/z = 525.4 [M + H]+ RT (min) = 0.553 (method C) 1H NMR (400 MHz, methanol-d4) δ = 8.03 (s, 1H), 7.51 (s, 1H), 7.42- 7.39 (m, 1H), 5.76-5.64 (m, 2H), 4.62-4.52 (m, 1H), 3.98-3.92 (m, 5H), 3.83-3.75 (m, 1H), 3.69- 3.63 (m, 3H), 3.19-3.11 (m, 2H), 3.10-3.05 (m, 1H), 2.98-2.92 (m, 1H), 2.26 (br d, J = 11.5 Hz, 1H),, 2.01-2.01 (m, 2H), 1.92-1.80 (m, 8H), 1.94-1.79 (m, 1H), 1.49- 1.43 (m, 2H), 0.99 (t, J = 7.3 Hz, 3H). |
| | (3S)-3-{[5-amino-1-({3-methoxy-5-[1- | | | |
| | (oxolan-3-yl)piperidin-4-yl]pyridin-2- | | | |
| | yl}methyl)-1H-pyrazolo[4,3-d]pyrimidin- | | | |
| | 7-yl]amino}hexan-1-ol | | | |
| 212 | 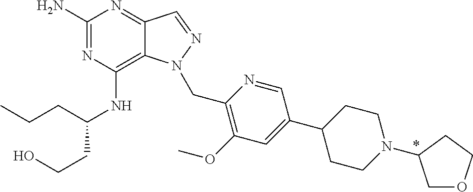 [SMILES: CCC[C@@H](CCO)Nc1nc(N)nc2cnn(Cc3ncc(C4CCN(C5CCOC5)CC4)cc3OC)c12] | 6.5 | | LC/MS m/z = 525.4 [M + H]⁺ RT (min) = 0.563 (method C) 1H NMR (400 MHz, methanol-d4) δ = 8.03 (s, 1H), 7.51 (s, 1H), 7.42- 7.39 (m, 1H), 5.76-5.64 (m, 2H), 4.62-4.52 (m, 1H), 3.98-3.92 (m, 5H), 3.83-3.75 (m, 1H), 3.69- 3.63 (m, 3H), 3.19-3.11 (m, 2H), 3.10-3.05 (m, 1H), 2.98-2.92 (m, 1H), 2.26 (br d, J = 11.5 Hz, 1H), , 2.01-2.01 (m, 2H), 1.92-1.80 (m, 8H), 1.94-1.79 (m, 1H), 1.49- 1.43 (m, 2H), 0.99 (t, J = 7.3 Hz, 3H). |
| | (3S)-3-{[5-amino-1-({3-methoxy-5-[1- | | | |
| | (oxolan-3-yl)piperidin-4-yl]pyridin-2- | | | |
| | yl}methyl)-1H-pyrazolo[4,3-d]pyrimidin- | | | |
| | 7-yl]amino}hexan-1-ol | | | |
| 213 | 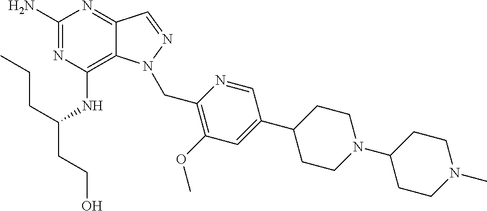 [SMILES: CCC[C@@H](CCO)Nc1nc(N)nc2cnn(Cc3ncc(C4CCN(C5CCN(C)CC5)CC4)cc3OC)c12] | 56.6 | | LC/MS m/z = 552.3 [M + H]+ RT (min) = 0.769 (method B) 1H NMR (400 MHz, DMSO-d6) δ = 7.96 (d, J = 1.5 Hz, 1H), 7.80 (d, J = 8.3 Hz, 1H), 7.47-7.41 (m, 2H), 5.67-5.51 (m, 4H), 4.49-4.33 (m, 2H), 3.88 (s, 3H), 3.45 (br d, J = 3.5 Hz, 2H), 3.00-2.90 (m, 2H), 2.82- 2.72 (m, 2H), 2.54 (s, 1H), 2.24- 2.15 (m, 3H), 2.12 (s, 3H), 1.89- 1.52 (m, 13H), 1.49-1.24 (m, 4H), 0.87 (t, J = 7.4 Hz, 3H). |
| | (3S)-3-({5-amino-1-[(3-methoxy-5-{1′- | | | |
| | methyl-[1,4′-bipiperidine]-4-yl}pyridin-2- | | | |
| | yl)methyl]-1H-pyrazolo[4,3-d]pyrimidin- | | | |
| | 7-yl}amino)hexan-1-ol | | | |
| 214 | 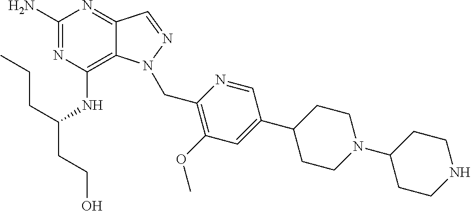 [SMILES: CCC[C@@H](CCO)Nc1nc(N)nc2cnn(Cc3ncc(C4CCN(C5CCNCC5)CC4)cc3OC)c12] | 315.00 | | LC/MS m/z = 538.3 [M + H]+ RT (min) = 0.769 (method B) ¹H NMR (400 MHz, DMSO-d6) 9.23- 9.12 (m, 1H), 7.94 (s, 1H), 7.88- 7.76 (m, 2H), 7.73 (s, 1H), 7.44 (s, 1H), 5.78 (s, 2H), 4.61-4.43 (m, 2H), 3.91 (s, 3H), 3.65-3.43 (m, 8H), 3.17-2.87 (m, 7H), 2.29-2.17 (m, 2H), 2.14-1.76 (m, 2H), 1.63 (q, J = 7.5 Hz, 3H), 1.38-1.22 (m, 2H), 1.16 (t, J = 7.3 Hz, 2H), 0.87 (t, J = 7.4 Hz, 3H). |
| | (3S)-3-([5-amino-1-[(5-{[1,4′- | | | |
| | bipiperidine]-4-yl}-3-methoxypyridin-2- | | | |
| | yl)methyl]-1H-pyrazolo[4,3-d]pyrimidin- | | | |
| | 7-yl}amino)hexan-1-ol | | | |
| 215 | 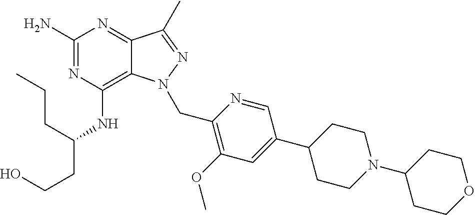 [SMILES: CCC[C@@H](CCO)Nc1nc(N)nc2c(C)nn(Cc3ncc(C4CCN(C5CCOCC5)CC4)cc3OC)c12] | 29.77 | | LC/MS m/z = 553.3 [M + H]+ RT (min) = 0.913( method B) ¹H NMR (400 MHz, DMSO-d6) δ = 7.95 (d, J = 1.5 Hz, 1H), 7.76 (d, J = 8.3 Hz, 1H), 7.43 (d, J = 1.5 Hz, 1H), 5.61-5.50 (m, 4H), 4.51- 4.35 (m, 2H), 3.91 (s, 1H), 3.90 (s, 3H), 3.50-3.46 (m, 2H), 3.04- 2.92 (m, 3H), 2.21 (br s, 1H), 2.19 (s, 3H), 2.19-2.16 (m, 1H), 2.16 (d, J = 2.4 Hz, 1H), 1.81-1.65 (m, 7H), 1.62-1.54 (m, 2H), 1.51-1.38 (m, 2H), 1.36-1.22 (m, 3H), 0.87 (t, J = 7.3 Hz, 3H). |
| | (3S)-3-{[5-amino-1-({3-methoxy-5-[1- | | | |
| | (oxan-4-yl)piperidin-4-yl]pyridin-2- | | | |
| | yl}methyl)-3-methyl-1H-pyrazolo[4,3- | | | |
| | d]pyrimidin-7-yl]amino}hexan-1-ol | | | |
| 216 | 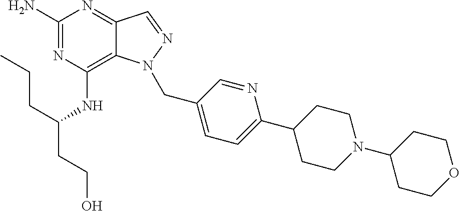 [SMILES: CCC[C@@H](CCO)Nc1nc(N)nc2cnn(Cc3ccc(C4CCN(C5CCOCC5)CC4)nc3)c12] | 1537 | | LC/MS m/z = 509.3 [M + H]+ RT (min) = 0.653 (method B) ¹H NMR (400 MHz, DMSO-d6) δ = 8.24-8.19 (m, 1H), 7.61 (s, 1H), 7.19 (d, J = 1.5 Hz, 2H), 6.18 (s, 1H), 5.82-5.69 (m, 2H), 5.65 (s, 2H), 4.09 (br d, J = 14.4 Hz, 1H), 3.91-3.85 (m, 2H), 3.27-3.17 (m, 6H), 3.01-2.95 (m, 2H), 2.61- 2.55 (m, 1H), 2.48-2.43 (m, 1H), 2.26-2.18 (m, 2H), 1.81-1.74 (m, 2H), 1.72-1.61 (m, 4H), 1.50- 1.39 (m, 4H), 1.07-1.00 (m, 2H), 0.77 (t, J = 7.2 Hz, 3H). |
| | (3S)-3-{[5-amino-1-({6-[1-(oxan-4- | | | |
| | yl)piperidin-4-yl]pyridin-3-yl}methyl)-1H- | | | |
| | pyrazolo[4,3-d]pyrimidin-7- | | | |
| | yl]amino}hexan-1-ol | | | |
| 217 | 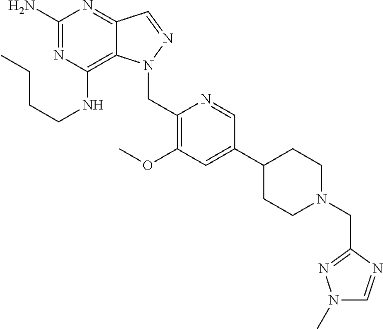 [SMILES: CCCCNc1nc(N)nc2cnn(Cc3ncc(C4CCN(Cc5ncn(C)n5)CC4)cc3OC)c12] | 80.08 | | LC-MS m/z 506.3 [M + H]⁺ RT (min) = 1.323. (Method D) ¹H NMR (400 MHz, DMSO-d6) δ = 8.34 (s, 1H), 8.03-7.98 (m, 1H), 7.96 (d, J = 1.5 Hz, 1H), 7.45 (s, 1H), 7.41 (d, J = 2.0 Hz, 1H), 5.70- 5.65 (m, 2H), 5.63 (s, 2H), 3.87 (s, 3H), 3.81 (s, 3H), 3.51 (s, 2H), 3.47- 3.44 (m, 2H), 3.00-2.95 (m, 2H), 2.54 (br d, J = 1.8 Hz, 2H), 2.15- 2.08 (m, 2H), 1.73-1.66 (m, 3H), 1.65-1.58 (m, 2H), 1.39-1.32 (m, 2H), 0.91 (t, J = 7.3 Hz, 3H); |
| | N7-butyl-1-[(3-methoxy-5-{1-[(1-methyl- | | | |
| | 1H-1,2,4-triazol-3-yl)methyl]piperidin-4- | | | |
| | yl}pyridin-2-yl)methyl]-1H-pyrazolo[4,3- | | | |
| | d]pyrimidine-5,7-diamine | | | |
| 218 | 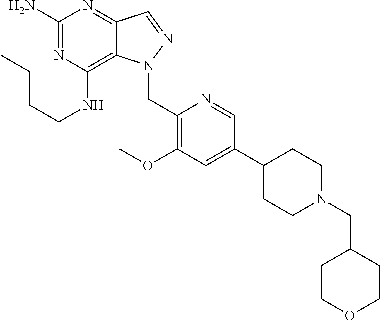 [SMILES: CCCCNc1nc(N)nc2cnn(Cc3ncc(C4CCN(CC5CCOCC5)CC4)cc3OC)c12] | 25.94 | | LC-MS m/z: 509.3 [M + H]+. RT (min) = 1.43. (Method D). ¹H NMR (400 MHz, DMSO-d6) δ = 8.09-8.03 (m, 1H), 7.98 (d, J = 1.5 Hz, 1H), 7.47 (s, 1H), 7.42 (d, J = 1.5 Hz, 1H), 5.80-5.70 (m, 2H), 5.64 (s, 2H), 3.89 (s, 3H), 3.83 (br dd, J = 2.1, 9.2 Hz, 2H), 3.47 (br d, J = 5.4 Hz, 1H), 3.28-3.25 (m, 3H), 2.98-2.93 (m, 2H), 2.63-2.55 (m, 2H), 2.24-2.15 (m, 2H), 2.08- 1.92 (m, 3H), 1.84-1.53 (3H), 1.59- 1.65 (m, 4H), 1.41-1.30 (m, 2H), 1.18-1.06 (m, 2H), 0.92 (t, J = 7.3 Hz, 3H) |
| | N7-butyl-1-[(3-methoxy-5-{1-[(oxan-4- | | | |
| | yl)methyl]piperidin-4-yl}pyridin-2- | | | |
| | yl)methyl]-1H-pyrazolo[4,3-d]pyrimidine- | | | |
| | 5,7-diamine | | | |
| 219 | 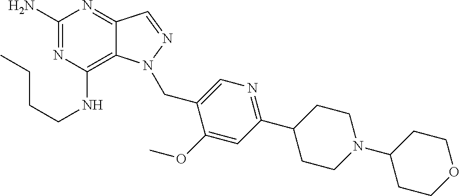 [SMILES: CCCCNc1nc(N)nc2cnn(Cc3cnc(C4CCN(C5CCOCC5)CC4)cc3OC)c12] | 21.9 | | LC/MS m/z = 495.3 [M + H]+ RT (min) = 1.083 (method D) ¹H NMR (400 MHz, DMSO-d6) δ = 7.59 (s, 1H), 7.54 (s, 1H), 6.93 (s, 1H), 6.69 (t, J = 5.4 Hz, 1H), 5.65 (s, 2H), 5.59 (s, 2H), 3.91-3.83 (m, 5H), 3.43 (br d, J = 6.1 Hz, 2H), 3.27 (br t, J = 10.9 Hz, 2H), 2.97 (br d, J = 11.2 Hz, 2H), 2.60-2.54 (m, 1H), 2.47-2.38 (m, 1H), 2.17 (dt, J = 2.1, 11.3 Hz, 2H), 1.89 (br s, 2H), 1.82-1.62 (m, 4H), 1.57-1.37 (m, 4H), 1.23 (sxt, J = 7.4 Hz, 2H), 0.86 (t, J = 7.3 Hz, 3H). |
| | N7-butyl-1-({4-methoxy-6-[1-(oxan-4- | | | |
| | yl)piperidin-4-yl]pyridin-3-yl}methyl)-1H- | | | |
| | pyrazolo[4,3-d]pyrimidine-5,7-diamine | | | |
| 220 | 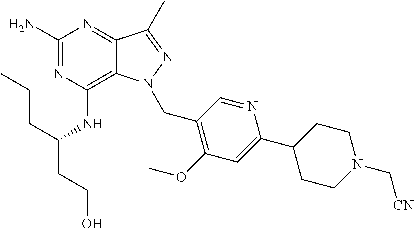 [SMILES: CCC[C@@H](CCO)Nc1nc(N)nc2c(C)nn(Cc3cnc(C4CCN(CC#N)CC4)cc3OC)c12] | 436.38 | | LC/MS [M + H]+ = 508.2 RT (min) = 1.172 (method D) 1H NMR (400 MHz, DMSO-d6) δ = 7.45 (s, 1H), 6.96 (s, 1H), 6.06- 6.04 (m, 1H), 5.67-5.44 (m, 4H), 4.46-4.40 (m, 2H), 4.10-4.08 (m, 1H), 3.88-3.73 (s, 3H), 3.45 (s, 2H), 3.17-3.16 (m, 2H), 2.88-2.85 (m, 2H), 2.5 (s, 3H), 2.10-2.06 (m, 4H), 1.78-1.75 (m, 2H), 1.46- 1.44 (m, 3H), 1.09-1.06 (m, 2H), 0.79-0.75 (t, 3H). |
| | 2-(4-{5-[(5-amino-7-{[(3S)-1- | | | |
| | hydroxyhexan-3-yl]amino}-3-methyl-1H- | | | |
| | pyrazolo[4,3-d]pyrimidin-1-yl)methyl]-4- | | | |
| | methoxypyridin-2-yl}piperidin-1- | | | |
| | yl)acetonitrile | | | |
| 221 | 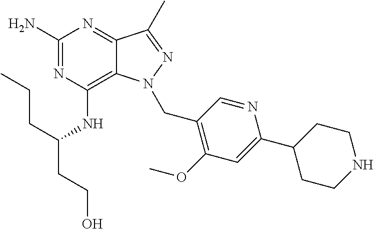 [SMILES: CCC[C@@H](CCO)Nc1nc(N)nc2c(C)nn(Cc3cnc(C4CCNCC4)cc3OC)c12] | 57.11 | | LC/MS [M + H]+ = 469.3 RT (min) = 0.818 (method D) 1H NMR (400 MHz, DMSO-d6) δ = 7.48-7.45 (m, 1H), 6.91 (s, 1H), 6.06-6.01 (m, 1H), 5.66-5.62 (m, 2H), 5.61-5.42 (m, 2H), 4.40- 4.31 (m, 2H), 3.88-3.84 (m, 3H), 3.10-3.02 (m, 2H), 2.75-2.60 (m, 3H), 2.25-2.21 (m, 3H), 1.76- 1.57 (m, 7H), 1.50-1.42 (m, 2H), 1.15-1.06 (m, 2H), 0.82-0.74 (m, 3H). |
| | (3S)-3-[(5-amino-1-{[4-methoxy-6- | | | |
| | (piperidin-4-yl)pyridin-3-yl]methyl}-3- | | | |
| | methyl-1H-pyrazolo[4,3-d]pyrimidin-7- | | | |
| | yl)amino]hexan-1-ol | | | |
| 222 | 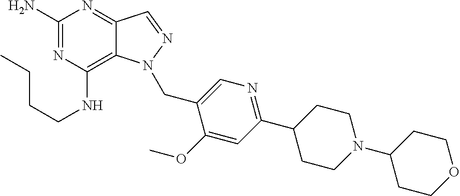 | 170 | | LC/MS m/z = 553.5 [M + H]+ RT (min) = 0.991 (method D). 1H NMR (400 MHz, DMSO-d6) δ = 7.47-7.45 (m, 1H), 6.93 (s, 1H), 6.03 (s, 1H), 5.67-5.63 (m, 2H), 5.59-5.43 (m, 2H), 4.47 (br d, J = 2.0 Hz, 1H), 4.40-4.31 (m, 1H), 3.90-3.85 (m, 7H), 2.98 (ddd, J = 1.6, 5.4, 9.1 Hz, 4H), 2.23 (s, 3H), 2.21-2.16 (m, 3H), 1.81-1.73 (m, 4H), 1.72-1.66 (m, 6H), 1.50- 1.41 (m, 4H), 1.15-1.08 (m, 2H), 0.77 (br d, J = 2.0 Hz, 3H). |
| | (3S)-3-{[5-amino-1-({4-methoxy-6-[1- | | | |
| | (oxan-4-yl)piperidin-4-yl]pyridin-3- | | | |
| | yl}methyl)-3-methyl-1H-pyrazolo[4,3- | | | |
| | d]pyrimidin-7-yl]amino}hexan-1-ol | | | |

### Pharmaceutical Compositions and Administration

In another aspect, there is provided a pharmaceutical composition comprising a compound of as disclosed herein, or of a conjugate thereof, formulated together with a pharmaceutically acceptable carrier or excipient. It may optionally contain one or more additional pharmaceutically active ingredients, such as a biologic or a small molecule drug. The pharmaceutical compositions can be administered in a combination therapy with another therapeutic agent, especially an anti-cancer agent.

The pharmaceutical composition may comprise one or more excipients. Excipients that may be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003).

Preferably, a pharmaceutical composition is suitable for intravenous, intramuscular, subcutaneous, parenteral, spinal or epidermal administration (e.g., by injection or infusion). Depending on the route of administration, the active compound may be coated in a material to protect it from the action of acids and other natural conditions that may inactivate it. The phrase “parenteral administration” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical composition can be administered via a non-parenteral route, such as a topical, epidermal or mucosal route of administration, for example, intranasally, orally, vaginally, rectally, sublingually or topically.

Pharmaceutical compositions can be in the form of sterile aqueous solutions or dispersions. They can also be formulated in a microemulsion, liposome, or other ordered structure suitable to achieve high drug concentration. The compositions can also be provided in the form of lyophilates, for reconstitution in water prior to administration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide a therapeutic response. For example, a single bolus may be administered, several divided doses may be administered over time, or the dose may be proportionally reduced or increased as indicated by the exigencies of the situation. It is especially advantageous to formulate parenteral compositions in dosage unit form for ease of administration and uniformity of dosage. “Dosage unit form” refers to physically discrete units suited as unitary dosages for the subjects to be treated; each unit containing a predetermined quantity of active compound calculated to produce the desired therapeutic response, in association with the required pharmaceutical carrier.

The dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg, or alternatively 0.1 to 5 mg/kg. Exemplary treatment regimens are administration once per week, once every two weeks, once every three weeks, once every four weeks, once a month, once every 3 months, or once every three to 6 months. Preferred dosage regimens include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/mL and in some methods about 25-300 μg/mL.

A “therapeutically effective amount” of a compound of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective amount” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal. Where two or more therapeutic agents are administered in a combination treatment, “therapeutically effective amount” refers to the efficacy of the combination as a whole, and not each agent individually.

The pharmaceutical composition can be a controlled or sustained release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices; (2) micro-infusion pumps; (3) transdermal devices; (4) infusion devices; and (5) osmotic devices.

In certain embodiments, the pharmaceutical composition can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs.

### INDUSTRIAL APPLICABILITY AND USES

TLR7 agonist compounds disclosed herein can be used for the treatment of a disease or condition that can be ameliorated by activation of TLR7.

In one embodiment, the TLR7 agonist is used in combination with an anti-cancer immunotherapy agent—also known as an immuno-oncology agent. An anti-cancer immunotherapy agent works by stimulating a body's immune system to attack and destroy cancer cells, especially through the activation of T cells. The immune system has numerous checkpoint (regulatory) molecules, to help maintain a balance between its attacking legitimate target cells and preventing it from attacking healthy, normal cells. Some are stimulators (up-regulators), meaning that their engagement promotes T cell activation and enhances the immune response. Others are inhibitors (down-regulators or brakes), meaning that their engagement inhibits T cell activation and abates the immune response. Binding of an agonistic immunotherapy agent to a stimulatory checkpoint molecule can lead to the latter's activation and an enhanced immune response against cancer cells. Reciprocally, binding of an antagonistic immunotherapy agent to an inhibitory checkpoint molecule can prevent down-regulation of the immune system by the latter and help maintain a vigorous response against cancer cells. Examples of stimulatory checkpoint molecules are B7-1, B7-2, CD28, 4-1BB (CD137), 4-1BBL, ICOS, CD40, ICOS-L, OX40, OX40L, GITR, GITRL, CD70, CD27, CD40, DR3 and CD28H. Examples of inhibitory checkpoint molecules are CTLA-4, PD-1, PD-L1, PD-L2, LAG-3, TIM-3, Galectin 9, CEACAM-1, BTLA, CD69, Galectin-1, CD113, GPR56, VISTA, 2B4, CD48, GARP, PD1H, LAIR1, TIM-1, CD96 and TIM-4.

Whichever the mode of action of an anti-cancer immunotherapy agent, its effectiveness can be increased by a general up-regulation of the immune system, such as by the activation of TLR7. Thus, in one embodiment, this specification provides a method of treating a cancer, comprising administering to a patient suffering from such cancer a therapeutically effective combination of an anti-cancer immunotherapy agent and a TLR7 agonist as disclosed herein. The timing of administration can be simultaneous, sequential, or alternating. The mode of administration can systemic or local. The TLR7 agonist can be delivered in a targeted manner, via a conjugate.

Cancers that could be treated by a combination treatment as described above include acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, testicular cancer, throat cancer, thyroid cancer, urethral cancer, uterine cancer, vaginal cancer, and vulvar cancer.

Anti-cancer immunotherapy agents that can be used in combination therapies as disclosed herein include: AMG 557, AMP-224, atezolizumab, avelumab, BMS 936559, cemiplimab, CP-870893, dacetuzumab, durvalumab, enoblituzumab, galiximab, IMP321, ipilimumab, lucatumumab, MEDI-570, MEDI-6383, MEDI-6469, muromonab-CD3, nivolumab, pembrolizumab, pidilizumab, spartalizumab, tremelimumab, urelumab, utomilumab, varlilumab, vonlerolizumab. Table B below lists their alternative name(s) (brand name, former name, research code, or synonym) and the respective target checkpoint molecule.

TABLE B

| Immunotherapy Agent | Alternative Name(s) | Target |
|---|---|---|
| AMG 557 | | B7RP-1 (ICOSL) |
| AMP-224 | | PD-1 |
| Atezolizumab | MPDL3280A, RO5541267, | PD-L1 |
| | TECENTRIQ ® | |
| Avelumab | BAVENCIO ® | PD-L1 |
| BMS 936559 | | PD-L1 |
| Cemiplimab | LIBTAYO ® | PD-1 |
| CP-870893 | | CD40 |
| Dacetuzumab | | CD40 |
| Durvalumab | IMFINZI ® | PD-L1 |
| Enoblituzumab | MGA271 | B7-H3 |
| Galiximab | | B7-1 (CD80) |
| IMP321 | | LAG-3 |
| Ipilimumab | YERVOY ® | CTLA-4 |
| Lucatumumab | | CD40 |
| MEDI-570 | | ICOS (CD278) |
| MEDI-6383 | | OX40 |
| MEDI-6469 | | OX40 |
| Muromonab-CD3 | | CD3 |
| Nivolumab | OPDIVO ® | PD-1 |
| Pembrolizumab | KEYTRUDA ® | PD-1 |
| Pidilizumab | MDV9300 | PD-1 |
| Spartalizumab | PDR001 | PD-1 |
| Tremelimumab | Ticilimumab, CP-675, CP- | CTLA-4 |
| | 675,206 | |
| Urelumab | BMS-663513 | CD137 |
| Utomilumab | PF-05082566 | CD137 |
| Varlilumab | CDX 1127 | CD27 |
| Vonlerolizumab | RG7888, MOXR0916, | OX40 |
| | pogalizumab | |

In one embodiment of a combination treatment with a TLR7 agonist, the anti-cancer immunotherapy agent is an antagonistic anti-CTLA-4, anti-PD-1, or anti-PD-L1 antibody. The cancer can be lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

In another embodiment of a combination treatment with a TLR7 agonist, the anti-cancer immunotherapy agent is an antagonistic anti-CTLA-4 antibody, preferably ipilimumab.

In another embodiment of a combination treatment with a TLR7 agonist, the anti-cancer immunotherapy agent is an antagonistic anti-PD-1 antibody, preferably nivolumab or pembrolizumab.

The TLR7 agonists disclosed herein also are useful as vaccine adjuvants.

The practice of this invention can be further understood by reference to the following examples, which are provided by way of illustration and not of limitation.

### Analytical Procedures

### NMR

The following conditions were used for obtaining proton nuclear magnetic resonance (NMR) spectra: NMR spectra were taken in either 400 Mz or 500 Mhz Bruker instrument using either DMSO-d6 or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following preparative and analytical (LC/MS) liquid chromatography procedures were used, as noted in Table A:

LC/MS Procedure A: Analytical LC/MS was used to determine the final purity and retention times. Injection conditions: Column: Waters XBridge C18, 2.1 mm×50 mm, 1.7 μm particles; Mobile Phase A: 5:95 acetonitrile:water with 0.1% TFA; Mobile Phase B: 95:5 acetonitrile:water with 0.1% TFA; Temperature: 50° C.; Gradient: 0% B to 100% B over 3 min, then a 0.50 min hold at 100% B; Flow: 1 mL/min; Detection: MS and UV (220 nm).

LCMS Method B: Column: Xbridge BEH C18 XP (50×2.1 mm), 2.5 μm; mobile phase A: 5:95 CH₃CN:H₂O with 0.1% CF₃CO₂H; mobile phase B: 95:5 CH₃CN:H₂O with 0.1% CF₃CO₂H; temperature: 50° C.; gradient: 0-100% B over 3 minutes; flow rate: 1.1 mL/min).

LCMS Method C: Column: Kinetex XB-C18 (75×3 mm), 2.6 μm; mobile phase A: 10 mM HCO₂NH₄ in water (pH 3.3); mobile phase B: CH₃CN; temperature: 50° C.; gradient: 0-100% B over 3 minutes; flow rate: 1.1 mL/min).

LCMS Method D: Column: Xbridge BEH C18 XP (50×2.1 mm), 2.5 am; mobile phase A: 5:95 CH₃CN:H₂O with 10 mM NH₄OAc; mobile phase B: 95:5 CH₃CN:H₂O with 10 mM NH₄OAc; temperature: 50° C.; gradient: 0-100% B over 3 minutes; flow rate: 1.1 mL/min).

### Synthesis—General Procedures

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). For brevity, the N2 regioisomers are not shown for convenience, but it is to be understood that they are present in the initial product mixture and separated at a later time, for example by preparative HPLC.

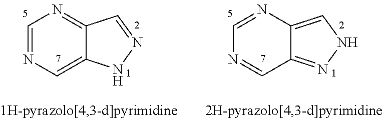 [SMILES: c1ncc2[nH]ncc2n1 ; c1ncc2n[nH]cc2n1]

The mixture of regioisomers can be separated at an early stage of the synthesis and the remaining synthetic steps carried out with the 1H regioisomer or, alternatively, the synthesis can be progressed carrying the mixture of regioisomers and separation effected at a later stage, as desired.

The compounds of the present disclosure can be prepared by a number of methods well known to one skilled in the art of synthetic organic chemistry. These methods include those described below, or variations thereof. Preferred methods include, but are not limited to, those described below in the Schemes below.

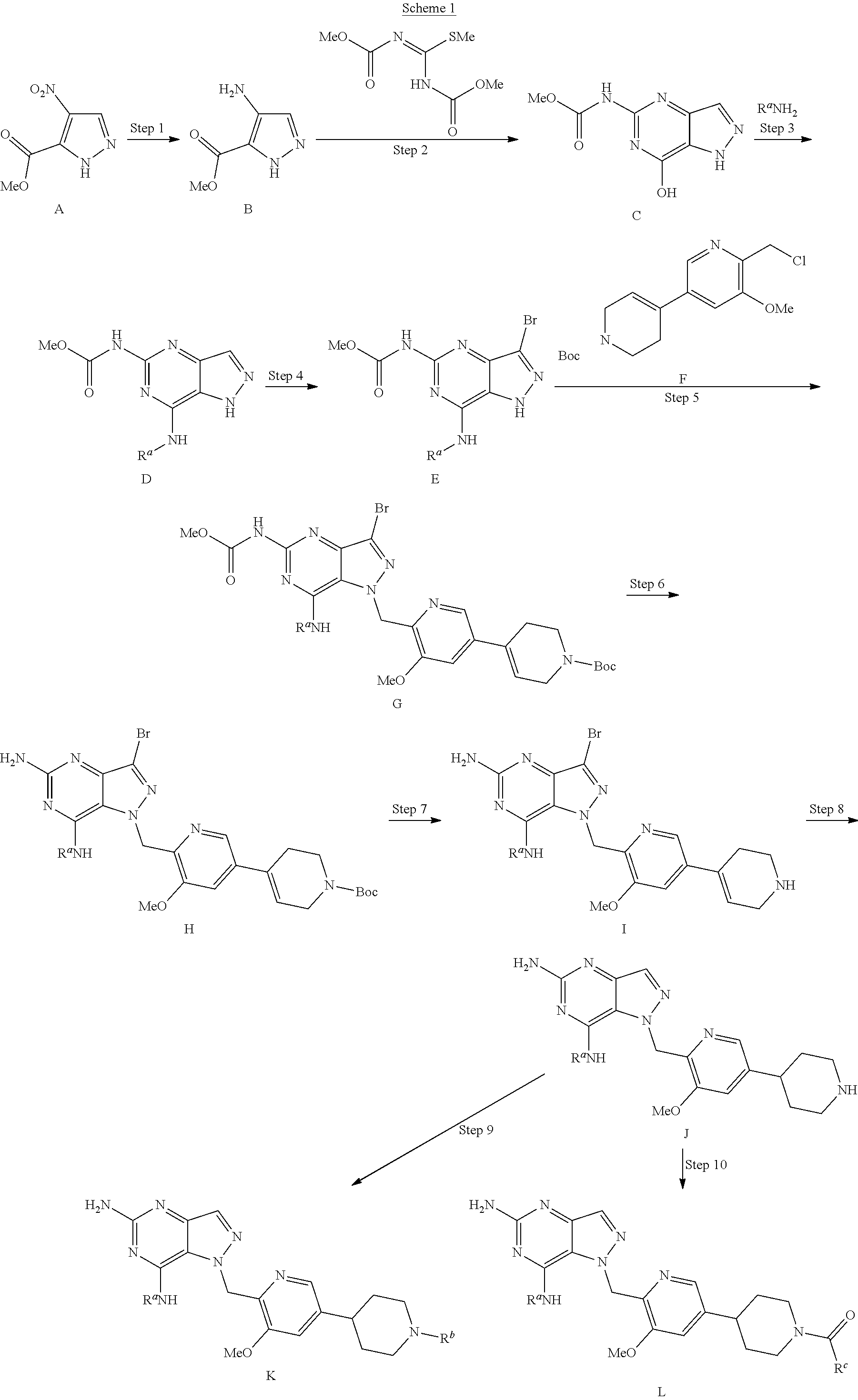

Compound K can be prepared by the synthetic sequence outlined in Scheme 1 above. Reduction of nitropyrazole A to afford compound B followed by cyclization with 1,3-bis(methoxycarbonyl)-2-methyl-2-thiopseudourea gives the hydroxypyrazolopyrimidine C. The amine R^{a}NH₂ is introduced using BOP/DBU coupling conditions, and the subsequent bromination using NBS (Step 4) gives the bromopyrazolopyrimidine E. Alkylation using a benzyl halide F gives a mixture of N1 and N2 products, which are separated, giving N1 intermediate G. Methyl carbamate deprotection (step 6) followed by tert-butyl carbamate deprotection gives intermediate I. Catalytic hydrogenation gives target molecule J. Alkylation or reductive amination of molecule J gives target molecule K (step 9). Coupling of the amine J with the acid using BOP (or HATU) conditions gives the target compound L.

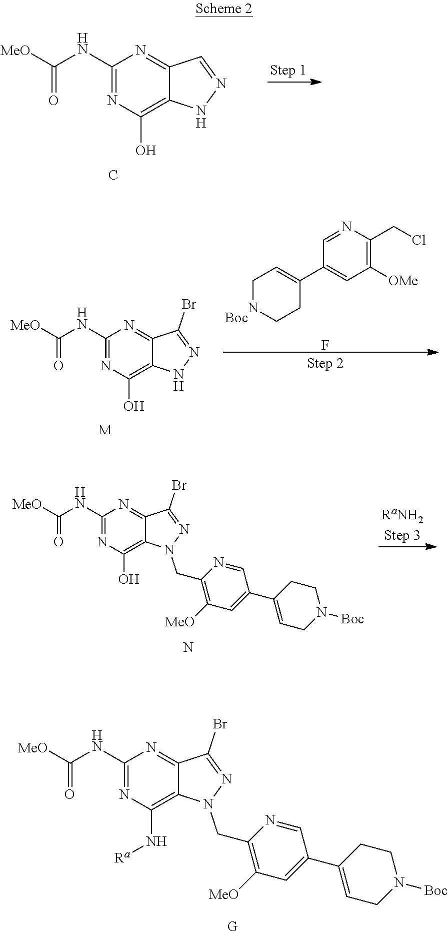

Alternatively, intermediate G may be accessed using the route described in Scheme 2 above. Intermediate C is brominated using NBS, then alkylated to give the intermediate N. Amination then follows, using BOP coupling conditions to give intermediate G.

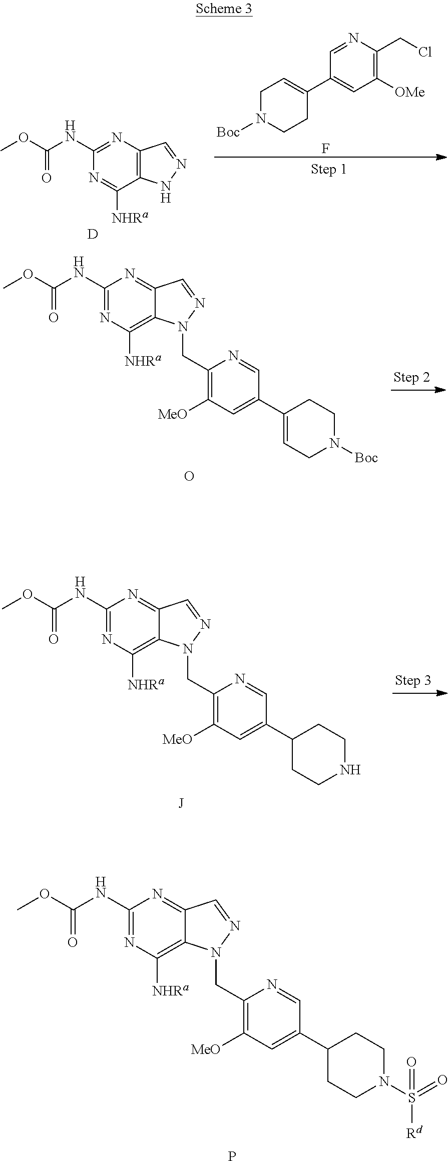

Another alternative route to the compound J is shown in scheme 3 above. Alkylation of intermediate D with benzyl halide F gives intermediate O. However, in this method the ratio of N1 isomer to N2 isomer is generally less favorable. Deprotection of tert-butyl carbamate followed by catalytic hydrogenation gives target molecule J. Sulfonylation of amine J gives target molecule P.

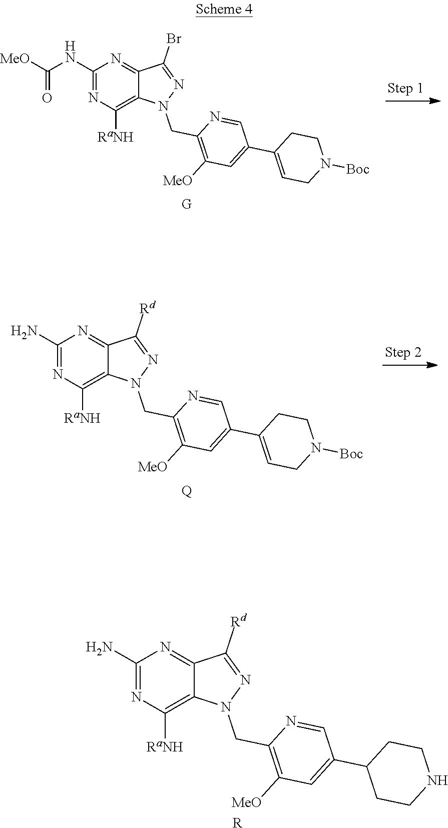

Compound R can be prepared by the synthetic sequence outlined in Scheme 4 above. Suzuki coupling of bromide G with boronic acids (or borane) gives intermediate Q. Deprotection of tert-butyl carbamate followed by catalytic hydrogenation gives target molecule R.

### Synthesis—Specific Examples

To further illustrate the foregoing, the following non-limiting, the following exemplary synthetic schemes are included. Variations of these examples within the scope of the the claims are within the purview of one skilled in the art and are considered to fall within the scope of this disclosure. The reader will recognize that the skilled artisan, provided with the present disclosure and skilled in the relevant art, will be able to prepare and use the compounds disclosed herein without exhaustive examples.

Analytical data for compounds numbered 100 and higher is found in Table A.

### Example 1—Compound 124

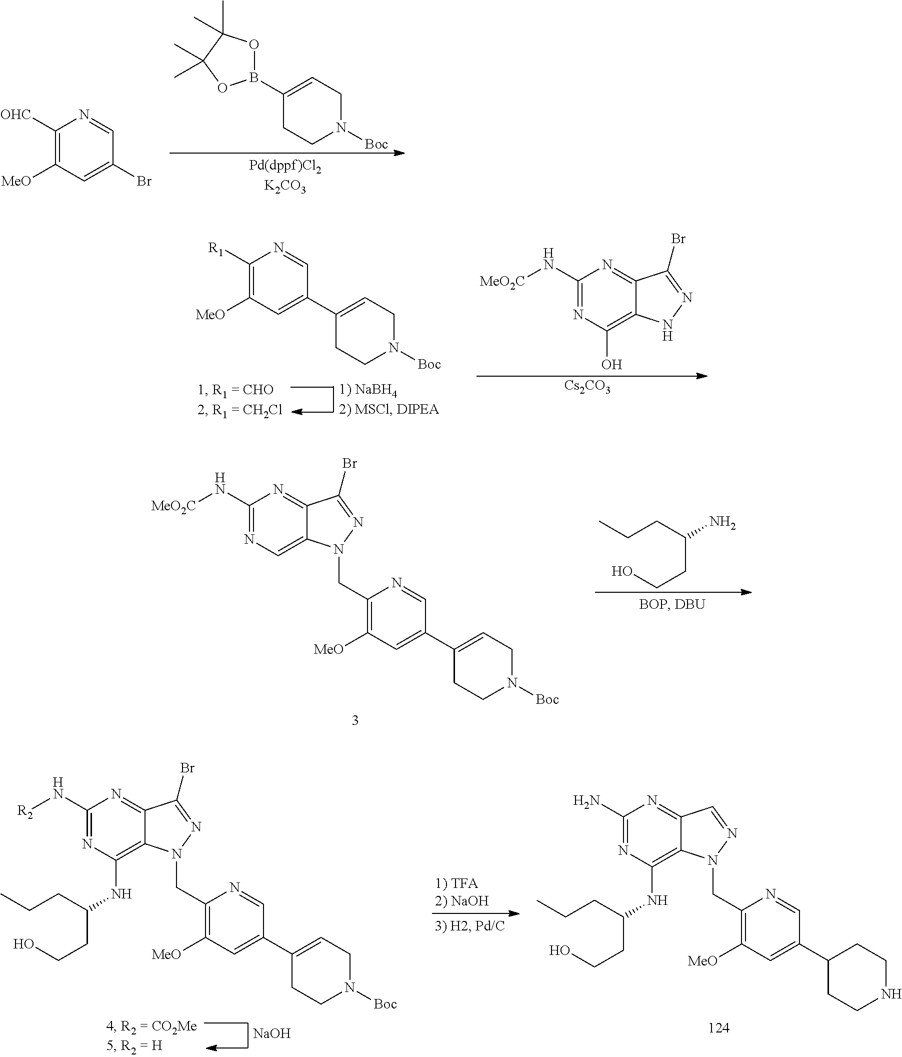

Preparation of (1): A stream of N₂ was bubbled through a mixture of 5-bromo-3-methoxypicolinaldehyde (1.85 g, 8.56 mmol), tert-butyl 4-(4,4,5,5-tetramethyl-1,3,2-dioxaborolan-2-yl)-3,6-dihydropyridine-1(2H)-carboxylate (3.44 g, 11.13 mmol) and K₂CO₃ (4.14 g, 30.0 mmol) in DMF (25 mL) for 3 min. PdCl₂(dppf)-CH₂Cl₂ adduct (0.699 g, 0.856 mmol) was added. An N₂ stream was bubbled through for another 2 min. The reaction mixture was stirred at 80° C. for 5 h and diluted with EtOAc (20 mL). Solids were removed by filtration through a CELITE™ pad. The filtrate was concentrated. The residue was purified using a silica column (80 g), eluting with EtOAc:hexanes, 0-100% gradient. The desired fractions were concentrated to yield tert-butyl 6-formyl-5-methoxy-3′,6′-dihydro-[3,4′-bipyridine]-1′(2′H)-carboxylate 1 (2.7 g, 8.48 mmol, 99% yield).

LCMS ESI: calculated for C₁₇H₂₃N₂O₄=319.2 (M+H⁺), found 319.1 (M+H⁺).

¹H NMR (400 MHz, DMSO-d6) δ 10.14 (s, 1H), 8.48 (d, J=1.5 Hz, 1H), 7.65 (d, J=1.3 Hz, 1H), 6.55 (br s, 1H), 4.08 (br d, J=2.4 Hz, 2H), 3.97 (s, 3H), 3.58 (br t, J=5.7 Hz, 3H), 2.57 (br d, J=1.8 Hz, 2H), 1.44 (s, 9H).

Preparation of (2), part 1: NaBH₄ (0.321 g, 8.48 mmol) was added to a mixture of tert-butyl 6-formyl-5-methoxy-3′,6′-dihydro-[3,4′-bipyridine]-1′(2′H)-carboxylate 1 (2.7 g, 8.48 mmol) in MeOH (50 mL). After stirring at RT for 5 min, the reaction was quenched with water (10 ml) and extracted with 10% MeOH in DCM (6×20 ml). The combined organic extracts were dried over Na₂SO₄, filtered and concentrated. The crude product was purified by silica column (80 g), eluted with EtOAc:hexanes, 0-100% gradient. The desired fractions were concentrated to yield tert-butyl 6-(hydroxymethyl)-5-methoxy-3′,6′-dihydro-[3,4′-bipyridine]-1′(2′H)-carboxylate (1.72 g, 5.37 mmol, 63.3% yield).

LCMS ESI: calculated for C₁₇H₂₅N₂O₄=321.2 (M+H⁺), found 321.2 (M+H⁺).

¹H NMR (400 MHz, DMSO-d6) δ 8.18 (d, J=1.5 Hz, 1H), 7.40 (d, J=1.5 Hz, 1H), 6.29 (br s, 1H), 4.80 (t, J=5.7 Hz, 1H), 4.52 (d, J=5.7 Hz, 2H), 4.03 (br d, J=2.4 Hz, 2H), 3.86 (s, 3H), 3.56 (t, J=5.6 Hz, 2H), 2.56-2.51 (m, 2H), 1.44 (s, 9H)

Preparation of (2), part 2. A solution of tert-butyl 6-(hydroxymethyl)-5-methoxy-3′,6′-dihydro-[3,4′-bipyridine]-1′(2′H)-carboxylate (2.2 g, 6.87 mmol) in DCM (25 mL) was treated with Hunig's base (1.439 mL, 8.24 mmol), followed by addition of Ms-Cl (0.589 mL, 7.55 mmol) dropwise at RT. The resulting reaction mixture was stirred at RT for 16 h. After quenching with water, the two resulting layers were separated. The aqueous layer was back-extracted with EtOAc (2×10 mL). The combined organic extracts were dried over Na₂SO₄, filtered and concentrated. The crude product was purified by silica column chromatography (80 g), EtOAc/hexanes 0-70% gradient. The desired fractions were concentrated to yield tert-butyl 6-(chloromethyl)-5-methoxy-3′,6′-dihydro-[3,4′-bipyridine]-1′(2′H)-carboxylate (1.75 g, 5.16 mmol, 75% yield) as a white solid.

LCMS ESI: calculated for C₁₇H₂₅ClN₂O₄=339.1 (M+H⁺), found 339.2 (M+H⁺).

¹H NMR (400 MHz, CHLOROFORM-d) δ 8.21 (d, J=1.5 Hz, 1H), 7.15 (d, J=1.5 Hz, 1H), 6.11 (br s, 1H), 4.74 (s, 2H), 4.11 (br d, J=3.3 Hz, 2H), 3.92 (s, 3H), 3.66 (t, J=5.6 Hz, 2H), 2.52 (br d, J=1.5 Hz, 2H), 1.50 (s, 9H).

Preparation of (3): Cs₂CO₃ (3.85 g, 11.81 mmol) was added to a stirred mixture of methyl (3-bromo-7-hydroxy-1H-pyrazolo[4,3-d]pyrimidin-5-yl)carbamate (1.700 g, 5.90 mmol) in DMF (60 mL), followed by tert-butyl 6-(chloromethyl)-5-methoxy-3′,6′-dihydro-[3,4′-bipyridine]-1′(2′H)-carboxylate 2 (2.0 g, 5.90 mmol). The resulting reaction mixture was stirred at RT for 16 h. After quenching with saturated NH₄Cl solution and stirring for 1 h, the resulting solid was collected by filtration, suspended in MeOH (10 mL), stirred for 1 hr, collected by filtration, and air dried. The filtrate was concentrated, and the residue was purified by silica column chromatography (80 g), eluting with EtOAc:hexanes=0-100% gradient. The desired fractions were concentrated and the resulting material was combined to yield tert-butyl 6-((3-bromo-7-hydroxy-5-((methoxycarbonyl)amino)-1H-pyrazolo[4,3-d]pyrimidin-1-yl)methyl)-5-methoxy-3′,6′-dihydro-[3,4′-bipyridine]-1′(2′H)-carboxylate 3 (2 g, 3.39 mmol, 57.4% yield) as a off-white solid.

LCMS ESI: calculated for C₂₄H₂₉BrN₇O₆=590.1 (M+H⁺), found 590.1, 592.0 (M+H⁺).

¹H NMR (400 MHz, DMSO-d6) δ 11.52-11.18 (m, 1H), 8.04 (br s, 1H), 7.47 (br s, 1H), 6.28 (br d, J=1.8 Hz, 1H), 5.78 (br s, 2H), 4.01 (br s, 2H), 3.91 (br s, 3H), 3.76 (s, 3H), 3.54 (br s, 2H), 2.50-2.40 (m, 2H), 1.43 (s, 9H). One exchangeable proton is not visible.

Methyl (3-bromo-7-hydroxy-1H-pyrazolo[4,3-d]pyrimidin-5-yl)carbamate can be prepared as follows: 1-Bromopyrrolidine-2,5-dione (N-bromo succinimide (NBS), 2.059 g, 11.57 mmol) is added to a solution of methyl (7-hydroxy-1H-pyrazolo[4,3-d]pyrimidin-5-yl)carbamate (2.2 g, 10.52 mmol) in DMF (20 mL). The reaction mixture is stirred at RT for 1 h. The reaction mixture is worked up with EtOAc, water and brine. The organic phases are combined, concentrated and dried under high vacuum for 1 h to yield methyl (3-bromo-7-hydroxy-1H-pyrazolo[4,3-d]pyrimidin-5-yl)carbamate (2.85 g, 9.89 mmol).

LC-MS m/z 288.0; 290.0 [M+H]⁺

Preparation of (4): DBU (1.225 mL, 8.13 mmol) was added to a suspension of tert-butyl 6-((3-bromo-7-hydroxy-5-((methoxycarbonyl)amino)-1H-pyrazolo[4,3-d]pyrimidin-1-yl)methyl)-5-methoxy-3′,6′-dihydro-[3,4′-bipyridine]-1′(2′H)-carboxylate 3 (960 mg, 1.626 mmol) and (S)-3-aminohexan-1-ol hydrochloride (625 mg, 4.06 mmol) in DMSO (8 mL), followed by stirring for 10 min, leading to a clear solution. BOP (1.8 g, 4.06 mmol) was added. The reaction mixture was stirred at 70° C. for 1 h, cooled, poured into saturated NH₄Cl solution and stirred for 30 min. The resulting solid was collected by filtration, washed with water, and dried in air to yield tert-butyl (S)-6-((3-bromo-7-((1-hydroxyhexan-3-yl)amino)-5-((methoxycarbonyl)-amino)-1H-pyrazolo[4,3-d]pyrimidin-1-yl)methyl)-5-methoxy-3′,6′-dihydro-[3,4′-bipyridine]-1′(2′H)-carboxylate 4 (1.2 g, 1.740 mmol, 107% yield) as a brown solid, which was directly carried over to the next step without purification.

LCMS ESI: calculated for C₃₁H₄₁BrN₈O₆=689.2 (M+H⁺), found 689.3, 691.3 (M+H⁺).

¹H NMR (400 MHz, DMSO-d6) δ 9.91 (s, 1H), 8.09 (d, J=1.5 Hz, 1H), 7.72 (d, J=8.4 Hz, 1H), 7.54 (d, J=1.5 Hz, 1H), 6.39-6.22 (m, 1H), 5.89-5.65 (m, 2H), 4.37 (t, J=5.6 Hz, 1H), 4.01 (br s, 2H), 3.97-3.92 (m, 3H), 3.63 (s, 3H), 3.58-3.52 (m, 2H), 3.48 (br t, J=6.1 Hz, 1H), 3.42 (br d, J=5.9 Hz, 2H), 2.55 (d, J=1.3 Hz, 2H), 1.82-1.70 (m, 2H), 1.61-1.55 (m, 2H), 1.42 (s, 9H), 1.31-1.15 (m, 2H), 0.82 (t, J=7.3 Hz, 3H).

Preparation of (5): A mixture of tert-butyl (S)-6-((3-bromo-7-((1-hydroxyhexan-3-yl)amino)-5-((methoxycarbonyl)amino)-1H-pyrazolo[4,3-d]pyrimidin-1-yl)methyl)-5-methoxy-3′,6′-dihydro-[3,4′-bipyridine]-1′(2′H)-carboxylate 4 (1.2 g, 1.740 mmol) in dioxane (5 mL) was treated with NaOH (5.0 M in water, 3.40 mL, 17.40 mmol). The reaction mixture was stirred at 70° C. for 16 h, cooled, quenched with saturated NH₄Cl solution, and further neutralized with HOAc to pH 6. The mixture was extracted with EtOAc (3×50 mL). The combined organic extracts were dried over Na₂SO₄, filtered and concentrated. The crude product was purified by silica column (80 g) chromatography, eluting with EtOAc/hexanes, 0-100% gradient. The desired fractions were collected and concentrated to yield tert-butyl (S)-6-((5-amino-3-bromo-7-((1-hydroxyhexan-3-yl)amino)-1H-pyrazolo[4,3-d]pyrimidin-1-yl)methyl)-5-methoxy-3′,6′-dihydro-[3,4′-bipyridine]-1′(2′H)-carboxylate 5 (663.2 mg, 1.050 mmol, 60.3% yield) as a off-white solid.

LCMS ESI: calculated for C₂₈H₄₀BrN₈O₅=631.2 (M+H⁺), found 631.3, 633.3 (M+H⁺).

¹H NMR (400 MHz, DMSO-d6) δ 8.11 (d, J=1.3 Hz, 1H), 7.54 (d, J=1.1 Hz, 1H), 6.32 (br s, 1H), 5.91 (s, 2H), 5.75-5.57 (m, 2H), 4.41 (br d, J=3.3 Hz, 2H), 4.15-4.03 (m, 1H), 4.02 (br s, 2H), 3.94 (s, 3H), 3.55 (br t, J=5.5 Hz, 2H), 3.45 (br s, 2H), 2.50-2.44 (m, 2H), 1.86-1.65 (m, 2H), 1.61-1.49 (m, 2H), 1.43 (s, 9H), 1.33-1.18 (m, 2H), 0.84 (t, J=7.3 Hz, 3H).

Preparation of Compound 124: A solution of tert-butyl (S)-6-((5-amino-3-bromo-7-((1-hydroxyhexan-3-yl)amino)-1H-pyrazolo[4,3-d]pyrimidin-1-yl)methyl)-5-methoxy-3′,6′-dihydro-[3,4′-bipyridine]-1′(2′H)-carboxylate 5 (660 mg, 1.045 mmol) in DCM (5 mL) was treated with TFA (1.5 mL, 19.47 mmol) and stirred at RT for 1.5 h, after which LCMS indicated two major products: (S)-3-((5-amino-3-bromo-1-((5-methoxy-1′,2′,3′,6′-tetrahydro-[3,4′-bipyridin]-6-yl)methyl)-1H-pyrazolo[4,3-d]pyrimidin-7-yl)amino)hexan-1-ol and (S)-3-((5-amino-3-bromo-1-((5-methoxy-1′,2′,3′,6′-tetrahydro-[3,4′-bipyridin]-6-yl)methyl)-1H-pyrazolo[4,3-d]pyrimidin-7-yl)amino)hexyl 2,2,2-trifluoroacetate. The reaction mixture was concentrated to dryness. The crude material was suspended in THF (5 mL). NaOH (5.0 N in water, 0.84 mL, 4.18 mmol) was added. The reaction mixture was stirred at RT for 1 h, neutralized with HOAc to pH 6-7, and concentrated to dryness. The resulting semi-solid residue was suspended in EtOAc and stirred for 5 h. The solid was filtered off. The filtrate was concentrated to yield (S)-3-((5-amino-1-((3-methoxy-5-(piperidin-4-yl)pyridin-2-yl)methyl)-1H-pyrazolo[4,3-d]pyrimidin-7-yl)amino)hexan-1-ol, which was suspended in MeOH (20 ml) and transferred into a Parr shaker bottle containing Pd—C (91 mg, 0.852 mmol), which was purged with H₂ and shaked under H₂ (45 psi) for 16 h. The catalyst was removed by filtration through a syringe filter disc. The filtrate was concentrated to dryness to yield (S)-3-((5-amino-1-((3-methoxy-5-(piperidin-4-yl)pyridin-2-yl)methyl)-1H-pyrazolo[4,3-d]pyrimidin-7-yl)amino)hexan-1-ol, (500 mg, 0.732 mmol, 105% yield). 40 mg of the crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 0% B, 0-40% B over 20 min, then a 0-minute hold at 100% B; Flow Rate: 20 mL/min; Column Temperature: 25° C. Fraction collection was triggered by MS and UV signals. Fractions containing the desired product were combined and dried via centrifugal evaporation to yield 18.4 mg of pure Compound 124, which was used in subsequent syntheses without further purification.

Compound 137 and Compound 152 were analogously prepared by reacting Compound 3 with (5-methylisoxazol-3-yl)methanamine and spiro[2.3]hexan-5-ylmethanamine, respectively, instead of (S)-3-aminohexan-1-ol.

### Example 2—Compound 133

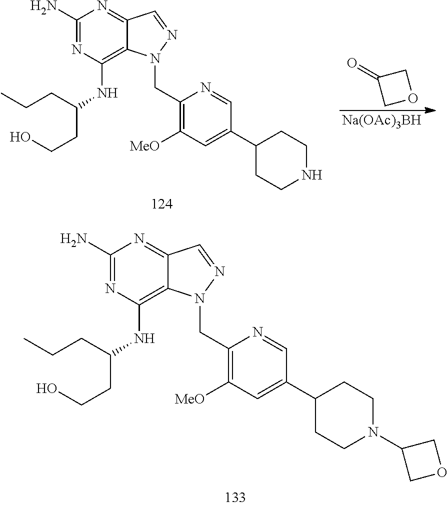

A solution of Compound 124 (40 mg, 0.059 mmol) in DMF (1 mL) was treated with Molecular Sieves, oxetan-3-one (12.67 mg, 0.176 mmol) and 2 drops of HOAc, followed by sodium triacetoxyborohydride (49.7 mg, 0.234 mmol). The reaction mixture was stirred at RT for 16 h. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 13% B, 13-53% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield Compound 133 (4.1 mg, 7.87 μmol, 13.43% yield).

The following compounds were analogously prepared per this Example, using precursors such as Compound 101, Compound 137, Compound 152, or Compound 186, as applicable, instead of Compound 124, for reaction with the complementary carbonyl compound: Compound 102, Compound 108, Compound 111, Compound 112, Compound 113, Compound 115, Compound 116, Compound 126, Compound 127, Compound 129, Compound 131, Compound 134, Compound 141, Compound 142, Compound 143, Compound 147, Compound 148, Compound 150, Compound 154, Compound 156, Compound 157, Compound 158, Compound 159, Compound 163, Compound 189, Compound 190, and Compound 191.

### Example 3—Compound 125

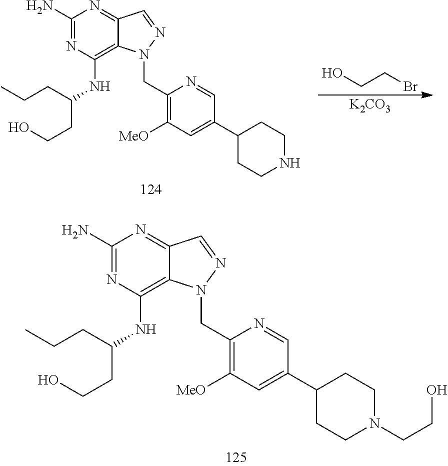

A solution of Compound 124 (60 mg, 0.088 mmol) in DMF (1 mL) was treated with K₂CO₃ (42.5 mg, 0.308 mmol), followed by 2-bromoethan-1-ol (43.9 mg, 0.352 mmol). The reaction mixture was stirred at 50° C. for 1.5 h. The solid was filtered off and the filtrate was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 5% B, 5-45% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield yield Compound 125 (16.8 mg, 0.033 mmol, 37.3% yield).

The following compounds were analogously prepared per this Example, using precursors such as Compound 101, Compound 137, Compound 152, or Compound 186, as applicable, instead of Compound 124, for reaction with the complementary alkyl halide: Compound 114, Compound 119, Compound 120, Compound 144, Compound 145, Compound 146, Compound 151, Compound 153, Compound 164, Compound 165, Compound 188, Compound 204, Compound 205, Compound 206, Compound 207, and Compound 209.

### Example 4—Compound 135

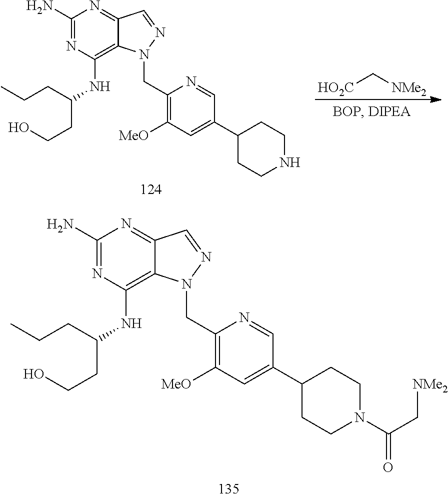

A stirred mixture of (S)-3-((5-amino-1-((3-methoxy-5-(piperidin-4-yl)pyridin-2-yl)methyl)-1H-pyrazolo[4,3-d]pyrimidin-7-yl)amino)hexan-1-ol Compound 124 (30 mg, 0.044 mmol) in DMF (1 mL) was treated with dimethylglycine (5.44 mg, 0.053 mmol), Hunig's base (0.023 mL, 0.132 mmol) and BOP (38.9 mg, 0.088 mmol). The reaction mixture was stirred at RT for 4 h. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 7% B, 7-47% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield Compound 135 (9.6 mg, 0.017 mmol, 39.1% yield).

The following compounds were analogously prepared per this Example, using precursors such as Compound 101, Compound 137, Compound 152, Compound 160, or Compound 186, as applicable, instead of Compound 124, for reaction with the complementary acid: Compound 104, Compound 105, Compound 109, Compound 110, Compound 128, Compound 136, Compound 140, Compound 155, Compound 162, Compound 187, and Compound 208.

### Example 5—Compound 106 and Compound 121

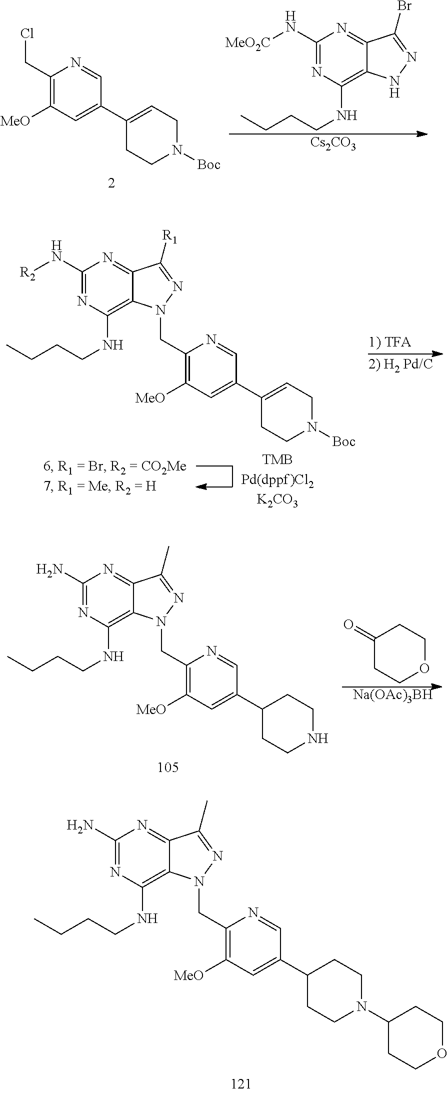

Preparation of 6. To a stirred mixture of methyl (3-bromo-7-(butylamino)-1H-pyrazolo[4,3-d]pyrimidin-5-yl)carbamate (1.215 g, 3.54 mmol) in DMF (20 mL) was added K₂CO₃ (1.428 g, 10.33 mmol), followed by tert-butyl 6-(chloromethyl)-5-methoxy-3′,6′-dihydro-[3,4′-bipyridine]-1′(2′H)-carboxylate 2 (1 g, 2.95 mmol). The reaction mixture was stirred at RT for 5 h, quenched by addition of saturated NH₄Cl solution, and extracted with EtOAc (3×20 mL). The combined organic extracts were dried over Na₂SO₄ and filtered. The filtrate was concentrated. The crude product was purified by silica column (80 g), eluting with EtOAc:hexanes, 0-100% gradient. The desired fractions were concentrated to yield tert-butyl 6-((3-bromo-7-(butylamino)-5-((methoxycarbonyl)amino)-1H-pyrazolo[4,3-d]pyrimidin-1-yl)methyl)-5-methoxy-3′,6′-dihydro-[3,4′-bipyridine]-1′(2′H)-carboxylate 6 (1.0 g, 1.549 mmol, 52.5% yield).

LCMS ESI: calculated for C₂₈H₃₈BrN₈O₅=645.2 (M+H⁺), found 645.3, 647.3 (M+H⁺).

¹H NMR (400 MHz, DMSO-d6) δ 9.82 (s, 1H), 8.10 (d, J=1.5 Hz, 1H), 7.91 (t, J=5.4 Hz, 1H), 7.52 (d, J=1.5 Hz, 1H), 6.31 (br s, 1H), 5.79 (s, 2H), 4.01 (br d, J=2.0 Hz, 2H), 3.93 (s, 3H), 3.64-3.60 (m, 3H), 3.58-3.48 (m, 4H), 2.54-2.50 (m, 2H), 1.66-1.53 (m, 2H), 1.42 (s, 9H), 1.34-1.21 (m, 2H), 0.91-0.80 (m, 3H).

Preparation of 7. A mixture of compound 6 (100 mg, 0.155 mmol), K₂CO₃ (74.9 mg, 0.542 mmol) and PdCl₂(dppf)-CH₂Cl₂ adduct (12.65 mg, 0.015 mmol) in dioxane (0.5 mL) and water (0.1 mL) was bubbled with a stream of N₂ for 2 min. 2,4,6-Trimethyl-1,3,5,2,4,6-trioxatriborinane (TMB, 0.217 mL, 1.549 mmol) was added, followed by bubbling with N₂ for another 2 min. The reaction mixture was stirred at 110° C. for 16 h, cooled, and diluted with EtOAc (5 mL). The solid was removed by filtration through a syringe filter disc. The filtrate was concentrated. The crude product was purified by ISCO silica column (24 g), eluted with 20% MeOH in DCM; DCM 0-30% gradient. The desired fractions were concentrated to yield tert-butyl 6-((5-amino-7-(butylamino)-3-methyl-1H-pyrazolo[4,3-d]pyrimidin-1-yl)methyl)-5-methoxy-3′,6′-dihydro-[3,4′-bipyridine]-1′(2′H)-carboxylate (35.2 mg, 0.067 mmol, 43.5% yield).

LCMS ESI: calculated for C₂₇H₃₉N₈O₃=523.3 (M+H⁺), found 523.4 (M+H⁺).

¹H NMR (400 MHz, DMSO-d6) δ 8.12 (d, J=1.8 Hz, 1H), 7.67 (br d, J=4.6 Hz, 1H), 7.51 (d, J=1.8 Hz, 1H), 6.31 (br d, J=4.4 Hz, 1H), 5.60 (s, 4H), 4.02 (br d, J=1.3 Hz, 2H), 3.92 (s, 3H), 3.54 (t, J=5.5 Hz, 2H), 3.49-3.39 (m, 2H), 3.18 (d, J=5.3 Hz, 2H), 2.19 (s, 3H), 1.68-1.54 (m, 2H), 1.43 (s, 9H), 1.39-1.30 (m, 2H), 0.91 (t, J=7.4 Hz, 3H).

Preparation of Compound 106. To a mixture of compound 7 (35 mg, 0.067 mmol) in DCM (1 mL) was added TFA (0.5 ml, 6.49 mmol). The reaction mixture was stirred at RT for 1 h and concentrated to dryness. The residue was dissolved into MeOH (5 mL), purged with N₂. Pd—C (7.13 mg, 0.067 mmol) was added. The reaction mixture was allowed to stirred at RT under an H₂ balloon for 16 h. The catalyst was removed by filtering through a syringe filter disc. The filtrate was concentrated. The crude product was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 2% B, 2-32% B over 30 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield Compound 106 (9.7 mg, 0.023 mmol, 33.7% yield).

Preparation of Compound 121. A mixture of Compound 106, TFA salt (25 mg, 0.046 mmol) in DMF (1 mL) was treated with tetrahydro-4H-pyran-4-one (46.5 mg, 0.464 mmol), followed by sodium triacetoxyborohydride (29.5 mg, 0.139 mmol) and 1 drop of acetic acid. The reaction mixture was stirred at RT for 16 h and quenched with a small amount of water. The precipitated solid was filtered off via a syringe filter disc, and filtrate was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 11% B, 11-51% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield Compound 121 (6.5 mg, 0.012 mmol, 25.4% yield).

Compound 122 and Compound 123 were analogously prepared.

### Example 6—Compound 101

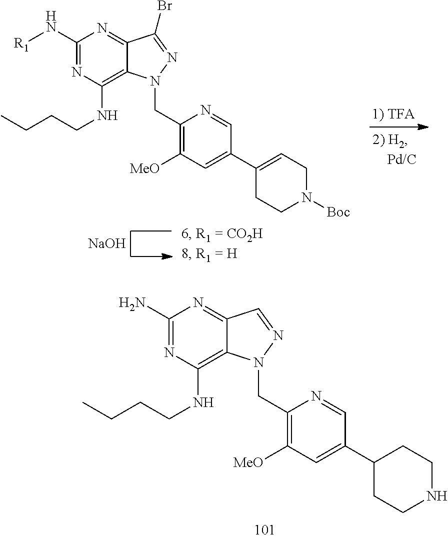

Preparation of 8. A solution of compound 6 (862 mg, 1.335 mmol) in dioxane (5 mL) was treated with NaOH (5.0 N in water; 2.136 mL, 10.68 mmol). The reaction mixture was stirred at 70° C. for 5 h, cooled, and quenched with saturated NH₄Cl, followed by stirring at RT for 1 h. The resulting solid was collected by filtration, washed with water and air dried to yield tert-butyl 6-((5-amino-3-bromo-7-(butylamino)-1H-pyrazolo[4,3-d]pyrimidin-1-yl)methyl)-5-methoxy-3′,6′-dihydro-[3,4′-bipyridine]-1′(2′H)-carboxylate 8 (690 mg, 1.174 mmol, 88% yield).

LCMS ESI: calculated for C₂₆H₃₆BrN₈O₃=587.2 (M+H⁺), found 587.2, 589.2 (M+H⁺).

¹H NMR (400 MHz, DMSO-d6) δ 8.12 (d, J=1.8 Hz, 1H), 7.68 (t, J=5.3 Hz, 1H), 7.52 (d, J=1.5 Hz, 1H), 6.31 (br d, J=2.2 Hz, 1H), 5.91 (s, 2H), 5.68 (s, 2H), 4.02 (br s, 2H), 3.93 (s, 3H), 3.54 (br t, J=5.6 Hz, 2H), 3.45 (br d, J=5.5 Hz, 2H), 2.50-2.49 (m, 2H), 1.64-1.53 (m, 2H), 1.43 (s, 9H), 1.37-1.21 (m, 2H), 0.89 (t, J=7.4 Hz, 3H).

Preparation of Compound 101; Part 1. A mixture of compound 8 (450 mg, 0.766 mmol) in DCM (5 mL) was treated with TFA (3 mL, 38.9 mmol). The reaction mixture was stirred at RT for 45 min. The solvent was removed and residue was triturated with Et₂O twice and dried in vacuo to yield 3-bromo-N7-butyl-1-((5-methoxy-1′,2′,3′,6′-tetrahydro-[3,4′-bipyridin]-6-yl)methyl)-1H-pyrazolo[4,3-d]pyrimidine-5,7-diamine, TFA salt (400 mg, 0.665 mmol, 87% yield).

LCMS ESI: calculated for C₂₁H₂₈BrN₈O=487.1 (M+H⁺), found 487.2, 489.2 (M+H⁺).

Part 2. A solution of 3-bromo-N7-butyl-1-((5-methoxy-1′,2′,3′,6′-tetrahydro-[3,4′-bipyridin]-6-yl)methyl)-1H-pyrazolo[4,3-d]pyrimidine-5,7-diamine, TFA salt (300 mg, 0.499 mmol) in MeOH (10 mL) was purged with N₂, then Pd—C (10%; 53.1 mg, 0.499 mmol) was added. The reaction mixture was stirred under a H₂ balloon for 16 h. The catalyst was removed by filtering through a syringe disc. The filtrate was concentrated to yield 30 mg of crude product which was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 0% B, 0-40% B over 25 min, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield Compound 101 (19.7 mg, 0.048 mmol, 9.62% yield).

The following compounds were analogously prepared: Compound 160, Compound 169, and Compound 170.

In the instance of compound 160: instead of Compound 2, the following compound,

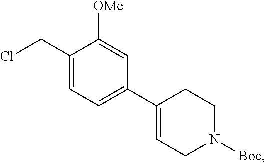 [SMILES: COc1cc(C2=CCN(C(=O)OC(C)(C)C)CC2)ccc1CCl]

which can be prepared from 4-bromo-2-methoxybenzaldehyde per Example 1, was used to generate an analog of compound 6.

Compound 169 was prepared using the this compound instead of Compound 6:

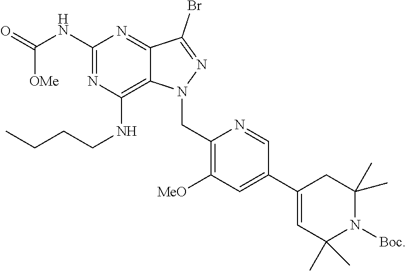 [SMILES: CCCCNc1nc(NC(=O)OC)nc2c(Br)nn(Cc3ncc(C4=CC(C)(C)N(C(=O)OC(C)(C)C)C(C)(C)C4)cc3OC)c12]

This compound can be prepared from

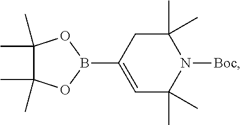 [SMILES: CC(C)(C)OC(=O)N1C(C)(C)C=C(B2OC(C)(C)C(C)(C)O2)CC1(C)C]

generally following Examples 1 and 5 hereinabove.

### Example 7—Compound 130 and Compound 132

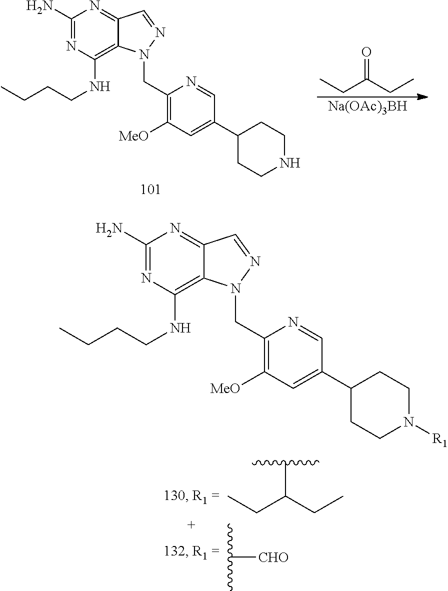

A mixture of N7-butyl-1-((3-methoxy-5-(piperidin-4-yl)pyridin-2-yl)methyl)-1H-pyrazolo[4,3-d]pyrimidine-5,7-diamine (Compound 124) (30 mg, 0.046 mmol) in DMF (1 mL) was treated with 1 drop of HOAc, followed by pentan-3-one (23.65 mg, 0.275 mmol) and Na(OAc)₃BH (97 mg, 0.458 mmol). The resulting reaction mixture was stirred at 60° C. for 24 h. The precipitated solid was filtered off. The filtrate was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 12% B, 12-62% B over 30 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield Compound 130 (1.5 mg, 0.003 mmol, 6.45% yield) and Compound 132 (12.2 mg, 0.028 mmol, 60.4% yield).

### Example 8—Compound 103

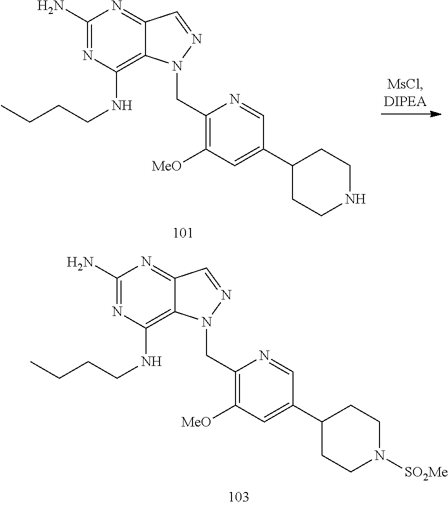 [SMILES: CCCCNc1nc(N)nc2cnn(Cc3ncc(C4CCNCC4)cc3OC)c12 ; CCCCNc1nc(N)nc2cnn(Cc3ncc(C4CCN(S(C)(=O)=O)CC4)cc3OC)c12]

Compound 101 (25 mg, 0.061 mmol) in DCM (0.5 mL) was treated with Hunig's base (0.011 mL, 0.064 mmol). Ms-Cl (4.75 μl, 0.061 mmol) was added dropwise at 0° C. The reaction mixture was stirred at 0° C. for 4 h. The DCM solvent was removed. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-min hold at 17% B, 17-57% B over 25 min, then a 0-min hold at 100% B; Flow Rate: 20 mL/min; Column Temperature: 25° C. Fraction collection was triggered by MS and UV signals. Fractions containing Compound 103 were combined and dried via centrifugal evaporation (6.0 mg, 0.012 mmol, 20.16% yield).

Compound 161 was analogously prepared, using Compound 160 instead of Compound 101.

### Example 9—Compound 117

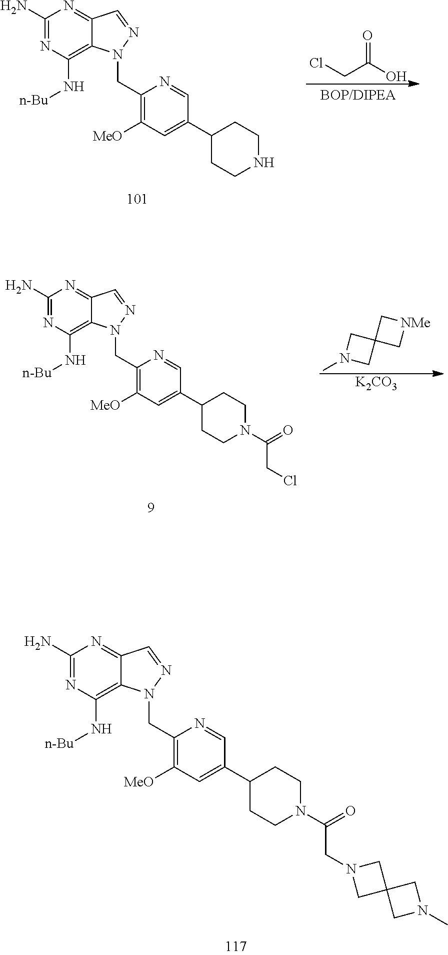

Preparation of 9. A mixture of compound 101, TFA salt (150 mg, 0.286 mmol), 2-chloroacetic acid (40.5 mg, 0.429 mmol) in DMF (4 mL) was treated with Hunig's base (0.100 mL, 0.572 mmol), followed by addition of BOP (190 mg, 0.429 mmol). The reaction mixture was stirred for 4 h, quenched with water, and stirred for 1 hr. The resulting solid was collected by filtration and dried in vacuo to yield 1-(4-(6-((5-amino-7-(butylamino)-1H-pyrazolo[4,3-d]pyrimidin-1-yl)methyl)-5-methoxypyridin-3-yl)piperidin-1-yl)-2-chloroethan-1-one 9 (220 mg, 0.316 mmol, 111% yield) which was directly carried over to the next step without further purification.

LCMS ESI: calculated for C₂₃H₃₂N₈O₂=487.2 (M+H⁺), found 487.2 (M+H⁺).

Preparation of Compound 117. A mixture of compound 9 (25 mg, 0.051 mmol) in DMA (1 mL) was treated with 2-methyl-2,6-diazaspiro[3.3]heptane, HCl salt (38.2 mg, 0.257 mmol), followed by DBU (0.054 mL, 0.359 mmol), The reaction mixture was stirred at 60° C. for 16 h. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 6% B, 6-46% B over 30 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield Compound 117 (4.0 mg, 6.03 μmol, 11.74% yield). See Table A for analytical data.

Compound 118 was analogously prepared.

### Example 10—Compound 139

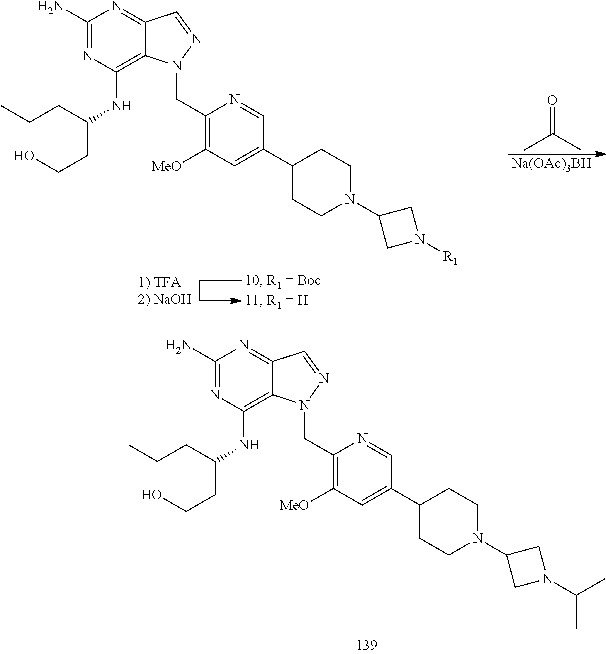

Preparation of 11. A solution of compound 10 prepared analogously to the procedure above for Compound 133 (120 mg, 0.197 mmol) in DCM (2 mL) was treated with TFA (0.5 ml, 6.49 mmol). The reaction mixture was stirred at RT for 1.5 h and concentrated to dryness. The crude material was dissolved into THF (2 mL) and a couple drops of 10 N NaOH in water was added to adjust the pH to 10-11. This resulting mixture was stirred at 60° C. for 1 h and neutralized with HOAc to pH 6-7. The mixture was concentrated and then triturated with 20% DCM in MeOH (20 ml). The solid was filter off. The filtrate was concentrated to dryness to provide compound 11 which was carried over to the next step without further purification.

LCMS ESI: calculated for C₂₆H₄₀N₉O₂=510.3 (M+H⁺), found 510.2 (M+H⁺).

Preparation of Compound 139. A solution of compound 11 was treated with Molecular Sieves, propan-2-one (13.78 mg, 0.237 mmol) and 2 drops of HOAc, followed by Na(OAc)₃BH (40.2 mg, 0.190 mmol). The reaction mixture was stirred at 60° C. for 16 h. The solid was filtered off. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 5% B, 5-45% B over 25 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield Compound 139 (11.9 mg, 0.021 mmol, 44.6% yield).

Compound 138 was analogously prepared.

### Example 11—Compound 180

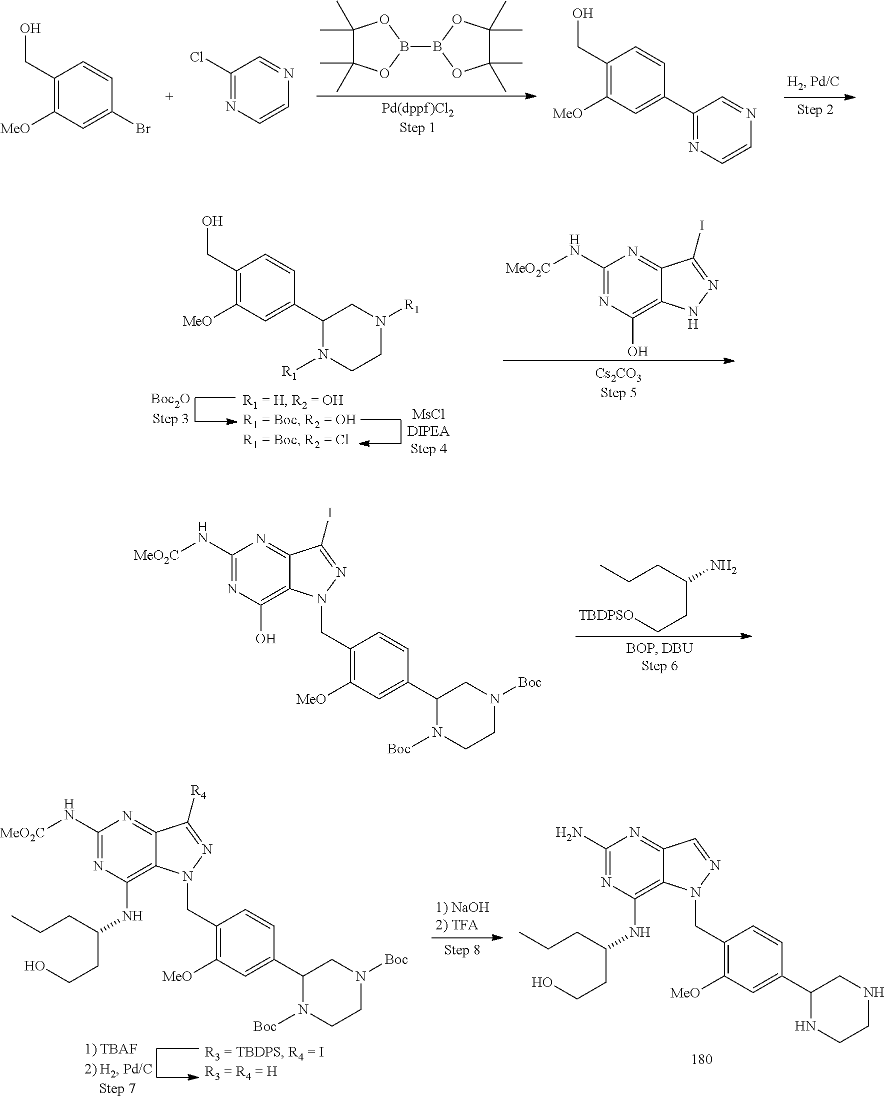

Step 1. A purged suspension of 4-bromo-2-methoxybenzyl alcohol (1 g, 4.61 mmol), PdCl₂(dppf) (0.270 g, 0.369 mmol), bispinacol diborane (1.228 g, 4.84 mmol), and potassium acetate (0.904 g, 9.21 mmol) in dioxane (10 mL) was heated at 95° C. for 16 h. The cooled suspension was diluted with water (2 mL). Potassium phosphate tribasic (2.445 g, 11.52 mmol) and 2-chloropyrazine (0.405 mL, 4.61 mmol) were added. PdCl2(dppf) (0.270 g, 0.369 mmol) was added, while purging with nitrogen. After heating at 95° C. for 16 h, the reaction mixture was diluted with ethyl acetate (50 mL). The organic layer was dried with Na₂SO₄, filtered and concentrated. The crude product was purified on silicagel with a gradient of 0% to 100% of ethyl acetate in hexanes to provide (2-methoxy-4-(pyrazin-2-yl)phenyl)methanol (812 mg).

LC-MS m/z 216.9 [M+H]⁺.

¹H NMR (400 MHz, DMSO-d₆) δ 9.35-9.25 (m, 1H), 8.77-8.67 (m, 1H), 8.65-8.54 (m, 1H), 7.81-7.72 (m, 1H), 7.72-7.65 (m, 1H), 7.60-7.47 (m, 1H), 5.17-5.06 (m, 1H), 4.62-4.51 (m, 2H), 3.95-3.85 (m, 3H)

Step 2. A suspension of (2-methoxy-4-(pyrazin-2-yl)phenyl)methanol (2.51 g, 11.61 mmol), Pd—C (2.5 g, 1.175 mmol), in ethanol (75 mL) was purged 3 times with argon and evacuated and then placed under 50 psi of H₂ for 16 h. To the reaction mixture was added Pd—C (2.5 g, 1.175 mmol). The flask was purged 3 times with argon and evacuated, then placed under 50 psi of H₂. After 16 h, the reaction mixture was diluted with ethyl acetate (50 mL) filtered through CELITE™ and evaporated under reduced pressure to provide (2-methoxy-4-(piperazin-2-yl)phenyl)methanol (2.58 g). This product was used without further purification.

LC-MS m/z 223.0 [M+H]⁺.

¹H NMR (400 MHz, DMSO-d₆) δ 7.30-7.22 (m, 1H), 6.96-6.93 (m, 1H), 6.92-6.88 (m, 1H), 4.50-4.42 (m, 2H), 3.79-3.74 (m, 3H), 3.65-3.55 (m, 1H), 2.95-2.69 (m, 4H), 2.66-2.54 (m, 2H), 2.45-2.34 (m, 1H).

Step 3. A solution of (2-methoxy-4-(piperazin-2-yl)phenyl)methanol (513 mg, 2.31 mmol), DIPEA (1.210 mL, 6.93 mmol) and Boc-anhydride (Boc₂O, 2.145 mL, 9.24 mmol) in DCM (50 mL) was stirred at RT for 80 h. The reaction mixture was evaporated under reduced pressure and dried under high vacuum. The crude product was purified on silicagel with a gradient of 0% to 100% of ethyl acetate in hexanes to provide di-tert-butyl 2-(4-(hydroxymethyl)-3-methoxyphenyl)piperazine-1,4-dicarboxylate (664 mg).

¹H NMR (400 MHz, DMSO-d₆) δ 7.37-7.29 (m, 1H), 6.90-6.72 (m, 2H), 5.20-5.08 (m, 1H), 5.00-4.90 (m, 1H), 4.50-4.42 (m, 2H), 4.40-4.30 (m, 1H), 3.91-3.77 (m, 2H), 3.76-3.71 (m, 3H), 3.31-3.20 (m, 1H), 3.11-2.81 (m, 2H), 1.44-1.38 (m, 9H), 1.38-1.25 (m, 9H).

Step 4. To a solution of di-tert-butyl 2-(4-(hydroxymethyl)-3-methoxyphenyl)-piperazine-1,4-dicarboxylate (3.24 g, 7.67 mmol) and DIPEA (2.009 mL, 11.50 mmol) in DCM (100 mL) cooled to 0° C. was added methanesylfonyl chlor (MsCl, 0.896 mL, 11.50 mmol). The reaction mixture was warmed to RT and stirred for 16 h, concentrated, and dried under high vacuum to provide di-tert-butyl 2-(4-(chloromethyl)-3-methoxyphenyl)piperazine-1,4-dicarboxylate (3.38 g). This product was used without further purification.

LC-MS m/z 441.1 [M+H]⁺.

Step 5. A mixture of di-tert-butyl 2-(4-(chloromethyl)-3-methoxyphenyl)piperazine-1,4-dicarboxylate (3.38 g, 7.67 mmol), methyl (7-hydroxy-3-iodo-2H-pyrazolo[4,3-d]pyrimidin-5-yl)carbamate (2.313 g, 6.90 mmol) and Cs₂CO₃ (7.50 g, 23.01 mmol) in DMF (100 mL) was stirred at RT. After 16 h the reaction mixture was partitioned between EtOAc (300 mL) and 10% aqueous LiCl (200 mL). The organic layer was dried with Na₂SO₄, filtered and concentrated. The crude product was purified on silica gel with a gradient of 0% to 100% of EtOAc in DCM to provide di-tert-butyl 2-(4-((7-hydroxy-3-iodo-5-((methoxycarbonyl)amino)-1H-pyrazolo[4,3-d]pyrimidin-1-yl)methyl)-3-methoxyphenyl)piperazine-1,4-dicarboxylate (5.26 g).

LC-MS m/z 740.0 [M+H]⁺.

Step 6. A solution of di-tert-butyl 2-(4-((7-hydroxy-3-iodo-5-((methoxycarbonyl)-amino)-1H-pyrazolo[4,3-d]pyrimidin-1-yl)methyl)-3-methoxyphenyl)piperazine-1,4-dicarboxylate (5.26 g, 7.11 mmol), (S)-1-((tert-butyldiphenylsilyl)oxy)hexan-3-amine (3.03 g, 8.53 mmol), BOP (6.29 g, 14.22 mmol), and DBU (4.29 ml, 28.4 mmol) in DMSO (10 ml) was stirred for 16 h at RT. The reaction mixture was evaporated under reduced pressure and dried under high vacuum. The crude product was purified on silica gel with a gradient of 0% to 100% of ethyl acetate in dichloromethane to provide di-tert-butyl 2-(4-((7-(((S)-1-((tert-butyl-diphenylsilyl)oxy)hexan-3-yl)amino)-3-iodo-5-((methoxycarbonyl)amino)-1H-pyrazolo[4,3-d]pyrimidin-1-yl)methyl)-3-methoxyphenyl)piperazine-1,4-dicarboxylate (1.12 g).

LC-MS m/z 1077.8 [M+H]⁺.

Step 7. To a solution of di-tert-butyl 2-(4-((7-(((S)-1-((tert-butyldiphenylsilyl)-oxy)hexan-3-yl)amino)-3-iodo-5-((methoxycarbonyl)amino)-1H-pyrazolo[4,3-d]pyrimidin-1-yl)methyl)-3-methoxyphenyl)piperazine-1,4-dicarboxylate (1.12 g, 1.040 mmol) in MeOH (40 mL) was added TBAF (1M in THF, 2.080 mL, 2.080 mmol). After 30 min the reaction mixture concentrated under reduced pressure amd dried under high vacuum. The residue was diluted with MeOH (40 mL). Pd—C (0.221 g, 0.104 mmol) was added. The reaction vessel was purged 3 times with vacuum and nitrogen then purged three times with vacuum and hydrogen. The mixture was stirred under hydrogen for 70 h, filtered through CELITE™ and evaporated under reduced pressure. The crude product was purified on silicagel with a gradient of 0% to 20% of methanol in dichloromethane to provide di-tert-butyl 2-(4-((7-(((S)-1-hydroxyhexan-3-yl)amino)-5-((methoxycarbonyl)amino)-1H-pyrazolo[4,3-d]pyrimidin-1-yl)methyl)-3-methoxyphenyl)piperazine-1,4-dicarboxylate LC-MS m/z 713.3 [M+H]⁺.

The two diastereoisomers of di-tert-butyl 2-(4-((7-(((S)-1-hydroxyhexan-3-yl)amino)-5-((methoxycarbonyl)amino)-1H-pyrazolo[4,3-d]pyrimidin-1-yl)methyl)-3-methoxyphenyl)piperazine-1,4-dicarboxylate (239 mg) were separated via SCF with the following conditions: Column OX 21×250 mm ID, 5 μm; Modile phase: 80/20 CO₂/MeOH; Flow rate: 50 mL/min; Column Temperature 40° C.

Fractions of the first eluting peak were collected and evaporated under reduced pressure (P1; 73 mg). 1H NMR (400 MHz, DMSO-d₆) δ 7.05-6.81 (m, 1H), 6.77-6.65 (m, 1H), 6.61-6.42 (m, 1H), 6.39-6.24 (m, 1H), 5.86-5.56 (m, 2H), 5.18-5.03 (m, 1H), 4.56-4.43 (m, 1H), 4.39-4.35 (m, 1H), 4.35-4.25 (m, 1H), 3.82-3.79 (m, 3H), 3.62 (s, 3H), 3.40-3.33 (m, 2H), 1.77-1.43 (m, 5H), 1.42-1.24 (m, 19H), 1.20-1.01 (m, 4H), 0.97-0.93 (m, 1H), 0.89-0.81 (m, 2H), 0.81-0.74 (m, 3H).

Fractions of the second eluting peak were collected and evaporated under reduced pressure (P2; 80 mg). ¹H NMR (400 MHz, DMSO-d₆) δ 7.05-6.78 (m, 1H), 6.77-6.63 (m, 1H), 6.61-6.43 (m, 1H), 6.39-6.18 (m, 1H), 5.87-5.59 (m, 2H), 5.23-5.01 (m, 1H), 4.57-4.41 (m, 1H), 4.39-4.20 (m, 2H), 3.82-3.78 (m, 3H), 3.64-3.60 (m, 3H), 3.38-3.32 (m, 2H), 1.75-1.42 (m, 5H), 1.40-1.21 (m, 19H), 1.20-1.01 (m, 4H), 0.99-0.92 (m, 1H), 0.89-0.82 (m, 2H), 0.82-0.75 (m, 3H).

Step 8. A solution of di-tert-butyl 2-(4-((7-(((S)-1-hydroxyhexan-3-yl)amino)-5-((methoxycarbonyl)amino)-1H-pyrazolo[4,3-d]pyrimidin-1-yl)methyl)-3-methoxyphenyl)-piperazine-1,4-dicarboxylate (53 mg, 0.074 mmol) and NaOH (0.149 ml, 1.487 mmol) in dioxane (5 mL) was heated at 60° C. for 16 h. The cooled reaction mixture was evaporated under reduced pressure and dried under high vacuum. The crude mixture was diluted with DCM (5 mL). TFA (1 ml, 12.98 mmol) was added. After 16 h, the reaction mixture was partitioned between ethyl acetate 25 mL and saturated NaHCO₃ (25 mL). The organic layer was dried with Na₂SO₄, filtered and concentrated. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 4% B, 4-44% B over 20 minutes, then a 0-minute hold at 100% B; Flow Rate: 20 mL/min; Column Temperature: 25° C. Fraction collection was triggered by MS signals. Fractions containing the desired product Compound 180 were combined and dried via centrifugal evaporation.

### Example 12—Compound 184

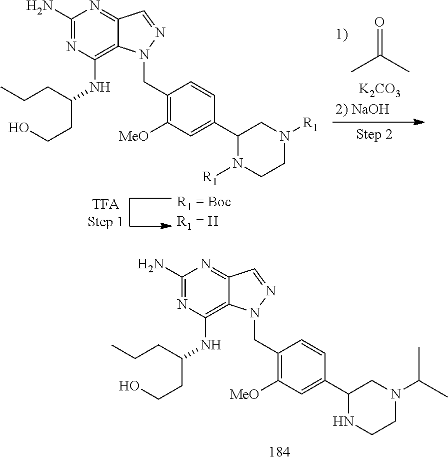

Step 1. To a solution of di-tert-butyl 2-(4-((7-(((S)-1-hydroxyhexan-3-yl)amino)-5-((methoxycarbonyl)amino)-1H-pyrazolo[4,3-d]pyrimidin-1-yl)methyl)-3-methoxyphenyl)-piperazine-1,4-dicarboxylate (see previous example; 80 mg, 0.112 mmol) in DCM (5 mL) was added TFA (500 μl, 6.49 mmol). After 2 h the reaction mixture was concentrated under reduced pressure and dried under high vacuum. The reaction mixture was diluted with methanol (20 mL). K₂CO₃ (50 mg, 0.362 mmol) was added. After 2 h the reaction mixture was diluted with ethyl acetate (25 mL) and filtered through CELITE™ and evaporated under reduced pressure to provide methyl (7-(((S)-1-hydroxyhexan-3-yl)amino)-1-(2-methoxy-4-(piperazin-2-yl)benzyl)-1H-pyrazolo[4,3-d]pyrimidin-5-yl)carbamate. This product was used without further purification.

LC-MS m/z 513.3 [M+H]⁺.

Step 2. A suspension of methyl (7-(((S)-1-hydroxyhexan-3-yl)amino)-1-(2-methoxy-4-(piperazin-2-yl)benzyl)-1H-pyrazolo[4,3-d]pyrimidin-5-yl)carbamate (57.4 mg, 0.112 mmol), 2-bromopropane (55.1 mg, 0.448 mmol), and K₂CO₃ (155 mg, 1.120 mmol) in NMP (1 mL) was heated to 50° C. After 16 h, NaOH (0.112 mL, 1.120 mmol) was added and heating continued for 16 h. The reaction mixture was diluted with methanol (20 mL), filtered and evaporated under reduced pressure. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 5% B, 5-45% B over 25 min, then a 0-minute hold at 100% B; Flow Rate: 20 mL/min; Column Temperature: 25° C. Fraction collection was triggered by MS signals. Fractions containing Compound 184 were combined and dried via centrifugal evaporation.

Compound 181 and Compound 182 were analogously prepared.

### Example 13—Compound 175

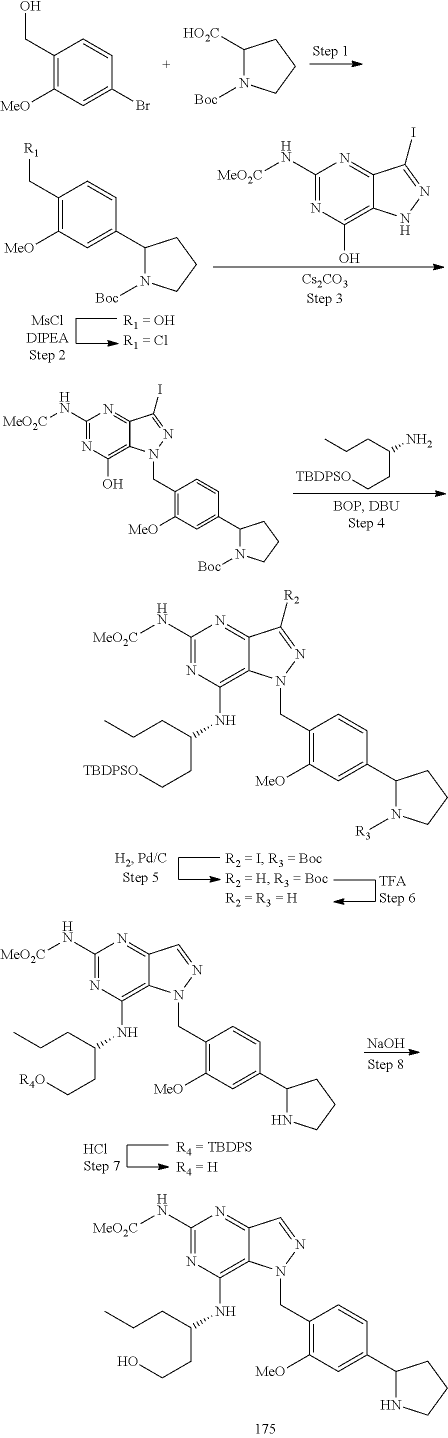

Step 1. In two identical vials were placed (tert-butoxycarbonyl)-L-proline (793 mg, 3.69 mmol), (4-bromo-2-methoxyphenyl)methanol (400 mg, 1.843 mmol), Ir(dF(CF₃)ppy)₂(dtbbpy)PF₆ (20.67 mg, 0.018 mmol), NiBr₂.dttbpy (44.9 mg, 0.092 mmol) and Cs₂CO₃ (1201 mg, 3.69 mmol) with DMA (20 mL). The suspensions were degassed (cap on) with nitrogen for 10 minutes. The caps were sealed with parafilm. The resulting suspensions were placed in a block with stirring and Kessil PR160 427 (427 nm) purple lamps, with a cooling fan. After 16 h, the reaction mixtures were diluted with DCM (50 mL), combined, filtered through CELITE™ and evaporated under reduced pressure. The crude product was purified on silica gel with a gradient of 0% to 100% of ethyl acetate in hexanes to provide a 2:3 mixture of tert-butyl 2-(4-(hydroxymethyl)-3-methoxyphenyl)pyrrolidine-1-carboxylate and tert-butyl 2-(4-formyl-3-methoxyphenyl)pyrrolidine-1-carboxylate.

The mixture was dissolved in EtOH (50 mL) and NaBH₄ (100 mg, 2.64 mmol) was added portion wise. After 4 h the reaction mixture was partitioned between ethyl acetate (100 mL) and saturated NaHCO₃ (50 mL). The organic layer was dried with Na₂SO₄, filtered and concentrated. The crude product was purified on silica gel with a gradient of 0% to 100% of ethyl acetate in hexanes to provide tert-butyl 2-(4-(hydroxymethyl)-3-methoxyphenyl)-pyrrolidine-1-carboxylate LC-MS m/z 252.0 [M+H-tBuOH]⁺.

Step 2. To a mixture of tert-butyl 2-(4-(hydroxymethyl)-3-methoxyphenyl)-pyrrolidine-1-carboxylate (380 mg, 1.236 mmol) and Hunig's base (0.130 mL, 0.742 mmol) in DCM (5 mL) was added Ms-Cl (0.053 mL, 0.680 mmol). After 16 h the reaction mixture was evaporated under reduced pressure and dried under high vacuum to provide tert-butyl 2-(4-(chloromethyl)-3-methoxyphenyl)pyrrolidine-1-carboxylate (201 mg). This product was used without further purification.

LC-MS m/z 326.1 [M+H]⁺.

Step 3. A mixture of tert-butyl 2-(4-(chloromethyl)-3-methoxyphenyl)pyrrolidine-1-carboxylate (401 mg, 1.23 mmol), methyl (7-hydroxy-3-iodo-2H-pyrazolo[4,3-d]pyrimidin-5-yl)carbamate (412 mg, 1.230 mmol), and Cs₂CO₃ (1202 mg, 3.69 mmol) in DMF (20 mL) was stirred at RT for 40 h and concentrated under reduced pressure. The crude product was purified on silica gel with a gradient of 0% to 100% of ethyl acetate in dichloromethane to provide tert-butyl 2-(4-((7-hydroxy-3-iodo-5-((methoxycarbonyl)amino)-1H-pyrazolo[4,3-d]pyrimidin-1-yl)methyl)-3-methoxyphenyl)pyrrolidine-1-carboxylate LC-MS m/z 625.0 [M+H]⁺.

Step 4. A mixture of tert-butyl 2-(4-((7-hydroxy-3-iodo-5-((methoxycarbonyl)-amino)-1H-pyrazolo[4,3-d]pyrimidin-1-yl)methyl)-3-methoxyphenyl)pyrrolidine-1-carboxylate (332 mg, 0.532 mmol), (S)-1-((tert-butyldiphenylsilyl)oxy)hexan-3-amine, HCl (292 mg, 0.744 mmol), BOP (470 mg, 1.063 mmol), and DBU (0.321 mL, 2.127 mmol) in DMSO (3 mL) was heated at 70° C. for 16 h. The reaction mixture was partitioned between ethyl acetate (100 mL) and water (50 mL). The organic layer was dried with Na₂SO₄, filtered and concentrated under reduced pressure. The crude product was purified on silicagel with a gradient of 0% to 100% of ethyl acetate in dichloromethane to provide tert-butyl 2-(4-((7-(((S)-1-((tert-butyldiphenylsilyl)-oxy)hexan-3-yl)amino)-3-iodo-5-((methoxycarbonyl)amino)-1H-pyrazolo[4,3-d]pyrimidin-1-yl)methyl)-3-methoxyphenyl)pyrrolidine-1-carboxylate (146 mg).

LC-MS m/z 962.5 [M+H]⁺.

¹H NMR (400 MHz, DMSO-d₆) δ 7.62-7.53 (m, 2H), 7.51-7.29 (m, 6H), 7.25-7.14 (m, 2H), 6.78-6.73 (m, 1H), 5.73-5.58 (m, 2H), 4.82-4.52 (m, 2H), 3.77-3.65 (m, 3H), 3.64-3.46 (m, 5H), 3.44-3.39 (m, 1H), 2.25-2.04 (m, 1H), 1.88-1.77 (m, 2H), 1.74-1.29 (m, 8H), 1.29-1.13 (m, 6H), 1.14-0.97 (m, 6H), 0.97-0.72 (m, 13H)

Step 5. A suspension of tert-butyl 2-(4-((7-(((S)-1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-3-methoxyphenyl)pyrrolidine-1-carboxylate (146 mg, 0.152 mmol), Pd—C (32.3 mg, 0.015 mmol), and pyridine (0.012 mL, 0.152 mmol) in MeOH (10 mL) was purged 3 times with vacuum and nitrogen then purged three more times with vacuum and hydrogen. The mixture was then stirred under hydrogen for 16 h, filtered through CELITE™, and evaporated under reduced pressure. The crude product was purified on silica gel with a gradient of 0% to 100% of ethyl acetate in hexanes to provide tert-butyl 2-(4-((7-(((S)-1-((tert-butyldiphenylsilyl)oxy)hexan-3-yl)amino)-5-((methoxycarbonyl)amino)-1H-pyrazolo[4,3-d]pyrimidin-1-yl)methyl)-3-methoxyphenyl)pyrrolidine-1-carboxylate (100 mg).

LC-MS m/z 836.3 [M+H]⁺.

Step 6. To a solution of tert-butyl 2-(4-((7-(((S)-1-((tert-butyldiphenylsilyl)oxy)hexan-3-yl)amino)-5-((methoxycarbonyl)amino)-1H-pyrazolo[4,3-d]pyrimidin-1-yl)methyl)-3-methoxy-phenyl)pyrrolidine-1-carboxylate (100 mg, 0.120 mmol) in DCM (10 mL) was added TFA (500 μl, 6.49 mmol). After 16 h the solvent was evaporated under reduced pressure and dried under high vacuum to provide methyl (7-(((S)-1-((tert-butyldiphenylsilyl)oxy)hexan-3-yl)amino)-1-(2-methoxy-4-(pyrrolidin-2-yl)benzyl)-1H-pyrazolo[4,3-d]pyrimidin-5-yl)carbamate trifluoroacetate (102 mg). This product was used without further purification.

LC-MS m/z 736.3 [M+H]⁺.

Step 7. A mixture of methyl (7-(((S)-1-((tert-butyldiphenylsilyl)oxy)hexan-3-yl)amino)-1-(2-methoxy-4-(pyrrolidin-2-yl)benzyl)-1H-pyrazolo[4,3-d]pyrimidin-5-yl)carbamate (0.088 g, 0.12 mmol) and HCl (0.4 mL, 4.80 mmol) in MeOH (10 mL) was stirred at RT for 2 h, then concentrated under reduced pressure and dried under high vacuum to provide methyl (7-(((S)-1-hydroxyhexan-3-yl)amino)-1-(2-methoxy-4-(pyrrolidin-2-yl)benzyl)-1H-pyrazolo[4,3-d]pyrimidin-5-yl)carbamate (64 mg). This product was used without further purification.

LC-MS m/z 498.3 [M+H]⁺.

Step 8. A mixture of methyl (7-(((S)-1-hydroxyhexan-3-yl)amino)-1-(2-methoxy-4-(pyrrolidin-2-yl)benzyl)-1H-pyrazolo[4,3-d]pyrimidin-5-yl)carbamate (59.7 mg, 0.12 mmol) and NaOH (0.120 mL, 1.200 mmol) in dioxane (7 mL) was heated at 50° C. for 16 h. The reaction mixture was concentrated under reduced pressure, dried under high vacuum and diluted with DMF:Acetic acid 1:1 (2 mL). The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 4% B, 4-100% B over 20 minutes, then a 0-minute hold at 100% B; Flow Rate: 20 mL/min; Column Temperature: 25° C. Fraction collection was triggered by MS and UV signals. Fractions containing Compound 175 were combined and dried via centrifugal evaporation.

### Example 14—Compound 179

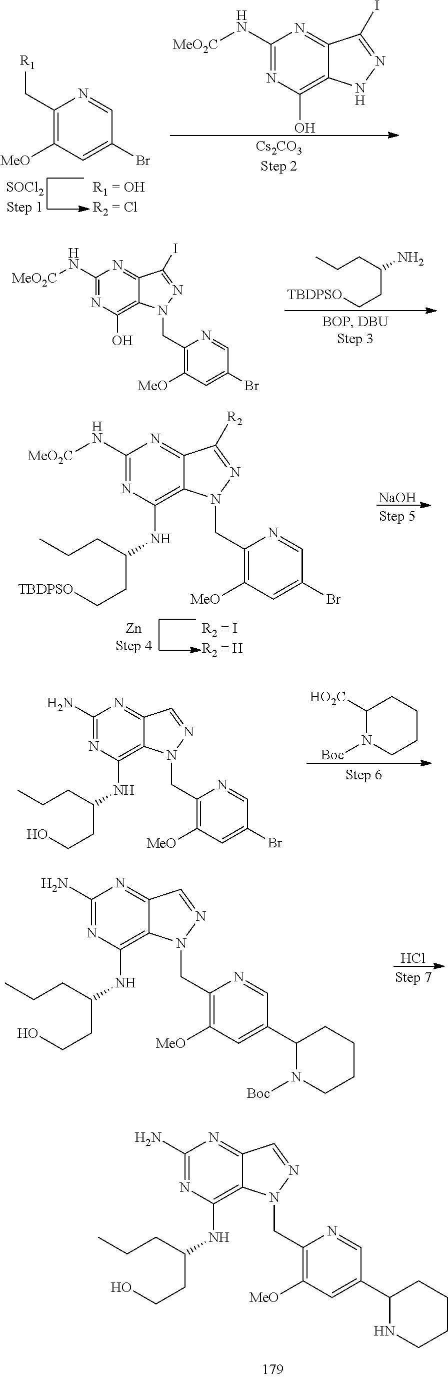

Step 1. To a 0° C. solution of (5-bromo-3-methoxypyridin-2-yl)methanol (Sigma-Aldrich) (2.462 g, 11.29 mmol) in CH₂Cl₂ (113 ml) was added SOCl₂ (1.235 ml, 16.94 mmol), dropwise. The reaction was stirred at RT for 1 h, then it was concentrated in vacuo. This material was mixed with CH₂Cl₂ and concentrated in vacuo (2×) to provide the crude product, 5-bromo-2-(chloromethyl)-3-methoxypyridine. This material was used without further purification.

LC-MS m/z 236/238 [M+H]⁺.

Step 2 To a RT suspension of methyl (7-hydroxy-3-iodo-1H-pyrazolo[4,3-d]pyrimidin-5-yl)carbamate (3.44 g, 10.26 mmol) in DMF (45.6 ml) was added Cs₂CO₃ (13.37 g, 41.0 mmol). The reaction mixture was stirred at 0° C. for 10 min; then a solution of the crude material from Step 1 in DMF (22.80 ml) was added. The reaction mixture was stirred at 0° C. for 1 h, then the cooling bath was removed and stirring was continued at RT for 20 h. The reaction mixture was added to H₂O (250 mL) and allowed to stand at RT. The solids were collected by vacuum filtration and washed with H₂O (3×15 mL), MeOH (2×15 mL), CH₂Cl₂ (15 mL), and hexanes (15 mL) to provide methyl (1-((5-bromo-3-methoxypyridin-2-yl)methyl)-7-hydroxy-3-iodo-1H-pyrazolo[4,3-d]pyrimidin-5-yl)carbamate (4.431 g, 81%).

LC-MS m/z 535/537 [M+H]⁺.

¹H NMR (400 MHz, DMSO-d₆) δ 13.19-12.96 (m, 1H), 8.95-8.80 (m, 1H), 8.06 (s, 1H), 7.71 (s, 1H), 5.87-5.65 (m, 2H), 3.89 (s, 3H), 3.53 (br s, 3H).

Step 3. To a RT suspension of methyl (1-((5-bromo-3-methoxypyridin-2-yl)methyl)-7-hydroxy-3-iodo-1H-pyrazolo[4,3-d]pyrimidin-5-yl)carbamate (0.990 g, 1.850 mmol) in DMSO (12.33 ml) was added (S)-1-((tert-butyldiphenylsilyl)oxy)hexan-3-amine, HCl salt (0.870 g, 2.220 mmol) (US 2020/0038403 A1, FIG. 8, compound 71a), followed by 1,8-diazabicyclo[5.4.0]undec-7-ene (1.245 ml, 8.33 mmol) and (benzotriazol-1-yloxy)tris(dimethylamino)phosphonium hexafluorophosphate (0.982 g, 2.220 mmol). The reaction mixture was stirred at RT for 1 h, diluted with EtOAc (100 mL), and washed with H₂O (100 mL). The layers were separated, and the aqueous layer was extracted with EtOAc (100 mL). The combined organic layers were washed with saturated aqueous NaCl (100 mL), dried over Na₂SO₄, filtered, and concentrated in vacuo. The crude material was purified by flash chromatography (80 g silica gel; linear gradient 0-100% EtOAc-hexanes) to provide methyl (S)-(1-((5-bromo-3-methoxypyridin-2-yl)methyl)-7-((1-((tert-butyldiphenylsilyl)oxy)hexan-3-yl)amino)-3-iodo-1H-pyrazolo[4,3-d]pyrimidin-5-yl)carbamate (810 mg, 50%) as a yellow foam.

LC-MS m/z 872/874 [M+H]⁺.

¹H NMR (400 MHz, DMSO-d₆) δ 9.69 (s, 1H), 7.92 (d, J=1.8 Hz, 1H), 7.79 (d, J=1.8 Hz, 1H), 7.57-7.53 (m, 2H), 7.50-7.46 (m, 2H), 7.42-7.31 (m, 4H), 7.25-7.20 (m, 2H), 7.12 (d, J=8.3 Hz, 1H), 5.78-5.69 (m, 2H), 4.64-4.55 (m, 1H), 3.91 (s, 3H), 3.70-3.64 (m, 2H), 3.58 (s, 3H), 1.90-1.82 (m, 2H), 1.57-1.48 (m, 2H), 1.25-1.13 (m, 2H), 0.92 (s, 9H), 0.81 (t, J=7.3 Hz, 3H).

Step 4. To a 0° C. solution of methyl (S)-(1-((5-bromo-3-methoxypyridin-2-yl)methyl)-7-((1-((tert-butyldiphenylsilyl)oxy)hexan-3-yl)amino)-3-iodo-1H-pyrazolo[4,3-d]pyrimidin-5-yl)carbamate (0.810 g, 0.928 mmol) in a mixture of MeOH (9.28 ml) and AcOH (9.28 ml) was added zinc (0.607 g, 9.28 mmol). The reaction mixture was stirred at 0° C. for 30 min, filtered through CELITE™, and washed with MeOH (10 mL) and EtOAc (50 mL). The filtrate was diluted with EtOAc (200 mL). While stirring, saturated aqueous NaHCO₃ (250 mL) was slowly added to this solution (with care to control the rate of gas evolution). The layers were separated and the organic layer was washed with saturated aqueous NaCl (250 mL), dried over Na₂SO₄, filtered, and concentrated in vacuo to provide the crude product, methyl (S)-(1-((5-bromo-3-methoxy-pyridin-2-yl)methyl)-7-((1-((tert-butyldiphenylsilyl)oxy)hexan-3-yl)amino)-1H-pyrazolo[4,3-d]pyrimidin-5-yl)carbamate. This material was used without further purification.

LC-MS m/z 746/748 [M+H]⁺.

Step 5. To a RT solution of the crude material from Step 4 in 1,4-dioxane (9518 l) and MeOH (4759 μl) was added 10 M aqueous NaOH (928 μl, 9.28 mmol). The reaction mixture was stirred at 65° C. for 16 h, cooled to RT, diluted with H₂O (100 mL), and extracted with EtOAc (3×100 mL). The combined organic layers were washed with saturated aqueous NaCl (100 mL), dried over Na₂SO₄, filtered, and concentrated in vacuo. The crude material was purified by flash chromatography (24 g silica gel; linear gradient 0-10% MeOH—CH₂Cl₂) to provide (S)-3-((5-amino-1-((5-bromo-3-methoxypyridin-2-yl)methyl)-1H-pyrazolo[4,3-d]pyrimidin-7-yl)amino)hexan-1-ol (368 mg, 88%) as a light yellow foam.

LC-MS m/z 450/452 [M+H]⁺.

¹H NMR (400 MHz, DMSO-d₆) δ 8.13 (d, J=1.9 Hz, 1H), 7.82 (d, J=1.9 Hz, 1H), 7.47 (s, 1H), 6.86 (d, J=8.4 Hz, 1H), 5.75-5.68 (m, 1H), 5.66-5.60 (m, 1H), 5.57 (s, 2H), 4.43 (t, J=5.3 Hz, 1H), 4.41-4.34 (m, 1H), 3.91 (s, 3H), 3.47-3.41 (m, 2H), 1.78-1.62 (m, 2H), 1.54-1.46 (m, 2H), 1.27-1.16 (m, 2H), 0.83 (t, J=7.3 Hz, 3H).

Step 6. A mixture of (S)-3-((5-amino-1-((5-bromo-3-methoxypyridin-2-yl)methyl)-1H-pyrazolo[4,3-d]pyrimidin-7-yl)amino)hexan-1-ol (28 mg, 0.062 mmol), 1-(tert-butoxycarbonyl)-piperidine-2-carboxylic acid (28.5 mg, 0.124 mmol), NiBr₂.dtbbpy (1.514 mg, 3.11 μmol), and Ir[dF(CF₃)ppy]₂(dtbbpy)PF₆ (0.698 mg, 0.622 μmol) was evacuated and back-filled with N₂. DMA (1244 μl) was added, followed by 1,8-diazabicyclo[5.4.0]undec-7-ene (18.60 μl, 0.124 mmol). The reaction mixture was sparged with N₂ for 20 min, then sealed and stirred under irradiation with purple LEDs (395-405 nm) with a cooling fan for 18 h. This reaction mixture was combined with the reaction mixture from a repeated run of this reaction under similar conditions. The resulting mixture was diluted with H₂O (20 mL) and extracted with EtOAc (2×20 mL). The combined organic layers were washed with saturated aqueous NaCl (20 mL), dried over Na₂SO₄, filtered, and concentrated in vacuo. The crude material was purified by flash chromatography (12 g silica gel; linear gradient 0-10% MeOH—CH₂Cl₂) to provide tert-butyl 2-(6-((5-amino-7-(((S)-1-hydroxyhexan-3-yl)amino)-1H-pyrazolo[4,3-d]pyrimidin-1-yl)methyl)-5-methoxypyridin-3-yl)piperidine-1-carboxylate (mixture of diastereomers) as a mixture with additional impurities. This material was used without further purification.

LC-MS m/z 555 [M+H]⁺.

Step 7. To a RT solution of the material from Step 6 in 1,4-dioxane (894 l) and MeOH (224 μL) was added 4 M HCl in 1,4-dioxane (279 μL, 1.118 mmol). The reaction mixture was stirred at RT for 3 h and concentrated. The crude material was dissolved in MeOH and concentrated. The crude material was re-dissolved in DMF (2 mL), filtered (0.45 am nylon syringe filter), and purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 2 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 3% B, 3-43% B over 20 minutes, then a 0-min hold at 100% B; Flow Rate: 20 mL/min; Column Temperature: 25° C. Fraction collection was triggered by MS signals. Fractions containing the desired product were combined and dried via centrifugal evaporation to provide Compound 179 (14.5 mg).

Compound 178 was analogously prepared.

### Example 15—Compound 183

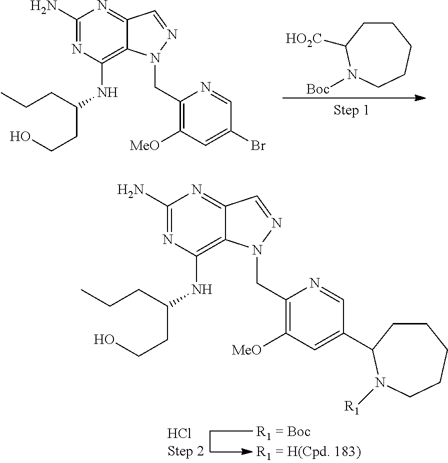

Step 1. A mixture of (S)-3-((5-amino-1-((5-bromo-3-methoxypyridin-2-yl)methyl)-1H-pyrazolo[4,3-d]pyrimidin-7-yl)amino)hexan-1-ol (40 mg, 0.089 mmol), 1-(tert-butoxycarbonyl)-azepane-2-carboxylic acid (32.4 mg, 0.133 mmol), NiBr₂.dtbbpy (2.162 mg, 4.44 μmol), and Ir[dF(Me)ppy]₂(dtbbpy)PF₆ (0.901 mg, 0.888 μmol) was dissolved in DMA (1776 l), and 1,8-diazabicyclo[5.4.0]undec-7-ene (19.92 μl, 0.133 mmol) was added. The reaction flask was sparged with N₂ for 10 min and sealed The reaction mixture was stirred under irradiation by purple LEDs (395-405 nm) with a cooling fan for 22 h. The reaction mixture was diluted with saturated aqueous NaHCO₃ (20 mL) and extracted with EtOAc (3×20 mL). The combined organic layers were washed with saturated aqueous NaCl (20 mL), dried over Na₂SO₄, filtered, and concentrated in vacuo. The crude material was purified by flash chromatography (12 g silica gel; linear gradient 0-10% MeOH—CH₂Cl₂) to provide tert-butyl 2-(6-((5-amino-7-(((S)-1-hydroxyhexan-3-yl)amino)-1H-pyrazolo[4,3-d]pyrimidin-1-yl)methyl)-5-methoxypyridin-3-yl)azepane-1-carboxylate (mixture of diastereomers) (24.7 mg, 49%).

LC-MS m/z 569 [M+H]⁺.

Step 2. To a RT solution of tert-butyl 2-(6-((5-amino-7-(((S)-1-hydroxyhexan-3-yl)amino)-1H-pyrazolo[4,3-d]pyrimidin-1-yl)methyl)-5-methoxypyridin-3-yl)azepane-1-carboxylate (mixture of diastereomers) (24.7 mg) in 1,4-dioxane (347 μl) and MeOH (87 μL) was added 4 M HCl in dioxane (109 μl, 0.434 mmoL). The reaction mixture was stirred at RT for 3 h and concentrated. The crude material was dissolved in MeOH and concentrated. The crude material was re-dissolved in DMF (2 mL), filtered (0.45 am nylon syringe filter), and purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to provide Compound 183 (13.4 mg).

### Example 16—Compound 185

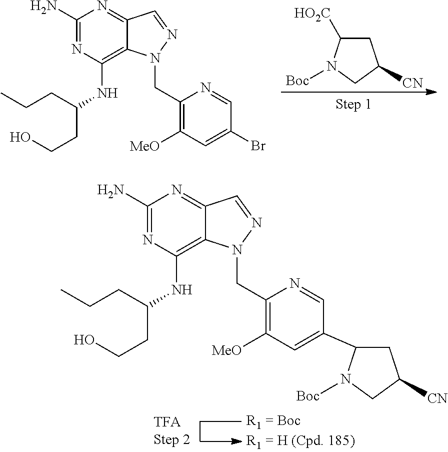

Step 1. A mixture of (S)-3-((5-amino-1-((5-bromo-3-methoxypyridin-2-yl)methyl)-1H-pyrazolo[4,3-d]pyrimidin-7-yl)amino)hexan-1-ol (40 mg, 0.089 mmol), (2S,4R)-1-(tert-butoxycarbonyl)-4-cyanopyrrolidine-2-carboxylic acid (42.7 mg, 0.178 mmol), NiBr₂.dtbbpy (2.162 mg, 4.44 μmol), and Ir[dF(Me)ppy]₂(dtbbpy)PF₆ (0.901 mg, 0.888 μmol) was dissolved in DMA (1776 μL), and 1,8-diazabicyclo[5.4.0]undec-7-ene (26.6 μl, 0.178 mmol) was added. The reaction mixture was sparged with N₂ for 10 min, then it was sealed and stirred under irradiation by purple LEDs (395-405 nm) with a cooling fan for 17 h. The reaction was diluted with saturated aqueous NaHCO₃ (20 mL) and extracted with EtOAc (3×20 mL). The combined organic layers were washed with saturated aqueous NaCl (20 mL), dried over Na₂SO₄, filtered, and concentrated in vacuo. The crude material was purified by flash chromatography (12 g silica gel; linear gradient 0-20% MeOH—CH₂Cl₂) to provide tert-butyl (4R)-2-(6-((5-amino-7-(((S)-1-hydroxyhexan-3-yl)amino)-1H-pyrazolo[4,3-d]pyrimidin-1-yl)methyl)-5-methoxypyridin-3-yl)-4-cyanopyrrolidine-1-carboxylate (mixture of diastereomers) (25.4 mg, 51%).

LC-MS m/z 566 [M+H]⁺.

Step 2. To a RT solution of tert-butyl (4R)-2-(6-((5-amino-7-(((S)-1-hydroxyhexan-3-yl)amino)-1H-pyrazolo[4,3-d]pyrimidin-1-yl)methyl)-5-methoxypyridin-3-yl)-4-cyanopyrrolidine-1-carboxylate (mixture of diastereomers) (25.4 mg, 0.045 mmol) in CH₂Cl₂ (808 al) was added TFA (90 al). The reaction was stirred at RT for 5 h. The reaction was concentrated in vacuo. The crude material was dissolved in CH₂Cl₂ and concentrated in vacuo (2×).

The crude material was dissolved in a mixture of CH₂Cl₂ (225 μL) and MeOH (225 μL), and triethylamine (31.3 μl, 0.225 mmol) was added. The solution was stirred at RT for 20 min. The reaction was concentrated. The crude material was dissolved in CH₂Cl₂ and concentrated. The crude material was dissolved in DMF (2 mL), filtered (0.45 am nylon syringe filter), and purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to provide Compound 185 (mixture of diastereomers), TFA salt (19.3 mg).

### Example 17—Compound 171 and Compound 172

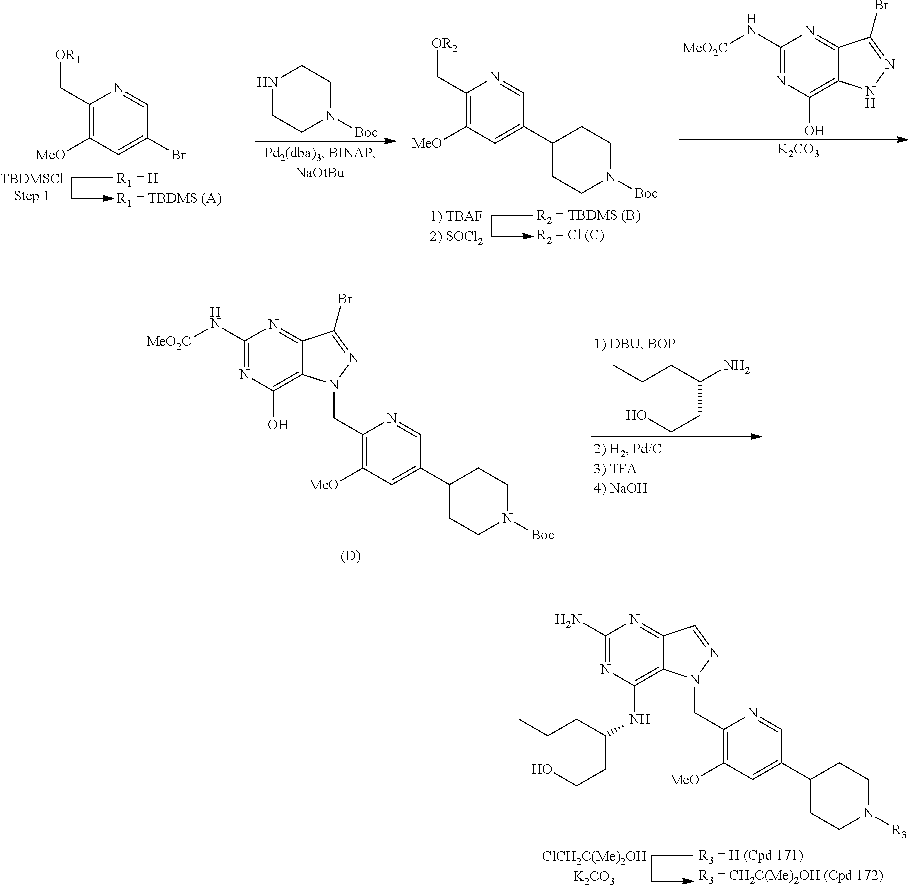

Compound A. A solution of (5-bromo-3-methoxypyridin-2-yl)methanol (3.75 g, 17.20 mmol) in DMF (20 mL) was treated with TBDMS-Cl (6.48 g, 43.0 mmol), followed by imidazole (2.58 g, 37.8 mmol). The resulting reaction mixture was stirred at RT for 3 h, quenched with water, and extracted with ethyl acetate (3×50 mL). The combined organic extracts were dried over Na₂SO₄ and filtered. The filtrate was concentrated. The residue was purified by silica column (80 g), eluted with ethyl acetate:hexanes, 0-30% gradient. The desired fractions were concentrated to yield 5-bromo-2-(((tert-butyldimethylsilyl)oxy)methyl)-3-methoxypyridine A (5.55 g, 16.70 mmol, 97% yield).

LCMS ESI: calculated for C₁₃H₂₃BrNO₂Si=332.1, 334.1 (M+H⁺), found 332.0, 334.0 (M+H⁺).

¹H NMR (400 MHz, CHLOROFORM-d) δ 8.14 (d, J=2.0 Hz, 1H), 7.19 (d, J=2.0 Hz, 1H), 4.69 (s, 2H), 3.75 (s, 3H), 0.83-0.75 (m, 9H), 0.02-−0.03 (m, 6H).

Compound B. A stirred solution of 5-bromo-2-(((tert-butyldimethylsilyl)oxy)methyl)-3-methoxypyridine A (2.0 g, 6.02 mmol) in toluene (25 mL) was treated with sodium tert-butoxide (1.157 g, 12.04 mmol), tert-butyl piperazine-1-carboxylate (1.681 g, 9.03 mmol), and BINAP (1.874 g, 3.01 mmol). A stream of N₂ was bubbled into the reaction mixture for 5 min; then Pd₂(dba)₃ (0.551 g, 0.602 mmol) was added to the mixture. The reaction mixture was heated at 100° C. for 16 h, cooled, diluted with EtOAc, and filtered through CELITE™, washing with EtOAC. The filtrate was concentrated. The crude compound was purified by flash chromatography using 80 g column eluted with EA:hexanes 0-80% gradient, the desired fractions were concentrated to yield tert-butyl 4-(6-(((tert-butyldimethylsilyl)oxy)methyl)-5-methoxypyridin-3-yl)piperazine-1-carboxylate B (1.22 g, 2.79 mmol, 46.3% yield).

LCMS ESI: calculated for C₂₂H₄₀N₃O₄Si=438.3 (M+H⁺), found 438.3 (M+H⁺).

¹H NMR (400 MHz, DMSO-d6) δ 7.76 (d, J=2.2 Hz, 1H), 6.92 (d, J=2.2 Hz, 1H), 4.58 (s, 2H), 3.78 (s, 3H), 3.49-3.38 (m, 4H), 3.17 (br s, 4H), 1.40 (s, 9H), 0.82 (s, 9H), 0.00 (s, 6H).

Compound C, Part 1. A suspension of tert-butyl 4-(6-(((tert-butyldimethylsilyl)-oxy)methyl)-5-methoxypyridin-3-yl)piperazine-1-carboxylate B (550 mg, 1.257 mmol) in THF (5 mL) was treated with TBAF (1.0 M in THF) (1.257 mL, 1.257 mmol) at RT. The reaction mixture was stirred at RT for 16 h and concentrated. The crude residue was purified by silica column (40 g), eluted with 20% MeOH in DCM:DCM, 0-25% gradient, the desired fraction was concentrated to yield tert-butyl 4-(6-(hydroxymethyl)-5-methoxypyridin-3-yl)piperazine-1-carboxylate (405 mg, 1.277 mmol, 100% yield) as a brown oil.

LCMS ESI: calculated for C₁₆H₂₆N₃O₄=324.2 (M+H⁺), found 324.1 (M+H⁺).

¹H NMR (400 MHz, DMSO-d6) δ 7.84 (d, J=2.2 Hz, 1H), 6.98 (d, J=2.2 Hz, 1H), 5.01 (s, 2H), 3.83 (s, 3H), 3.58-3.41 (m, 4H), 3.26-3.17 (m, 4H), 1.43 (s, 9H), one exchangeable proton was not visible.

Compound C, Part 2. A mixture of tert-butyl 4-(6-(hydroxymethyl)-5-methoxy-pyridin-3-yl)piperazine-1-carboxylate (230 mg, 0.711 mmol) in anhydrous THF (5 mL) was treated with SOCl₂ (0.260 mL, 3.56 mmol) at 0° C. for 5 min. The reaction mixture was concentrated. The excess SOCl₂ was azeotropically removed with DCM (3×). The resulting crude material tert-butyl 4-(6-(chloromethyl)-5-methoxypyridin-3-yl)piperazine-1-carboxylate C was directly carried over to the next step without purification.

LCMS ESI: calculated for C₁₆H₂₆N₃O₄=338.2 (M+H⁺), found 338.2 (M+H⁺).

Compound D. To a stirred mixture of methyl (3-bromo-7-hydroxy-2H-pyrazolo[4,3-d]pyrimidin-5-yl)carbamate (0.219 g, 0.761 mmol) in DMF (5 mL) was added K₂CO₃ (0.162 g, 1.170 mmol), followed by tert-butyl 4-(6-(chloromethyl)-5-methoxypyridin-3-yl)piperazine-1-carboxylate (0.2 g, 0.585 mmol) at 0° C. After 5 mins, LCMS indicated reaction was completed with major desired product. The reaction mixture was quenched with saturated NH₄Cl solution and stirred at RT for 1 h The resulting solid was collected by filtration and purified by ISCO silica column (40 g), eluting with ethyl acetate:hexanes 0-100% gradient. The desired fractions were concentrated to yield tert-butyl 4-(6-((3-bromo-7-hydroxy-5-((methoxycarbonyl)amino)-1H-pyrazolo[4,3-d]pyrimidin-1-yl)methyl)-5-methoxypyridin-3-yl)piperazine-1-carboxylate D (150 mg, 0.253 mmol, 43.2% yield) as a white solid.

LCMS ESI: calculated for C₂₃H₃₀BrN₈O₂=593.1 595.1 (M+H⁺), found 593.1, 595.1 (M+H⁺).

¹H NMR (400 MHz, DMSO-d6) δ 8.03-7.83 (m, 1H), 7.68 (d, J=2.4 Hz, 1H), 7.00 (d, J=2.2 Hz, 1H), 5.68 (s, 2H), 3.83 (s, 3H), 3.75 (s, 3H), 3.44 (br d, J=5.3 Hz, 4H), 3.18 (br t, J=5.0 Hz, 4H), 1.42 (s, 9H). One exchangeable proton was not visible.

Compound 171. (S)-3-((5-amino-1-((3-methoxy-5-(piperazin-1-yl)pyridin-2-yl)methyl)-1H-pyrazolo[4,3-d]pyrimidin-7-yl)amino)hexan-1-ol (Compound 171) was prepared from Compound D analogously following the procedure above for Compound 124. See Table A for analytical data.

Compound 172. A solution of (S)-3-((5-amino-1-((3-methoxy-5-(piperazin-1-yl)pyridin-2-yl)methyl)-1H-pyrazolo[4,3-d]pyrimidin-7-yl)amino)hexan-1-ol, (25 mg, 0.052 mmol) in DMF (1 mL) was treated with 1-chloro-2-methylpropan-2-ol (34.0 mg, 0.313 mmol), and catalytic amount of NaI. The reaction mixture was then stirred at 100° C. for 20 h. Upon cooling, the solid was filtered off, and filtrate was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 16% B, 16-56% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to yield Compound 172 (6.6 mg, 0.012 mmol, 23.58% yield).

### Example 18—Compound 186

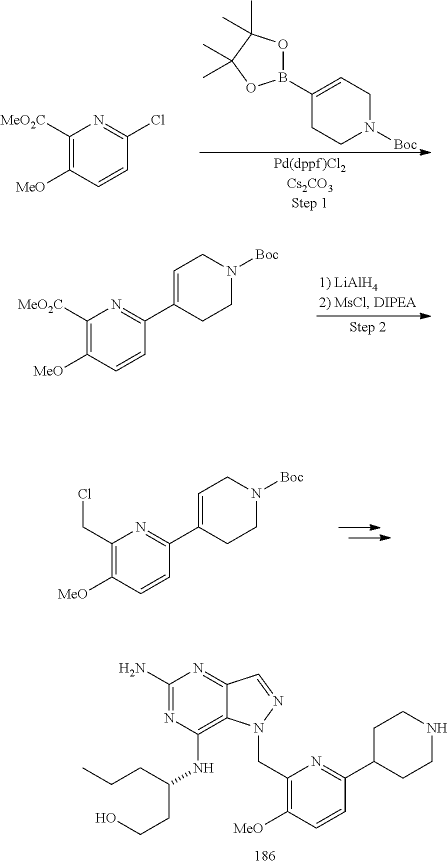

Step 1. A mixture of tert-butyl 4-(4,4,5,5-tetramethyl-1,3,2-dioxaborolan-2-yl)-3,6-dihydropyridine-1(2H)-carboxylate (1252 mg, 4.05 mmol) in dioxane (4 mL) and water (1 mL) was treated with methyl 6-chloro-3-methoxypicolinate (680 mg, 3.37 mmol), K₂CO₃ (1.63 g, 11.81 mmol). After a stream of N₂ gas was bubbled into the mixture for 2 min; then PdCl₂(dppf)-CH₂Cl₂ adduct (275 mg, 0.337 mmol) was added. The resulting mixture was stirred at 70° C. for 2.5 h. Upon cooling, the reaction mixture was diluted with EtOAc (5 mL). The solid material was removed by filtration through a CELITE™ pad. The filtrate was dried over Na₂SO₄, filtered and concentrated. The crude material was purified by silica column (40 g), eluting with EtOAc/hexanes 0-100% gradient. The desired fractions were concentrated to yield 1′-(tert-butyl) 6-methyl 5-methoxy-3′,6′-dihydro-[2,4′-bipyridine]-1′,6(2′H)-dicarboxylate (compound 1) (700 mg, 2.009 mmol, 59.6% yield).

LCMS ESI: calculated for C₁₈H₂₅N₂O₅=349.2 (M+H⁺), found 349.1 (M+H⁺).

¹H NMR (400 MHz, DMSO-d6) δ 7.74-7.66 (m, 1H), 7.67-7.42 (m, 1H), 6.55 (br s, 1H), 4.03 (br d, J=2.2 Hz, 2H), 3.86 (s, 3H), 3.84 (s, 3H), 3.53 (t, J=5.7 Hz, 2H), 2.52 (br s, 2H), 1.43 (s, 9H).

Step 2, Part 1. A solution of 1′-(tert-butyl) 6-methyl 5-methoxy-3′,6′-dihydro-[2,4′-bipyridine]-1′,6(2′H)-dicarboxylate (1.2 g, 3.44 mmol) in THF (30 mL) was treated with LiAlH₄ (3.44 mL, 3.44 mmol) in portions under N₂ at 0° C. The reaction mixture was stirred for 30, carefully quenched with Rochelle salt solution, and stirred at RT for 16 h. The two liquid phases were separated. The aqueous phase was back extracted with EtOAc (2×25 mL). The combined organic extracts were dried over Na₂SO₄, filtered and concentrated to yield tert-butyl 6-(hydroxymethyl)-5-methoxy-3′,6′-dihydro-[2,4′-bipyridine]-1′(2′H)-carboxylate (1.1 g, 3.43 mmol, 100% yield) which was pure enough for use in the next step.

LCMS ESI: calculated for C₁₇H₂₅N₂O₄=321.2 (M+H⁺), found 321.2 (M+H⁺).

¹H NMR (400 MHz, CHLOROFORM-d) δ 7.30-7.25 (m, 1H), 7.12 (d, J=8.4 Hz, 1H), 6.54 (br s, 1H), 4.73 (s, 2H), 4.43 (br s, 1H), 4.14-4.11 (m, 2H), 3.86 (s, 3H), 3.65 (br t, J=5.7 Hz, 2H), 2.70-2.58 (m, 2H), 1.49 (s, 9H).

Step 2, Part 2. A mixture of tert-butyl 6-(hydroxymethyl)-5-methoxy-3′,6′-dihydro-[2,4′-bipyridine]-1′(2′H)-carboxylate (1.1 g, 3.43 mmol) in DCM (20 mL) was treated with Hunig's base (0.899 mL, 5.15 mmol), followed by Ms-Cl (0.321 mL, 4.12 mmol). The reaction mixture was stirred at RT for 16 h, diluted with DCM (10 mL), and washed with water. The two layers were separated. The organic layer was dried over Na₂SO₄, filtered and concentrated. The crude material was purified by silica column (40 g), eluting with EtOAc:hexanes 0-70% gradient. The desired fractions were concentrated to yield tert-butyl 6-(chloromethyl)-5-methoxy-3′,6′-dihydro-[2,4′-bipyridine]-1′(2′H)-carboxylate compound 2 (724 mg, 2.137 mmol, 62.2% yield).

LCMS ESI: calculated for C₁₇H₂₅N₂O₄=321.2 (M+H⁺), found 321.2 (M+H⁺).

¹H NMR (400 MHz, CHLOROFORM-d) δ 7.31 (d, J=8.6 Hz, 1H), 7.18 (d, J=8.6 Hz, 1H), 6.54 (br s, 1H), 4.73 (s, 2H), 4.12 (br d, J=2.9 Hz, 2H), 3.90 (s, 3H), 3.64 (br t, J=5.5 Hz, 2H), 2.62 (br dd, J=4.4, 2.6 Hz, 2H), 1.49 (s, 9H)

tert-Butyl 6-(chloromethyl)-5-methoxy-3′,6′-dihydro-[2,4′-bipyridine]-1′(2′H)-carboxylate was taken over multiple steps, performed analogously to those for Compound 2, to Compound 186.

### Example 19—Compound 192 and Compound 196

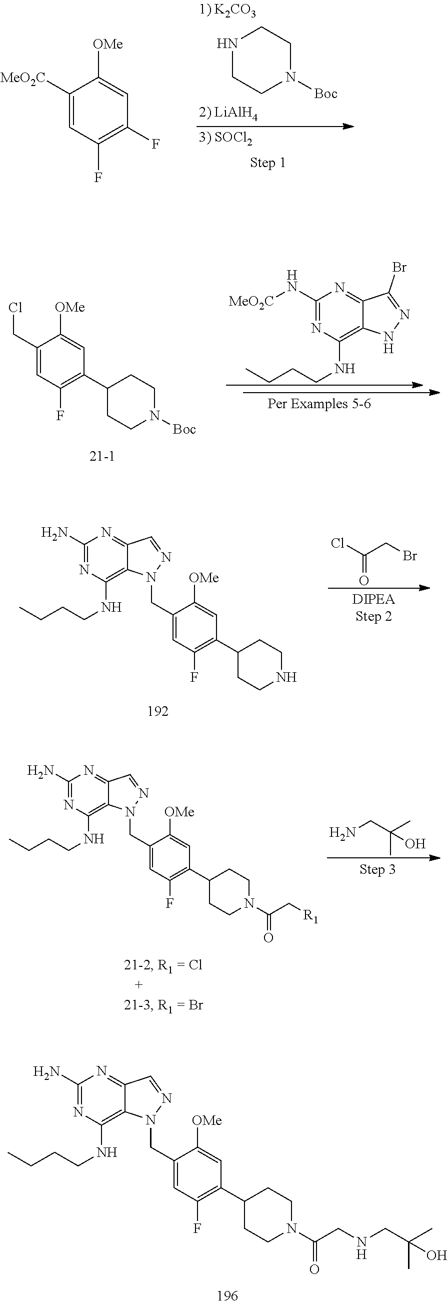

Step 1, Part 1. A mixture of methyl 4,5-difluoro-2-methoxybenzoate (1.0 g, 4.95 mmol), tert-butyl piperazine-1-carboxylate (1.013 g, 5.44 mmol) and K₂CO₃ (1.367 g, 9.89 mmol) in DMF (20 mL) was stirred at 90° C. for 16 h, cooled, and quenched with water. The reaction mixture was extracted with EtOAc (3×25 mL). The combined organic extracts were dried over Na₂SO₄, filtered and concentrated. The resulting crude material was purified by ISCO silica column chromatography (80 g), eluting with EtOAc-hexanes 0-80% gradient. The desired fractions were concentrated to yield tert-butyl 4-(2-fluoro-5-methoxy-4-(methoxycarbonyl)-phenyl)piperazine-1-carboxylate (1.31 g, 3.56 mmol, 71.9% yield).

LCMS ESI: calculated for C₁₈H₂₆FN₂O₅=369.2 (M+H⁺), found 369.1 (M+H⁺).

¹H NMR (400 MHz, CHLOROFORM-d) δ 7.57 (d, J=13.4 Hz, 1H), 6.48 (d, J=7.0 Hz, 1H), 3.88 (s, 3H), 3.85 (s, 3H), 3.65-3.58 (m, 4H), 3.22-3.11 (m, 4H), 1.49 (s, 9H).

Step 1, Part 2. A solution of tert-butyl 4-(2-fluoro-5-methoxy-4-(methoxycarbonyl)-phenyl)piperazine-1-carboxylate (1.3 g, 3.53 mmol) in THF (30 mL) was treated with LiAlH₄ (2.0 M in THF) (1.764 mL, 3.53 mmol) in portions under N₂ at RT. After 10 min, the reaction was carefully quenched with Rochelle salt solution. After stirring for 16 h at RT, the two layers were separated. The organic layer was dried over Na₂SO₄, filtered and concentrated to yield tert-butyl 4-(2-fluoro-4-(hydroxymethyl)-5-methoxyphenyl)piperazine-1-carboxylate (1.20 g, 3.53 mmol, 100% yield) which was pure enough to use in the next step.

LCMS ESI: calculated for C₁₇H₂₆FN₂O₄=341.2 (M+H⁺), found 341.2 (M+H⁺).

¹H NMR (400 MHz, CHLOROFORM-d) δ 7.00 (d, J=12.3 Hz, 1H), 6.46 (d, J=6.8 Hz, 1H), 4.59 (br s, 2H), 3.88-3.82 (m, 3H), 3.69-3.40 (m, 4H), 3.17-2.85 (m, 4H), 1.48 (s, 9H).

Step 1, Part c. A solution of tert-butyl 4-(2-fluoro-4-(hydroxymethyl)-5-methoxy-phenyl)piperazine-1-carboxylate (1.2 g, 3.53 mmol) in THF (20 mL) was treated with SOCl₂ (0.334 mL, 4.58 mmol) at 0° C. After 10 min, LCMS only showed starting material. However, NMR indicated the crude product was the desired tert-butyl 4-(4-(chloromethyl)-2-fluoro-5-methoxyphenyl)piperazine-1-carboxylate (Compound 21-1), which was directly carried over to the next step.

¹H NMR (400 MHz, CHLOROFORM-d) δ 8.30 (d, J=6.4 Hz, 1H), 7.33 (d, J=12.5 Hz, 1H), 4.59 (s, 2H), 4.11 (br t, J=4.8 Hz, 4H), 3.93 (s, 3H), 3.61 (br t, J=5.1 Hz, 4H), 1.50 (s, 9H).

Compound 192 was prepared from methyl (3-bromo-7-(butylamino)-1H-pyrazolo[4,3-d]pyrimidin-5-yl)carbamate and tert-butyl 4-(4-(chloromethyl)-2-fluoro-5-methoxyphenyl)piperazine-1-carboxylate (Compound 21-1) by reactions analogous to those used to prepare Compound 101 (Examples 5 and 6 above).

LCMS ESI: calculated for C₂₁H₃₀FN₈O=429.2 (M+H⁺), found 429.1 (M+H⁺).

¹H NMR (500 MHz, DMSO-d₆) δ 9.54-9.23 (m, 1H), 7.97 (s, 1H), 7.87 (br s, 2H), 7.01 (br d, J=12.5 Hz, 1H), 6.69 (br d, J=7.0 Hz, 1H), 5.41 (s, 2H), 3.82 (s, 3H), 3.26 (s, 4H), 1.68-1.51 (m, 2H), 1.41-1.23 (m, 2H), 0.91 (br t, J=7.3 Hz, 3H). Seven protons were not visible, most likely due to the water suppression and the overlap with the DMSO-d6 peak.

Step 2. A mixture of N7-butyl-1-(5-fluoro-2-methoxy-4-(piperazin-1-yl)benzyl)-1H-pyrazolo[4,3-d]pyrimidine-5,7-diamine and TFA (150 mg, 0.276 mmol) in THF (5 mL) was treated with Hunig's base (0.145 mL, 0.829 mmol), followed by 2-bromoacetyl chloride (43.5 mg, 0.276 mmol) dropwise at RT. After 5 min, the reaction was quenched with water and extracted with EtOAc (3×15 mL). The combined organic extracts was dried over Na₂SO₄, filtered and concentrated. The crude product was purified by ISCO silica column (12 g), eluting with 20% MeOH in DCM:DCM 0-60% gradient. The desired fractions were concentrated to yield 153 mg of a mixture of 1-(4-(4-((5-amino-7-(butylamino)-1H-pyrazolo[4,3-d]pyrimidin-1-yl)methyl)-2-fluoro-5-methoxyphenyl)piperazin-1-yl)-2-chloroethan-1-one (Compound 21-2) and 1-(4-(4-((5-amino-7-(butylamino)-1H-pyrazolo[4,3-d]pyrimidin-1-yl)methyl)-2-fluoro-5-methoxyphenyl)-piperazin-1-yl)-2-bromoethan-1-one (Compound 21-3).

LCMS ESI (Compound 21-2): calculated for C₂₃H₃₁ClFN₈O₂=505.2 (M+H⁺), found 505.2 (M+H⁺).

LCMS ESI (Compound 22-3): calculated for C₂₃H₃₁BrFN₈O₂=548.2, 550.2 (M+H⁺), found 548.2, 550.2 (M+H⁺).

Step 3. A mixture of Compound 21-2 and compound 21-3 (25 mg,) in N,N-dimethylacetamide (0.5 mL) was treated with 1-amino-2-methylpropan-2-ol (44.1 mg, 0.495 mmol). After stirring at 60° C. for 16 hrs, LCMS indicated that reaction was complete. The mixture was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 8% B, 8-48% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to yield Compound 196) (22 mg).

The following compounds were analogously prepared: Compound 194, Compound 195, Compound 197, Compound 198, and Compound 199.

Compound 193 was prepared from Compound 192 using a procedure analogous to that used in Example 8 above to prepare Compound 103.

### Example 20—Compound 149

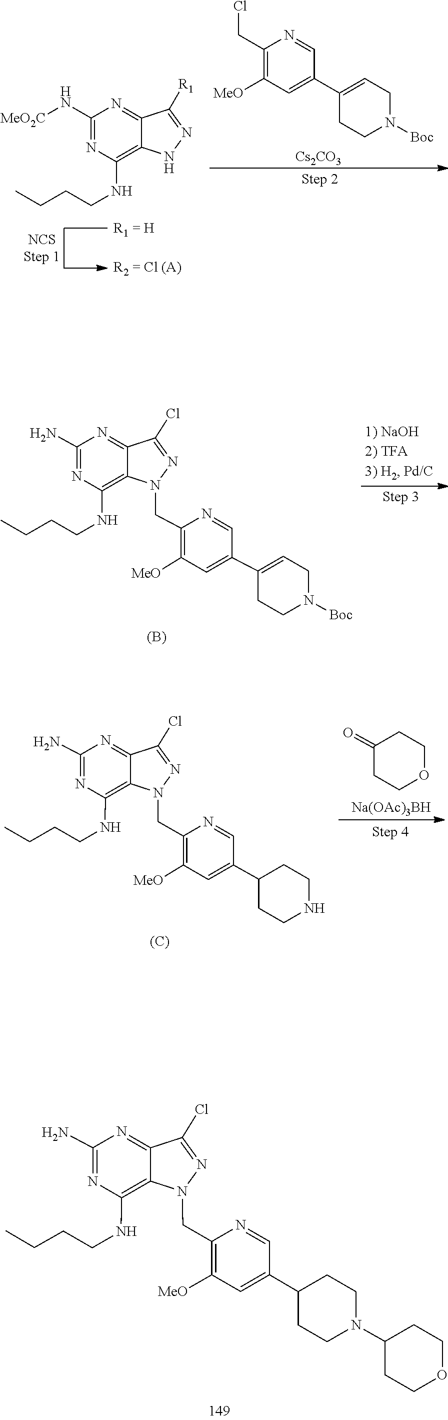

Step 1. A suspension of methyl (7-(butylamino)-1H-pyrazolo[4,3-d]pyrimidin-5-yl)carbamate (500 mg, 1.892 mmol) in DMF (9.5 ml) was treated with N-chlorosuccinimide (NCS, 303 mg, 2.270 mmol) in one portion at RT. After stirring for 10 min, the reaction mixture was diluted with DCM (100 mL), washed with water (3×75 mL), dried over Na₂SO₄, and filtered. The filtrate was concentrated. The crude material was purified by silica column chromatography (24 g), eluting with DCM:10% MeOH in DCM 0-10% gradient. The desired fractions were concentrated to yield methyl (7-(butylamino)-3-chloro-2H-pyrazolo[4,3-d]pyrimidin-5-yl)carbamate (Compound A) (536.8 mg, 1.80 mmol, 95% yield).

LCMS ESI: calculated for C₁₁H₁₆ClN₆O₂=299.7 (M+H⁺), found 299.1 (M+H⁺).

¹H NMR (400 MHz, DMSO-d6) δ 9.81 (s, 1H), 3.62 (s, 3H), 3.54 (br d, J=5.7 Hz, 2H), 1.71-1.54 (m, 2H), 1.40 (br dd, J=15.0, 7.5 Hz, 2H), 0.94 (br t, J=7.4 Hz, 3H). Two exchangeable protons were not visible.

Step 2. tert-Butyl 6-((5-amino-7-(butylamino)-3-chloro-1H-pyrazolo[4,3-d]pyrimidin-1-yl)methyl)-5-methoxy-3′,6′-dihydro-[3,4′-bipyridine]-1′(2′H)-carboxylate (Compound B) was prepared from Compound A, by a procedure analogous to that used for Compound 6.

LCMS ESI: calculated for C₂₆H₃₆ClN₈O₃=543.3 (M+H⁺), found 543.2 (M+H⁺).

Step 3. N7-butyl-3-chloro-1-((3-methoxy-5-(piperidin-4-yl)pyridin-2-yl)methyl)-1H-pyrazolo[4,3-d]pyrimidine-5,7-diamine (Compound C) was prepared from Compound B analogously to the procedures above for from compound B.

LCMS ESI: calculated for C₂₆H₃₀ClN₈O=445.2 (M+H⁺), found 445.2 (M+H⁺).

Step 4. (Compound 149 was prepared from compound C, using a procedure analogous to that employed for Compound 121.

### Example 21—Compound 107

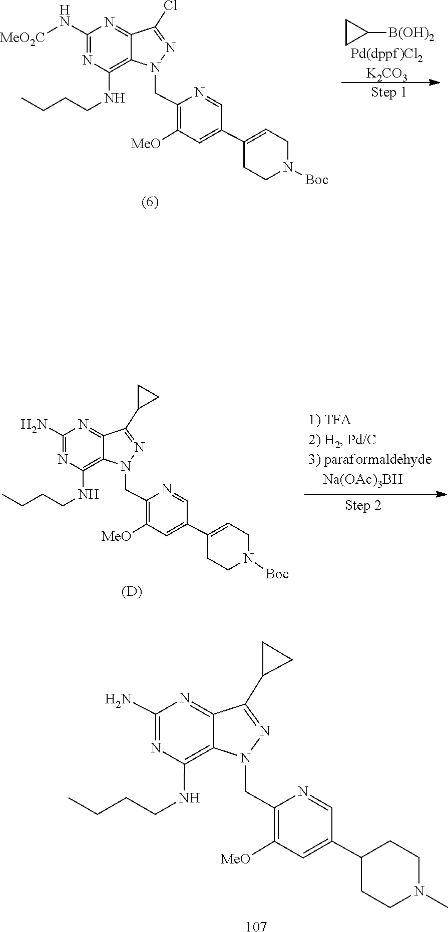

Step 1. A mixture of Compound 6 (100 mg, 0.155 mmol), K₂CO₃ (74.9 mg, 0.542 mmol) and PdCl₂(dppf)-CH₂Cl₂ adduct (12.65 mg, 0.015 mmol) in dioxane (0.5 mL) and water (0.1 mL) was bubbled with a stream of N₂ for 3 min. Cyclopropylboronic acid (133 mg, 1.549 mmol) was added. A stream of N₂ was bubbled for another 1 min. The reaction vessel was sealed and the reaction mixture was stirred at 110° C. for 24. The mixture mixture was diluted with EtOAc (15 ml). The resulting solid was removed by filtering through a CELITE™ pad. The filtrate was concentrated. The crude product was purified by ISCO silica column (12 g) chromatography, eluting with 20% MeOH in DCM:DCM, 0-30% gradient. The desired fractions were concentrated to yield tert-butyl 6-((5-amino-7-(butylamino)-3-cyclopropyl-1H-pyrazolo[4,3-d]pyrimidin-1-yl)methyl)-5-methoxy-3′,6′-dihydro-[3,4′-bipyridine]-1′(2′H)-carboxylate (compound D) (20 mg, 0.036 mmol, 23.53% yield).

LCMS ESI: calculated for C₂₉H₄₁N₈O₃=549.3 (M+H⁺), found 549.4 (M+H⁺).

Step 2. Compound 107 was prepared from Compound D analogously to the synthetic procedure employed above for compound 121.

### Example 22—Compound 166 and Compound 167

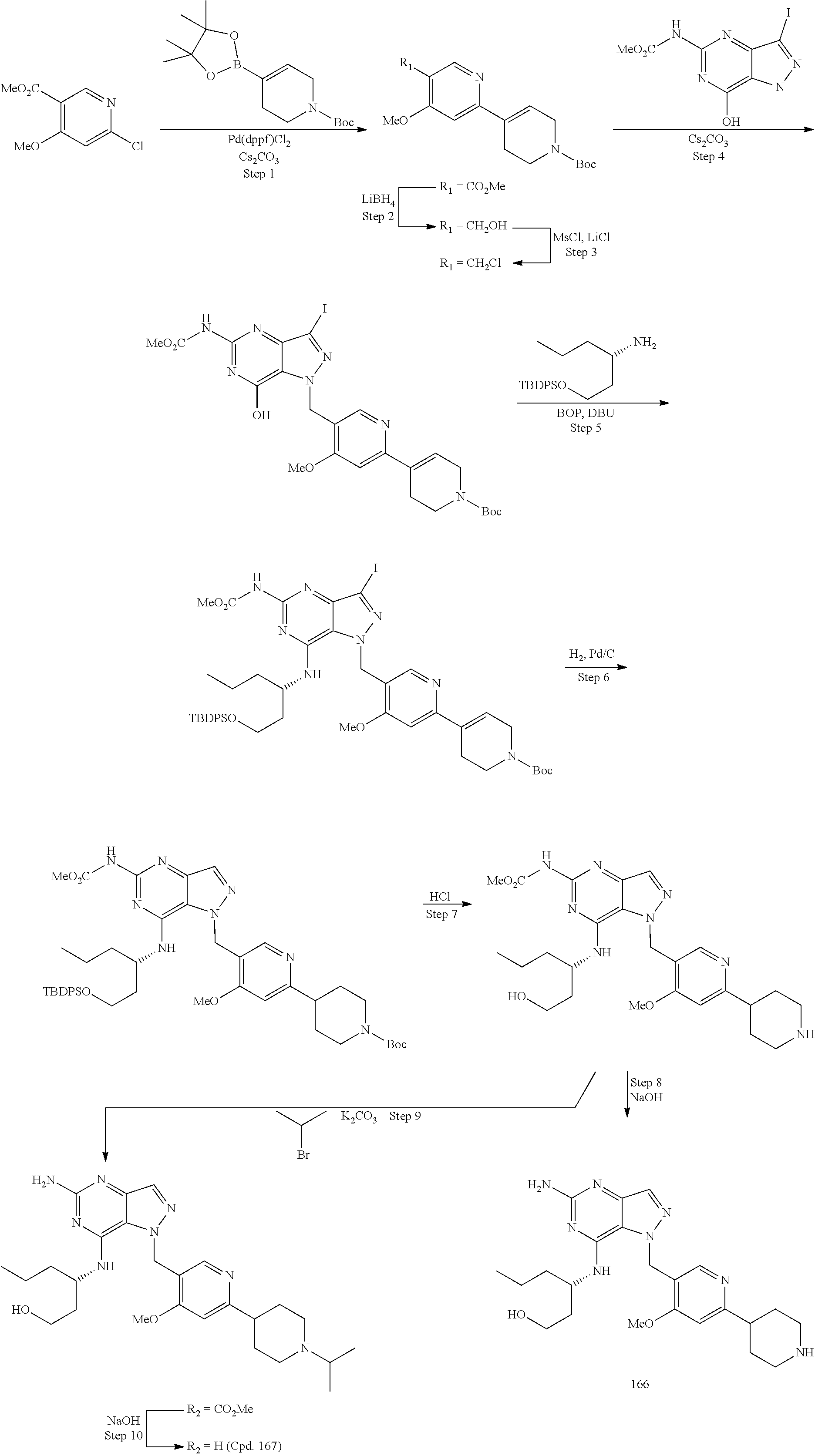

Step 1. To a stirred solution of methyl 6-chloro-4-methoxynicotinate (4.0 g, 19.84 mmol) in 1,4-Dioxane (40.0 mL), water (10.0 mL), Cs₂CO₃ (19.39 g, 59.5 mmol), tert-butyl 4-(4,4,5,5-tetramethyl-1,3,2-dioxaborolan-2-yl)-3,6-dihydropyridine-1(2H)-carboxylate (9.20 g, 29.8 mmol) and PdCl₂(dppf)-CH₂Cl₂ adduct (1.620 g, 1.984 mmol) were added while purging with N₂. The reaction mixture was stirred at 100° C. for 4 h, filtered through CELITE™ and washed with EtOAc. The filtrate was concentrated under reduced pressure. The crude product was purified on silicagel with a gradient of 0% to 100% of ethyl acetate in pet ether to provide 1′-(tert-butyl) 5-methyl 4-methoxy-3′,6′-dihydro-[2,4′-bipyridine]-1′,5(2′H)-dicarboxylate (5.19 g, 14.15 mmol, 71.3% yield) as a light brown oil.

LC-MS m/z 349.3 [M+H]⁺.

¹H NMR (300 MHz, DMSO-d6) d=8.72-8.66 (m, 1H), 7.27-7.18 (m, 1H), 6.94-6.81 (m, 1H), 5.78-5.73 (m, 1H), 4.12-4.05 (m, 2H), 3.95 (d, J=3.0 Hz, 6H), 3.80 (s, 3H), 3.57-3.49 (m, 2H), 2.58 (br d, J=1.5 Hz, 2H), 1.45-1.41 (m, 9H), 1.09-1.05 (m, 15H).

Step 2. To a stirred solution of 1′-(tert-butyl) 5-methyl 4-methoxy-3′,6′-dihydro-[2,4′-bipyridine]-1′,5(2′H)-dicarboxylate (1.5 g, 4.31 mmol) in THF (12.0 mL), MeOH (3.0 mL), LiBH₄ (in THF; 5.38 mL, 10.76 mmol) were added. After 16 h, the reaction mixture was quenched with 10% NaOH solution and partitioned between EtOAc and water. The organic layer was washed with brine and dried over Na₂SO₄, filtered and concentrated under reduced pressure to provide tert-butyl 5-(hydroxymethyl)-4-methoxy-3′,6′-dihydro-[2,4′-bipyridine]-1′(2′H)-carboxylate (1.1 g, 3.36 mmol, 78% yield) as a brown thick oil. The product was used as-is.

LC-MS m/z 321.2 [M+H]⁺.

¹H NMR (300 MHz, DMSO-d6) d=8.32 (s, 1H), 7.10 (s, 1H), 6.67 (br s, 1H), 5.77-5.76 (m, 1H), 5.77 (s, 1H), 5.09 (t, J=5.5 Hz, 1H), 4.48 (d, J=5.7 Hz, 2H), 4.07-4.02 (m, 2H), 3.95 (s, 1H), 3.88 (s, 3H), 3.57-3.49 (m, 2H), 2.62-2.54 (m, 2H), 2.01-1.98 (m, 1H), 1.43 (s, 9H), 1.09-1.05 (m, 3H).

Step 3. To a stirred solution of tert-butyl 5-(hydroxymethyl)-4-methoxy-3′,6′-dihydro-[2,4′-bipyridine]-1′(2′H)-carboxylate (1.2 g, 3.75 mmol) in DCM (15.0 mL), TEA (1.044 mL, 7.49 mmol), Ms-Cl (0.584 mL, 7.49 mmol) and lithium chloride (0.318 g, 7.49 mmol) were added at 0° C. The reaction mixture was stirred at same temperature for 30 min and then at RT for 4 h and partitioned between DCM and water. The organic layer was washed with brine, dried over Na₂SO₄, filtered and concentrated under reduced pressure to afford tert-butyl 5-(chloromethyl)-4-methoxy-3′,6′-dihydro-[2,4′-bipyridine]-1′(2′H)-carboxylate (1.3 g, 3.68 mmol, 98% yield) as a brown oil. The product was used without further purification.

LC-MS m/z 339.2 [M+H]⁺

¹H NMR (300 MHz, DMSO-d6) d=8.74-8.65 (m, 1H), 7.58-7.48 (m, 1H), 6.98-6.86 (m, 1H), 5.77-5.74 (m, 1H), 4.82 (s, 2H), 4.19-4.12 (m, 5H), 3.62-3.54 (m, 2H), 2.67-2.58 (m, 2H), 2.36-2.32 (m, 2H), 1.50-1.48 (m, 1H), 1.44 (s, 9H), 1.31 (s, 1H).

Step 4. To a stirred solution of methyl (7-hydroxy-3-iodo-1H-pyrazolo[4,3-d]pyrimidin-5-yl)carbamate (1.0 g, 2.98 mmol) in DMF (10.0 mL), Cs₂CO₃ (1.945 g, 5.97 mmol) was added. To this mixture tert-butyl 5-(chloromethyl)-4-methoxy-3′,6′-dihydro-[2,4′-bipyridine]-1′(2′H)-carboxylate (1.011 g, 2.98 mmol) in DMF (5.0 mL) was added at 0° C. The reaction mixture was stirred at 0° C. for 1 h and at RT for 1 h and partitioned between EtOAc and water. The organic layer was washed with brine, dried over Na₂SO₄, filtered and concentrated under reduced pressure. The crude product was purified on silica gel with a gradient of 0% to 100% of ethyl acetate in pet ether to provide tert-butyl 5-((7-hydroxy-3-iodo-5-((methoxycarbonyl)-amino)-1H-pyrazolo[4,3-d]pyrimidin-1-yl)methyl)-4-methoxy-3′,6′-dihydro-[2,4′-bipyridine]-1′(2′H)-carboxylate (718 mg, 0.946 mmol, 31.7% yield) as a light brown solid.

LC-MS m/z 638.0 [M+H]⁺

¹H NMR (300 MHz, DMSO-d6) d=11.74-11.66 (m, 1H), 11.44-11.36 (m, 1H), 8.06-7.98 (m, 1H), 7.19-7.09 (m, 1H), 6.75-6.63 (m, 1H), 5.74-5.67 (m, 2H), 4.07-4.01 (m, 2H), 3.85 (s, 3H), 3.78-3.71 (m, 5H), 3.52 (br t, J=5.3 Hz, 2H), 2.59-2.53 (m, 2H), 1.42 (s, 13H), 1.07 (s, 1H).

Step 5. To a stirred solution of tert-butyl 5-((7-hydroxy-3-iodo-5-((methoxy-carbonyl)amino)-1H-pyrazolo[4,3-d]pyrimidin-1-yl)methyl)-4-methoxy-3′,6′-dihydro-[2,4′-bipyridine]-1′(2′H)-carboxylate (0.5 g, 0.784 mmol) in DMSO (5.0 mL), DBU (0.355 mL, 2.353 mmol), BOP (0.520 g, 1.177 mmol) and (S)-1-((tert-butyldiphenylsilyl)oxy)hexan-3-amine (0.335 g, 0.941 mmol) were added. The reaction mixture was stirred at 45° C. for 16 h and partitioned between EtOAc and water. The organic layer was washed with brine, dried over Na₂SO₄, filtered and concentrated under reduced pressure. The crude product was purified on silica gel with a gradient of 0% to 100% of ethyl acetate in petroleum ether to provide tert-butyl (S)-5-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-4-methoxy-3′,6′-dihydro-[2,4′-bipyridine]-1′(2′H)-carboxylate (0.16 g, 0.100 mmol, 12.76% yield) as a brown solid.

LC-MS m/z 975.3 [M+H]⁺.

¹H NMR (300 MHz, DMSO-d6) d=9.77 (s, 1H), 7.83-7.80 (m, 1H), 7.59-7.52 (m, 3H), 7.51-7.44 (m, 3H), 7.42-7.30 (m, 6H), 7.28-7.18 (m, 3H), 7.11-7.08 (m, 1H), 6.70-6.61 (m, 2H), 5.75-5.64 (m, 2H), 4.69-4.60 (m, 1H), 4.05-3.97 (m, 2H), 3.74 (s, 3H), 3.70-3.59 (m, 4H), 3.57 (s, 3H), 3.50-3.40 (m, 2H), 2.02-1.97 (m, 1H), 1.91-1.78 (m, 3H), 1.46-1.40 (m, 11H), 1.26-1.16 (m, 4H), 0.95-0.88 (m, 12H), 0.84-0.75 (m, 5H).

Step 6. To a stirred solution of tert-butyl (S)-5-((7-((1-((tert-butyldiphenylsilyl)oxy)-hexan-3-yl)amino)-3-iodo-5-((methoxycarbonyl)amino)-1H-pyrazolo[4,3-d]pyrimidin-1-yl)methyl)-4-methoxy-3′,6′-dihydro-[2,4′-bipyridine]-1′(2′H)-carboxylate (0.35 g, 0.359 mmol) in MeOH (5.0 mL), was added Pd—C (0.191 g, 0.179 mmol). The reaction mixture was stirred at 50° C. under 100 psi hydrogen for 16 h and filtered through a CELITE™ bed, washing with methanol. The filtrate was concentrated under reduced pressure to provide tert-butyl (S)-4-(5-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-4-methoxypyridin-2-yl)piperidine-1-carboxylate (0.23 g, 0.208 mmol, 58.0% yield) as a light brown solid. The product was used without further purification.

LC-MS m/z 851.5 [M+H]⁺.

Step 7. To a stirred solution of tert-butyl (S)-4-(5-((7-((1-((tert-butyldiphenyl-silyl)oxy)hexan-3-yl)amino)-5-((methoxycarbonyl)amino)-1H-pyrazolo[4,3-d]pyrimidin-1-yl)methyl)-4-methoxypyridin-2-yl)piperidine-1-carboxylate (0.1 g, 0.117 mmol) in MeOH (2.0 mL), was added HCl (0.2 mL, 2.304 mmol) at 0° C. The reaction mixture was stirred at RT for 4 h, concentrated under reduced pressure, and co-distilled with DCM and diethyl ether to provide methyl (S)-(7-((1-hydroxyhexan-3-yl)amino)-1-((4-methoxy-6-(piperidin-4-yl)pyridin-3-yl)methyl)-1H-pyrazolo[4,3-d]pyrimidin-5-yl)carbamate, HCl (103 mg, 0.116 mmol, 99% yield) as a light brown solid. The product was used without further purification.

LC-MS m/z 513.3 [M+H]⁺.

Step 8. To a stirred solution of methyl (S)-(7-((1-hydroxyhexan-3-yl)amino)-1-((4-methoxy-6-(piperidin-4-yl)pyridin-3-yl)methyl)-1H-pyrazolo[4,3-d]pyrimidin-5-yl)carbamate, HCl (0.1 g, 0.182 mmol) in dioxane (1.5 mL), water (1.5 mL), NaOH (0.073 g, 1.821 mmol) were added. The reaction mixture was stirred at 70° C. for 4 h. The layers were separated and the aqueous layer was extracted with EtOAc. The combined organic layers were washed with brine, dried over Na₂SO₄, filtered and concentrated under reduced pressure. The crude material was purified via preparative LC/MS with the following conditions: Column: Waters XBridge C18, 150 mm×19 mm, 5-μm particles; Mobile Phase A: 5:95 acetonitrile:water with 10-mM NH₄OAc; Mobile Phase B: 95:5 acetonitrile:water with 10-mM NH₄OAc; Gradient: a 0-min hold at 7% B, 7-25% B over 20 min, then a 5-min hold at 100% B; Flow Rate: 20 mL/min; Column Temperature: 25° C. Fraction collection was triggered by MS and UV signals. Fractions containing the desired product were combined and dried via centrifugal evaporation to provide (16.3 mg, 0.035 mmol, 19.49% yield).

Step 9. To a stirred solution of methyl (S)-(7-((1-hydroxyhexan-3-yl)amino)-1-((4-methoxy-6-(piperidin-4-yl)pyridin-3-yl)methyl)-1H-pyrazolo[4,3-d]pyrimidin-5-yl)carbamate (50.0 mg, 0.098 mmol) in DMF (2.0 mL), were added K₂CO₃ (40.4 mg, 0.293 mmol) and 2-bromopropane (0.027 mL, 0.293 mmol). The reaction mixture was stirred at 50° C. for 6 h and partitioned between EtOAc and water. The organic layer was washed with brine, dried over Na₂SO₄, filtered and concentrated under reduced pressure to provide methyl (S)-(7-((1-hydroxyhexan-3-yl)amino)-1-((6-(1-isopropylpiperidin-4-yl)-4-methoxypyridin-3-yl)methyl)-1H-pyrazolo[4,3-d]pyrimidin-5-yl)carbamate (60 mg, 0.089 mmol, 91% yield) as a light brown semi solid. The product was used without further purification.

LC-MS m/z 555.2 [M+H]⁺.

Step 10. To a stirred solution of methyl (S)-(7-((1-hydroxyhexan-3-yl)amino)-1-((6-(1-isopropylpiperidin-4-yl)-4-methoxypyridin-3-yl)methyl)-1H-pyrazolo[4,3-d]pyrimidin-5-yl)carbamate (80.0 mg, 0.144 mmol) in 1,4-dioxane (1.0 mL), water (1.0 mL), was added NaOH (28.8 mg, 0.721 mmol). The reaction mixture was stirred at 70° C. for 6 h. The organic layer separated and concentrated under reduced pressure. The crude material was purified via preparative LC/MS with the following conditions: Column: Waters XBridge C18, 19×150 mm, 5-μm particles; Mobile Phase A: 10-mM NH₄OAc; Mobile Phase B: acetonitrile; Gradient: 9-27% B over 22 minutes, then a 5-minute hold at 100% B; Flow: 20 mL/min. Fractions containing the desired product were combined and dried via centrifugal evaporation to provide Compound 167 (14.4 mg, 0.028 mmol, 19.70% yield).

The following compounds were analogously prepared: Compound 168, Compound 173, Compound 174, and Compound 176.

### Example 23—Compound 177

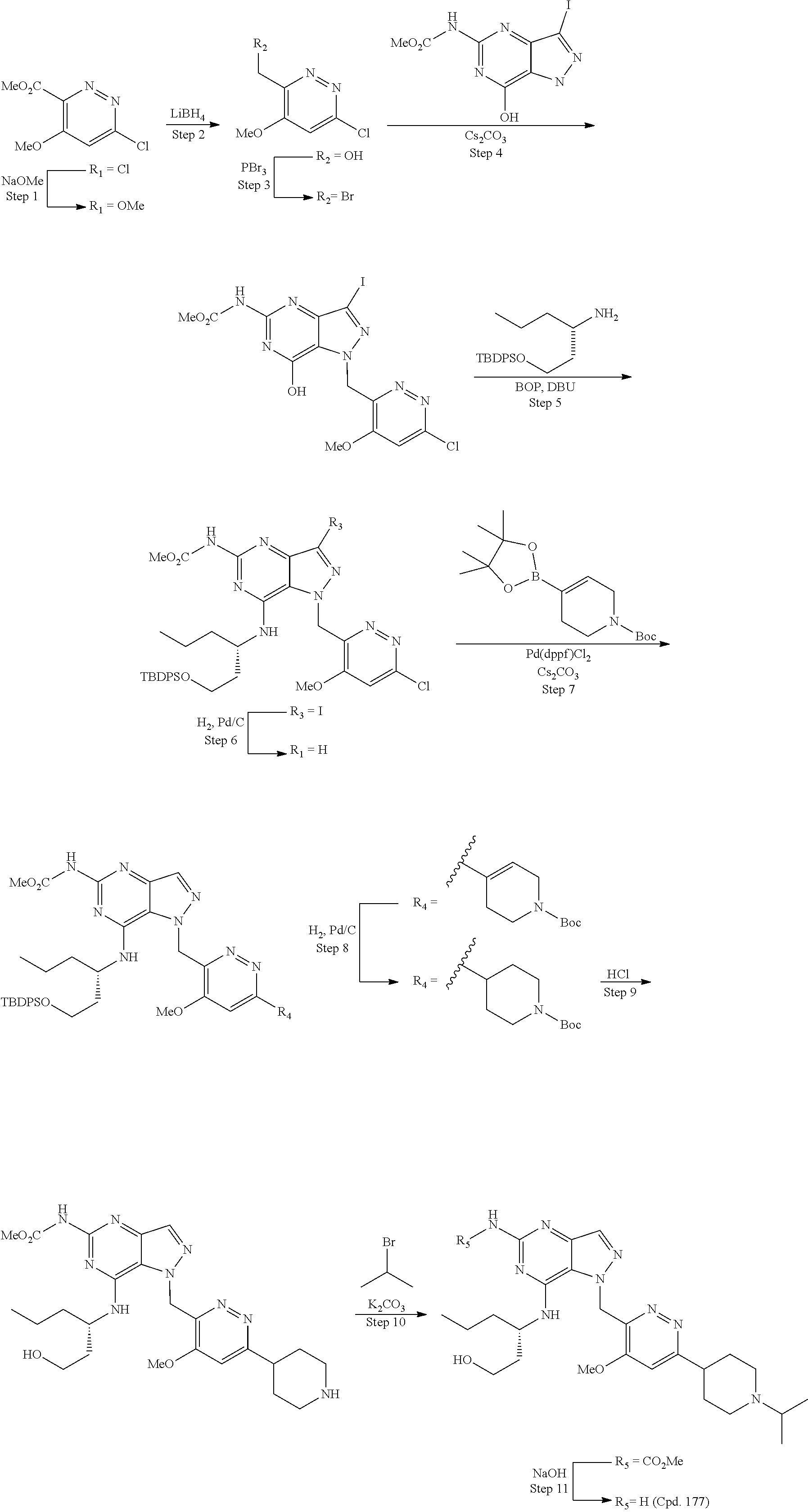

Step 1. To a stirred solution of methyl 4,6-dichloropyridazine-3-carboxylate (7 g, 33.8 mmol) in anhydrous THF (70 mL) at 0° C., was added drop-wise 25% NaOMe in methanol (2.009 g, 37.2 mmol). Ice bath was removed. The reaction mixture was stirred for 16 h at RT, diluted with 1.5N HCl (40 mL), and added to a saturated NaHCO₃ solution. The mixture was extracted with DCM. The organic layer was washed with H₂O and saturated NaCl solution, dried over anhydrous Na₂SO₄, filtered and concentrated under reduced pressure. The crude product was purified on silicagel with 15% ethyl acetate in pet ether to provide methyl 6-chloro-4-methoxypyridazine-3-carboxylate (3.8 g, 17.82 mmol, 52.7% yield) as an off-white solid.

LCMS(ES): m/z=205.1 [M+H]⁺.

Step 2. To a stirred solution of methyl 6-chloro-4-methoxypyridazine-3-carboxylate (4.7 g, 23.20 mmol) in a mixture of THF (40 mL) and methanol (8 mL) at 0° C., was added drop-wise lithium borohydride (29.0 mL, 58.0 mmol). The ice bath was removed. The reaction mixture was stirred for 2 h at RT and cooled to 0° C. Ice cold water was added drop-wise. The reaction mixture was partitioned with ethyl acetate and the organic layer was washed with H₂O, saturated NaCl solution, dried over anhydrous Na₂SO₄, filtered and concentrated under reduced pressure to provide (6-chloro-4-methoxypyridazin-3-yl)methanol (3.5 g, 18.04 mmol, 78% yield) as a brown solid. The product was used without further purification.

LCMS(ES): m/z=175.0 [M+H]⁺.

Step 3. To a stirred solution of (6-chloro-4-methoxypyridazin-3-yl)methanol (1 g, 5.73 mmol) in anhydrous DCM (15 mL) at 0° C., was added PBr₃ (0.810 mL, 8.59 mmol). The ice bath was removed. The reaction mixture was stirred for 1 h at RT and partitioned between saturated NaHCO₃ and DCM. The organic layer was washed with H₂O, saturated NaCl, dried over anhydrous Na₂SO₄, filtered and concentrated under reduced pressure to provide 3-(bromomethyl)-6-chloro-4-methoxypyridazine (1 g, 3.37 mmol, 58.8% yield) as a brown solid.

LCMS(ES): m/z=238.9 [M+H]⁺.

Step 4. To a stirred solution of methyl (7-hydroxy-3-iodo-1H-pyrazolo[4,3-d]pyrimidin-5-yl)carbamate (1.25 g, 3.73 mmol) in anhydrous DMF (25 mL) at 0° C., were added Cs₂CO₃ (2.431 g, 7.46 mmol) and 3-(bromomethyl)-6-chloro-4-methoxypyridazine (0.886 g, 3.73 mmol). After stirring for 1 h at 0° C., the reaction mixture was partitioned between water and DCM. The organic layer was washed with H₂O, saturated NaCl solution, dried over anhydrous Na₂SO₄, filtered and concentrated under reduced pressure. The crude product was purified on silicagel with 5% methanol in chloroform to provide methyl (1-((6-chloro-4-methoxypyridazin-3-yl)methyl)-7-hydroxy-3-iodo-1H-pyrazolo[4,3-d]pyrimidin-5-yl)carbamate (1.1 g, 2.014 mmol, 54.0% yield) as a brown solid.

LC-MS (ES): m/z=492.0 [M+H]⁺.

Step 5. To a stirred solution of methyl (1-((6-chloro-4-methoxypyridazin-3-yl)methyl)-7-hydroxy-3-iodo-1H-pyrazolo[4,3-d]pyrimidin-5-yl)carbamate (1.1 g, 2.237 mmol) in anhydrous DMSO (8 mL), were added DBU (0.675 mL, 4.47 mmol), BOP (1.979 g, 4.47 mmol) and lastly (S)-1-((tert-butyldiphenylsilyl)oxy)hexan-3-amine (0.875 g, 2.461 mmol) at RT. The reaction mixture was heated to 45° C. for 1 h and partitioned between water and ethyl acetate. The organic layer was washed with H₂O, saturated NaCl, dried over anhydrous Na₂SO₄, filtered and concentrated under reduced pressure. The crude product was purified on silicagel with 55% ethyl acetate in proleum ether to provide methyl (S)-(7-((1-((tert-butyldiphenylsilyl)oxy)-hexan-3-yl)amino)-1-((6-chloro-4-methoxypyridazin-3-yl)methyl)-3-iodo-1H-pyrazolo[4,3-d]pyrimidin-5-yl)carbamate (1.1 g, 1.327 mmol, 59.3% yield) as a pale yellow semi solid.

LCMS(ES): m/z=829.1 [M+H]⁺.

Step 6. To a stirred solution of methyl (S)-(7-((1-((tert-butyldiphenylsilyl)oxy)hexan-3-yl)amino)-1-((6-chloro-4-methoxypyridazin-3-yl)methyl)-3-iodo-1H-pyrazolo[4,3-d]pyrimidin-5-yl)carbamate (900 mg, 1.085 mmol) in a mixture of anhydrous ethyl acetate (25 mL) and EtOH (5 mL), was added Pd/C (866 mg, 0.814 mmol) at RT. The reaction mixture was stirred under hydrogen for 16 h. The suspension was filtered through a CELITE™ bed, which was washed with ethyl acetate. The filtrate was concentrated under reduced pressure to provide methyl (S)-(7-((1-((tert-butyldiphenylsilyl)oxy)hexan-3-yl)amino)-1-((6-chloro-4-methoxypyridazin-3-yl)methyl)-1H-pyrazolo[4,3-d]pyrimidin-5-yl)carbamate (650 mg, 0.739 mmol, 68.1% yield) as a pale yellow semi solid. The product was used without further purification.

LCMS(ES): m/z=703.3 [M+H]⁺.

Step 7. To a stirred solution of methyl (S)-(7-((1-((tert-butyldiphenylsilyl)oxy)hexan-3-yl)amino)-1-((6-chloro-4-methoxypyridazin-3-yl)methyl)-1H-pyrazolo[4,3-d]pyrimidin-5-yl)carbamate (650 mg, 0.924 mmol) in anhydrous dioxane (15 mL) and water (0.2 mL), were added tert-butyl 4-(4,4,5,5-tetramethyl-1,3,2-dioxaborolan-2-yl)-3,6-dihydropyridine-1(2H)-carboxylate (572 mg, 1.848 mmol), Cs₂CO₃ (903 mg, 2.77 mmol) and PdCl₂(dppf)-CH₂Cl₂ adduct (75 mg, 0.092 mmol) at RT. The reaction mixture was purged with nitrogen and heated to 100° C. After 16 h the suspension was filtered through CELITE™ and washed with ethyl acetate. The filtrate was concentrated under reduced. The crude product was purified on silicagel with 35% ethyl acetate in pet ether to provide tert-butyl (S)-4-(6-((7-((1-((tert-butyldiphenylsilyl)oxy)-hexan-3-yl)amino)-5-((methoxycarbonyl)amino)-1H-pyrazolo[4,3-d]pyrimidin-1-yl)methyl)-5-methoxypyridazin-3-yl)-3,6-dihydropyridine-1(2H)-carboxylate (380 mg, 0.358 mmol, 38.7% yield) as a pale yellow solid.

LCMS(ES): m/z=850.4 [M+H]⁺.

Step 8. To a stirred solution of tert-butyl (S)-4-(6-((7-((1-((tert-butyldiphenylsilyl)-oxy)hexan-3-yl)amino)-5-((methoxycarbonyl)amino)-1H-pyrazolo[4,3-d]pyrimidin-1-yl)methyl)-5-methoxypyridazin-3-yl)-3,6-dihydropyridine-1(2H)-carboxylate (370 mg, 0.435 mmol) in anhydrous Methanol (15 mL) was added Pd/C (232 mg, 0.218 mmol) at RT. The reaction mixture was heated to 50° C. in an autoclave under 5 kPa of hydrogen. After 24 h, the suspension was filtered through a CELITE™ pad, which was washed with ethyl acetate. The filtrate was concentrated under reduced pressure to provide tert-butyl (S)-4-(6-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-5-methoxypyridazin-3-yl)piperidine-1-carboxylate (350 mg, 0.370 mmol, 85% yield) as a pale yellow semi solid. The product was used without further purification.

LCMS(ES): m/z=552.3 [M+H]⁺.

Step 9. To a stirred solution of tert-butyl (S)-4-(6-((7-((1-((tert-butyldiphenylsilyl)-oxy)hexan-3-yl)amino)-5-((methoxycarbonyl)amino)-1H-pyrazolo[4,3-d]pyrimidin-1-yl)methyl)-5-methoxypyridazin-3-yl)piperidine-1-carboxylate (70 mg, 0.082 mmol) in Methanol (2 mL), was added HCl (0.5 mL, 16.46 mmol) at RT. After 4 h, the reaction mixture was concentrated to dryness under high vacuum to provide methyl (S)-(7-((1-hydroxyhexan-3-yl)amino)-1-((4-methoxy-6-(piperidin-4-yl)pyridazin-3-yl)methyl)-1H-pyrazolo[4,3-d]pyrimidin-5-yl)carbamate HCl (crude 60 mg) as a brown semi solid. The product was used without further purification.

LCMS(ES): m/z=514.3 [M+H]⁺

Step 10. To a stirred solution of methyl (S)-(7-((1-hydroxyhexan-3-yl)amino)-1-((4-methoxy-6-(piperidin-4-yl)pyridazin-3-yl)methyl)-1H-pyrazolo[4,3-d]pyrimidin-5-yl)carbamate (60 mg, 0.117 mmol) in anhydrous DMF (1 mL), were added K₂CO₃ (81 mg, 0.584 mmol) and 2-bromopropane (0.055 mL, 0.584 mmol) at RT. The reaction mixture was heated to 50° C. After 16 h, the reaction mixture was concentrated to dryness under high vacuum to provide methyl (S)-(7-((1-hydroxyhexan-3-yl)amino)-1-((6-(1-isopropylpiperidin-4-yl)-4-methoxypyridazin-3-yl)methyl)-1H-pyrazolo[4,3-d]pyrimidin-5-yl)carbamate (60 mg). The product was used without further purification.

LCMS(ES): m/z=556.3 [M+H]⁺.

Step 11. To a stirred solution of methyl (S)-(7-((1-hydroxyhexan-3-yl)amino)-1-((6-(1-isopropylpiperidin-4-yl)-4-methoxypyridazin-3-yl)methyl)-1H-pyrazolo[4,3-d]pyrimidin-5-yl)carbamate (60 mg, 0.108 mmol) in a mixture of dioxane (1.5 mL) and water, was added NaOH (43.2 mg, 1.080 mmol). The reaction mixture was heated to 75° C. After 3 h, the dioxane layer from the reaction mixture was separated and concentrated to dryness to get the crude product. The crude material was purified via preparative LC/MS with the following conditions: Column: Waters XBridge C18, 19×150 mm, 5-μm particles; Mobile Phase A: 10-mM NH₄OAc; Mobile Phase B: acetonitrile; Gradient: 7-22% B over 20 minutes, then a 5-minute hold at 100% B; Flow: 20 mL/min. Fractions containing the desired product were combined and dried via centrifugal evaporation to provide Compound 177 (12.7 mg, 0.025 mmol, 24%).

### Example 24—Compound 200

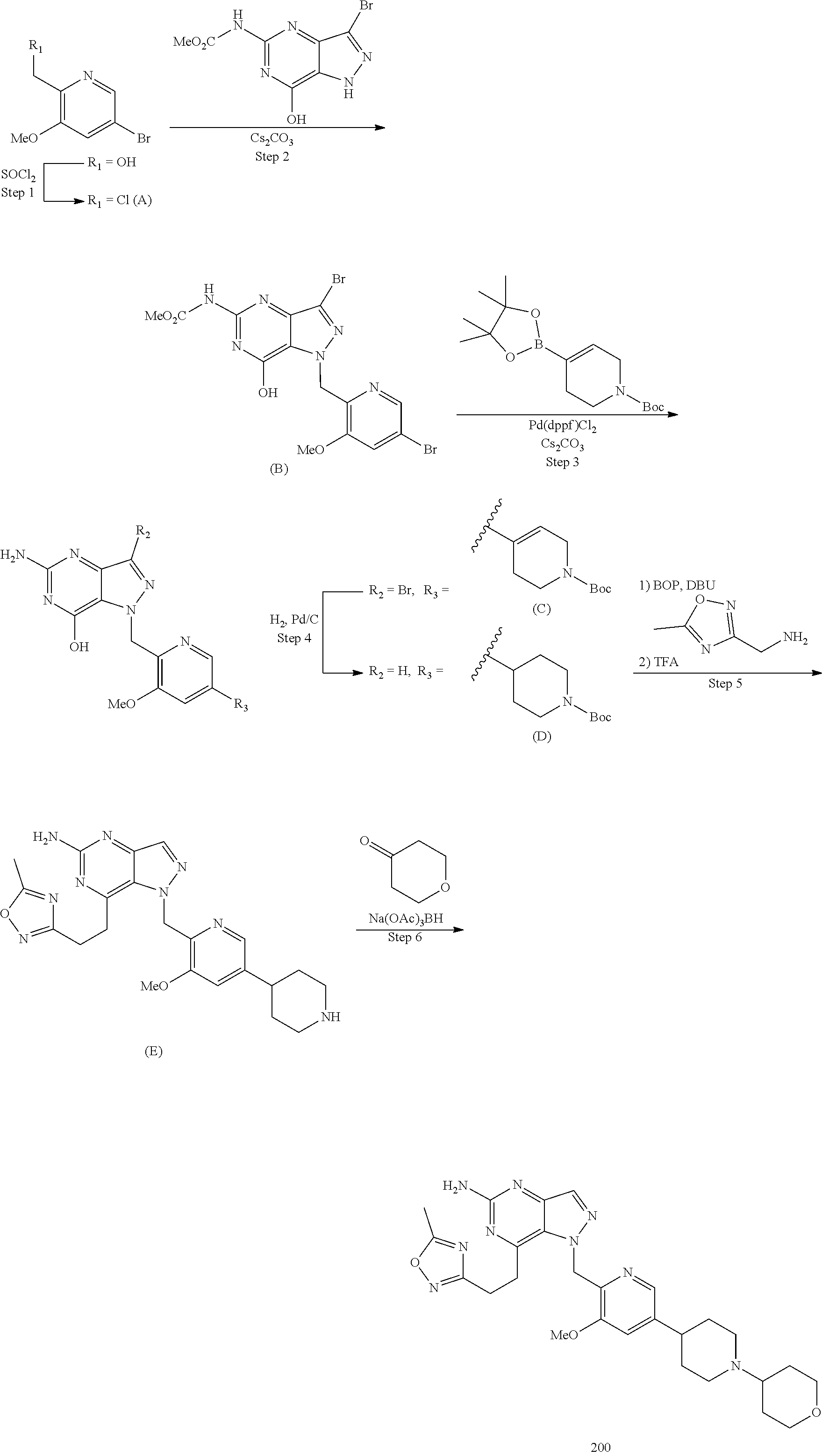

Step 1. A mixture of (5-bromo-3-methoxypyridin-2-yl)methanol (1 g, 4.59 mmol) in DCM (10 mL) was treated with SOCl₂ (0.669 mL, 9.17 mmol) at 0° C. After stirring for 2 hrs, the reaction mixture was concentrated. The excess SOCl₂ was azeotropically removed with DCM three times. The resulting Compound A was directly carried over to the next step.

LCMS ESI: calculated for C₇H₈BrClNO=235.9, 237.9 (M+H⁺), found 235.9, 237.9 (M+H⁺).

¹H NMR (400 MHz, DMSO-d6) δ 8.27 (d, J=1.8 Hz, 1H), 7.82 (d, J=1.8 Hz, 1H), 4.70 (s, 2H), 3.93 (s, 3H).

Step 2. Compound B was made from compound A and methyl (3-bromo-7-hydroxy-2H-pyrazolo[4,3-d]pyrimidin-5-yl)carbamate, using a procedure analogous to that in Example 1.

LCMS ESI: calculated for C₁₂H₁₃Br₂N₆O₄=488.0 (M+H⁺), found 488.9 (M+H⁺).

¹H NMR (400 MHz, DMSO-d₆) δ 8.10 (d, J=1.8 Hz, 1H), 7.78 (d, J=1.8 Hz, 1H), 5.75 (s, 2H), 3.91 (s, 3H), 3.75 (s, 3H).

Step 3. A suspension of compound B (750 mg, 1.537 mmol), tert-butyl 4-(4,4,5,5-tetramethyl-1,3,2-dioxaborolan-2-yl)-3,6-dihydropyridine-1(2H)-carboxylate (713 mg, 2.305 mmol), and K₂CO₃ (849 mg, 6.15 mmol) in DMF (10 mL) was bubbled with a stream of N₂ for 2 min. PdCl₂(dppf)-CH₂Cl₂ adduct (125 mg, 0.154 mmol) was added. The reaction mixture was bubbled with a stream of N₂ for another 1 min, then stirred at 100° C. for 16 h under N₂. Upon cooling, the reaction mixture was diluted with DCM. The catalyst was removed by filtration through a CELITE™ pad. The filtrate was neutralized with HOAc to pH 6-7 and concentrated on a rotary evaporator. The crude product was purified by silica column (80 g) chromatography, eluting with 20% MeOH in DCM:DCM, 0-40% gradient. The desired fractions were combined and concentrated. The residue, which contained DMF, was triturated with water and stirred at RT for 1 hr. The resulting solid was collected by filtration and air dried to yield Compound C (350 mg, 0.655 mmol, 42.8% yield).

LCMS ESI: calculated for C₂₂H₂₇BrN₇O₄=532., 534.1 (M+H⁺), found 532.1, 534.1 (M+H⁺).

¹H NMR (400 MHz, DMSO-d6) δ 11.23-10.87 (m, 1H), 8.04 (d, J=1.5 Hz, 1H), 7.45 (d, J=1.5 Hz, 1H), 6.33 (br s, 2H), 6.28 (br s, 1H), 4.01 (br d, J=2.0 Hz, 2H), 3.90 (s, 3H), 3.52-3.50 (m, 2H), 2.48 (br d, J=1.8 Hz, 2H), 1.43 (s, 9H).

Step 4. A Parr shaking bottle charged with a mixture of compound C (70 mg, 0.131 mmol) in MeOH (10 mL) was purged with N₂. Pd—C 10% (13.99 mg, 0.131 mmol) was added. The bottle was purged with H₂ and then shaken under H₂ (20 psi) for 3 days. The catalyst was removed by filtering through a syringe filter disc. The filtrate was concentrated to yield compound D (46.4 mg, 0.102 mmol, 77% yield).

LCMS ESI: calculated for C₂₂H₃₀N₇O₄=456.2 (M+H⁺), found 456.2 (M+H⁺).

¹H NMR (400 MHz, DMSO-d6) δ 10.98-10.76 (m, 1H), 7.84 (d, J=1.5 Hz, 1H), 7.48 (s, 1H), 7.33 (d, J=1.5 Hz, 1H), 5.68 (s, 2H), 3.86 (s, 3H), 3.37-3.32 (m, 4H), 2.74-2.66 (m, 1H), 1.81-1.70 (m, 2H), 1.63-1.53 (m, 2H), 1.42 (s, 9H).

Step 5, Part 1. To a solution of compound D (45 mg, 0.099 mmol) and (5-methyl-1,2,4-oxadiazol-3-yl)methanamine hydrochloride (29.6 mg, 0.198 mmol) in DMSO (2 mL) was added DBU (0.074 mL, 0.494 mmol), followed by BOP (87 mg, 0.198 mmol). The reaction mixture was stirred at 50° C. for 45 min. Upon cooling, the reaction mixture was poured onto sat NH₄Cl solution and stirred at RT for 30 min. The solid was collected by filtration and purified by silica column (12 g) chromatography, eluting with 5% MeOH in DCM:DCM=0-25%. The desired fractions were concentrated to yield tert-butyl 4-(6-((5-amino-7-(((5-methyl-1,2,4-oxadiazol-3-yl)methyl)amino)-1H-pyrazolo[4,3-d]pyrimidin-1-yl)methyl)-5-methoxypyridin-3-yl)piperidine-1-carboxylate (20 mg, 0.036 mmol, 36.8% yield) as a off-white solid solid.

LCMS ESI: calculated for C₂₆H₃₅N₁₀O₂=551.3 (M+H⁺), found 551.3 (M+H⁺).

Step 5, Part 2. The above solid was dissolved into DCM (0.5 mL). TFA (0.5 mL, 6.49 mmol) was added. After stirring at RT for 1 h, the reaction was complete. The mixture was concentrated to dryness to yield compound E as the TFA salt (18 mg, 0.032 mmol, 32.3% yield) which is directly carried over to the next step.

LCMS ESI: calculated for C₂₁H₂₇N₁₀O₄=451.2 (M+H⁺), found 451.3 (M+H⁺).

Step 6. Compound 200 was prepared from Compound E and tetrahydro-4H-pyran-4-one using sodium triacetoxyborohydride, analogously following the procedure of Example 2.

### Example 25—Compound 201

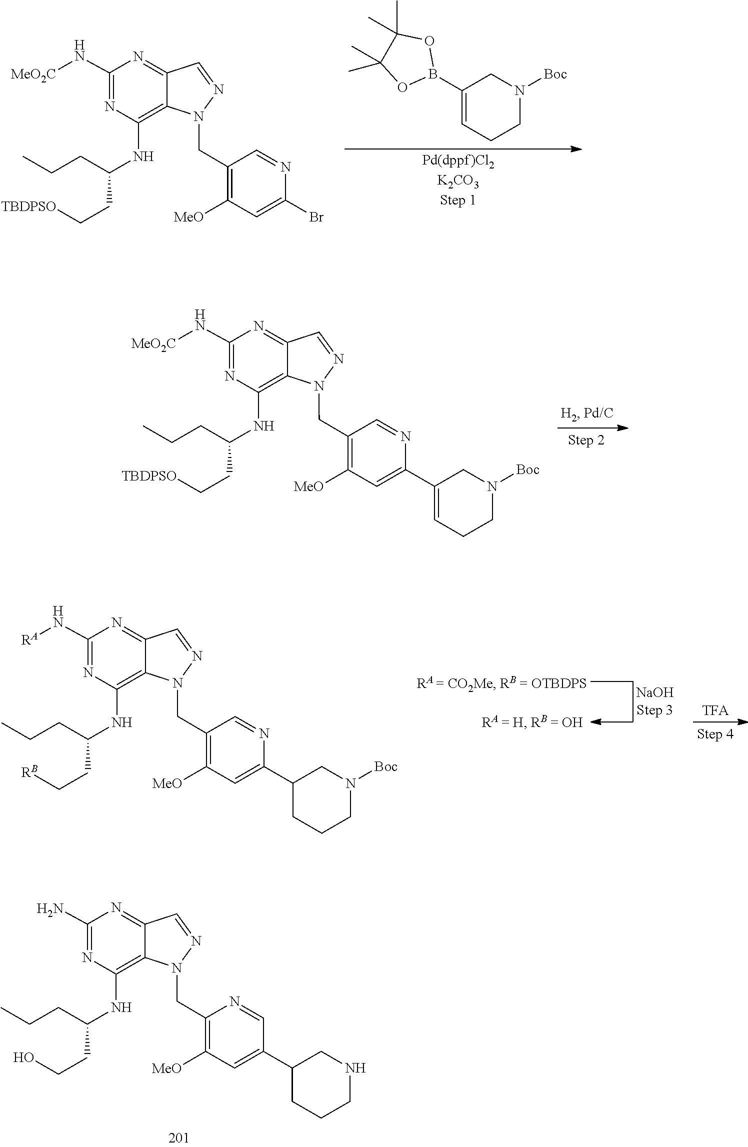

Step 1. A two phase mixture of methyl (S)-(1-((5-bromo-3-methoxypyridin-2-yl)methyl)-7-((1-((tert-butyldiphenylsilyl)oxy)hexan-3-yl)amino)-1H-pyrazolo[4,3-d]pyrimidin-5-yl)carbamate (130 mg, 0.174 mmol), tert-butyl 5-(4,4,5,5-tetramethyl-1,3,2-dioxaborolan-2-yl)-3,6-dihydropyridine-1(2H)-carboxylate (108 mg, 0.348 mmol), PdCl2(dppf) (25.5 mg, 0.035 mmol), and K₂CO₃ (96 mg, 0.696 mmol) in dioxane (4 mL) and water (300 μL) was prepared. The reaction mixture was purged 3 times with vacuum and nitrogen before heating to 80° C. for 3 h. The reaction mixture was diluted with ethyl acetate (50 mL). The organic layer was dried with Na₂SO₄, filtered and concentrated. The crude product was purified on silica gel with a gradient of 0% to 100% ethyl acetate in petroleum ether to provide tert-butyl (S)-6′-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-5′-methoxy-5,6-dihydro-[3,3′-bipyridine]-1(2H)-carboxylate (100 mg, 67.7% yield) as a light brown solid.

LC-MS m/z 849.6 [M+H]⁺.

¹H NMR (400 MHz, DMSO-d₆) δ 9.49-9.41 (m, 1H), 7.96-7.88 (m, 1H), 7.82-7.79 (m, 1H), 7.78-7.72 (m, 1H), 7.63-7.53 (m, 4H), 7.50-7.43 (m, 4H), 7.41-7.35 (m, 1H), 7.35-7.26 (m, 3H), 7.23-7.16 (m, 2H), 6.37-6.28 (m, 1H), 5.79-5.57 (m, 2H), 4.67-4.53 (m, 1H), 4.18-4.11 (m, 2H), 3.95-3.88 (m, 3H), 3.79-3.68 (m, 2H), 3.63-3.56 (m, 3H), 3.45-3.38 (m, 2H), 2.26-2.16 (m, 2H), 2.00-1.84 (m, 2H), 1.67-1.54 (m, 2H), 1.47-1.38 (m, 9H), 1.35-1.27 (m, 2H), 0.94-0.89 (m, 9H)

Step 2. A suspension of tert-butyl (S)-6′-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-5′-methoxy-5,6-dihydro-[3,3′-bipyridine]-1(2H)-carboxylate (100 mg, 0.118 mmol) and Pd/C (50.1 mg, 0.024 mmol) in MeOH (10 mL) was purged three times with vacuum and nitrogen then purged three times with vacuum and hydrogen. It was then stirred under hydrogen for 16 hrs. After purging with nitrogen the reaction mixture was filtered through a CELITE™ pad and evaporated under reduced pressure and dried under high vacuum to give tert-butyl 3-(6-((7-(((S)-1-((tert-butyldiphenylsilyl)oxy)hexan-3-yl)amino)-5-((methoxycarbonyl)amino)-1H-pyrazolo[4,3-d]pyrimidin-1-yl)methyl)-5-methoxypyridin-3-yl)piperidine-1-carboxylate, which was used as-is without further purification.

LC-MS m/z 851.7 [M+H]⁺

Step 3. A solution of tert-butyl 3-(6-((7-(((S)-1-((tert-butyldiphenylsilyl)oxy)hexan-3-yl)amino)-5-((methoxycarbonyl)amino)-1H-pyrazolo[4,3-d]pyrimidin-1-yl)methyl)-5-methoxy-pyridin-3-yl)piperidine-1-carboxylate (100 mg, 0.118 mmol) and NaOH (0.15 mL, 1.500 mmol) in MeOH (1.000 mL) and dioxane (2 mL) was heated at 50° C. for 16 h. The reaction mixture was partitioned between ethyl acetate (50 mL) and saturated NH₄Cl (10 mL). The organic layer was dried with Na₂SO₄, filtered and concentrated. The crude product was purified on silica gel with a gradient of 0% to 20% of methanol in dichloromethane to provide tert-butyl 3-(6-((5-amino-7-(((S)-1-hydroxyhexan-3-yl)amino)-1H-pyrazolo[4,3-d]pyrimidin-1-yl)methyl)-5-methoxypyridin-3-yl)piperidine-1-carboxylate (50 mg, 76% yield) as a light brown solid.

LC-MS m/z 555.4 [M+H]⁺.

Step 4. A solution of tert-butyl 3-(6-((5-amino-7-(((S)-1-hydroxyhexan-3-yl)amino)-1H-pyrazolo[4,3-d]pyrimidin-1-yl)methyl)-5-methoxypyridin-3-yl)piperidine-1-carboxylate (50 mg, 0.090 mmol) and TFA (0.069 mL, 0.901 mmol) in DCM (1 mL) was stirred at RT for 16 h. The reaction mixture was diluted with methanol 2 mL and K₂CO₃ (276 mg, 2 mmol) was added. After 2 h the reaction mixture was diluted with methanol (10 mL), filter and evaporated under reduced pressure. The crude material was purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 1% B, 1-41% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The second eluting diastereoisomer of Compound 201 (4.9 mg, 23% yield) was isolated via SCF with the following conditions: Column OD 30×250 mm ID, 5 μm; Modile phase: 85/15 CO₂/MeOH w/0.1% DEA; Flow rate: 100 mL/min; Column Temperature 40° C.

### Example 26—Compound 202

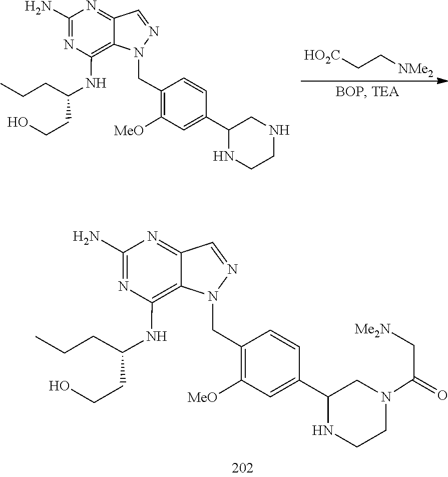 [SMILES: CCC[C@@H](CCO)Nc1nc(N)nc2cnn(Cc3ccc(C4CNCCN4)cc3OC)c12 ; CN(C)CCC(=O)O ; CCC[C@@H](CCO)Nc1nc(N)nc2cnn(Cc3ccc(C4CN(C(=O)CN(C)C)CCN4)cc3OC)c12]

To a solution of (3S)-3-((5-amino-1-(2-methoxy-4-(piperazin-2-yl)benzyl)-1H-pyrazolo[4,3-d]pyrimidin-7-yl)amino)hexan-1-ol (0.107 g, 0.236 mmol), 3-(dimethylamino)-propanoic acid (0.028 g, 0.236 mmol), and TEA (0.164 mL, 1.180 mmol) in NMP (1 mL) was added BOP (0.157 g, 0.354 mmol). After 3 h the reaction mixture was filtered. The crude material was purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 1% B, 1-41% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to provide 1-(3-{4-[(5-amino-7-{[(3S)-1-hydroxyhexan-3-yl]amino}-1H-pyrazolo[4,3-d]pyrimidin-1-yl)methyl]-3-methoxyphenyl}piperazin-1-yl)-3-(dimethylamino)propan-1-one (2.2 mg, 3.6% yield). See Table A for analytical data.

### Example 27—Compound 203

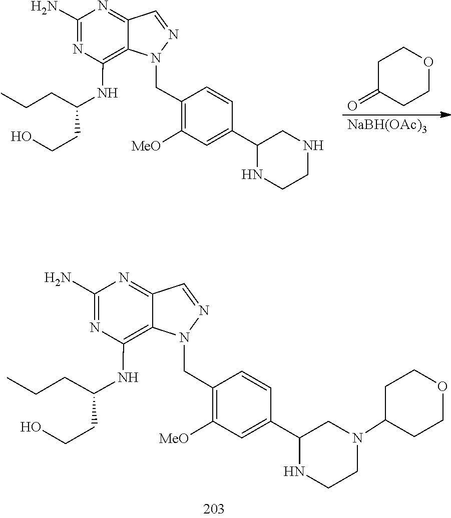 [SMILES: CCC[C@@H](CCO)Nc1nc(N)nc2cnn(Cc3ccc(C4CNCCN4)cc3OC)c12 ; O=C1CCOCC1 ; CCC[C@@H](CCO)Nc1nc(N)nc2cnn(Cc3ccc(C4CN(C5CCOCC5)CCN4)cc3OC)c12 ; "NaBH(OAc)3"]

To a solution of (3S)-3-((5-amino-1-(2-methoxy-4-(piperazin-2-yl)benzyl)-1H-pyrazolo[4,3-d]pyrimidin-7-yl)amino)hexan-1-ol (0.107 g, 0.236 mmol) and tetrahydro-4H-pyran-4-one (40 mg, 0.400 mmol) in NMP (1 mL), were added 15 mg Molecular Sieve powder and sodium triacetoxyborohydride (0.250 g, 1.180 mmol). After 16 h at RT the reaction mixture was diluted with DMF:acetic acid 1:11 mL. The crude material was purified via preparative LC/MS with the following conditions: Column: XBridge C18, 200 mm×19 mm, 5-μm particles; Mobile Phase A: 5:95 acetonitrile:water with NH₄OAc; Mobile Phase B: 95:5 acetonitrile:water with NH₄OAc; Gradient: a 0-minute hold at 8% B, 8-48% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile:water with 0.05% TFA; Mobile Phase B: 95:5 acetonitrile:water with 0.05% TFA; Gradient: a 0-minute hold at 0% B, 0-40% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. to provide Compound 203 (10.1 mg, 7.8% yield).

### Example 28—Compound 210

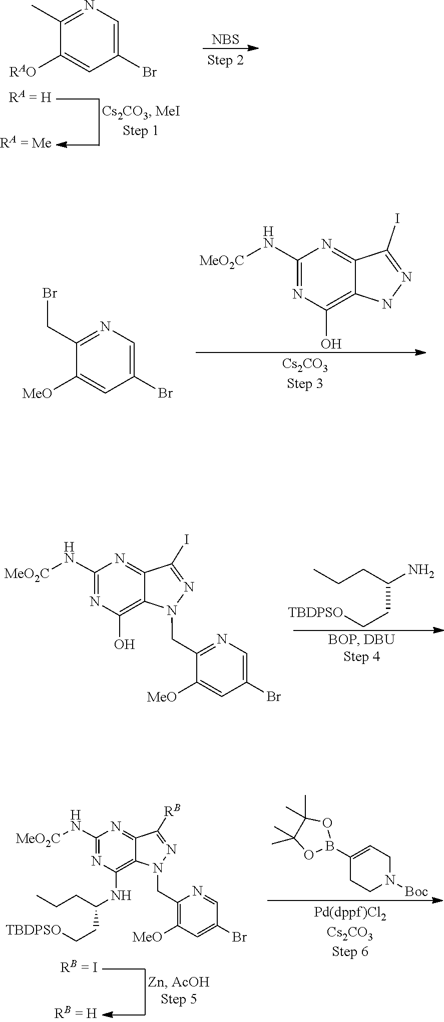

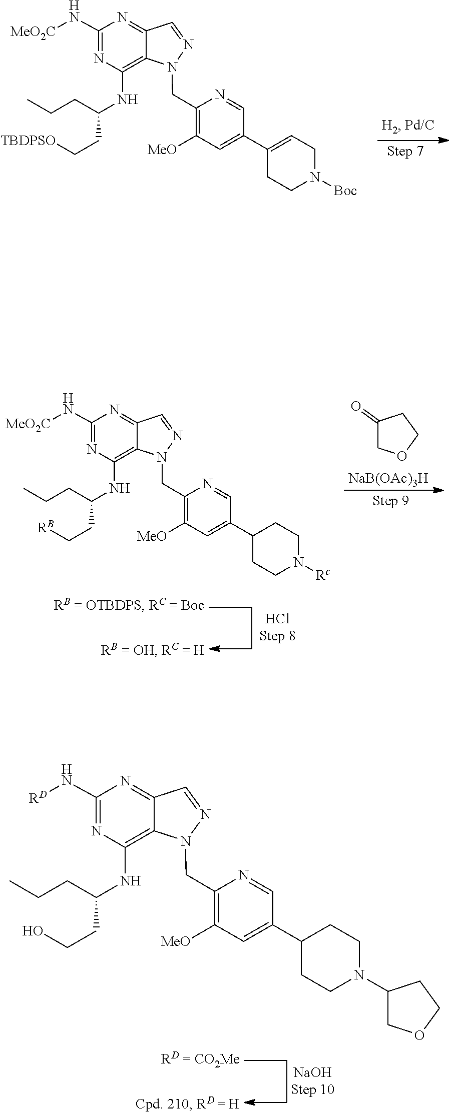

Step 1. To a stirred solution of 5-bromo-2-methylpyridin-3-ol (5 g, 26.6 mmol) in anhydrous acetonitrile (100 mL), were added Cs₂CO₃ (8.66 g, 26.6 mmol) and methyl iodide (4.16 mL, 66.5 mmol) at RT. The reaction mixture was stirred for 2 h at RT. The reaction mixture was partitioned between water and ethyl acetate. The organic layer was washed with H₂O and saturated NaCl solution, dried over anhydrous Na₂SO₄, filtered and concentrated under reduced pressure to provide the crude product. The residue was purified using CombiFlash (silica gel 60-120 mesh; 25% ethyl acetate in petroleum ether as eluent) to afford 5-bromo-3-methoxy-2-methylpyridine (3.5 g, 15.59 mmol, 58.6% yield) as an off-white solid.

¹H NMR (400 MHz, methanol-d₄) δ=8.07 (d, J=2.0 Hz, 1H), 7.55-7.54 (m, 1H), 3.90 (s, 3H), 2.39 (s, 3H).

LCMS (ES): m/z=204.1 [M+H]⁺

Step 2. To a stirred solution of 5-bromo-3-methoxy-2-methylpyridine (7 g, 34.6 mmol) in anhydrous CCl₄ (70 mL), were added NBS (6.47 g, 36.4 mmol) and AIBN (1.138 g, 6.93 mmol) at RT. The reaction mixture was heated to 65° C. and stirred for 16 h. The reaction mixture was concentrated to dryness under high vacuum to provide the crude product. The residue was purified using CombiFlash (silica gel 60-120 mesh; 25% ethyl acetate in petroleum ether as eluent) to afford 5-bromo-2-(bromomethyl)-3-methoxypyridine (7.2 g, 21.78 mmol, 62.9% yield) as a pale yellow solid.

¹H NMR (400 MHz, chloroform-d) δ=8.26 (d, J=2.0 Hz, 1H), 7.36 (d, J=1.5 Hz, 1H), 4.60 (s, 2H), 3.95 (s, 3H).

LCMS (ES): m/z=281.9 [M+H]⁺

Step 3. To a stirred solution of methyl (7-hydroxy-3-iodo-1H-pyrazolo[4,3-d]pyrimidin-5-yl)carbamate (1.1 g, 3.28 mmol) in anhydrous DMF (10 mL) at 0° C., were added Cs₂CO₃ (2.139 g, 6.57 mmol) and 5-bromo-2-(bromomethyl)-3-methoxypyridine (0.922 g, 3.28 mmol). The reaction mixture was stirred for 1 h at 0° C. The reaction mixture was partitioned between water and DCM. The organic layer was washed with H₂O and saturated NaCl solution, dried over anhydrous Na₂SO₄, filtered and concentrated under reduced pressure to get the crude product. The residue was purified using CombiFlash (silica gel 60-120 mesh; 5% methanol in chloroform as eluent) to afford methyl (1-((5-bromo-3-methoxypyridin-2-yl)methyl)-7-hydroxy-3-iodo-1H-pyrazolo[4,3-d]pyrimidin-5-yl)carbamate (450 mg, 0.757 mmol, 23.05% yield) as a brown solid.

¹H NMR (400 MHz, DMSO-d₆) δ=11.65 (br s, 1H), 11.33 (br s, 1H), 8.08 (d, J=1.5 Hz, 1H), 7.76 (d, J=2.0 Hz, 1H), 5.77-5.73 (m, 2H), 3.90 (s, 3H), 3.74 (s, 3H).

LCMS (ES): m/z=537.0 [M+H]⁺.

Step 4. To a stirred solution of methyl (1-((5-bromo-3-methoxypyridin-2-yl)methyl)-7-hydroxy-3-iodo-1H-pyrazolo[4,3-d]pyrimidin-5-yl)carbamate (3 g, 5.61 mmol) in anhydrous DMSO (40 mL), were added BOP (4.96 g, 11.21 mmol), DBU (1.268 mL, 8.41 mmol) and (S)-1-((tert-butyldiphenylsilyl)oxy)hexan-3-amine (2.193 g, 6.17 mmol) at RT. The reaction mixture was heated to 40° C. and stirred for 4 h. The reaction mixture was cooled to 0° C. Ice cold water was added drop-wise. The mixture was extracted with DCM. The organic layer was washed with H₂O, and saturated NaCl solution, dried over anhydrous Na₂SO₄, filtered and concentrated under reduced pressure to provide the crude product. The residue was purified using CombiFlash (silica gel 60-120 mesh; 25% ethyl acetate in petroleum ether as eluent) to afford methyl (S)-(1-((5-bromo-3-methoxypyridin-2-yl)methyl)-7-((1-((tert-butyldiphenylsilyl)oxy)hexan-3-yl)amino)-3-iodo-1H-pyrazolo[4,3-d]pyrimidin-5-yl)carbamate (2.7 g, 2.63 mmol, 46.9% yield) as a pale yellow semi-solid.

¹H NMR (400 MHz, methanol-d₄) δ=8.02 (d, J=1.5 Hz, 1H), 7.73-7.71 (m, 1H), 7.58-7.53 (m, 2H), 7.47-7.43 (m, 2H), 7.37-7.30 (m, 1H), 7.29-7.20 (m, 3H), 7.13-7.06 (m, 2H), 5.64-5.46 (m, 2H), 3.99 (s, 3H), 3.88 (d, J=7.5 Hz, 1H), 3.76 (s, 3H), 2.17-2.04 (m, 1H), 1.98-1.82 (m, 1H), 1.75-1.62 (m, 2H), 1.50-1.35 (m, 3H), 1.33-1.27 (m, 1H), 1.00-0.91 (m, 13H).

LCMS (ES): m/z=872.5 [M+H]⁺.

Step 5. To a stirred solution of methyl (S)-(1-((5-bromo-3-methoxypyridin-2-yl)methyl)-7-((1-((tert-butyldiphenylsilyl)oxy)hexan-3-yl)amino)-3-iodo-1H-pyrazolo[4,3-d]pyrimidin-5-yl)carbamate (5.5 g, 6.30 mmol) in a mixture of methanol (40 mL) and acetic acid (10 mL) at 0° C., was added zinc powder (4.12 g, 63.0 mmol). After being stirred for 2 h at 0° C., the reaction mixture was allowed cool to RT, diluted with water and extracted with DCM. The organic layer was washed with H₂O, and saturated NaCl solution, dried over anhydrous Na₂SO₄, filtered and concentrated under reduced pressure to provide methyl (S)-(1-((5-bromo-3-methoxypyridin-2-yl)methyl)-7-((1-((tert-butyldiphenylsilyl)oxy)hexan-3-yl)amino)-1H-pyrazolo-[4,3-d]pyrimidin-5-yl)carbamate (4.7 g, 5.03 mmol, 80% yield) as a brown solid.

¹H NMR (400 MHz, methanol-d₄) δ=8.02 (d, J=2.0 Hz, 1H), 7.85 (s, 1H), 7.59-7.55 (m, 2H), 7.48-7.43 (m, 6H), 7.30-7.26 (m, 2H), 7.11-7.10 (m, 1H), 5.70-5.49 (m, 2H), 4.76-4.72 (m, 1H), 3.96 (s, 3H), 3.79 (s, 3H), 2.14-2.05 (m, 1H), 1.75-1.62 (m, 3H), 1.46-1.39 (m, 3H), 1.05-1.03 (m, 5H), 0.98 (s, 9H).

LCMS (ES): m/z=748.5 [M+H]⁺.

Step 6. To a stirred solution of methyl (S)-(1-((5-bromo-3-methoxypyridin-2-yl)methyl)-7-((1-((tert-butyldiphenylsilyl)oxy)hexan-3-yl)amino)-1H-pyrazolo[4,3-d]pyrimidin-5-yl)carbamate (4.6 g, 6.16 mmol) in a mixture of dioxane (50 mL) and water (1 mL), were added tert-butyl 4-(4,4,5,5-tetramethyl-1,3,2-dioxaborolan-2-yl)-3,6-dihydropyridine-1(2H)-carboxylate (3.05 g, 9.86 mmol), PdCl₂(dppf)-CH₂Cl₂ adduct (0.503 g, 0.616 mmol) and Cs₂CO₃ (6.02 g, 18.48 mmol) at RT. The reaction mixture was purged with nitrogen, heated to 100° C. and stirred for 16 h. The residue was purified using CombiFlash (silica gel 60-120 mesh; 4% methanol in chloroform as eluent) to afford tert-butyl (S)-6-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-5-methoxy-3′,6′-dihydro-[3,4′-bipyridine]-1′(2′H)-carboxylate (4.6 g, 4.60 mmol, 74.8% yield) as a pale yellow semi solid.

LCMS (ES): m/z=849.7 [M+H]⁺

Step 7. To a stirred solution of tert-butyl (S)-6-((7-((1-((tert-butyldiphenylsilyl)-oxy)hexan-3-yl)amino)-5-((methoxycarbonyl)amino)-1H-pyrazolo[4,3-d]pyrimidin-1-yl)methyl)-5-methoxy-3′,6′-dihydro-[3,4′-bipyridine]-1′(2′H)-carboxylate (4.6 g, 5.42 mmol) in anhydrous methanol (100 mL), was added Pd/C (2.88 g, 2.71 mmol) at RT. The reaction mixture was heated to 50° C. in an autoclave under 5 Kg hydrogen gas pressure for 24 h. The black suspension was filtered through a CELITE™ bed and the bed was washed with ethyl acetate. The filtrate was concentrated under reduced pressure to afford tert-butyl (S)-4-(6-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-5-methoxypyridin-3-yl)piperidine-1-carboxylate (4.1 g, 3.85 mmol, 71.1% yield) as a brown semi-solid.

LCMS (ES): m/z=851.4 [M+H]⁺

Step 8. To a stirred solution of tert-butyl (S)-4-(6-((7-((1-((tert-butyldiphenylsilyl)-oxy)hexan-3-yl)amino)-5-((methoxycarbonyl)amino)-1H-pyrazolo[4,3-d]pyrimidin-1-yl)methyl)-5-methoxypyridin-3-yl)piperidine-1-carboxylate (1 g, 1.175 mmol) in methanol (10 mL), was added conc. HCl (0.357 mL, 11.75 mmol) at RT. After being stirred for 4 h, the reaction mixture was concentrated to dryness under high vacuum to get the crude product. The crude product was triturated with diethyl ether and then the ether was decanted. The resultant residue was dried to provide methyl (S)-(7-((1-hydroxyhexan-3-yl)amino)-1-((3-methoxy-5-(piperidin-4-yl)pyridin-2-yl)methyl)-1H-pyrazolo[4,3-d]pyrimidin-5-yl)carbamate (550 mg, 0.858 mmol, 73.1% yield) as a brown solid.

LCMS (ES): m/z=513.4 [M+H]⁺

Step 9. To a stirred solution of methyl (S)-(7-((1-hydroxyhexan-3-yl)amino)-1-((3-methoxy-5-(piperidin-4-yl)pyridin-2-yl)methyl)-1H-pyrazolo[4,3-d]pyrimidin-5-yl)carbamate (250 mg, 0.488 mmol) in anhydrous DCE (5 mL), was added dihydrofuran-3(2H)-one (126 mg, 1.463 mmol) at RT. After being stirred for 30 min, was added sodium triacetoxyborohydride (517 mg, 2.438 mmol) at RT and stirred for 16 h. The reaction mixture was partitioned between saturated NaHCO₃ solution and DCM. The organic layer was washed with H₂O and saturated NaCl solution, dried over anhydrous Na₂SO₄, filtered and concentrated under reduced pressure to provide crude methyl (7-(((S)-1-hydroxyhexan-3-yl)amino)-1-((3-methoxy-5-(1-(tetrahydrofuran-3-yl)piperidin-4-yl)pyridin-2-yl)methyl)-1H-pyrazolo[4,3-d]pyrimidin-5-yl)carbamate (350 mg), which was used as such in the next reaction.

LCMS (ES): m/z=583.4 [M+H]⁺.

Step 10 To a stirred solution of methyl (7-(((S)-1-hydroxyhexan-3-yl)amino)-1-((3-methoxy-5-(1-(tetrahydrofuran-3-yl)piperidin-4-yl)pyridin-2-yl)methyl)-1H-pyrazolo[4,3-d]pyrimidin-5-yl)carbamate (350 mg, 0.601 mmol) in a mixture of dioxane (5 mL) and water (2 mL), was added NaOH (240 mg, 6.01 mmol) at RT. The reaction mixture was heated to 75° C. and stirred for 3 h. The organic layer from the reaction mixture was separated and the concentrated to dryness to get the residue. The crude compound was purified via preparative LC/MS with the following conditions: Column: Waters XBridge Phenyl C18, 19×250 mm, 5-μm particles; mobile phase A: 10 mM ammonium bicarbonate in water pH 9.5; mobile phase B: acetonitrile; gradient: 20-59% B over 15 minutes; flow rate: 19 mL/min. The crude product was purified in RP HPLC (using NH₄OAc as buffer). The prep. fraction was concentrated under high vacuum at 30° C. The residue was dissolved in a mixture of MeCN and water, frozen and lyophilized for 12 h to afford Compound 210, diastereomeric (65 mg, 0.124 mmol, 20.63% yield) as a white solid.

The diastereomeric compound 210 was subjected to chiral separation to obtain the individual diastereomers using the chiral SFC method (Column: Chiralpak IG (250×4.6) mm, 5 μm, mobile phase: 0.2% of DEA in CH₃CN+isopropanol (1:1), temperature: 30° C.). The first isomer, which was eluted at 7.51 min, was compound 211. The second isomer, which was eluted at 13.37 min, was compound 212. Compounds 211 and 212 thus are enantiomers differing in the stereochemistry at the asterisked (*) carbon.

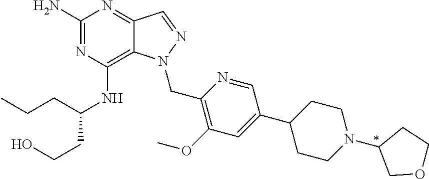 [SMILES: CCC[C@@H](CCO)Nc1nc(N)nc2cnn(Cc3ncc(C4CCN(C5CCOC5)CC4)cc3OC)c12]

### Example 29—Compound 213

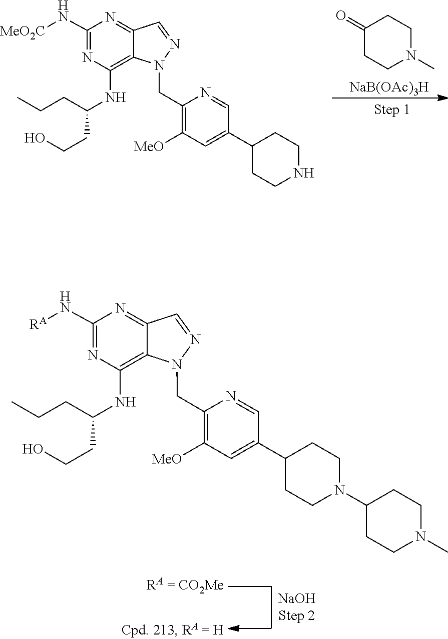

Step 1. To a stirred solution of methyl (S)-(7-((1-hydroxyhexan-3-yl)amino)-1-((3-methoxy-5-(piperidin-4-yl)pyridin-2-yl)methyl)-1H-pyrazolo[4,3-d]pyrimidin-5-yl)carbamate (100 mg, 0.195 mmol) in DCE (2.5 mL), were added 1-methylpiperidin-4-one (44.2 mg, 0.390 mmol) and sodium triacetoxyborohydride (124 mg, 0.585 mmol) at RT. The reaction mixture was stirred at ambient temperature for 16 h. The suspension was filtered through a CELITE™ bed and the bed was washed with ethyl acetate. The filtrate was concentrated under reduced pressure to afford crude methyl (S)-(7-((1-hydroxyhexan-3-yl)amino)-1-((3-methoxy-5-(1′-methyl-[1,4′-bipiperidin]-4-yl)pyridin-2-yl)methyl)-1H-pyrazolo[4,3-d]pyrimidin-5-yl)carbamate (130 mg) as a brown semi-solid, which was used as such in the next reaction.

LCMS (ES) m/z=611.3 [M+H]⁺

Step 2. To a stirred solution of methyl (S)-(7-((1-hydroxyhexan-3-yl)amino)-1-((3-methoxy-5-(1′-methyl-[1,4′-bipiperidin]-4-yl)pyridin-2-yl)methyl)-1H-pyrazolo[4,3-d]pyrimidin-5-yl)carbamate (130 mg, 0.213 mmol) in a mixture of dioxane (2 mL) and water (1 mL), was added NaOH (85 mg, 2.132 mmol) at RT. The reaction mixture was heated to 75° C. and stirred for 3 h. The dioxane layer from the reaction mixture was separated and then concentrated to dryness to provide the crude product. The crude material was purified via preparative LC/MS with the following conditions: Column: Waters XBridge C18, 19×150 mm, 5-μm particles; mobile phase A: 10 mM NH₄OAc; mobile phase B: acetonitrile; gradient: 10-35% B over 20 minutes, then a 5-minute hold at 100% B; flow rate: 15 mL/min. The fraction collection was triggered by MS and UV signals. Fractions containing the desired product were combined and dried via centrifugal evaporation to afford Compound 213 (6.9 mg, 0.013 mmol, 5.87% yield).

### Example 30—Compound 214

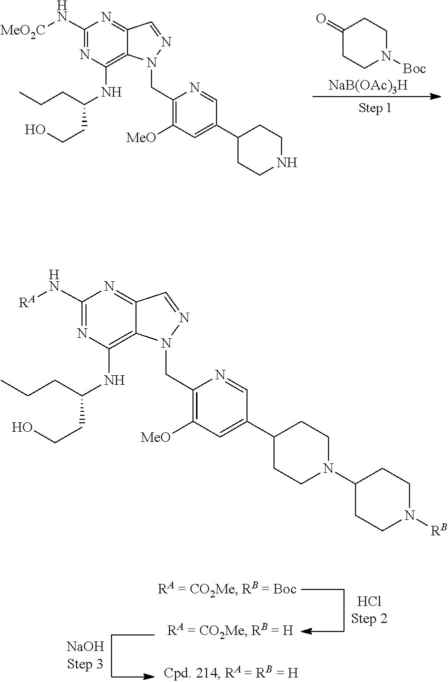

Step 1. To a stirred solution of methyl (S)-(7-((1-hydroxyhexan-3-yl)amino)-1-((3-methoxy-5-(piperidin-4-yl)pyridin-2-yl)methyl)-1H-pyrazolo[4,3-d]pyrimidin-5-yl)carbamate (100 mg, 0.195 mmol) in DCE (2.5 mL), were added tert-butyl 4-oxopiperidine-1-carboxylate (78 mg, 0.390 mmol) and sodium triacetoxyborohydride (124 mg, 0.585 mmol) at RT. The reaction mixture was stirred for 16 h. The suspension was filtered through a CELITE™ bed and the bed was washed with ethyl acetate. The filtrate was concentrated under reduced pressure to afford crude tert-butyl (S)-4-(6-((7-((1-hydroxyhexan-3-yl)amino)-5-((methoxycarbonyl)amino)-1H-pyrazolo[4,3-d]pyrimidin-1-yl)methyl)-5-methoxypyridin-3-yl)-[1,4′-bipiperidine]-1′-carboxylate as a brown semi-solid (150 mg), which was used as such in the next reaction.

LCMS (ES) m/z=696.4 [M+H]⁺

Step 2. To a stirred solution of tert-butyl (S)-4-(6-((7-((1-hydroxyhexan-3-yl)amino)-5-((methoxycarbonyl)amino)-1H-pyrazolo[4,3-d]pyrimidin-1-yl)methyl)-5-methoxypyridin-3-yl)-[1,4′-bipiperidine]-1′-carboxylate (150 mg, 0.216 mmol) in anhydrous DCM (2 mL), was added drop-wise 4 M HCl in dioxane (2.5 mL, 10.00 mmol) at RT. After being stirred for 1 h, the reaction mixture was concentrated to dryness under high vacuum to afford crude methyl (S)-(1-((5-([1,4′-bipiperidin]-4-yl)-3-methoxypyridin-2-yl)methyl)-7-((1-hydroxyhexan-3-yl)amino)-1H-pyrazolo[4,3-d]pyrimidin-5-yl)carbamate (130 mg), which was used as such in the next reaction.

LCMS (ES) m/z=596.4 [M+H]⁺.

Step 3. To a stirred solution of methyl (S)-(1-((5-([1,4′-bipiperidin]-4-yl)-3-methoxypyridin-2-yl)methyl)-7-((1-hydroxyhexan-3-yl)amino)-1H-pyrazolo[4,3-d]pyrimidin-5-yl)carbamate (130 mg, 0.218 mmol) in a mixture of dioxane (2 mL) and water (1 mL), was added NaOH (87 mg, 2.182 mmol) at RT. The reaction mixture was heated to 75° C. and stirred for 4 h. The dioxane layer from the reaction mixture was separated and the concentrated to dryness to provide the crude product. The crude material was purified via preparative LC/MS with the following conditions: Column: Waters XBridge C18, 19×150 mm, 5-μm particles; mobile phase A: 10 mM NH₄OAc; mobile phase B: acetonitrile; gradient: 10-40% B over 20 minutes, then a 5-minute hold at 100% B; flow rate: 15 mL/min. The fraction collection was triggered by MS and UV signals. Fractions containing the desired product were combined and dried via centrifugal evaporation to afford Compound 214 (5.6 mg, 9.89 μmol, 4.53% yield).

### Example 31—Compound 216

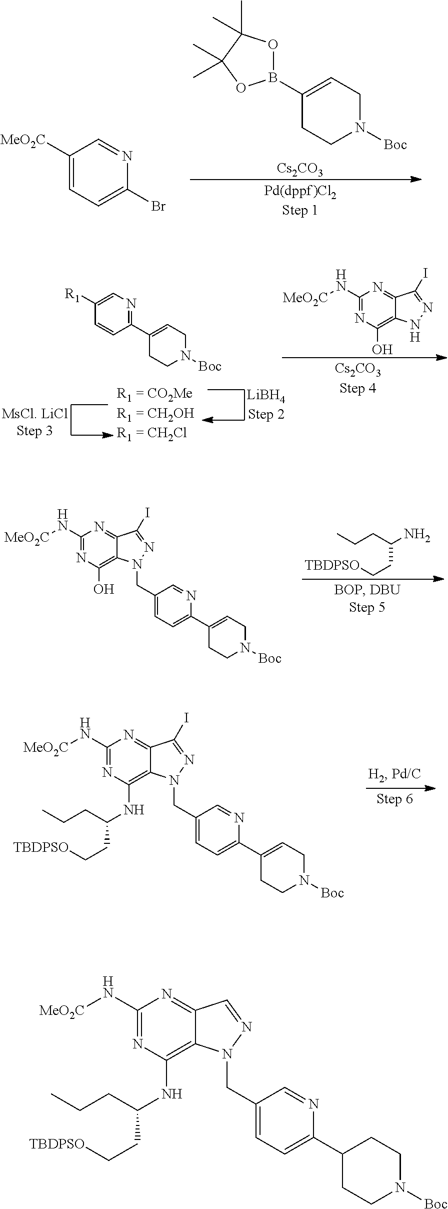

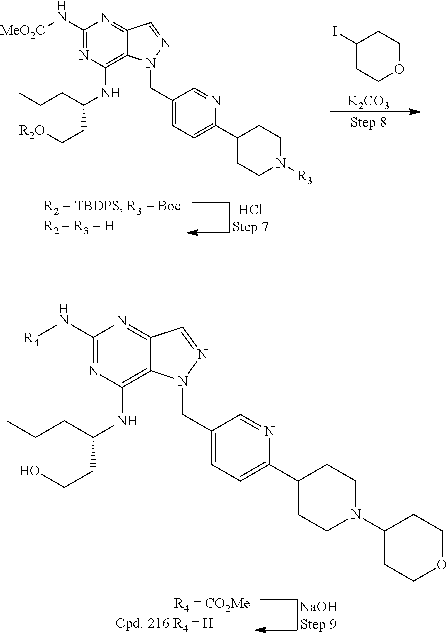

Step 1. To a stirred solution of methyl 6-bromonicotinate (5 g, 23.14 mmol) in 1,4-dioxane (100 mL), were added tert-butyl 4-(4,4,5,5-tetramethyl-1,3,2-dioxaborolan-2-yl)-3,6-dihydropyridine-1(2H)-carboxylate (10.73 g, 34.7 mmol), Cs₂CO₃ (15.08 g, 46.3 mmol) and PdCl₂(dppf)-CH₂Cl₂ adduct (1.890 g, 2.314 mmol). The mixture was purged with N₂ gas. After being stirred at 100° C. for 5 h, the reaction mixture was partitioned between ethyl acetate and ice cold water. The organic layer was washed with water and brine, dried over anhydrous Na₂SO₄ and concentrated in vacuo at 45° C. The crude product was purified by CombiFlash chromatography (60-120 silica gel; 20-60% ethyl acetate in petroleum ether as eluent) to afford 1′-(tert-butyl)₅-methyl 3′,6′-dihydro-[2,4′-bipyridine]-1′,5(2′H)-dicarboxylate (4.6 g, 14.45 mmol, 62.4% yield) as a light yellow oil.

¹H NMR (400 MHz, chloroform-d) δ=9.16 (s, 1H), 8.25 (d, J=8.6 Hz, 1H), 7.45 (d, J=8.5 Hz, 1H), 6.81-6.78 (m, 1H), 4.18 (br d, J=3.0 Hz, 2H), 3.96 (s, 3H), 3.69-3.65 (m, 2H), 2.67 (br d, J=2.0 Hz, 2H), 1.50 (s, 9H).

LCMS (ES) m/z=319.2 [M+H]⁺

Step 2. To a solution of 1′-(tert-butyl) 5-methyl 3′,6′-dihydro-[2,4′-bipyridine]-1′,5(2′H)-dicarboxylate (4 g, 12.56 mmol) in a mixture of THF (50 mL) and MeOH (10 mL), was added LiBH₄ (31.4 mL, 62.8 mmol) under nitrogen atmosphere and then partitioned between ammonium chloride solution and EtOAc. The organic layer was washed with water and brine, dried over anhydrous Na₂SO₄ and concentrated to get crude product as a brown oil, which was purified by CombiFlash chromatography (60-120 silica gel; 10-100% ethyl acetate in petroleum ether to afford tert-butyl 5-(hydroxymethyl)-3′,6′-dihydro-[2,4′-bipyridine]-1′(2′H)-carboxylate (3.6 g, 12.40 mmol, 99% yield) as a light yellow oil.

¹H NMR (400 MHz, DMSO-d₆) δ=8.47 (d, J=2.0 Hz, 1H), 7.70 (dd, J=2.0, 8.0 Hz, 1H), 7.53 (d, J=8.5 Hz, 1H), 6.66 (br s, 1H), 5.30 (t, J=5.5 Hz, 1H), 4.52 (d, J=5.5 Hz, 2H), 4.10-3.99 (m, 2H), 3.54 (t, J=5.8 Hz, 2H), 2.58-2.52 (m, 2H), 1.43 (s, 9H).

LCMS (ES) m/z=291.3 [M+H]⁺

Step 3. To a stirred solution of tert-butyl 5-(hydroxymethyl)-3′,6′-dihydro-[2,4′-bipyridine]-1′(2′H)-carboxylate (3.5 g, 12.05 mmol) in dry DCM (30 mL), were added TEA (3.36 mL, 24.11 mmol), MsCl (1.878 mL, 24.11 mmol) and lithium chloride (1.022 g, 24.11 mmol) at 0° C. The reaction mixture was allowed to cool to RT and stirred for 16 h. The reaction mixture was partitioned between ethyl acetate and water. The organic layer was washed with brine, dried over anhydrous Na₂SO₄ and concentrated in vacuo to get crude product, which was purified by CombiFlash chromatography (60-120 silica gel; 10-30% ethyl acetate in petroleum ether to afford tert-butyl 5-(chloromethyl)-3′,6′-dihydro-[2,4′-bipyridine]-1′(2′H)-carboxylate (3.7 g, 11.98 mmol, 99% yield) as a white solid.

¹H NMR (400 MHz, chloroform-d) δ=8.58 (d, J=2.0 Hz, 1H), 7.72 (dd, J=2.3, 8.3 Hz, 1H), 7.41 (d, J=8.0 Hz, 1H), 6.66 (td, J=1.6, 3.4 Hz, 1H), 4.61 (s, 2H), 4.16 (br d, J=3.0 Hz, 2H), 3.67 (br t, J=5.5 Hz, 2H), 2.70-2.63 (m, 2H), 1.58 (s, 9H).

LCMS (ES) m/z=309.2 [M+H]⁺.

Step 4. To a stirred solution of methyl (7-hydroxy-3-iodo-1H-pyrazolo[4,3-d]pyrimidin-5-yl)carbamate (3.8 g, 11.34 mmol) in dry DMF (20 mL), were added Cs₂CO₃ (7.39 g, 22.68 mmol) and tert-butyl 5-(chloromethyl)-3′,6′-dihydro-[2,4′-bipyridine]-1′(2′H)-carboxylate (3.50 g, 11.34 mmol) at RT. After being stirred for 12 h, the reaction mixture was partitioned between ethyl acetate and ice cold water. The organic layer was washed with water and brine, dried over anhydrous Na₂SO₄ and concentrated in vacuo. The crude product was purified by CombiFlash chromatography (60-120 silica gel; 20-60% ethyl acetate in CHCl₃ as eluent) to afford tert-butyl 5-((7-hydroxy-3-iodo-5-((methoxycarbonyl)amino)-1H-pyrazolo[4,3-d]pyrimidin-1-yl)methyl)-3′,6′-dihydro-[2,4′-bipyridine]-1′(2′H)-carboxylate (1.5 g, 2.470 mmol, 21.77% yield) as an off-white solid.

¹H NMR (400 MHz, DMSO-d₆) δ=11.72 (br s, 1H), 11.36 (br s, 1H), 8.50 (d, J=1.5 Hz, 1H), 7.65 (d, J=7.9 Hz, 1H), 7.53 (d, J=8.0 Hz, 1H), 6.70-6.62 (m, 1H), 5.79-5.70 (m, 2H), 4.07-3.98 (m, 2H), 3.75 (s, 3H), 3.52 (t, J=5.8 Hz, 2H), 2.56-2.53 (m, 2H), 1.48 (s, 1H), 1.42 (s, 9H).

LCMS (ES): m/z=608.2 [M+H]⁺.

Step 5. To a stirred solution of tert-butyl 5-((7-hydroxy-3-iodo-5-((methoxycarbonyl)-amino)-1H-pyrazolo[4,3-d]pyrimidin-1-yl)methyl)-3′,6′-dihydro-[2,4′-bipyridine]-1′(2′H)-carboxylate (1.50 g, 2.470 mmol) in anhydrous DMSO (15 mL), were added DBU (1.117 mL, 7.41 mmol), BOP (1.638 g, 3.70 mmol) and (S)-1-((tert-butyldiphenylsilyl)oxy)hexan-3-amine (0.878 g, 2.470 mmol) at RT. The reaction mixture was heated to 45° C., stirred for 4 h, and partitioned between water and ethyl acetate. The organic layer was washed with brine solution, dried over anhydrous Na₂SO₄, filtered and concentrated under vacuum to get the crude product as a light yellow oil. The crude product was purified using CombiFlash (silica gel 60-120 mesh; 25% ethyl acetate in chloroform as eluent). The fraction was concentrated using high vacuum at 50° C. to afford tert-butyl (S)-5-((7-((1-((tert-butyldiphenylsilyl)oxy)hexan-3-yl)amino)-3-iodo-5-((methoxycarbonyl)amino)-1H-pyrazolo[4,3-d]pyrimidinyl)methyl)3′,6′dihydro[2,4′-bipyridine]-1′(2′H)-carboxylate (0.900 g, 0.867 mmol, 35.1% yield) as a yellow solid.

¹H NMR (400 MHz, DMSO-d₆) δ=8.24 (d, J=2.0 Hz, 1H), 7.62-7.58 (m, 3H), 7.51-7.48 (m, 2H), 7.34 (br d, J=7.0 Hz, 4H), 7.15-7.08 (m, 3H), 6.42 (br s, 1H), 5.93-5.78 (m, 2H), 4.65 (br dd, J=4.0, 8.0 Hz, 1H), 4.01 (q, J=7.0 Hz, 1H), 3.89 (br s, 2H), 3.55 (s, 3H), 3.46-3.39 (m, 1H), 3.35 (t, J=5.8 Hz, 2H), 2.30 (br s, 2H), 1.77 (br d, J=4.5 Hz, 2H), 1.62-1.52 (m, 2H), 1.39 (s, 9H), 1.16 (t, J=7.0 Hz, 3H).

LCMS (ES) m/z=945.2 [M+H]⁺

Step 6. To a stirred solution of tert-butyl (S)-5-((7-((1-((tert-butyldiphenyl-silyl)oxy)hexan-3-yl)amino)-3-iodo-5-((methoxycarbonyl)amino)-1H-pyrazolo[4,3-d]pyrimidin-1-yl)methyl)-3′,6′-dihydro-[2,4′-bipyridine]-1′(2′H)-carboxylate (930 mg, 0.984 mmol) in anhydrous methanol (25 mL), was added Pd/C (524 mg, 0.492 mmol) at RT. The reaction mixture was heated to 50° C. in an autoclave under 5 Kg hydrogen gas pressure for 24 h. The black suspension was filtered through a CELITE™ bed and the bed was washed with ethyl acetate. The filtrate was concentrated under reduced pressure to afford tert-butyl (S)-4-(5-((7-((1-((tert-butyldiphenylsilyl)oxy)hexan-3-yl)amino)-5-((methoxycarbonyl)amino)-1H-pyrazolo[4,3-d]pyrimidin-1-yl)methyl)pyridin-2-yl)piperidine-1-carboxylate (800 mg, 0.779 mmol, 79% yield) as a pale yellow semi-solid.

LCMS (ES): m/z=821.5 [M+H]⁺

Step 7. To a stirred solution of tert-butyl (S)-4-(5-((7-((1-((tert-butyldiphenyl-silyl)oxy)hexan-3-yl)amino)-5-((methoxycarbonyl)amino)-1H-pyrazolo[4,3-d]pyrimidin-1-yl)methyl)pyridin-2-yl)piperidine-1-carboxylate (200 mg, 0.244 mmol) in methanol (2 mL), was added conc. HCl (4 mL, 132 mmol) at RT. After stirring for 3 h, the reaction mixture was concentrated to dryness under high vacuum to provide the crude product. The crude product was triturated with diethyl ether and the ether layer was decanted. The residue was dried to afford methyl (S)-(7-((1-hydroxyhexan-3-yl)amino)-1-((6-(piperidin-4-yl)pyridin-3-yl)methyl)-1H-pyrazolo[4,3-d]pyrimidin-5-yl)carbamate (170 mg, assumed 100% yield) as a brown solid.

LCMS (ES) m/z=483.3 [M+H]⁺

Step 8. To a stirred solution of methyl (S)-(7-((1-hydroxyhexan-3-yl)amino)-1-((6-(piperidin-4-yl)pyridin-3-yl)methyl)-1H-pyrazolo[4,3-d]pyrimidin-5-yl)carbamate (130 mg, 0.269 mmol) in anhydrous DMF (2 mL), were added K₂CO₃ (112 mg, 0.808 mmol) and 4-iodotetra-hydro-2H-pyran (86 mg, 0.404 mmol) at RT. The reaction mixture was heated to 55° C., stirred for 24 h, and concentrated to dryness under high vacuum to provide crude methyl (S)-(7-((1-hydroxyhexan-3-yl)amino)-1-((6-(1-(tetrahydro-2H-pyran-4-yl)piperidin-4-yl)pyridin-3-yl)methyl)-1H-pyrazolo[4,3-d]pyrimidin-5-yl)carbamate (160 mg), which was used as such in the next reaction.

LC-MS (ES) m/z=567.3 [M+H]⁺

Step 9. To a stirred solution of methyl (S)-(7-((1-hydroxyhexan-3-yl)amino)-1-((6-(1-(tetrahydro-2H-pyran-4-yl)piperidin-4-yl)pyridin-3-yl)methyl)-1H-pyrazolo[4,3-d]pyrimidin-5-yl)carbamate (160 mg, 0.282 mmol) in a mixture of dioxane (2 mL) and water (1 mL), was added NaOH (113 mg, 2.82 mmol) at RT. The reaction mixture was heated to 70° C. and stirred for 3 h. The organic layer from the reaction mixture was separated and the concentrated to dryness to provide the crude product. The residue was purified by reversed phase preparative LC/MS (Column: Waters XBridge C18, 19×150 mm, 5-μm particles; mobile phase A: 10 mM NH₄OAc; mobile phase B: acetonitrile; gradient: 10-45% B over 20 minutes, then a 5-minute hold at 100% B; flow rate: 15 mL/min). The fraction collection was triggered by MS and UV signals. Fractions containing the desired product were combined and dried via centrifugal evaporation using Genevac to afford Compound 216 (20.7 mg, 0.040 mmol, 14.05% yield).

### Example 32—Compound 215

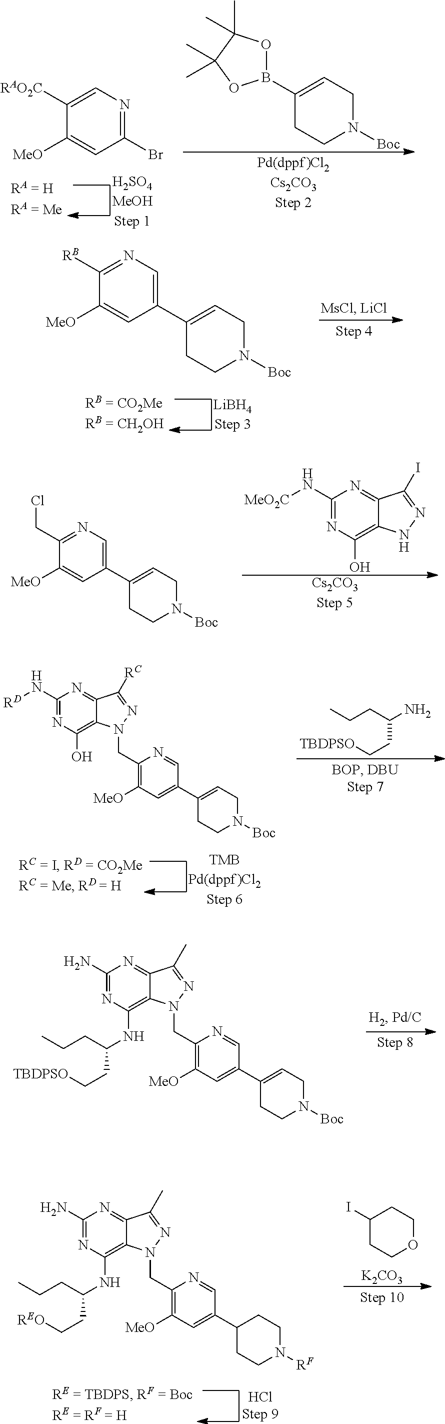

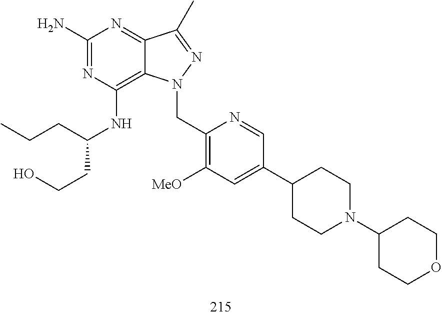 [SMILES: CCC[C@@H](CCO)Nc1nc(N)nc2c(C)nn(Cc3ncc(C4CCN(C5CCOCC5)CC4)cc3OC)c12]

Step 1. To a stirred solution of 5-bromo-3-methoxypicolinic acid (10 g, 43.1 mmol) in anhydrous methanol (100 mL), was added H₂SO₄ (2.297 mL, 43.1 mmol) at RT. The reaction mixture was heated to 70° C., stirred for 16 h, and concentrated to dryness under high vacuum to provide the crude product. The residue was partitioned between saturated NaHCO₃ solution and DCM. The organic layer was washed with H₂O and saturated NaCl solution, dried over anhydrous Na₂SO₄, filtered and concentrated under reduced pressure to afford methyl 5-bromo-3-methoxypicolinate (10.1 g, 41.0 mmol, 95% yield) as a pale yellow oil.

¹H NMR (400 MHz, chloroform-d) δ=8.35 (d, J=1.8 Hz, 1H), 7.53 (d, J=1.8 Hz, 1H), 3.98 (s, 3H), 3.95 (s, 3H).

LCMS (ES): m/z=246.0 [M+H]⁺.

Step 2. To a stirred solution of methyl 5-bromo-3-methoxypicolinate (5 g, 20.32 mmol) in a mixture of dioxane (75 mL) and water (1 mL), were added tert-butyl 4-(4,4,5,5-tetramethyl-1,3,2-dioxaborolan-2-yl)-3,6-dihydropyridine-1(2H)-carboxylate (10.05 g, 32.5 mmol), Cs₂CO₃ (19.86 g, 61.0 mmol) and PdCl₂(dppf)-CH₂Cl₂ adduct (1.659 g, 2.032 mmol) at RT. The reaction mixture was purged with nitrogen, heated to 100° C. and stirred for 16 h. The black suspension was filtered through a CELITE™ bed and the bed was washed with ethyl acetate. The filtrate was concentrated under reduced pressure to afford the crude product as a brown semi solid. The residue was purified using CombiFlash (silica gel 60-120 mesh; 35% ethyl acetate in petroleum ether as eluent) to afford 1′-(tert-butyl) 6-methyl 5-methoxy-3′,6′-dihydro-[3,4′-bipyridine]-1′,6(2′H)-dicarboxylate (4.4 g, 12.63 mmol, 62.2% yield) as a brown semi-solid.

¹H NMR (400 MHz, chloroform-d) δ=8.33 (s, 1H), 6.23 (br s, 1H), 4.17-4.14 (m, 2H), 4.00 (s, 3H), 3.97 (s, 3H), 3.70 (t, J=5.5 Hz, 2H), 2.59-2.55 (m, 2H), 1.52-1.52 (m, 9H).

LCMS (ES): m/z=349.3 [M+H]⁺.

Step 3. To a stirred solution of 1′-(tert-butyl) 6-methyl 5-methoxy-3′,6′-dihydro-[3,4′-bipyridine]-1′,6(2′H)-dicarboxylate (3.7 g, 10.62 mmol) in a mixture of THF (40 mL) and methanol (5 mL) at 0° C., was added 2 M LiBH₄ in THF (21.24 mL, 42.5 mmol). The ice bath was removed and the reaction mixture was stirred for 6 h at RT and then cooled to 0° C. Ice cold water was added drop-wise. The mixture was extracted with DCM. The organic layer was washed with H₂O, and saturated NaCl solution, dried over anhydrous Na₂SO₄, filtered and concentrated under reduced pressure to afford tert-butyl 6-(hydroxymethyl)-5-methoxy-3′,6′-dihydro-[3,4′-bipyridine]-1′(2′H)-carboxylate (3.1 g, 8.22 mmol, 77% yield) as a brown solid.

¹H NMR (400 MHz, DMSO-d₆) δ=8.18 (d, J=1.0 Hz, 1H), 7.40 (d, J=1.0 Hz, 1H), 6.32-6.25 (m, 1H), 4.80 (t, J=5.8 Hz, 1H), 4.52 (d, J=6.0 Hz, 2H), 4.03 (br s, 2H), 3.86 (s, 3H), 3.56 (t, J=5.5 Hz, 2H), 1.46-1.42 (m, 11H).

LCMS (ES): m/z=321.0 [M+H]⁺.

Step 4. To a stirred solution of tert-butyl 6-(hydroxymethyl)-5-methoxy-3′,6′-dihydro-[3,4′-bipyridine]-1′(2′H)-carboxylate (2.8 g, 8.74 mmol) in anhydrous DCM (40 mL), were added TEA (3.65 mL, 26.2 mmol) and mesyl chloride (1.362 mL, 17.48 mmol) at RT. After being stirred for 5 min at RT, lithium chloride (0.741 g, 17.48 mmol) was added. After being stirred for 16 h, the reaction mixture was partitioned between saturated NaHCO₃ solution and DCM. The organic layer was washed with H₂O and saturated NaCl solution, dried over anhydrous Na₂SO₄, filtered and concentrated under reduced pressure to afford tert-butyl 6-(chloromethyl)-5-methoxy-3′,6′-dihydro-[3,4′-bipyridine]-1′(2′H)-carboxylate (3.2 g, 8.03 mmol, 92% yield) as a brown semi-solid.

¹H NMR (400 MHz, methanol-d₄) δ=8.23-8.21 (m, 1H), 7.64 (s, 1H), 6.38-6.33 (m, 1H), 4.77-4.75 (m, 2H), 4.20 (s, 2H), 4.02 (s, 3H), 3.71-3.67 (m, 2H), 2.64-2.58 (m, 2H), 2.60 (br s, 2H), 1.51 (s, 9H).

LCMS (ES): m/z=339.2 [M+H]⁺

Step 5. To a stirred solution of methyl (7-hydroxy-3-iodo-1H-pyrazolo[4,3-d]pyrimidin-5-yl)carbamate (2.7 g, 8.06 mmol) in a mixture of DMF (30 mL) at 0° C., were added Cs₂CO₃ (5.25 g, 16.12 mmol) and tert-butyl 6-(bromomethyl)-5-methoxy-3′,6′-dihydro-[3,4′-bipyridine]-1′(2′H)-carboxylate (3.09 g, 8.06 mmol). The ice bath was removed and the reaction mixture was stirred for 16 h at RT and then partitioned between water and DCM. The organic layer was washed with H₂O and saturated NaCl solution, dried over anhydrous Na₂SO₄, filtered and concentrated under reduced pressure to get the crude product. The residue was purified using CombiFlash (silica gel 60-120 mesh; 5% methanol in chloroform as eluent) to afford tert-butyl 6-((7-hydroxy-3-iodo-5-((methoxycarbonyl)amino)-1H-pyrazolo[4,3-d]pyrimidin-1-yl)methyl)-5-methoxy-3′,6′-dihydro-[3,4′-bipyridine]-1′(2′H)-carboxylate (1.95 g, 2.294 mmol, 28.5% yield) as a brown solid.

¹H NMR (400 MHz, methanol-d₄) δ=8.03 (s, 1H), 7.44 (s, 1H), 6.27-6.20 (m, 1H), 5.91-5.88 (m, 2H), 4.20 (s, 2H), 4.10 (br s, 3H), 3.93 (s, 3H), 3.69-3.64 (m, 2H), 2.58-2.52 (m, 2H), 1.31 (s, 9H).

LCMS (ES): m/z=638.2 [M+H]⁺.

Step 6. To a stirred solution of tert-butyl 6-((7-hydroxy-3-iodo-5-((methoxycarbonyl)-amino)-1H-pyrazolo[4,3-d]pyrimidin-1-yl)methyl)-5-methoxy-3′,6′-dihydro-[3,4′-bipyridine]-1′(2′H)-carboxylate (1.8 g, 2.82 mmol) in a mixture of dioxane (50 mL) and water (0.1 mL), were added K₂CO₃ (1.171 g, 8.47 mmol), PdCl₂(dppf)-CH₂Cl₂ adduct (0.231 g, 0.282 mmol) and 2,4,6-trimethyl-1,3,5,2,4,6-trioxatriborinane (1.974 mL, 14.12 mmol) at RT. The reaction mixture was purged with nitrogen, heated to 100° C., and stirred for 16 h. The black suspension was filtered through a CELITE™ bed and the bed was washed with ethyl acetate. The filtrate was concentrated under reduced pressure to afford the crude product as a semi-solid. The residue was purified using CombiFlash (silica gel 60-120 mesh; 3% methanol in chloroform as eluent) to afford tert-butyl 6-((5-amino-7-hydroxy-3-methyl-1H-pyrazolo[4,3-d]pyrimidin-1-yl)methyl)-5-methoxy-3′,6′-dihydro-[3,4′-bipyridine]-1′(2′H)-carboxylate (550 mg, 0.765 mmol, 27.1% yield) as a pale yellow semi-solid.

¹H NMR (400 MHz, DMSO-d₆) δ=10.85-10.76 (m, 1H), 8.02 (s, 1H), 7.42 (br s, 1H), 6.26 (br s, 1H), 6.03-5.98 (m, 2H), 4.01 (br s, 2H), 3.89 (s, 3H), 3.54 (br t, J=5.5 Hz, 2H), 2.18 (s, 3H), 1.43 (s, 9H), 1.28-1.21 (m, 2H).

LCMS (ES): m/z=468.2 [M+H]⁺

Step 7. To a stirred solution of tert-butyl 6-((5-amino-7-hydroxy-3-methyl-1H-pyrazolo[4,3-d]pyrimidin-1-yl)methyl)-5-methoxy-3′,6′-dihydro-[3,4′-bipyridine]-1′(2′H)-carboxylate (450 mg, 0.963 mmol) in anhydrous DMSO (3 mL), were added BOP (851 mg, 1.925 mmol), DBU (0.290 mL, 1.925 mmol) and (S)-1-((tert-butyldiphenylsilyl)oxy)hexan-3-amine (376 mg, 1.059 mmol) at RT. The reaction mixture was heated to 40° C. and stirred for 3 h. The reaction mixture was allowed cool to RT and diluted with water and extracted with DCM. The organic layer was washed with H₂O, and saturated NaCl solution, dried over anhydrous Na₂SO₄, filtered and concentrated under reduced pressure to provide the crude product. The residue was purified using CombiFlash (silica gel 60-120 mesh; 3% methanol in chloroform as eluent) to afford tert-butyl (S)-6-((5-amino-7-((1-((tert-butyldiphenylsilyl)oxy)hexan-3-yl)amino)-3-methyl-1H-pyrazolo[4,3-d]pyrimidin-1-yl)methyl)-5-methoxy-3′,6′-dihydro-[3,4′-bipyridine]-1′(2′H)-carboxylate (800 mg, 0.894 mmol, 93% yield) as a pale yellow semi-solid.

¹H NMR (400 MHz, methanol-d₄) δ=7.96 (s, 1H), 7.59 (d, J=7.0 Hz, 2H), 7.48-7.40 (m, 7H), 7.32-7.26 (m, 2H), 6.10 (br d, J=2.5 Hz, 1H), 5.47 (s, 2H), 4.73-4.65 (m, 1H), 4.18-4.09 (m, 2H), 4.01 (br s, 2H), 3.97 (s, 3H), 3.86-3.78 (m, 2H), 1.72-1.67 (m, 2H), 1.51 (s, 9H), 1.33 (br d, J=7.5 Hz, 5H), 1.07-1.01 (m, 4H), 0.99 (s, 9H).

LCMS (ES): m/z=805.4 [M+H]⁺.

Step 8. To a stirred solution of tert-butyl (S)-6-((5-amino-7-((1-((tert-butyldiphenyl-silyl)oxy)hexan-3-yl)amino)-3-methyl-1H-pyrazolo[4,3-d]pyrimidin-1-yl)methyl)-5-methoxy-3′,6′-dihydro-[3,4′-bipyridine]-1′(2′H)-carboxylate (800 mg, 0.994 mmol) in anhydrous methanol (20 mL), was added Pd/C (529 mg, 0.497 mmol). The reaction mixture was stirred under 5 kg hydrogen gas pressure at 50° C. for 16 h. The black suspension was filtered through a CELITE™ bed and the bed was washed with ethyl acetate. The filtrate was concentrated under reduced pressure to afford tert-butyl (S)-4-(6-((5-amino-7-((1-((tert-butyldiphenylsilyl)oxy)hexan-3-yl)amino)-3-methyl-1H-pyrazolo[4,3-d]pyrimidin-1-yl)methyl)-5-methoxypyridin-3-yl)piperidine-1-carboxylate (700 mg, 0.867 mmol, 87% yield) as pale yellow semi-solid, which was used as such in the next reaction.

LCMS (ES): m/z=807.4 [M+H]⁺

Step 9. To a stirred solution of tert-butyl (S)-4-(6-((5-amino-7-((1-((tert-butyl-diphenylsilyl)oxy)hexan-3-yl)amino)-3-methyl-1H-pyrazolo[4,3-d]pyrimidin-1-yl)methyl)-5-methoxypyridin-3-yl)piperidine-1-carboxylate (700 mg, 0.867 mmol) in methanol (2 mL), was added concentrated HCl (5 mL, 165 mmol) at RT. After being stirred for 4 h, the reaction mixture was concentrated to dryness under high vacuum to provide the crude product. The crude product was triturated with diethyl ether and then the ether was decanted. The residue was dried to afford (S)-3-((5-amino-1-((3-methoxy-5-(piperidin-4-yl)pyridin-2-yl)methyl)-3-methyl-1H-pyrazolo[4,3-d]pyrimidin-7-yl)amino)hexan-1-ol (500 mg) as a brown semi-solid.

LCMS (ES): m/z=469.4 [M+H]⁺

Step 10. To a stirred solution of (S)-3-((5-amino-1-((3-methoxy-5-(piperidin-4-yl)pyridin-2-yl)methyl)-3-methyl-1H-pyrazolo[4,3-d]pyrimidin-7-yl)amino)hexan-1-ol (80 mg, 0.171 mmol) in anhydrous DMF (1 mL), were added K₂CO₃ (70.8 mg, 0.512 mmol) and 4-iodotetrahydro-2H-pyran (181 mg, 0.854 mmol) at RT. The reaction mixture was heated to 50° C. and stirred for 16 h. The reaction mixture was partitioned between water and ethyl acetate. The organic layer was washed with H₂O and saturated NaCl solution, dried over anhydrous Na₂SO₄, filtered and concentrated under reduced pressure to provide the crude product. The residue was purified by reversed phase preparative LC/MS (Column: Waters XBridge C18, 19×150 mm, 5-μm particles; mobile phase A: 10 mM NH₄OAc; mobile phase B: acetonitrile; gradient: 10-45% B over 20 minutes, then a 5-minute hold at 100% B; flow rate: 15 mL/min). The fraction collection was triggered by MS and UV signals. Fractions containing the desired product were combined and dried via centrifugal evaporation using Genevac to afford Compound 215 (8.1 mg, 0.014 mmol, 8.17% yield).

### Example 33—Compound 217

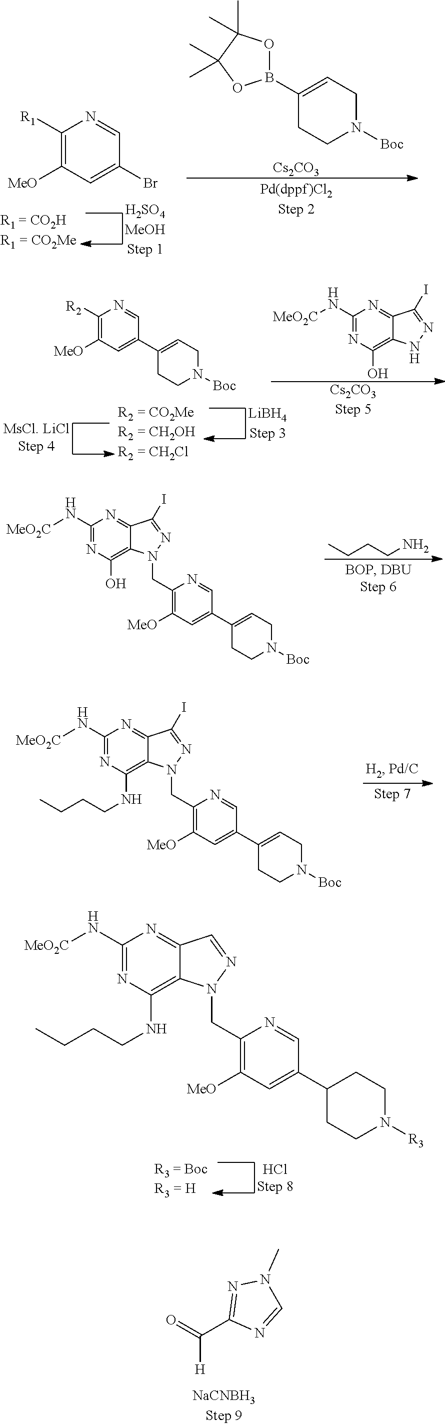

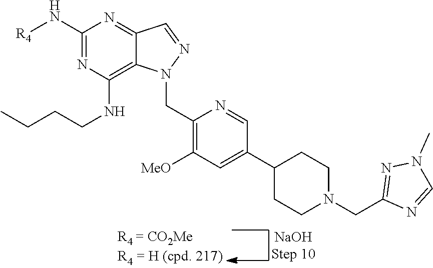

Step 1: To a solution of 5-bromo-3-methoxypicolinic acid (10 g, 43.1 mmol) in dry methanol (180 mL), was added slowly sulphuric acid (18.38 mL, 345 mmol). The reaction mixture was stirred at reflux for 16 h. Heating was stopped and the reaction mixture was allowed to cool to RT. Methanol was evaporated to a residue that was dissolved in DCM (200 mL) and washed with saturated aqueous NaHCO₃ solution (2×100 mL). The organic layer was dried over Na₂SO₄, filtered and evaporated to dryness to provide crude methyl 5-bromo-3-methoxypicolinate (9.8 g, 39.8 mmol, 92% yield) as a colourless oil, which was taken to the next step without further purification.

¹H NMR (400 MHz, CDCl₃) δ=8.36 (d, J=2.0 Hz, 1H), 7.54 (d, J=1.5 Hz, 1H), 3.99 (s, 3H), 3.96 (s, 3H).

LC-MS m/z 247.9 [M+H]⁺.

Step 2. To a stirred solution of methyl 5-bromo-3-methoxypicolinate (4 g, 16.26 mmol) in 1,4-dioxane (90 mL) and H₂O (10 mL), were added tert-butyl-4-(4,4,5,5-tetramethyl-1,3,2-dioxaborolan-2-yl)-3,6-dihydropyridine-1(2H)-carboxylate (10.05 g, 32.5 mmol) and Cs₂CO₃ (13.24 g, 40.6 mmol), followed by tetrakis(triphenylphosphine)palladium(0) (1.879 g, 1.626 mmol). The reaction mixture was purged with argon gas for about 5 minutes and then stirred for 6 h at 100° C. The reaction mixture was allowed to slowly cool to RT and was then filtered through a bed of CELITE™. The filtrate was evaporated to get crude product which was purified by flash column chromatography (SiO₂, 0-2% methanol in chloroform) to provide 1′-(tert-butyl) 6-methyl 5-methoxy-3′,6′-dihydro-[3,4′-bipyridine]-1′,6(2′H)-dicarboxylate (4.5 g, 12.92 mmol, 79% yield) as a pale yellow oil.

¹H NMR (400 MHz, DMSO-d₆) δ=8.30-8.25 (m, 1H), 7.60-7.58 (m, 1H), 6.50-6.34 (m, 1H), 4.09-4.00 (m, 2H), 3.89 (s, 3H), 3.83 (s, 3H), 3.61-3.53 (m, 2H), 2.58-2.53 (m, 2H), 1.44 (s, 9H).

LC-MS m/z 348.2 [M+H]⁺.

Step 3. To a stirred solution of 1′-(tert-butyl) 6-methyl 5-methoxy-3′,6′-dihydro-[3,4′-bipyridine]-1′,6(2′H)-dicarboxylate (5 g, 14.35 mmol) in THF (90 mL) and methanol (10 mL), was carefully added a 2 M solution of lithium borohydride in THF (35.9 mL, 71.8 mmol) dropwise at 0° C. Once the addition was completed, the reaction mixture was allowed to attain RT and was stirred at 45° C. for 6 h. Crushed ice was added to the reaction mixture followed by the addition of ethyl acetate. A white precipitate came out. The clear liquid was decanted and evaporated to yield tert-butyl 6-(hydroxymethyl)-5-methoxy-3′,6′-dihydro-[3,4′-bipyridine]-1′(2′H)-carboxylate (4.3 g, 13.43 mmol, 94% yield) as a pale yellow oil.

¹H NMR (400 MHz, DMSO-d6) δ=8.18 (d, J=1.0 Hz, 1H), 7.40 (d, J=1.0 Hz, 1H), 6.32-6.25 (m, 1H), 4.80 (t, J=5.8 Hz, 1H), 4.52 (d, J=6.0 Hz, 2H), 4.03 (br s, 2H), 3.86 (s, 3H), 3.56 (t, J=5.5 Hz, 2H), 1.46-1.42 (m, 11H).

LC-MS m/z 321.4 [M+H]⁺.

Step 4. To a solution of tert-butyl 6-(hydroxymethyl)-5-methoxy-3′,6′-dihydro-[3,4′-bipyridine]-1′(2′H)-carboxylate (4.3 g, 13.42 mmol) in DCM (30 mL), were added successively methanesulfonyl chloride (2.092 mL, 26.8 mmol), triethylamine (3.74 mL, 26.8 mmol) and lithium chloride (1.138 g, 26.8 mmol) at 0° C. The reaction mixture was stirred for 4 h and slowly allowed to reach RT. Crushed ice was added. The reaction mixture was extracted with DCM (2×150 mL). The combined organic layers were dried over Na₂SO₄ and evaporated on a rotary evaporator, keeping the water bath temperature below 35° C. to obtain tert-butyl 6-(chloromethyl)-5-methoxy-3′,6′-dihydro-[3,4′-bipyridine]-1′(2′H)-carboxylate (3.3 g, 9.74 mmol, 72.6% yield) as a yellowish oil.

¹H NMR (400 MHz, methanol-d₄) δ=8.23-8.21 (m, 1H), 7.64 (s, 1H), 6.38-6.33 (m, 1H), 4.20 (s, 2H), 4.02 (s, 3H), 3.71-3.67 (m, 2H), 2.64-2.58 (m, 2H), 2.60 (br s, 2H), 1.51 (s, 9H).

LC-MS m/z 339.4 [M+H]⁺.

Step 5. To a stirred solution of methyl (7-hydroxy-3-iodo-1H-pyrazolo[4,3-d]pyrimidin-5-yl)carbamate (3.2 g, 9.55 mmol) in DMF (20 mL), were added tert-butyl 6-(chloromethyl)-5-methoxy-3′,6′-dihydro-[3,4′-bipyridine]-1′(2′H)-carboxylate (3.24 g, 9.55 mmol) followed by Cs₂CO₃ (6.22 g, 19.10 mmol) at 0° C. The reaction mixture was slowly allowed to reach 25° C. and stirred for 6 h at the same temperature. After adding crushed ice, the reaction mixture was extracted with DCM (2×100 mL). The combined organic layers were dried over Na₂SO₄ and evaporated to yield crude product which was purified by flash column chromatography (SiO₂, 0-5% methanol in chloroform) to provide tert-butyl 6-((7-hydroxy-3-iodo-5-((methoxycarbonyl)amino)-1H-pyrazolo[4,3-d]pyrimidin-1-yl)methyl)-5-methoxy-3′,6′-dihydro-[3,4′-bipyridine]-1′(2′H)-carboxylate (3.5 g, 5.49 mmol, 57.5% yield) as a pale brown semi-solid.

¹H NMR (400 MHz, methanol-d₄) δ=8.03 (s, 1H), 7.44 (s, 1H), 6.27-6.20 (m, 1H), 5.91-5.88 (m, 2H), 4.20 (s, 2H), 4.10 (br s, 3H), 3.93 (s, 3H), 3.69-3.64 (m, 2H), 2.58-2.52 (m, 2H), 1.31 (s, 9H).

LC-MS m/z 638.2 [M+H]⁺.

Step 6. To a mixture of tert-butyl 6-((7-hydroxy-3-iodo-5-((methoxycarbonyl)amino)-1H-pyrazolo[4,3-d]pyrimidin-1-yl)methyl)-5-methoxy-3′,6′-dihydro-[3,4′-bipyridine]-1′(2′H)-carboxylate (2 g, 3.14 mmol) and butan-1-amine (1.639 mL, 15.69 mmol) in THF (5 mL) were added BOP (2.082 g, 4.71 mmol) and DBU (1.419 mL, 9.41 mmol) at 25° C. The reaction mixture was stirred for 12 h at the same temperature. Crushed ice was added. The mixture was then extracted by DCM (2×50 mL). The combined organic layer was dried over Na₂SO₄, filtered and evaporated to get the residue which was purified by flash column chromatography (SiO₂, 0-5% methanol in chloroform) to yield tert-butyl 6-((5-amino-7-(butylamino)-3-iodo-1H-pyrazolo[4,3-d]pyrimidin-1-yl)methyl)-5-methoxy-3′,6′-dihydro-[3,4′-bipyridine]-1′(2′H)-carboxylate (1.5 g, 2.166 mmol, 69.0% yield) as a yellow oil.

LC-MS m/z 693.4 [M+H]⁺.

Step 7. To a suspension of tert-butyl 6-((7-(butylamino)-3-iodo-5-((methoxy-carbonyl)amino)-1H-pyrazolo[4,3-d]pyrimidin-1-yl)methyl)-5-methoxy-3′,6′-dihydro-[3,4′-bipyridine]-1′(2′H)-carboxylate (2.5 g, 3.61 mmol) in methanol (100 mL) under an inert atmosphere, was added dry palladium on carbon (0.384 g, 3.61 mmol). The reaction mixture was stirred in an autoclave for 18 h at 50° C. under a hydrogen gas atmosphere at a pressure of 10 bar. The reaction mixture was filtered through a bed of CELITE™ and the filtrate was evaporated to get the residue which was purified by flash column chromatography (SiO₂, 0-5% methanol in chloroform) to yield tert-butyl 4-(6-((7-(butylamino)-5-((methoxycarbonyl)amino)-1H-pyrazolo[4,3-d]pyrimidin-1-yl)methyl)-5-methoxypyridin-3-yl)piperidine-1-carboxylate (1.5 g, 2.64 mmol, 73.1% yield) as a colourless oil.

¹H NMR (400 MHz, DMSO-d₆) δ=11.63 (s, 1H), 9.49 (s, 1H), 8.00 (s, 1H), 7.97-7.95 (m, 1H), 7.52-7.42 (m, 1H), 5.84 (s, 2H), 4.14-4.03 (m, 2H), 3.91 (s, 3H), 3.82 (s, 3H), 3.68-3.61 (m, 2H), 2.74-2.65 (m, 2H), 2.39-2.30 (m, 2H), 1.73-1.70 (m, 1H), 1.67-1.61 (m, 2H), 1.61-1.54 (m, 2H), 1.41 (s, 9H), 1.37-1.30 (m, 2H), 0.94-0.89 (m, 3H).

LC-MS m/z 569.5 [M+H]⁺.

Step 8. To a stirred solution of tert-butyl 4-(6-((7-(butylamino)-5-((methoxy-carbonyl)amino)-1H-pyrazolo[4,3-d]pyrimidin-1-yl)methyl)-5-methoxypyridin-3-yl)piperidine-1-carboxylate (1.5 g, 2.64 mmol) in THF (15 mL), was added 4 N hydrochloric acid in 1,4-dioxane (6.59 mL, 26.4 mmol). The reaction mixture was stirred for 6 h at RT. The solvent was evaporated to provide a residue that was washed with diethyl ether (2×30 mL). The residue was dissolved in methanol and solid Na₂CO₃ (1 g) was added. After stirring for 30 minutes, the suspension was filtered through a CELITE™ pad. The filtrate was evaporated to yield methyl (7-(butylamino)-1-((3-methoxy-5-(piperidin-4-yl)pyridin-2-yl)methyl)-1H-pyrazolo[4,3-d]pyrimidin-5-yl)carbamate (400 mg, 0.854 mmol, 32.4% yield) as a pale brown solid.

LC-MS m/z 469.4 [M+H]⁺.

Step 9. To a stirred solution of methyl (7-(butylamino)-1-((3-methoxy-5-(piperidin-4-yl)pyridin-2-yl)methyl)-1H-pyrazolo[4,3-d]pyrimidin-5-yl)carbamate (100 mg, 0.213 mmol) and 1-methyl-1H-1,2,4-triazole-3-carbaldehyde (47.4 mg, 0.427 mmol) in DMF (1 mL) and THF (1 mL), was added acetic acid (2.444 μLl, 0.043 mmol). The reaction mixture was stirred for 12 h at RT, after which NaCNBH₃ (40.2 mg, 0.64 mmol) was added. After stirring the reaction mixture for 1.5 h, crushed ice was added. The reaction mixture was extracted with DCM (2×15 mL). The combined organic layer was dried over Na₂SO₄ and evaporated to get methyl (7-(butylamino)-1-((3-methoxy-5-(1-((1-methyl-1H-1,2,4-triazol-3-yl)methyl)piperidin-4-yl)pyridin-2-yl)methyl)-1H-pyrazolo[4,3-d]pyrimidin-5-yl)carbamate (100 mg, 0.177 mmol, 83% yield) as a pale solid.

LC-MS m/z 564.4 [M+H]⁺.

Step 10. To a stirred solution of methyl (7-(butylamino)-1-((3-methoxy-5-(1-((1-methyl-1H-1,2,4-triazol-3-yl)methyl)piperidin-4-yl)pyridin-2-yl)methyl)-1H-pyrazolo[4,3-d]pyrimidin-5-yl)carbamate (100 mg, 0.177 mmol) in 1,4-dioxane (2 mL), was added a solution of NaOH (0.355 mL, 0.887 mmol) in water and the reaction mixture was stirred for 4 h at 75° C. The reaction mixture was slowly cooled to RT. The separated upper layer was taken out and evaporated to get the residue which was purified via preparative LC/MS with the following conditions: Column: Waters XBridge C18, 19×150 mm, 5-μm particles; mobile phase A: 10-mM NH₄OAc; mobile phase B: acetonitrile; gradient: 12-37% B over 22 minutes, then a 5-minute hold at 100% B; flow rate: 20 mL/min. The fraction collection was triggered by MS and UV signals. Fractions containing the desired product were combined and dried via centrifugal evaporation to yield Compound 217 (26.1 mg, 5.16 mmol, 28%).

### Example 34—Compound 218

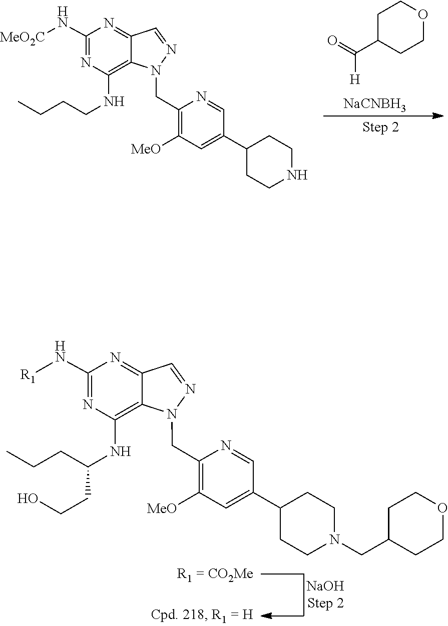

Step 1. To a stirred solution of methyl (7-(butylamino)-1-((3-methoxy-5-(piperidin-4-yl)pyridin-2-yl)methyl)-1H-pyrazolo[4,3-d]pyrimidin-5-yl)carbamate (100 mg, 0.213 mmol) and tetrahydro-2H-pyran-4-carbaldehyde (48.7 mg, 0.427 mmol) in DMF (1 mL) and THF (1 mL), was added acetic acid (2.444 μl, 0.043 mmol). The reaction mixture was stirred for 12 h at RT after which NaCNBH₃ (40.2 mg, 0.64 mmol) was added. After being stirred for 1.5 h, crushed ice was added. The mixture was extracted with DCM (15×2 mL). The combined organic layer was dried over Na₂SO₄ and evaporated to yield methyl (7-(butylamino)-1-((3-methoxy-5-(1-((tetrahydro-2H-pyran-4-yl)methyl)piperidin-4-yl)pyridin-2-yl)methyl)-1H-pyrazolo[4,3-d]pyrimidin-5-yl)carbamate (100 mg, 0.176 mmol, 83% yield).

LC-MS m/z 567.5 [M+H]⁺.

Step 2. To a stirred solution of methyl (7-(butylamino)-1-((3-methoxy-5-(1-((tetrahydro-2H-pyran-4-yl)methyl)piperidin-4-yl)pyridin-2-yl)methyl)-1H-pyrazolo[4,3-d]pyrimidin-5-yl)carbamate (100 mg, 0.176 mmol) in 1,4-dioxane (2 mL), was added NaOH (0.353 mL, 0.882 mmol) and the reaction mixture was stirred for 4 h at 75° C. The reaction mixture was slowly cooled to RT. The separated upper layer was taken out and evaporated to get the residue which was purified via preparative LC/MS (Column: Waters XBridge C18, 19×150 mm, 5-μm particles; mobile phase A: 10-mM NH₄OAc; mobile phase B: acetonitrile; gradient: 10-35% B over 20 minutes, then a 5-minute hold at 100% B; flow rate: 20 mL/min). Fractions containing the desired product were combined and dried via centrifugal evaporation to provide Compound 218 (7.4 mg, 1.4 mmol, 8.2%).

### Example 35—Compound 219

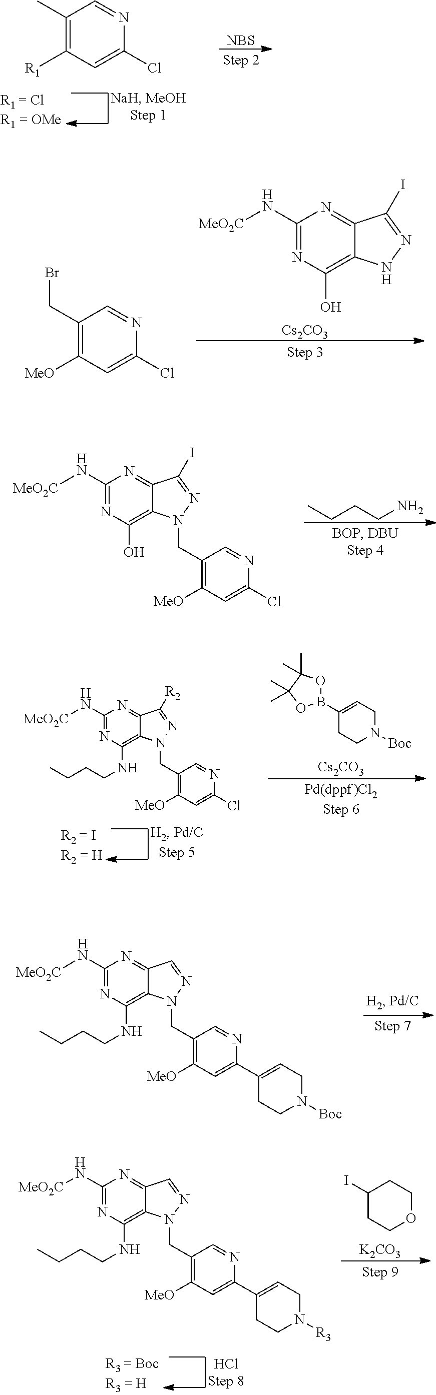

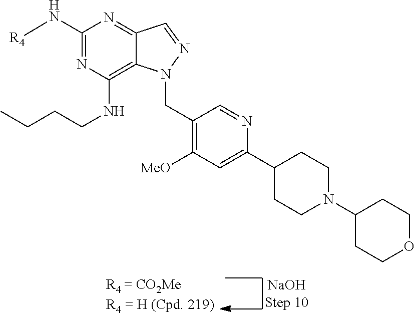

Step 1. To a stirred solution of sodium hydride (2.96 g, 74.1 mmol) in DMF (30.0 mL) and diethyl ether (30.0 mL), was added methanol (3.25 mL, 80 mmol) at 0° C. The reaction mixture was stirred at 0° C. for 20 minutes. To this mixture, 2,4-dichloro-5-methylpyridine (7.58 mL, 61.7 mmol) in diethyl ether (30.0 mL) was added at same temperature. The reaction mixture was stirred at 0° C. to RT for 1 h and then at RT for 16 h. The reaction mixture was quenched with ice cold water and it was partitioned between ethyl acetate and water. The organic layer was washed with brine solution, dried over Na₂SO₄, filtered and concentrated under reduced pressure to afford the residue, which was triturated with diethyl ether and petroleum ether. The solid was dried under vacuum to afford 2-chloro-4-methoxy-5-methylpyridine (10.4 g, 60.7 mmol, 98% yield) as a light brown solid.

¹H NMR (300 MHz, DMSO-d₆) δ=8.10-7.95 (m, 1H), 7.12-7.02 (m, 1H), 3.90 (s, 3H), 2.12-2.03 (m, 3H).

LC-MS m/z 158.0 [M+H]⁺.

Step 2. To a stirred solution of 2-chloro-4-methoxy-5-methylpyridine (5.0 g, 31.7 mmol) in CCl₄ (50.0 mL), NBS (6.78 g, 38.1 mmol) and AIBN (1.042 g, 6.35 mmol) were added. The reaction mixture was stirred at 60° C. for 18 h. The reaction mixture was filtered through a CELITE™ bed and washed with CCl₄. The filtrate was concentrated to dryness under reduced pressure to afford a residue. The crude product was purified by ISCO combiflash chromatography by eluting with 0-100% ethyl acetate in petroleum ether to afford 5-(bromomethyl)-2-chloro-4-methoxypyridine (4.8 g, 16.85 mmol, 53.1% yield) as a light brown solid.

¹H NMR (300 MHz, DMSO-d₆) δ=8.34-8.30 (m, 1H), 7.26-7.22 (m, 1H), 4.61 (s, 2H), 4.00-3.96 (m, 3H).

LC-MS m/z 236.0 [M+H]⁺.

Step 3. To a stirred solution of methyl (7-hydroxy-3-iodo-1H-pyrazolo[4,3-d]pyrimidin-5-yl)carbamate (9.0 g, 26.9 mmol) in DMF (100.0 mL), Cs₂CO₃ (17.50 g, 53.7 mmol) was added. To this mixture 5-(bromomethyl)-2-chloro-4-methoxypyridine (6.35 g, 26.9 mmol) was added at 0° C. The reaction mixture was stirred at 0° C. for 1 h and water was added. The precipitated solid was filtered and washed with excess of water followed by petroleum ether. The solid was dried under vacuum to afford crude compound, which was purified by ISCO combiflash chromatography by eluting with 0-100% ethyl acetate in petroleum ether to afford methyl (1-((6-chloro-4-methoxypyridin-3-yl)methyl)-7-hydroxy-3-iodo-1H-pyrazolo[4,3-d]pyrimidin-5-yl)carbamate (5.8 g, 10.28 mmol, 38.3% yield) as an off-white solid.

¹H NMR (300 MHz, DMSO-d₆) δ=11.81-11.30 (m, 2H), 7.99-7.92 (m, 2H), 7.23-7.18 (m, 1H), 5.68 (s, 2H), 3.85 (s, 3H), 3.75 (s, 3H).

LC-MS m/z 491.0 [M+H]⁺.

Step 4. To a stirred solution of methyl (1-((6-chloro-4-methoxypyridin-3-yl)methyl)-7-hydroxy-3-iodo-1H-pyrazolo[4,3-d]pyrimidin-5-yl)carbamate (2.5 g, 5.10 mmol) in DMSO (10.0 mL), BOP (3.38 g, 7.64 mmol) and butan-1-amine (0.755 mL, 7.64 mmol) were added. The reaction mixture was stirred at RT for 2 h. The reaction mixture was partitioned between EtOAc and water. The organic layer was washed with brine solution, dried over Na₂SO₄, filtered and concentrated under reduced pressure to afford a residue. The residue was purified by ISCO combiflash chromatography by eluting with 0-100% ethyl acetate in chloroform to afford methyl (7-(butylamino)-1-((6-chloro-4-methoxypyridin-3-yl)methyl)-3-iodo-1H-pyrazolo[4,3-d]pyrimidin-5-yl)carbamate (1.02 g, 1.813 mmol, 35.6% yield) as a light brown solid.

¹H NMR (300 MHz, DMSO-d₆) δ=9.84 (s, 1H), 7.85 (s, 1H), 7.44 (br t, J=5.1 Hz, 1H), 7.21 (s, 1H), 5.70 (s, 2H), 3.81 (s, 3H), 3.62 (s, 3H), 3.59-3.50 (m, 2H), 3.33 (s, 2H), 1.67-1.55 (m, 2H), 1.40-1.26 (m, 1H), 1.35-1.23 (m, 1H), 1.36-1.15 (m, 1H), 0.89 (t, J=7.4 Hz, 3H).

LC-MS m/z 546.0 [M+H]⁺.

Step 5. To a stirred solution of methyl (7-(butylamino)-1-((6-chloro-4-methoxypyridin-3-yl)methyl)-3-iodo-1H-pyrazolo[4,3-d]pyrimidin-5-yl)carbamate (1.0 g, 1.832 mmol) in ethyl acetate (10.0 mL) and ethanol (10.0 mL), Pd—C (0.975 g, 0.916 mmol) was added. The reaction mixture was stirred under hydrogen atmosphere at RT for 16 h. The reaction mixture was filtered through CELITE™ bed and washed with excess of methanol. The filtrate was concentrated under reduced pressure to afford methyl (7-(butylamino)-1-((6-chloro-4-methoxypyridin-3-yl)methyl)-1H-pyrazolo[4,3-d]pyrimidin-5-yl)carbamate (0.8 g, 1.810 mmol, 99% yield) as a light brown solid.

LC-MS m/z 420.2 [M+H]⁺.

Step 6. To a stirred solution of methyl (7-(butylamino)-1-((6-chloro-4-methoxypyridin-3-yl)methyl)-1Hpyrazolo[4,3-d]pyrimidin-5-yl)carbamate (730 mg, 1.739 mmol) in anhydrous dioxane (15 mL) and water (0.4 mL), were added tert-butyl 4-(4,4,5,5-tetramethyl-1,3,2-dioxaborolan-2-yl)-3,6-dihydropyridine-1(2H)-carboxylate (1075 mg, 3.48 mmol), Cs₂CO₃ (1699 mg, 5.22 mmol) and PdCl₂(dppf).CH₂Cl₂ adduct (142 mg, 0.174 mmol) at RT. The reaction mixture was purged with nitrogen, heated to 100° C., and stirred for 16 h. The black suspension was filtered through CELITE™ bed and the bed was washed with ethyl acetate. The filtrate was concentrated under reduced pressure to afford the crude product. The residue was purified using combiflash (silica gel 60-120 mesh; 35% ethyl acetate in petroleum ether as eluent) to afford tert-butyl 5-((7-(butylamino)-5-((methoxycarbonyl)amino)-1H-pyrazolo[4,3-d]pyrimidin-1-yl)methyl)-4-methoxy-3′,6′-dihydro-[2,4′-bipyridine]-1′(2′H)-carboxylate (650 mg, 1.147 mmol, 66.0% yield) as a pale yellow solid.

LC-MS m/z 567.3 [M+H]⁺.

Step 7. To a stirred solution of tert-butyl 5-((7-(butylamino)-5-((methoxycarbonyl)-amino)-1H-pyrazolo[4,3-d]pyrimidin-1-yl)methyl)-4-methoxy-3′,6′-dihydro-[2,4′-bipyridine]-1′(2′H)-carboxylate (0.770 g, 1.359 mmol) in tetrahydrofuran (10 mL):methanol (10 mL) and was added 10% palladium on carbon (0.723 g, 0.679 mmol). The reaction mixture was stirred at 50° C. under H₂ (5 kg pressure) for 14 h. The mixture was filtered through CELITE™ bed and which was washed with methanol and DCM (200 mL). The filtrate was concentrated under vacuum below 50° C. to give tert-butyl 4-(5-((7-(butylamino)-5-((methoxycarbonyl)amino)-1H-pyrazolo[4,3-d]pyrimidin-1-yl)methyl)-4-methoxypyridin-2-yl)piperidine-1-carboxylate (0.600 g, 0.591 mmol, 43.5% yield) as a brown solid.

LC-MS m/z 569.4 [M+H]⁺.

Step 8. To a stirred solution of tert-butyl 4-(5-((7-(butylamino)-5-((methoxy-carbonyl)amino)-1Hpyrazolo[4,3-d]pyrimidin-1-yl)methyl)-4-methoxypyridin-2-yl)piperidine-1-carboxylate (0.700 g, 1.231 mmol) in dichloromethane (3 mL), was added HCl in dioxane (6.15 mL, 24.62 mmol). The reaction mixture was stirred at RT for 2 h and concentrated under reduced temperature to give methyl (7-(butylamino)-1-((4-methoxy-6-(piperidin-4-yl)pyridin-3-yl)methyl)-1H-pyrazolo[4,3-d]pyrimidin-5-yl)carbamate (0.400 g, 0.666 mmol, 54.1% yield) as a brown solid.

LC-MS m/z 469.2 [M+H]⁺.

Step 9. To a stirred solution of methyl (7-(butylamino)-1-((4-methoxy-6-(piperidin-4-yl)pyridin-3-yl)methyl)-1H-pyrazolo[4,3-d]pyrimidin-5-yl)carbamate hydrochloride (0.100 g, 0.198 mmol) in DMF (2 mL), 4-iodotetrahydro-2H-pyran (0.084 g, 0.396 mmol) and K₂CO₃ (0.082 g, 0.594 mmol) were added. The reaction mixture was stirred at 50° C. for 14 h. The reaction mixture was concentrated under high vacuum to give methyl (7-(butylamino)-1-((4-methoxy-6-(1-(tetrahydro-2H-pyran-4-yl)piperidin-4-yl)pyridin-3-yl)methyl)-1H-pyrazolo[4,3-d]pyrimidin-5-yl)carbamate (0.100 g, 0.181 mmol) as a yellow oil.

LC-MS m/z 553.5 [M+H]⁺.

Step 10. To a stirred solution of methyl (7-(butylamino)-1-((4-methoxy-6-(1-(tetrahydro-2H-pyran-4-yl)piperidin-4-yl)pyridin-3-yl)methyl)-1H-pyrazolo[4,3-d]pyrimidin-5-yl)carbamate (0.100 g, 0.181 mmol) in 1,4-dioxane (1 mL), NaOH (0.036 g, 0.905 mmol) in water (1 mL) was added. The reaction mixture was stirred at 70° C. for 3 h. The reaction mixture was partitioned between water and ethyl acetate. The organic layer was washed with brine solution, dried over anhydrous Na₂SO₄, filtered and concentrated under vacuum to give crude compound. It was purified by reversed phase preparative LC/MS (Column: Waters XBridge C18, 150 mm×19 mm, 5-μm particles; mobile phase A: 5:95 acetonitrile:water with 10-mM NH₄OAc; mobile phase B: 95:5 acetonitrile:water with 10-mM NH₄OAc; gradient: a 0-minute hold at 10% B, 10-45% B over 25 minutes, then a 5-minute hold at 100% B; flow rate: 15 mL/min). LC-MS m/z 158.0 [M+H]⁺. Fraction collection was triggered by MS and UV signals. Fractions containing the desired product were combined and dried via centrifugal evaporation using Genevac to afford Compound 219 (0.008 g, 0.016 mmol, 8.58% yield).

### Example 36—Compound 220

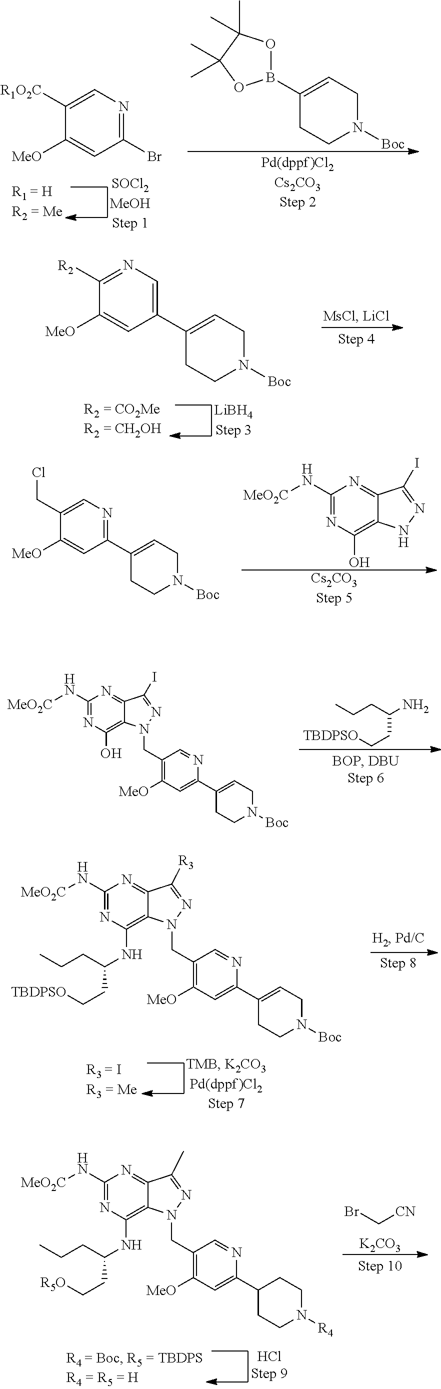

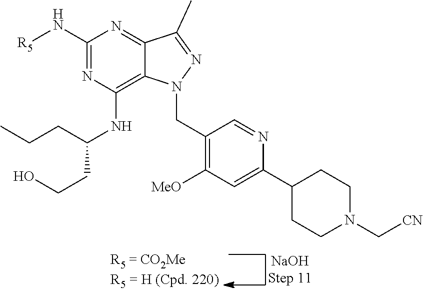

Step 1. To a stirred solution of 6-chloro-4-methoxynicotinic acid (4.5 g, 23.99 mmol) in MeOH (45.0 mL), SOCl₂ (2.63 mL, 36.0 mmol) was added. The reaction mixture was stirred at 75° C. for 16 h. The reaction mixture was concentrated under reduced pressure to afford a residue which was quenched with saturated NaHCO₃ solution and then reaction mixture was partitioned between DCM and water. The organic layer was washed with brine solution and dried over Na₂SO₄, filtered and concentrated under reduced pressure to afford crude compound. It was purified by ISCO combiflash chromatography by eluting with 0-100% ethyl acetate in petroleum ether to afford methyl 6-chloro-4-methoxynicotinate (3.86 g, 18.95 mmol, 79% yield) as an off white solid.

¹H NMR (300 MHz, DMSO-d₆) δ=8.57 (s, 1H), 7.38 (s, 1H), 3.96 (s, 3H), 3.83-3.79 (m, 3H).

LC-MS m/z 202.0 [M+H]⁺.

Step 2. To a stirred solution of methyl 6-chloro-4-methoxynicotinate (3.8 g, 18.85 mmol) in 1,4-dioxane (40.0 mL):water (10.0 mL), Cs₂CO₃ (18.42 g, 56.5 mmol), tert-butyl 4-(4,4,5,5-tetramethyl-1,3,2-dioxaborolan-2-yl)-3,6-dihydropyridine-1(2H)-carboxylate (8.74 g, 28.3 mmol) and PdCl₂(dppf).CH₂Cl₂ adduct (1.539 g, 1.885 mmol) were added under nitrogen purging. The reaction mixture was stirred at 100° C. for 4 h. The reaction mixture was filtered through CELITE™ bed and washed with excess of EtOAc. The filtrate was concentrated under reduced pressure to afford the residue. The crude compound was purified by ISCO combiflash chromatography by eluting with 0-100% ethyl acetate in petroleum ether to afford 1′-(tert-butyl) 5-methyl 4-methoxy-3′,6′-dihydro-[2,4′-bipyridine]-1′,5(2′H)-dicarboxylate (4.06 g, 10.72 mmol, 56.9% yield) as a light brown oil.

¹H NMR (300 MHz, DMSO-d₆) δ=8.73-8.66 (m, 1H), 7.27-7.19 (m, 1H), 6.92-6.84 (m, 1H), 4.13-4.03 (m, 2H), 3.97-3.93 (m, 4H), 3.82-3.77 (m, 3H), 3.59-3.48 (m, 2H), 2.63-2.53 (m, 2H), 1.47-1.39 (m, 9H).

LC-MS m/z 349.2 [M+H]⁺.

Step 3. To a stirred solution of 1′-(tert-butyl) 5-methyl 4-methoxy-3′,6′-dihydro-[2,4′-bipyridine]-1′,5(2′H)-dicarboxylate (4.0 g, 11.48 mmol) in THF (40.0 mL):MeOH (10.0 mL), LiBH₄ (2M in THF) (14.35 mL, 28.7 mmol) was added. The reaction mixture was stirred at RT for 16 h. The reaction mixture was treated with 10% NaOH solution, diluted with EtOAc, and filtered through a CELITE™ bed. The filtrate was partitioned between EtOAc and water. The organic layer was washed with brine solution, dried over Na₂SO₄, filtered and concentrated under reduced pressure to afford tert-butyl 5-(hydroxymethyl)-4-methoxy-3′,6′-dihydro-[2,4′-bipyridine]-1′(2′H)-carboxylate (2.69 g, 8.14 mmol, 70.9% yield) as a brown oil.

¹H NMR (300 MHz, DMSO-d₆) δ=8.32 (s, 1H), 7.10 (s, 1H), 6.67 (br s, 1H), 5.11-5.05 (m, 1H), 4.50-4.45 (m, 2H), 4.04 (br s, 2H), 3.95 (s, 1H), 3.88 (s, 3H), 3.57-3.48 (m, 2H), 2.61-2.54 (m, 2H), 1.43 (s, 9H).

LC-MS m/z 321.2 [M+H]⁺.

Step 4. To a stirred solution of tert-butyl 5-(hydroxymethyl)-4-methoxy-3′,6′-dihydro-[2,4′-bipyridine]-1′(2′H)-carboxylate (2.6 g, 8.12 mmol) in DCM (25.0 mL), TEA (2.262 mL, 16.23 mmol), MsCl (1.265 mL, 16.23 mmol) and lithium chloride (0.688 g, 16.23 mmol) were added at 0° C. The reaction mixture was stirred at same temperature for 30 minutes and then at RT for 5 h. The reaction mixture was partitioned between DCM and water. The organic layer was washed with brine solution, dried over Na₂SO₄, filtered and concentrated under reduced pressure to afford tert-butyl 5-(chloromethyl)-4-methoxy-3′,6′-dihydro-[2,4′-bipyridine]-1′(2′H)-carboxylate (2.68 g, 7.59 mmol, 94% yield) as a brown oil. The crude product was used as such.

¹H NMR (300 MHz, DMSO-d₆) δ=8.75-8.68 (m, 1H), 7.59-7.53 (m, 1H), 6.98-6.88 (m, 1H), 4.85-4.79 (m, 2H), 4.18 (s, 3H), 4.16-4.10 (m, 2H), 3.61-3.52 (m, 2H), 2.68-2.58 (m, 2H), 1.43 (s, 9H).

LC-MS m/z 339.2 [M+H]⁺.

Step 5. To a stirred solution of methyl (7-hydroxy-3-iodo-1H-pyrazolo[4,3-d]pyrimidin-5-yl)carbamate (1.0 g, 2.98 mmol) in DMF (10.0 mL), Cs₂CO₃ (1.945 g, 5.97 mmol) was added. To this mixture tert-butyl 5-(chloromethyl)-4-methoxy-3′,6′-dihydro-[2,4′-bipyridine]-1′(2′H)-carboxylate (1.011 g, 2.98 mmol) in DMF (5.0 mL) was added at 0° C. The reaction mixture was stirred at 0° C. for 1 h and at RT for 1 h. The reaction mixture was partitioned between EtOAc and water. The organic layer was washed with brine solution, dried over Na₂SO₄, filtered and concentrated under reduced pressure to afford the residue. The crude compound was purified by ISCO combiflash chromatography by eluting with 0-100% ethyl acetate in petroleum ether to afford tert-butyl 5-((7-hydroxy-3-iodo-5-((methoxycarbonyl)amino)-1H-pyrazolo[4,3-d]pyrimidin-1-yl)methyl)-4-methoxy-3′,6′-dihydro-[2,4′-bipyridine]-1′(2′H)-carboxylate (718 mg, 0.946 mmol, 31.7% yield) as a light brown solid.

¹H NMR (300 MHz, DMSO-d₆) δ=11.74-11.66 (m, 1H), 11.44-11.36 (m, 1H), 8.06-7.98 (m, 1H), 7.19-7.09 (m, 1H), 6.75-6.63 (m, 1H), 5.74-5.67 (m, 2H), 4.07-4.01 (m, 2H), 3.85 (s, 3H), 3.78-3.71 (m, 5H), 3.52 (br t, J=5.3 Hz, 2H), 2.59-2.53 (m, 2H), 1.42 (s, 9H).

LC-MS m/z 638.0 [M+H]⁺.

Step 6. To a stirred solution of tert-butyl 5-((7-hydroxy-3-iodo-5-((methoxycarbonyl)-amino)-1H-pyrazolo[4,3-d]pyrimidin-1-yl)methyl)-4-methoxy-3′,6′-dihydro-[2,4′-bipyridine]-1′(2′H)-carboxylate (0.5 g, 0.784 mmol) in DMSO (5.0 mL), DBU (0.355 mL, 2.353 mmol), BOP (0.520 g, 1.177 mmol) and (S)-1-((tert-butyldiphenylsilyl)oxy)hexan-3-amine (0.335 g, 0.941 mmol) were added. The reaction mixture was stirred at 45° C. for 4 h. The reaction mixture was partitioned between EtOAc and water. The organic layer was washed with brine solution, dried over Na₂SO₄, filtered and concentrated under reduced pressure to afford the residue, which was purified by ISCO combiflash chromatography by eluting with 0-100% ethyl acetate in petroleum ether to afford tert-butyl (S)-5-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-4-methoxy-3′,6′-dihydro-[2,4′-bipyridine]-1′(2′H)-carboxylate (0.16 g, 0.100 mmol, 12.76% yield) as a brown solid.

LC-MS m/z 975.3 [M+H]⁺.

Step 7. To a stirred solution of tert-butyl (S)-5-((7-((1-((tert-butyldiphenylsilyl)-oxy)hexan-3-yl)amino)-3-iodo-5-((methoxycarbonyl)amino)-1H-pyrazolo[4,3-d]pyrimidin-1-yl)methyl)-4-methoxy-3′,6′-dihydro-[2,4′-bipyridine]-1′(2′H)-carboxylate (2.1 g, 2.154 mmol) in 1,4-dioxane (20.0 mL), K₂CO₃ (0.595 g, 4.31 mmol), trimethylboroxine (0.541 g, 4.31 mmol) and PdCl₂(dppf).CH₂Cl₂ adduct (0.176 g, 0.215 mmol) were added under nitrogen purging. The reaction mixture was stirred at 100° C. for 6 h. The reaction mixture was partitioned between EtOAc and water. The organic layer was washed with brine solution, dried over Na₂SO₄, filtered and concentrated under reduced pressure to afford crude compound, which was purified by ISCO combiflash chromatography by eluting with 0-100% ethyl acetate in petroleum ether to afford tert-butyl (S)-5-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-4-methoxy-3′,6′-dihydro-[2,4′-bipyridine]-1′(2′H)-carboxylate (1.29 g, 1.495 mmol, 69.4% yield) as a light brown solid.

LC-MS m/z: 861.5 [M−H]⁺.

Step 8. To a stirred solution of tert-butyl (S)-5-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-4-methoxy-3′,6′-dihydro-[2,4′-bipyridine]-1′(2′H)-carboxylate (1.2 g, 1.390 mmol) in THF (15.0 mL):MeOH (15.0 mL), Pd—C (0.740 g, 0.695 mmol) was added. The reaction mixture was stirred at 50° C. under hydrogen atmosphere of 100 psi pressure in an autoclave for 16 h. The reaction mixture was filtered through CELITE™ bed and washed with excess of methanol. The filtrate was concentrated under reduced pressure to afford tert-butyl (S)-4-(5-((7-((1-((tert-butyldiphenylsilyl)oxy)hexan-3-yl)amino)-5-((methoxy-carbonyl)amino)-3-methyl-1H-pyrazolo[4,3-d]pyrimidin-1-yl)methyl)-4-methoxypyridin-2-yl)piperidine-1-carboxylate (1.0 g, 1.156 mmol, 83% yield) as a light brown solid.

LC-MS m/z 865.4 [M+H]⁺.

Step 9. To a stirred solution of tert-butyl (S)-4-(5-((7-((1-((tert-butyldiphenylsilyl)-oxy)hexan-3-yl)amino)-5-((methoxycarbonyl)amino)-3-methyl-1H-pyrazolo[4,3-d]pyrimidin-1-yl)methyl)-4-methoxypyridin-2-yl)piperidine-1-carboxylate (0.9 g, 1.040 mmol) in MeOH (10.0 mL), concentrated HCl (3.0 mL, 35.1 mmol) was added at 0° C. The reaction mixture was stirred at RT for 2 h. The reaction mixture was concentrated completely under reduced pressure to afford crude compound, which was triturated with diethyl ether and petroleum ether. The solvent was decanted and the residue was dried under vacuum to afford methyl (S)-(7-((1-hydroxyhexan-3-yl)amino)-1-((4-methoxy-6-(piperidin-4-yl)pyridin-3-yl)methyl)-3-methyl-1H-pyrazolo[4,3-d]pyrimidin-5-yl)carbamate (0.88 g, 0.969 mmol, 93% yield) as a brown solid.

LC-MS m/z 527.2 [M+H]⁺.

Step 10. To a stirred solution of methyl (S)-(7-((1-hydroxyhexan-3-yl)amino)-1-((4-methoxy-6-(piperidin-4-yl)pyridin-3-yl)methyl)-3-methyl-1H-pyrazolo[4,3-d]pyrimidin-5-yl)carbamate (0.15 g, 0.285 mmol) in DMF (2.5 mL), K₂CO₃ (0.079 g, 0.570 mmol) and 2-bromoacetonitrile (0.051 g, 0.427 mmol) were added. The reaction mixture was stirred at 50° C. for 1 h. The reaction mixture was filtered through CELITE™ bed and washed with excess of ethyl acetate, then filtrate was concentrated under reduced pressure to afford methyl (S)-(1-((6-(1-(cyanomethyl)piperidin-4-yl)-4-methoxypyridin-3-yl)methyl)-7-((1-hydroxyhexan-3-yl)amino)-3-methyl-1H-pyrazolo[4,3-d]pyrimidin-5-yl)carbamate (212 mg, 0.154 mmol, 53.9% yield) as a brown semi solid.

LC-MS m/z 566.5 [M+H]⁺.

Step 11. To a stirred solution of methyl (S)-(1-((6-(1-(cyanomethyl)piperidin-4-yl)-4-methoxypyridin-3-yl)methyl)-7-((1-hydroxyhexan-3-yl)amino)-3-methyl-1H-pyrazolo[4,3-d]pyrimidin-5-yl)carbamate (0.15 g, 0.265 mmol) in 1,4-dioxane (2.0 mL):water (2.0 mL), NaOH (0.053 g, 1.326 mmol) was added. The reaction mixture was stirred at 70° C. for 16 h. The layer separation of reaction mixture was observed. The organic layer was separated, dried over Na₂SO₄, filtered and concentrated under reduced pressure to afford crude compound. It was purified by reversed phase preparative LC/MS (Column: Waters XBridge C18, 150 mm×19 mm, 5-μm particles; mobile phase A: 5:95 acetonitrile:water with 10-mM NH₄OAc; mobile phase B: 95:5 acetonitrile:water with 10 mM NH₄OAc; gradient: a 0-minute hold at 5% B, 5-20% B over 20 minutes, then a 5-minute hold at 100% B; flow Rate: 20 mL/min; column temperature: 25° C.). Fraction collection was triggered by MS and UV signals. Fractions containing the desired product were combined and dried via centrifugal evaporation using Genevac to afford Compound 220 (10.7 mg, 0.020 mmol, 7.58% yield).

### Example 37—Compound 221

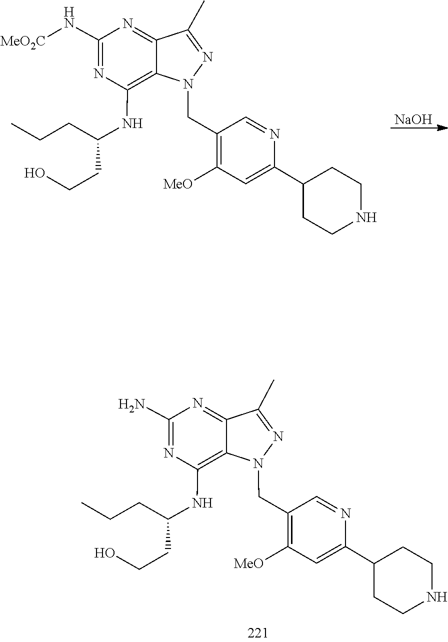 [SMILES: CCC[C@@H](CCO)Nc1nc(NC(=O)OC)nc2c(C)nn(Cc3cnc(C4CCNCC4)cc3OC)c12 ; "NaOH" ; CCC[C@@H](CCO)Nc1nc(N)nc2c(C)nn(Cc3cnc(C4CCNCC4)cc3OC)c12]

To a stirred solution of methyl (S)-(7-((1-hydroxyhexan-3-yl)amino)-1-((4-methoxy-6-(piperidin-4-yl)pyridin-3-yl)methyl)-3-methyl-1H-pyrazolo[4,3-d]pyrimidin-5-yl)carbamate (0.1 g, 0.190 mmol) in 1,4-dioxane (2.0 mL), NaOH (1.0 mL, 0.190 mmol) was added. The reaction mixture was stirred at 70° C. for 4 h. The layer separation of reaction mixture was observed. The organic layer was separated, dried over Na₂SO₄, filtered and concentrated under reduced pressure to afford crude compound. It was purified by reversed phase preparative LC/MS (Column: Waters XBridge C18, 19×150 mm, 5-μm particles; mobile phase A: 10-mM NH₄OAc; mobile phase B: acetonitrile; gradient: 15-55% B over 20 minutes, then a 5-minute hold at 100% B; flow rate: 15 mL/min). Fraction collection was triggered by MS and UV signals. Fractions containing the desired product were combined and dried via centrifugal evaporation using Genevac to afford Compound 221 (50.9 mg, 0.105 mmol, 55.5% yield).

### Example 38—Compound 222

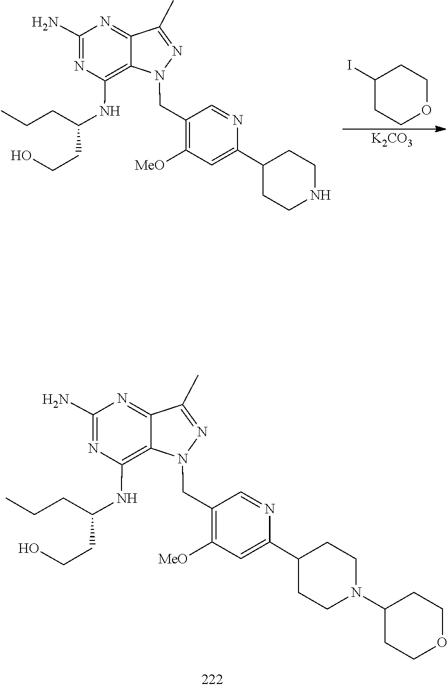

To a stirred solution of (S)-3-((5-amino-1-((4-methoxy-6-(piperidin-4-yl)pyridin-3-yl)methyl)-3-methyl-1H-pyrazolo[4,3-d]pyrimidin-7-yl)amino)hexan-1-ol (90.0 mg, 0.192 mmol) in DMF (1.5 mL), were added K₂CO₃ (80 mg, 0.576 mmol) and 4-iodotetrahydro-2H-pyran (0.034 mL, 0.288 mmol). The reaction mixture was stirred at 50° C. for 4 h. The reaction mixture was filtered through CELITE™ bed and washed with excess of ethyl acetate. The filtrate was concentrated completely under reduced pressure to afford the residue. The crude compound was purified by reversed phase preparative LC/MS (Preparative column: Gemini NX c18 (250×21.2 mm)×5 micron; mobile phase A:10 mM NH₄OAc, mobile phase B: CH₃CN/MeOH (1/1); flow rate: 20 mL/min; gradient T/% B: 0/30, 6/40, 16/60, 17/95, 22/95, 23/30, 25/30). The fraction collection was triggered by MS and UV signals. Fractions containing the desired product were combined and dried via centrifugal evaporation using Genevac to afford Compound 222 (27.1 mg, 0.049 mmol, 25.5% yield).

### Example 39—Starting Materials and Intermediates

Chart 1 below show schemes for making compounds that could be useful as starting materials or intermediates for the preparation of TLR7 agonists disclosed herein. The schemes can be adapted to make other, analogous compounds that could be used as starting materials or intermediates. The reagents employed are well known in the art and in many instances their use has been demonstrated in the preceding Examples.

### Chart 1

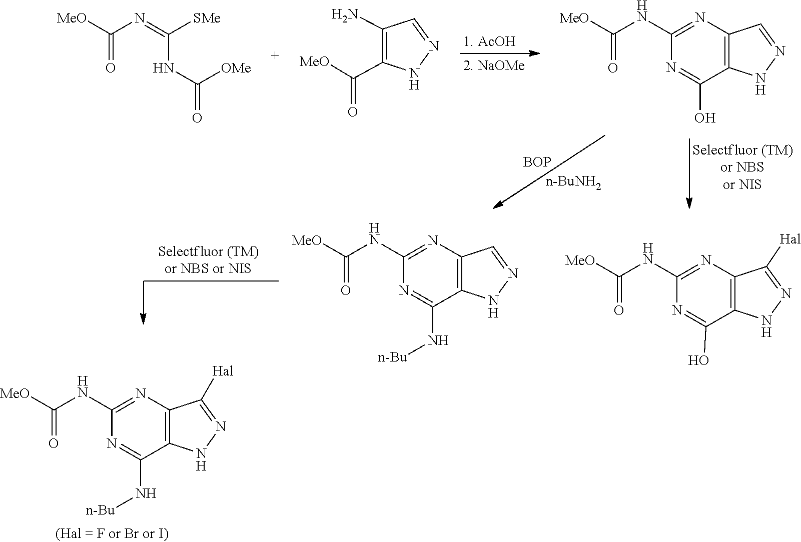

### Biological Activity

The biological activity of compounds disclosed herein as TLR7 agonists can be assayed by the procedures following.

### Human TLR7 Agonist Activity Assay

This procedure describes a method for assaying human TLR7 (hTLR7) agonist activity of the compounds disclosed in this specification.

Engineered human embryonic kidney blue cells (HEK-Blue™ TLR cells; Invivogen) possessing a human TLR7-secreted embryonic alkaline phosphatase (SEAP) reporter transgene were suspended in a non-selective, culture medium (DMEM high-glucose (Invitrogen), supplemented with 10% fetal bovine serum (Sigma)). HEK-Blue™ TLR7 cells were added to each well of a 384-well tissue-culture plate (15,000 cells per well) and incubated 16-18 h at 37° C., 5% CO₂. Compounds (100 nl) were dispensed into wells containing the HEK-Blue™ TLR cells and the treated cells were incubated at 37° C., 5% CO₂. After 18 h treatment ten microliters of freshly-prepared Quanti-Blue™ reagent (Invivogen) was added to each well, incubated for 30 min (37° C., 5% CO₂) and SEAP levels measured using an Envision plate reader (OD=620 nm). The half maximal effective concentration values (EC₅₀; compound concentration which induced a response halfway between the assay baseline and maximum) were calculated.

### Induction of Type I Interferon Genes (MX-1) and CD69 in Human Blood

The induction of Type I interferon (IFN) MX-1 genes and the B-cell activation marker CD69 are downstream events that occur upon activation of the TLR7 pathway. The following is a human whole blood assay that measures their induction in response to a TLR7 agonist.

Heparinized human whole blood was harvested from human subjects and treated with test TLR7 agonist compounds at 1 mM. The blood was diluted with RPMI 1640 media and Echo was used to predot 10 nL per well giving a final concentration of 1 uM (10 nL in 10 uL of blood). After mixing on a shaker for 30 sec, the plates were covered and placed in a 37° C. chamber for o/n=17 hrs. Fixing/lysis buffer was prepared (5×->1× in H₂O, warm at 37° C.; Cat #BD 558049) and kept the perm buffer (on ice) for later use.

For surface markers staining (CD69): prepared surface Abs: 0.045 ul hCD14-FITC (ThermoFisher Cat #MHCD1401)+0.6 ul hCD19-ef450 (ThermoFisher Cat #48-0198-42)+1.5 ul hCD69-PE (cat #BD555531)+0.855 ul FACS buffer. Added 3 ul/well, spin1000 rpm for 1 min and mixed on shaker for 30 sec, put on ice for 30 mins. Stop stimulation after 30 minutes with 70 uL of prewarmed 1× fix/lysis buffer and use Feliex mate to resuspend (15 times, change tips for each plate) and incubate at 37 C for 10 minutes.

Centrifuge at 2000 rpm for 5 minutes aspirate with HCS plate washer, mix on shaker for 30 sec and then wash with 70 uL in dPBS and pelleted 2×s (2000 rpm for 5 min) and 50 ul wash in FACS buffer pelleted 1×s (2000 rpm for 5 min). Mix on shaker for 30 sec. For Intracellular markers staining (MX-1): Add 50 ul of BD Perm buffer III and mix on shaker for 30 sec. Incubate on ice for 30 minutes (in the dark). Wash with 50 uL of FACS buffer 2×(spin @2300 rpm×5 min after perm) followed by mixing on shaker for 30 sec. Resuspended in 20 ul of FACS buffer containing MX1 antibody ( )(4812)-Alexa 647: Novus Biologicals #NBP2-43704AF647) 20 ul FACS bf+0.8 ul hIgG+0.04 ul MX-1. Spin 1000 rpm for 1 min, mix on shaker for 30 se and the samples were incubated at RT in the dark for 45 minutes followed by washing 2×FACS buffer (spin @2300 rpm×5 min after perm). Resuspend 20 ul (35 uL total per well) of FACS buffer and cover with foil and place in 4° C. to read the following day. Plates were read on iQuePlus. The results were loaded into toolset and IC50 curves are generated in curve master. The y-axis 100% is set to 1 uM of resiquimod.

### Induction of TNF-Alpha and Type I IFN Response Genes in Mouse Blood

The induction of TNF-alpha and Type I IFN response genes are downstream events that occur upon activation of the TLR7 pathway. The following is an assay that measures their induction in whole mouse blood in response to a TLR7 agonist.

Heparinized mouse whole blood was diluted with RPMI 1640 media with Pen-Strep in the ratio of 5:4 (50 uL whole blood and 40 uL of media). A volume of 90 uL of the diluted blood was transferred to wells of Falcon flat bottom 96-well tissue culture plates, and the plates were incubated at 4° C. for 1 h. Test compounds in 100% DMSO stocks were diluted 20-fold in the same media for concentration response assays, and then 10 uL of the diluted test compounds were added to the wells, so that the final DMSO concentration was 0.5%. Control wells received 10 uL media containing 5% DMSO. The plates were then incubated at 37° C. in a 5% CO₂ incubator for 17 h. Following the incubation, 100 uL of the culture medium as added to each well. The plates were centrifuged and 130 uL of supernatant was removed for use in assays of TNFa production by ELISA (Invitrogen, Catalog Number 88-7324 by Thermo-Fisher Scientific). A 70 uL volume of mRNA catcher lysis buffer (1×) with DTT from the Invitrogen mRNA Catcher Plus kit (Cat #K1570-02) was added to the remaining 70 uL sample in the well, and was mixed by pipetting up and down 5 times. The plate was then shaken at RT for 5-10 min, followed by addition of 2 uL of proteinase K (20 mg/mL) to each well. Plates were then shaken for 15-20 min at RT. The plates were then stored at −80° C. until further processing.

The frozen samples were thawed and mRNA was extracted using the Invitrogen mRNA Catcher Plus kit (Cat #K1570-02) according to the manufacturer's instructions. Half yield of mRNA from RNA extraction were used to synthesize cDNA in 20 μL reverse transcriptase reactions using Invitrogen SuperScript IV VILO Master Mix (Cat #11756500). TaqMan® real-time PCR was performed using QuantStudio Real-Time PCR system from ThermoFisher (Applied Biosystems). All real-time PCR reactions were run in duplicate using commercial predesigned TaqMan assays for mouse IFIT1, IFIT3, MX1 and PPIA gene expression and TaqMan Master Mix. PPIA was utilized as the housekeeping gene. The recommendations from the manufacturer were followed. All raw data (Ct) were normalized by average housekeeping gene (Ct) and then the comparative Ct (ΔΔCt) method were utilized to quantify relative gene expression (RQ) for experimental analysis.

### Definitions

“Aliphatic” means a straight- or branched-chain, saturated or unsaturated, non-aromatic hydrocarbon moiety having the specified number of carbon atoms (e.g., as in “C₃ aliphatic,” “C₁₋₅ aliphatic,” “C₁-C₅ aliphatic,” or “C₁ to C₅ aliphatic,” the latter three phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties). A similar understanding is applied to the number of carbons in other types, as in C₂₋₄ alkene, C₄-C₇ cycloaliphatic, etc. In a similar vein, a term such as “(CH₂)₁₋₃” is to be understand as shorthand for the subscript being 1, 2, or 3, so that such term represents CH₂, CH₂CH₂, and CH₂CH₂CH₂.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like. “Alkanediyl” (sometimes also referred to as “alkylene”) means a divalent counterpart of an alkyl group, such as

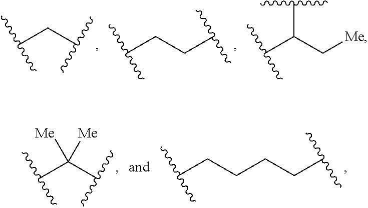

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E- (or Z-) 2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings, each ring having from 3 to 8 (preferably from 3 to 6) carbon atoms. “Cycloalkyl” means a cycloaliphatic moiety in which each ring is saturated. “Cycloalkenyl” means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl. “Cycloalkanediyl” (sometimes also referred to as “cycloalkylene”) means a divalent counterpart of a cycloalkyl group. Similarly, “bicycloalkanediyl” (osr “bicycloalkylene”) and “spiroalkanediyl” (or “spiroalkylene”) refer to divalent counterparts of a bicycloalkyl and spiroalkyl (or “spirocycloalkyl”) group.

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Preferred cycloaliphatic moieties consist of one ring, 5- to 6-membered in size. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like. “Heterocycloalkylene” means a divalent counterpart of a heterocycloalkyl group.

“Alkoxy,” “aryloxy,” “alkylthio,” and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine, unless a narrower meaning is indicated.

“Aryl” means a hydrocarbon moiety having a mono-, bi-, or tricyclic ring system (preferably monocyclic) wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl. “Arylene” means a divalent counterpart of an aryl group, for example 1,2-phenylene, 1,3-phenylene, or 1,4-phenylene.

“Heteroaryl” means a moiety having a mono-, bi-, or tricyclic ring system (preferably 5- to 7-membered monocyclic) wherein each ring has from 3 to 7 carbon atoms and at least one ring is an aromatic ring containing from 1 to 4 heteroatoms independently selected from from N, O, or S, where the N and S optionally may be oxidized and the N optionally may be quaternized. Such at least one heteroatom containing aromatic ring may be fused to other types of rings (as in benzofuranyl or tetrahydroisoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzofuranyl, indolyl, benzothiophenyl, oxadiazolyl, thiadiazolyl, phenothiazolyl, benzimidazolyl, benzotriazolyl, dibenzofuranyl, carbazolyl, dibenzothiophenyl, acridinyl, and the like. “Heteroarylene” means a divalent counterpart of a heteroaryl group.

Where it is indicated that a moiety may be substituted, such as by use of “unsubstituted or substituted” or “optionally substituted” phrasing as in “unsubstituted or substituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein. Where a moiety is identified as being “unsubstituted or substituted” or “optionally substituted,” in a preferred embodiment such moiety is unsubstituted.

“Arylalkyl,” (heterocycloaliphatic)alkyl,” “arylalkenyl,” “arylalkynyl,” “biarylalkyl,” and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl,” “alkenylcycloalkyl,” and the like mean an aryl, cycloalkyl, etc., moiety, as the case may be, substituted with an alkyl, alkenyl, etc., moiety, as the case may be, for example as in methylphenyl (tolyl) or allylcyclohexyl. “Hydroxyalkyl,” “haloalkyl,” “alkylaryl,” “cyanoaryl,” and the like mean an alkyl, aryl, etc., moiety, as the case may be, substituted with one or more of the identified substituent (hydroxyl, halo, etc., as the case may be).

For example, permissible substituents include, but are not limited to, alkyl (especially methyl or ethyl), alkenyl (especially allyl), alkynyl, aryl, heteroaryl, cycloaliphatic, heterocycloaliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF₃), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(═O)alkyl, —S(cycloalkyl), —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are phenyl, cyano, halo, hydroxyl, nitro, C₁-C₄ alkyoxy, O(C₂-C₄ alkanediyl)OH, and O(C₂-C₄ alkanediyl)halo.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(aryl), —O(cycloalkyl), —O(heterocycloalkyl), alkylthio, arylthio, —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are C₁-C₄ alkyl, cyano, nitro, halo, and C₁-C₄alkoxy.

Where a range is stated, as in “C₁-C₅ alkyl” or “5 to 10%,” such range includes the end points of the range, as in C₁ and C₅ in the first instance and 5% and 10% in the second instance.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature or symbols), all stereoisomers are included within the scope of the invention, as pure compounds as well as mixtures thereof. Unless otherwise indicated, racemates, individual enantiomers (whether optically pure or partially resolved), diastereomers, geometrical isomers, and combinations and mixtures thereof are all encompassed by this invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

“Pharmaceutically acceptable ester” means an ester that hydrolyzes in vivo (for example in the human body) to produce the parent compound or a salt thereof or has per se activity similar to that of the parent compound. Suitable esters include C₁-C₅ alkyl, C₂-C₅ alkenyl or C₂-C₅ alkynyl esters, especially methyl, ethyl or n-propyl.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methyl-sulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In the formulae of this specification, a wavy line ( [symbol] ) transverse to a bond or an asterisk (*) at the end of the bond denotes a covalent attachment site. For instance, a statement that R is

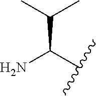 [SMILES: *[C@@H](N)C(C)C]

or that R is

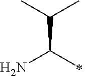

in the formula

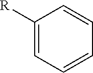

means

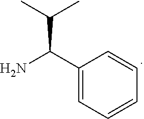

In the formulae of this specification, a bond traversing an aromatic ring between two carbons thereof means that the group attached to the bond may be located at any of the positions of the aromatic ring made available by removal of the hydrogen that is implicitly there (or explicitly there, if written out). By way of illustration:

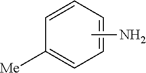

represents

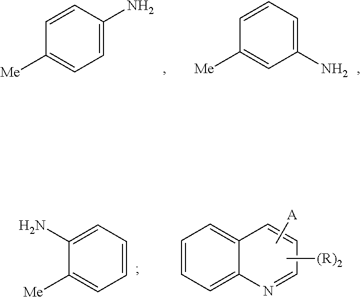

represents

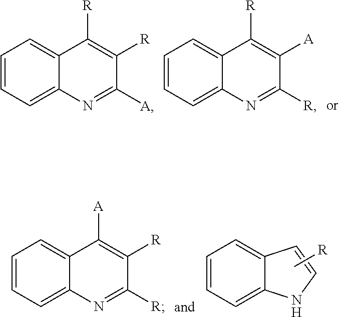

represents

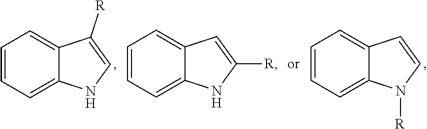

This disclosure includes all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. By way of example, a C₁-C₃ alkyl group can be undeuterated, partially deuterated, or fully deuterated and “CH₃” includes CH₃, ¹³CH₃, ¹⁴CH₃, CH₂T, CH₂D, CHD₂, CD₃, etc. In one embodiment, the various elements in a compound are present in their natural isotopic abundance.

Those skilled in the art will appreciate that certain structures can be drawn in one tautomeric form or another—for example, keto versus enol—and that the two forms are equivalent.

### Acronyms and Abbreviations

Table C provides a list of acronyms and abbreviations used in this specification, along with their meanings.

TABLE C

| ACRONYM OR | |
|---|---|
| ABBREVIATION | MEANING OR DEFINITION |
| AIBN | Azobisisobutyronitrile |
| Alloc | Allyloxycarbonyl |
| Aq. | Aqueous |
| Boc | t-Butyloxycarbonyl |
| BOP | (Benzotriazol-1-yloxy)tris(dimethylamino)- |
| | phosphonium hexafluorophosphate (V) |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIPEA, DIEA | N,N-diisopropylethylamine, also known as |
| | Hünig's base |
| DMA | N,N-Dimethylacetamide |
| DMAP | 4-(Dimethylamino) pyridine |
| DMF | N,N-dimethylformamide |
| DMSO | Dimethyl sulfoxide |
| DTDP | 2,2′-dithiodipyridine |
| DTPA | Diethylenetriaminepentaacetic acid |
| EEDQ | Ethyl 2-ethoxyquinoline-1(2H)-carboxylate |
| Fmoc | Fluorenyl methyloxycarbonyl |
| HATU | Hexafluorophosphate Azabenzotriazole |
| | Tetramethyl Uronium; 1-[Bis(dimethylamino)- |
| | methylene]-1H-1,2,3-triazolo[4,5-b]pyridinium |
| | 3-oxide hexafluorophosphate |
| HEPES | 4-(2-Hydroxyethyl)piperazine-1-ethanesulfonic |
| | acid, N-(2-Hydroxyethyl)piperazine-N′-(2- |
| | ethanesulfonic acid) |
| HPLC | High pressure liquid chromatography |
| Hunig's base | See DIPEA, DIEA |
| LCMS, LC-MS, | Liquid chromatography-mass spectrometry |
| LC/MS | |
| mCPBA | m-chloroperbenzoic acid |
| MS | Mass spectrometry |
| MsCI | Methanesylfonyl chloride, mesyl chloride |
| NBS | N-Bromosuccinimide |
| NMR | Nuclear magnetic resonance |
| PEG | Poly(ethylene glycol) |
| PTFE | Poly(tetrafluoroethylene) |
| RT (in context | Retention time, in min |
| of liquid | |
| chromatography) | |
| RT (in the context of | Room (ambient) temperature, circa 25° C. |
| reaction conditions) | |
| Sat. | Saturated |
| Soln | Solution |
| TBDPS | tert-Butyldiphenylsilyl |
| TBS | t-Butyldimethylsily group |
| TEA | Triethylamine |
| TEAA | Triethylammonium acetate |
| TFA | Trifluoroacetic acid |
| THF | Tetra hydrofuran |

### REFERENCES

Full citations for the following references cited in abbreviated fashion by first author (or inventor) and date earlier in this specification are provided below. Each of these references is incorporated herein by reference for all purposes.
Akinbobuyi et al., Tetrahedron Lett. 2015, 56, 458, “Facile syntheses of functionalized toll-like receptor 7 agonists”.
Akinbobuyi et al., Bioorg. Med. Chem. Lett. 2016, 26, 4246, “Synthesis and immunostimulatory activity of substituted TLR7 agonists.”
Barberis et al., US 2012/0003298 A1 (2012).
Beesu et al., J. Med. Chem. 2017, 60, 2084, “Identification of High-Potency Human TLR8 and Dual TLR7/TLR8 Agonists in Pyrimidine-2,4-diamines.”
Berghöfer et al., J. Immunol. 2007, 178, 4072, “Natural and Synthetic TLR7 Ligands Inhibit CpG-A- and CpG-C-Oligodeoxynucleotide-Induced IFN-α Production.”
Bonfanti et al., US 2014/0323441 A1 (2015) [2015a].
Bonfanti et al., US 2015/0299221 A1 (2015) [2015b].
Bonfanti et al., US 2016/0304531 A1 (2016).
Carson et al., US 2013/0202629 A1 (2013).
Carson et al., U.S. Pat. No. 8,729,088 B2 (2014).
Carson et al., U.S. Pat. No. 9,050,376 B2 (2015).
Carson et al., US 2016/0199499 A1 (2016).
Chan et al., Bioconjugate Chem. 2009, 20, 1194, “Synthesis and Immunological Characterization of Toll-Like Receptor 7 Agonistic Conjugates.”
Chan et al., Bioconjugate Chem. 2011, 22, 445, “Synthesis and Characterization of PEGylated Toll Like Receptor 7 Ligands.”
Chen et al., U.S. Pat. No. 7,919,498 B2 (2011).
Coe et al., U.S. Pat. No. 9,662,336 B2 (2017).
Cortez and Va, Medicinal Chem. Rev. 2018, 53, 481, “Recent Advances in Small-Molecule TLR7 Agonists for Drug Discovery”.
Cortez et al., US 2017/0121421 A1 (2017).
Cortez et al., U.S. Pat. No. 9,944,649 B2 (2018).
Dellaria et al., WO 2007/028129 A1 (2007).
Desai et al., U.S. Pat. No. 9,127,006 B2 (2015).
Ding et al., WO 2016/107536 A1 (2016).
Ding et al., US 2017/0273983 A1 (2017) [2017a].
Ding et al., WO 2017/076346 A1 (2017) [2017b].
Gadd et al., Bioconjugate Chem. 2015, 26, 1743, “Targeted Activation of Toll-Like Receptors: Conjugation of a Toll-Like Receptor 7 Agonist to a Monoclonal Antibody Maintains Antigen Binding and Specificity.”
Graupe et al., U.S. Pat. No. 8,993,755 B2 (2015).
Embrechts et al., J. Med. Chem. 2018, 61, 6236, “2,4-Diaminoquinazolines as Dual Toll Like Receptor (TLR) 7/8 Modulators for the Treatment of Hepatitis B Virus.”
Halcomb et al., U.S. Pat. No. 9,161,934 B2 (2015).
Hashimoto et al., US 2009/0118263 A1 (2009).
He et al., U.S. Pat. No. 10,487,084 B2 (2019) [2019a].
He et al., U.S. Pat. No. 10,508,115 B2 (2019) [2019b].
Hirota et al., U.S. Pat. No. 6,028,076 (2000).
Holldack et al., US 2012/0083473 A1 (2012).
Isobe et al., U.S. Pat. No. 6,376,501 B1 (2002).
Isobe et al., JP 2004137157 (2004).
Isobe et al., J. Med. Chem. 2006, 49 (6), 2088, “Synthesis and Biological Evaluation of Novel 9-Substituted-8-Hydroxyadenine Derivatives as Potent Interferon Inducers.”
Isobe et al., U.S. Pat. No. 7,521,454 B2 (2009) [2009a].
Isobe et al., US 2009/0105212 A1 (2009) [2009b].
Isobe et al., US 2011/0028715 A1 (2011).
Isobe et al., U.S. Pat. No. 8,148,371 B2 (2012).
Jensen et al., WO 2015/036044 A1 (2015).
Jones et al., U.S. Pat. No. 7,691,877 B2 (2010).
Jones et al., US 2012/0302598 A1 (2012).
Kasibhatla et al., U.S. Pat. No. 7,241,890 B2 (2007).
Koga-Yamakawa et al., Int. J. Cancer 2013, 132 (3), 580, “Intratracheal and oral administration of SM-276001: A selective TLR7 agonist, leads to antitumor efficacy in primary and metastatic models of cancer.”
Li et al., U.S. Pat. No. 9,902,730 B2 (2018).
Lioux et al., U.S. Pat. No. 9,295,732 B2 (2016).
Lund et al., Proc. Nat'l Acad. Sci (USA) 2004, 101 (15), 5598, “Recognition of single-stranded RNA viruses by Toll-like receptor 7.”
Maj et al., U.S. Pat. No. 9,173,935 B2 (2015).
McGowan et al., US 2016/0168150 A1 (2016) [2016a].
McGowan et al., U.S. Pat. No. 9,499,549 B2 (2016) [2016b].
McGowan et al., J. Med. Chem. 2017, 60, 6137, “Identification and Optimization of Pyrrolo[3,2-d]pyrimidine Toll-like Receptor 7 (TLR7) Selective Agonists for the Treatment of Hepatitis B.”
Musmuca et al., J. Chem. Information & Modeling 2009, 49 (7), 1777, “Small-Molecule Interferon Inducers. Toward the Comprehension of the Molecular Determinants through Ligand-Based Approaches.”
Nakamura et al., Bioorg. Med. Chem. Lett. 2013, 13, 669, “Synthesis and evaluation of 8-oxoadenine derivatives as potent Toll-like receptor agonists with high water solubility.”
Ogita et al., US 2007/0225303 A1 (2007).
Ota et al., WO 2019/124500 A1 (2019).
Pilatte et al., WO 2017/216293 A1 (2017).
Poudel et al., U.S. Pat. No. 10,472,361 B2 (2019) [2019a].
Poudel et al., U.S. Pat. No. 10,494,370 B2 (2019) [2019b].
Poudel et al., US 2020/0038403 A1 (2020) [2020a].
Poudel et al., US 2020/0039986 A1 (2020) [2020b].
Purandare et al., WO 2019/209811 A1 (2019).
Pryde, U.S. Pat. No. 7,642,350 B2 (2010).
Sato-Kaneko et al., JCI Insight 2017, 2, e93397, “Combination Immunotherapy with TLR Agonists and Checkpoint Inhibitors Suppresses Head and Neck Cancer”.
Smits et al., The Oncologist 2008, 13, 859, “The Use of TLR7 and TLR8 Ligands for the Enhancement of Cancer Immunotherapy”.
Vasilakos and Tomai, Expert Rev. Vaccines 2013, 12, 809, “The Use of Toll-like Receptor 7/8 Agonists as Vaccine Adjuvants”.
Vernejoul et al., US 2014/0141033 A1 (2014).
Young et al., U.S. Pat. No. 10,457,681 B2 (2019).
Yu et al., PLoS One 2013, 8 (3), e56514, “Toll-Like Receptor 7 Agonists: Chemical Feature Based Pharmacophore Identification and Molecular Docking Studies.”
Zhang et al., Immunity 2016, 45, 737, “Structural Analysis Reveals that Toll-like Receptor 7 Is a Dual Receptor for Guanosine and Single-Stranded RNA.”
Zhang et al., WO 2018/095426 A1 (2018)>
Zurawski et al., US 2012/0231023 A1 (2012).

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A compound having a structure according to formula I or formula (II) 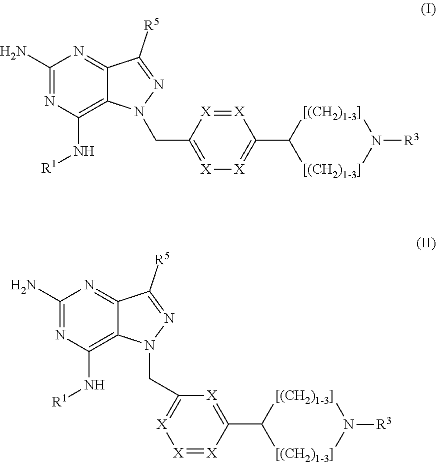
wherein
each X is independently N or CR²;
R¹ is H,
(C₁-C₅ alkyl),
(C₂-C₅ alkenyl),
(C₁-C₈ alkanediyl)₀₋₁(C₃-C₆ cycloalkyl),
(C₁-C₈ alkanediyl)₀₋₁(C₅-C₁₀ spiroalkyl),
(C₂-C₈ alkanediyl)OH,
(C₂-C₈ alkanediyl)O(C₁-C₃ alkyl),
(C₁-C₄ alkanediyl)₀₋₁(5-6 membered heteroaryl),
(C₁-C₄ alkanediyl)₀₋₁phenyl,
(C₁-C₄ alkanediyl)CF₃,
(C₂-C₈ alkanediyl)N[C(═O)](C₁-C₃ alkyl),
or
(C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN; or [C(═O)]₀₋₁NR^{x}R^{y};
R³ is [C(═O)]₀₋₁H,
[C(═O)]₀₋₁(C₁-C₅ alkyl),
[C(═O)]₀₋₁(C₁-C₄ alkanediyl)₀₋₁(C₃-C₅ cycloalkyl),
[C(═O)]₀₋₁(C₁-C₄ alkanediyl)₀₋₁(C₄-C₁₀ bicycloalkyl),
[C(═O)]₀₋₁(C₁-C₄ alkanediyl)₀₋₁(C₅-C₁₀ spiroalkyl),
[C(═O)]₀₋₁(C₁-C₄ alkanediyl)₀₋₁O(C₁-C₃ alkyl),
[C(═O)]₀₋₁(C₁-C₄ alkanediyl)₀₋₁OH,
[C(═O)]₀₋₁(C₁-C₄ alkanediyl)₀₋₁NR^{x}R^{y}, 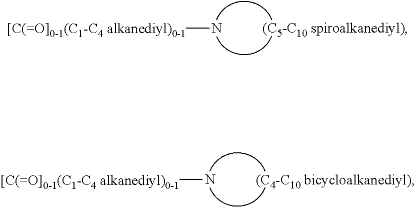
(C₁-C₄ alkanediyl)(C═O)NR^{x}R^{y},
(SO₂)(C₁-C₅ alkyl),
(SO₂)(C₁-C₄ alkanediyl)₀₋₁(C₃-C₅ cycloalkyl),
(SO₂)(C₁-C₄ alkanediyl)₀₋₁(C₄-C₁₀ bicycloalkyl),
(SO₂)(C₁-C₄ alkanediyl)₀₋₁(C₅-C₁₀ spiroalkyl),
a 6-membered aromatic or heteroaromatic moiety, or
a 5-membered heteroaromatic moiety;
R⁵ is H, C₁-C₅ alkyl, C₂-C₅ alkenyl, C₃-C₆ cycloalkyl, halo, O(C₁-C₅ alkyl),
(C₁-C₄ alkanediyl)OH, (C₁-C₄ alkanediyl)O(C₁-C₃ alkyl), phenyl, NH(C₁-C₅ alkyl), 5 or 6 membered heteroaryl, 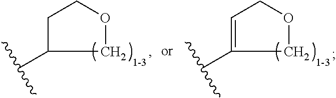
and
R^{x} and R^{y} are independently H or C₁-C₃ alkyl or R^{x} and R^{y} combine with the nitrogen to which they are bonded to form a 3- to 7-membered ring;
wherein in R¹, R², R³, and R⁵
an alkyl, cycloalkyl, alkanediyl, bicycloalkyl, spiroalkyl, cyclic amine, 6-membered aromatic or heteroaromatic moiety, 5-membered heteroaromatic moiety or a moiety of the formula 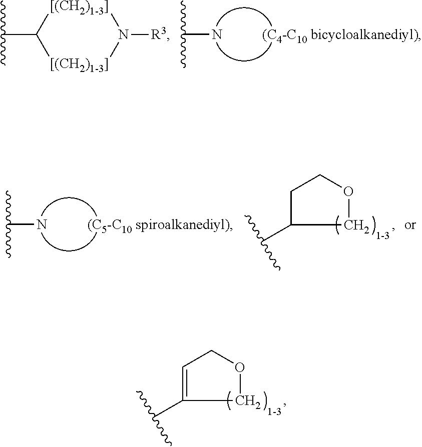
is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, bicycloalkyl, spiroalkyl, or a moiety of the formula 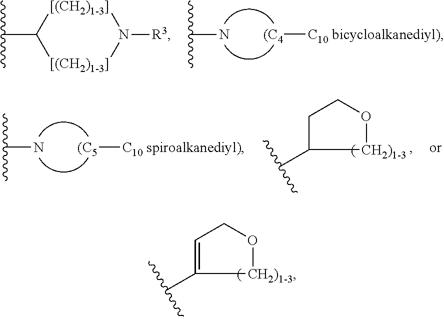
may have a CH₂ group replaced by O, SO₂, CF₂, C(═O), NH,
N[C(═O)]₀-1(C₁-C₃ alkyl),
N[C(═O)]₀-1(C₁-C₄ alkanediyl)₀₋₁CF₃,
or
N[C(═O)]₀-1(C₁-C₄ alkanediyl)₀₋₁(C₃-C₅ cycloalkyl).

2. A compound according to claim 1, wherein R¹ is

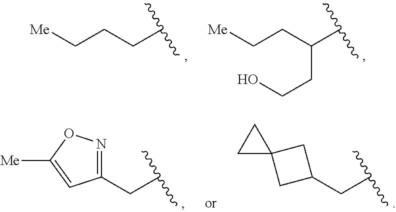

3. A compound according to claim 1, wherein R² is OMe.

4. A compound according to claim 1, wherein R³ is H, Me,

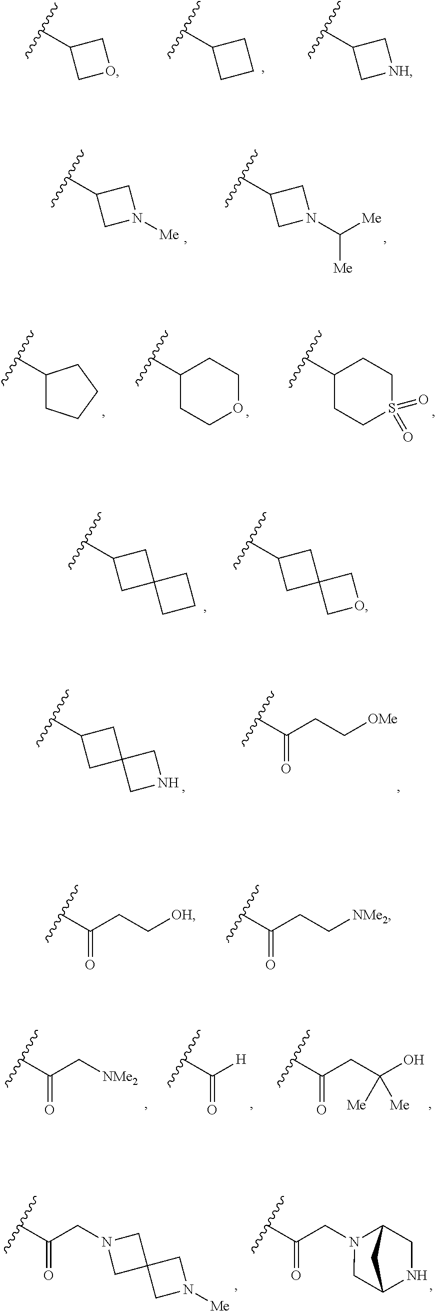

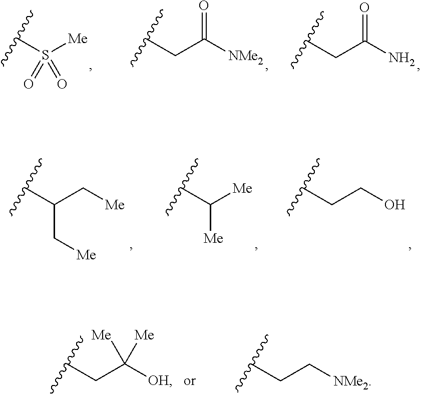

5. A compound according to claim 1, wherein R⁵ is H.

6. A compound having a structure represented by formula (Ia) 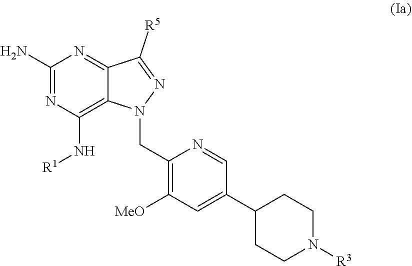
wherein
R¹ is 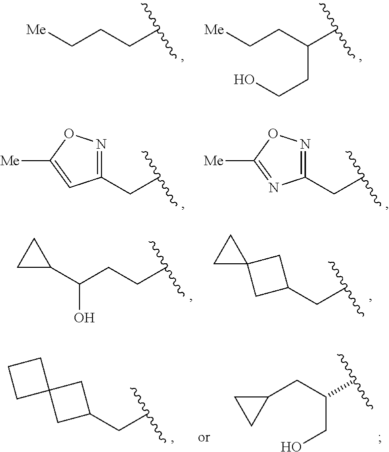
R³ is H, Me, 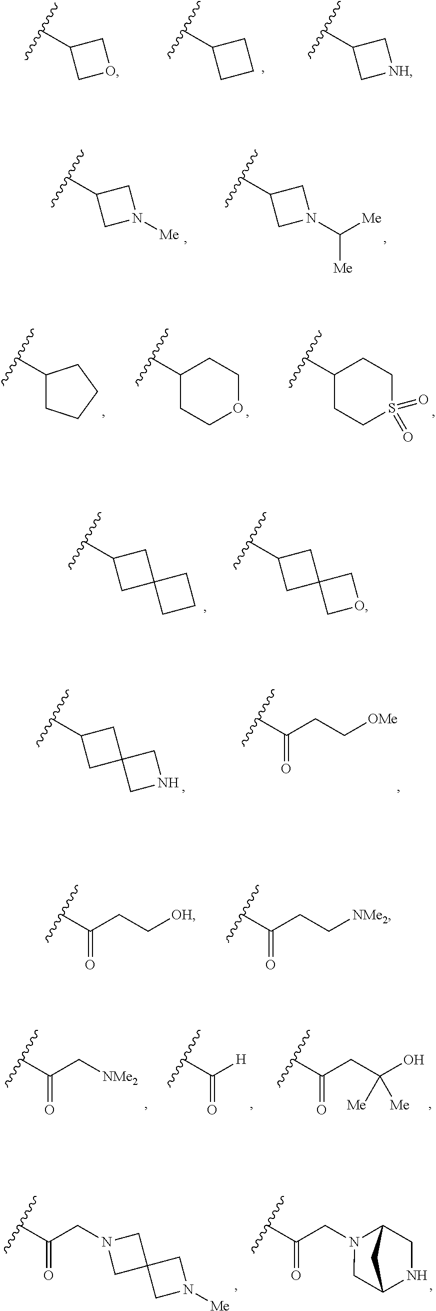 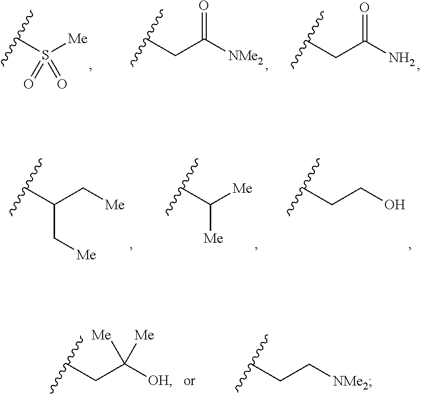
and
R⁵ is H, Me, cyclopropyl, or Cl.

7. A compound having a structure represented by formula (Ib) 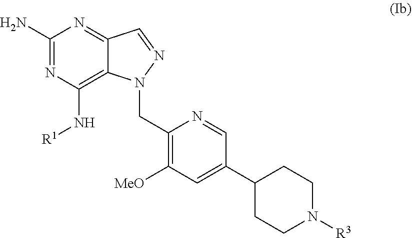
wherein
R¹ is 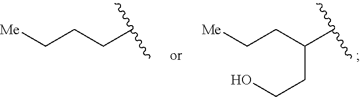 [SMILES: *CCCC ; *C(CCC)CCO]
and
R³ is H,

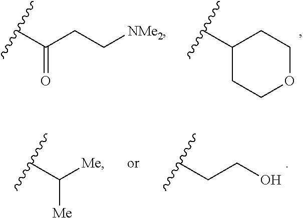

8. A method of treating a cancer, comprising administering to a patient suffering from such cancer a therapeutically effective combination of an anti-cancer immunotherapy agent and a compound according to claim 1.

9. A method according to claim 8, wherein the anti-cancer immunotherapy agent is an antagonistic anti-CTLA-4, anti-PD-1, or anti-PD-L1 antibody.

10. A method according to claim 9, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

11. A method according to claim 10, wherein the anti-cancer immunotherapy agent is ipilimumab, nivolumab, or pembrolizumab.

12. A compound according to claim 1, having a structure represented by formula (Ic)

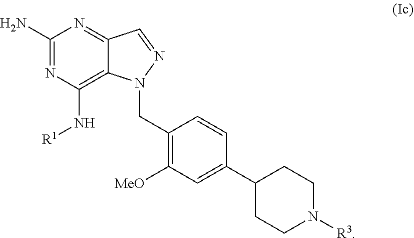

13. A compound according to claim 1, having a structure represented by formula (IIa)

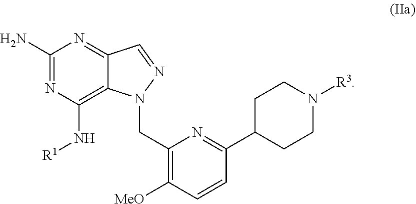

14. A compound according to formula (I′a) 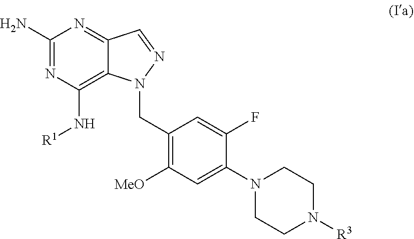
wherein
R¹ is 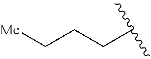 [SMILES: *CCCC]
and R³ is H,

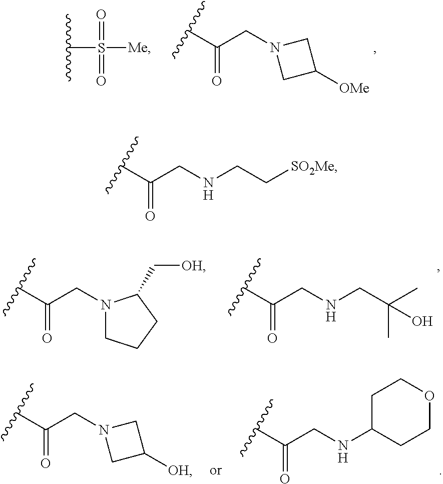

15. A compound having a structure according to formula (I″a), 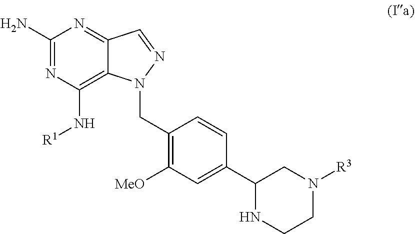
wherein
R¹ is 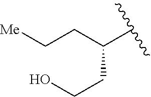 [SMILES: *[C@@H](CCC)CCO]
and
R³ is

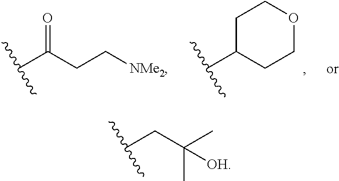

16. A method of treating a cancer, comprising administering to a patient suffering from such cancer a therapeutically effective combination of an anti-cancer immunotherapy agent and a compound according to claim 7.

17. A method according to claim 16, wherein the anti-cancer immunotherapy agent is an antagonistic anti-CTLA-4, anti-PD-1, or anti-PD-L1 antibody.

18. A method according to claim 16, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

19. A method according to claim 17, wherein the anti-cancer immunotherapy agent is ipilimumab, nivolumab, or pembrolizumab.
